(19) United States
(12) Patent Application Publication
(10) Pub. No.: US 2024/0016839 A1
(43) Pub. Date: Jan. 18, 2024

(54) BICISTRONIC INHIBITORY CHIMERIC ANTIGEN RECEPTOR (ICAR)/ACTIVATING CHIMERIC ANTIGEN RECEPTOR (ACAR) CONSTRUCTS FOR USE IN CANCER THERAPIES

(71) Applicant: ImmPACT BIO USA Inc., Camarillo, CA (US)

(72) Inventors: Rick Kendall, Camarillo, CA (US); Frank Calzone, Camarillo, CA (US); Orit Foord, Camarillo, CA (US); Gregor B. Adams, Camarillo, CA (US); Tanya Kim, Camarillo, CA (US); David Bassan, Camarillo, CA (US); Jason Yi, Camarillo, CA (US); Adi Sharbi-Yunger, Camarillo, CA (US)

(21) Appl. No.: 18/044,075

(22) PCT Filed: Sep. 7, 2021
(86) PCT No.: PCT/US2021/049315

§ 371 Date: Mar. 3, 2023

Related U.S. Application Data
(60) Provisional application No. 63/178,452, filed on Apr. 22, 2021.
(60) Provisional application No. 63/074,812, filed on Sep. 4, 2020.

(51) Int. Cl. A61K 35/17; A61K 39/00; C07K 14/725

(52) U.S. Cl. CPC A61K 35/17; A61K 39/4644; A61K 39/4631; C07K 14/7051; A61K 2039/5156; A61K 2039/5158; C07K 2317/622

(57) ABSTRACT

The invention relates to the field of cancer immunotherapy by employing bicistronic inhibitory chimeric antigen receptor (iCAR)/activating chimeric antigen receptor (aCAR) constructs for use in cancer treatment therapies.

### CROSS-REFERENCE TO RELATED APPLICATIONS

This application is a U.S. National Stage Application under 35 U.S.C. § 371 which claims the benefit of priority to International Application No. PCT/US2021/049315, filed Sep. 7, 2021, which claims priority under 35 U.S.C. § 119 to U.S. Patent Application Nos. 63/178,452, filed on Apr. 22, 2021, and 63/074,812, filed on Sep. 4, 2020, both of which are expressly incorporated herein by reference in their entireties.

### SEQUENCE LISTING

A Sequence Listing is being submitted electronically via EFS in the form of a text file, created Mar. 1, 2023, and named “194110560_1.txt” (690,086 bytes), the contents of which are incorporated herein by reference in their entirety.

### FIELD OF THE INVENTION

The invention relates to the field of cancer immunotherapy by employing inhibitory chimeric antigen receptors (iCARs) paired with activating chimeric antigen receptors (aCARs) for use in cancer treatment therapies.

### BACKGROUND OF THE INVENTION

The identification of targetable antigens that are exclusively expressed by tumor cells but not by healthy tissue is undoubtedly the major challenge in cancer immunotherapy today. Clinical evidence that T cells are capable of eradicating tumor cells comes from numerous studies evaluating highly diverse approaches for harnessing T cells to treat cancer (Rosenberg and Restifo, Science, 348(6230): 62-68 (2015)). These approaches employ bone marrow transplantation with donor lymphocyte infusion, adoptive transfer of tumor-infiltrating lymphocytes (TILs), treatment with T cells genetically redirected at pre-selected antigens via CARs (Gross and Eshhar, Annual Review of Pharmacology and Toxicology, 56:59-83, (2016)) or T cell receptors (TCRs), the use of immune checkpoint inhibitors, BiTEs (bispecific T-cell engager molecules) technologies; Einsele, H., et al., Cancer, 126(14):3192-3201 (2020)), or active vaccination. Of these, the use of genetically engineered T cells and different strategies for active immunization entail pre-existing information on candidate antigens which are likely to exert a durable clinical response but minimal adverse effects. Yet, as stated in the title of a review by S. Rosenberg, “Finding suitable targets is the major obstacle to cancer gene therapy” (Rosenberg, Cancer Gene Therapy, 21:45-47 (2014))).

The concept of using chimeric antigen receptors (or CARs) to genetically redirect T cells (or other killer cells of the immune system such as natural killer (NK) cells and cytokine-induced killer cells) against antigens of choice in an MHC-independent manner was first introduced by Gross and Eshhar in the late 1980s (Gross et al., PNAS, 86(24):10024-1002 (1989). They are produced synthetically from chimeric genes encoding an extracellular single-chain antibody variable fragment (scFv) fused through a flexible hinge and transmembrane domain to costimulatory domains and signaling components comprising immunoreceptor tyrosine-based activation motifs of CD3-ζ or FcRγ chains capable of T cell activation. At present, CARs are being examined in dozens of clinical trials and have shown exceptionally high efficacy in B cell malignancies (Dotti et al., 2014; Gill and June, 263(1): 68-89 (2015)); Gross and Eshhar, Annual Review of Pharmacology and Toxicology, 56:59-83, 2016). The safety of CAR-T cell therapy is determined, in large part, by its ability to discriminate between the tumor and healthy tissue. A major risk in targeting solid tumors, and the direct cause for adverse autoimmune effects that have been reported in clinical and preclinical studies, is off-tumor, on-target toxicity resulting from extra-tumor expression of the target antigen (dealt with in detail in the review (Gross and Eshhar, 2016b) and (Klebanoff, et al., Nature Medicine 22:26-36 (2016)).

While undoubtedly intriguing, these previous CAR-based approaches require tuning the affinity of CAR scFv's to selectively bind high antigen levels in tumors while minimizing recognition of lower antigen levels in healthy tissues. In addition, the magnitude of both the activating and costimulatory signals needs to be balanced to allow effective on-target, on-tumor T cell reactivity. It is worth noting that in B cell malignancies, CARs targeted antigen exclusive to B cells and did not require titration of affinity or T cell signaling. For solid tumors, whether such balance can be routinely attained in the clinical setting is questionable.

Off-tumor reactivity occurs when the tumor antigen targeted by CAR-redirected killer cells is shared with normal tissue. However, if the normal tissue expresses another surface antigen that is not present on the tumor, it can be targeted by inhibitory CARs (iCARs) that contains an inhibitory signaling moiety which when engaged prevents T-cell activation by the activating CAR (aCAR). Co-expression of aCAR and iCAR will therefore direct killer cells to target tumors while sparing normal tissue.

Instead of an activating domain (such as FcRγ or CD3-ζ), an iCAR possesses a signaling domain derived from an inhibitory receptor which can antagonize T cell activation, such as CTLA-4, PD-1, or NK inhibitory receptors.

There remains a need in the art for cancer therapies, in particular therapies that comprise iCARs in order to limit off-target effects. The present invention meets that need by providing either co-transduction of monocistronic aCAR and iCAR constructs, or bicistronic constructs comprising such iCARs and which find use in cancer treatment.

### BRIEF SUMMARY OF THE INVENTION

The present invention provides bicistronic iCAR/aCAR constructs or monocistronic aCAR and iCAR constructs for co-transduction and uses thereof.

The present invention provides a bicistronic iCAR/aCAR construct or monocistronic aCAR and iCAR constructs for co-transduction comprising:
1. an iCAR portion, wherein the iCAR portion comprises:
2. an iCAR single chain variable fragment (scFv) component optionally in the VH-VL or VL-VH orientation;
3. an iCAR hinge domain component;
4. an iCAR transmembrane (TM) domain component;
5. an iCAR inhibitory domain component; and
6. an aCAR portion, wherein the iCAR portion comprises:
7. an aCAR single chain variable fragment (scFv) component optionally in the VH-VL or VL-VH orientation;
8. an aCAR hinge domain component;
9. an aCAR co-stimulatory domain component an aCAR activation signaling domain; and
11. a linker that connects the iCAR portion in (i) and the aCAR portion in (ii).

In some embodiments of the bicistronic iCAR/aCAR construct or monocistronic aCAR and iCAR constructs for co-transduction, the linker connecting the VH-VL or VL-VH in either orientation comprises one or more linker selected from the group consisting of (G4S)X3 linker

(SEQ ID NO:81), G4S (SEQ ID NO:153), (G4S)X3 (SEQ ID NO:154), and Whitlow linker (SEQ ID NO:82).

In some embodiments of the bicistronic iCAR/aCAR construct or monocistronic aCAR and iCAR constructs for co-transduction, the iCAR scFv component targets an HLA antigen.

In some embodiments of the bicistronic iCAR/aCAR construct or monocistronic aCAR and iCAR constructs for co-transduction, the HLA antigen is selected from the group consisting of HLA-A2, HLA-A3, HLA-A, HLA-B, HLA-C, HLA-G, HLA-E, HLA-F, HLA-DPA1, HLA-DQA1, HLA-DQB1, HLA-DQB2, HLA-DRB1, and HLA-DRB5.

In some embodiments of the bicistronic iCAR/aCAR construct or monocistronic aCAR and iCAR constructs for co-transduction, the iCAR scFv component is selected from the group consisting of BB7.2, 3PF12, 3PF12/C4, 3PF12/F12, 3PF12/B11, W6/32, BBM.1, SN66E3, Ha5C2.A2, MWB1, MWB1-mod, Hz.BB7.2 VH1-69_A18VK, Hz.BB7.2 VH1-69 (27,30)_A18, Hz.BB7.2 VH1-69 (27,30,48)_A18, Hz.BB7.2 VH1-69 (27,30,67)_A18, Hz.BB7.2 VH1-69 (27,30,69)_A18, Hz.BB7.2 VH1-69 (27,30,67,69)_A18, Hz.BB7.2 VH1-3_A18, Hz.BB7.2 VH1-3(48)_A18, Hz.BB7.2 VH1-3(67)_A18, Hz.BB7.2 VH1-3(69)_A18, Hz.BB7.2 VH1-3(71)_A18, Hz.BB7.2 VH1-3(73)_A18, MWB1.2, SN66E3.2 and SN66E3.3.

In some embodiments of the bicistronic iCAR/aCAR construct or monocistronic aCAR and iCAR constructs for co-transduction, the iCAR scFv component is BB7.2.

In some embodiments of the bicistronic iCAR/aCAR construct or monocistronic aCAR and iCAR constructs for co-transduction, the iCAR scFv comprises the Vh and Vl from BB7.2 (SEQ ID NOs: 37 and 38) or vhCDR1, vhCDR2, vhCDR3, vlCDR1, vlCDR2, and vlCDR3 from SEQ ID NOs: 37 and 38.

In some embodiments of the bicistronic iCAR/aCAR construct or monocistronic aCAR and iCAR constructs for co-transduction, the iCAR scFv comprises the Vh and Vl from Hz.BB7.2 VH1-69_A18VK (SEQ ID NOs: 57 and 58) or vhCDR1, vhCDR2, vhCDR3, vlCDR1, vlCDR2, and vlCDR3 from SEQ ID NOs: 57 and 58.

In some embodiments of the bicistronic iCAR/aCAR construct or monocistronic aCAR and iCAR constructs for co-transduction, wherein the iCAR scFv comprises the Vh and Vl from Hz.BB7.2 VH1-69 (27,30) A18 (SEQ ID NOs: 59 and 60) or vhCDR1, vhCDR2, vhCDR3, vlCDR1, vlCDR2, and vlCDR3 from SEQ ID NOs: 59 and 60.

In some embodiments of the bicistronic iCAR/aCAR construct or monocistronic aCAR and iCAR constructs for co-transduction, the iCAR scFv comprises the Vh and Vl from Hz.BB7.2 VH1-69 (27,30,48) A18 (SEQ ID NOs: 61 and 62) or vhCDR1, vhCDR2, vhCDR3, vlCDR1, vlCDR2, and vlCDR3 from SEQ ID NOs: 61 and 62.

In some embodiments of the bicistronic iCAR/aCAR construct or monocistronic aCAR and iCAR constructs for co-transduction, the iCAR scFv comprises the Vh and Vl from Hz.BB7.2 VH1-69 (27,30,67) A18 (SEQ ID NOs: 63 and 64) or vhCDR1, vhCDR2, vhCDR3, vlCDR1, vlCDR2, and vlCDR3 from SEQ ID NOs: 63 and 64.

In some embodiments of the bicistronic iCAR/aCAR construct or monocistronic aCAR and iCAR constructs for co-transduction, the iCAR scFv comprises the Vh and Vl from Hz.BB7.2 VH1-69 (27,30,69) A18 (SEQ ID NOs: 65 and 66) or vhCDR1, vhCDR2, vhCDR3, vlCDR1, vlCDR2, and vlCDR3 from SEQ ID NOs: 65 and 66.

In some embodiments of the bicistronic iCAR/aCAR construct or monocistronic aCAR and iCAR constructs for co-transduction, the iCAR scFv comprises the Vh and Vl from Hz.BB7.2 VH1-69 (27,30,67,69) A18 (SEQ ID NOs: 67 and 68) or vhCDR1, vhCDR2, vhCDR3, vlCDR1, vlCDR2, and vlCDR3 from SEQ ID NOs: 67 and 68.

In some embodiments of the bicistronic iCAR/aCAR construct or monocistronic aCAR and iCAR constructs for co-transduction, the iCAR scFv comprises the Vh and Vl from Hz.BB7.2 VH1-3_A18 (SEQ ID NOs: 69 and 70) or vhCDR1, vhCDR2, vhCDR3, vlCDR1, vlCDR2, and vlCDR3 from SEQ ID NOs: 69 and 70.

In some embodiments of the bicistronic iCAR/aCAR construct or monocistronic aCAR and iCAR constructs for co-transduction, the iCAR scFv comprises the Vh and Vl from Hz.BB7.2 VH1-3(48) A18 (SEQ ID NOs: 71 and 72) or vhCDR1, vhCDR2, vhCDR3, vlCDR1, vlCDR2, and vlCDR3 from SEQ ID NOs: 71 and 72.

In some embodiments of the bicistronic iCAR/aCAR construct or monocistronic aCAR and iCAR constructs for co-transduction, the iCAR scFv comprises the Vh and Vl from Hz.BB7.2 VH1-3(67) A18 (SEQ ID NOs: 73 and 74) or vhCDR1, vhCDR2, vhCDR3, vlCDR1, vlCDR2, and vlCDR3 from SEQ ID NOs: 73 and 74.

In some embodiments of the bicistronic iCAR/aCAR construct or monocistronic aCAR and iCAR constructs for co-transduction, the iCAR scFv comprises the Vh and Vl from Hz.BB7.2 VH1-3(69) A18 (SEQ ID NOs: 75 and 76) or vhCDR1, vhCDR2, vhCDR3, vlCDR1, vlCDR2, and vlCDR3 from SEQ ID NOs: 75 and 76.

In some embodiments of the bicistronic iCAR/aCAR construct or monocistronic aCAR and iCAR constructs for co-transduction, the iCAR scFv comprises the Vh and Vl from Hz.BB7.2 VH1-3(71) A18 (SEQ ID NOs: 77 and 78) or vhCDR1, vhCDR2, vhCDR3, vlCDR1, vlCDR2, and vlCDR3 from SEQ ID NOs: 77 and 78.

In some embodiments of the bicistronic iCAR/aCAR construct or monocistronic aCAR and iCAR constructs for co-transduction, the iCAR scFv comprises the Vh and Vl from Hz.BB7.2 VH1-3(73) A18 (SEQ ID NOs: 79 and 80) or vhCDR1, vhCDR2, vhCDR3, vlCDR1, vlCDR2, and vlCDR3 from SEQ ID NOs: 79 and 80.

In some embodiments of the bicistronic iCAR/aCAR construct or monocistronic aCAR and iCAR constructs for co-transduction, the iCAR scFv is BB7.2 of SEQ ID NO:167.

In some embodiments of the bicistronic iCAR/aCAR construct or monocistronic aCAR and iCAR constructs for co-transduction, the iCAR scFv component is 3PF12.

In some embodiments of the bicistronic iCAR/aCAR construct or monocistronic aCAR and iCAR constructs for co-transduction, the iCAR scFv comprises the Vh and Vl from 3PF12/C4 (SEQ ID NOs: 39 and 40) or vhCDR1, vhCDR2, vhCDR3, vlCDR1, vlCDR2, and vlCDR3 from SEQ ID NOs: 39 and 40.

In some embodiments of the bicistronic iCAR/aCAR construct or monocistronic aCAR and iCAR constructs for co-transduction, the iCAR scFv comprises the Vh and Vl from 3PF12/F12 (SEQ ID NOs: 41 and 42) or vhCDR1, vhCDR2, vhCDR3, vlCDR1, vlCDR2, and vlCDR3 from SEQ ID NOs: 41 and 42.

In some embodiments of the bicistronic iCAR/aCAR construct or monocistronic aCAR and iCAR constructs for co-transduction, wherein the iCAR scFv comprises the Vh and Vl from 3PF12/B11 (SEQ ID NOs: 43 and 44) or vhCDR1, vhCDR2, vhCDR3, vlCDR1, vlCDR2, and vlCDR3 from SEQ ID NOs: 43 and 44.

In some embodiments of the bicistronic iCAR/aCAR construct or monocistronic aCAR and iCAR constructs for co-transduction, the iCAR scFv is 3PF12 of SEQ ID NO:168.

In some embodiments of the bicistronic iCAR/aCAR construct or monocistronic aCAR and iCAR constructs for co-transduction, the iCAR scFv component is SN66E3.

In some embodiments of the bicistronic iCAR/aCAR construct or monocistronic aCAR and iCAR constructs for co-transduction, the iCAR scFv comprises the Vh and Vl from SN66E3.1 (SEQ ID NOs: 49 and 50) or vhCDR1, vhCDR2, vhCDR3, vlCDR1, vlCDR2, and vlCDR3 from SEQ ID NOs: 49 and 50.

In some embodiments of the bicistronic iCAR/aCAR construct or monocistronic aCAR and iCAR constructs for co-transduction, the iCAR scFv is SN66E3.1 of SEQ ID NO:169.

In some embodiments of the bicistronic iCAR/aCAR construct or monocistronic aCAR and iCAR constructs for co-transduction, the iCAR scFv comprises the Vh and Vl from SN66E3.2 (SEQ ID NOs: 165 and 166) or vhCDR1, vhCDR2, vhCDR3, vlCDR1, vlCDR2, and vlCDR3 from SEQ ID NOs: 165 and 166.

In some embodiments of the bicistronic iCAR/aCAR construct or monocistronic aCAR and iCAR constructs for co-transduction, the iCAR scFv is SN66E3.2 of SEQ ID NO:285.

In some embodiments of the bicistronic iCAR/aCAR construct or monocistronic aCAR and iCAR constructs for co-transduction, the iCAR scFv comprises the Vh and Vl from SN66E3.3 (SEQ ID NOs: 283 and 284) or vhCDR1, vhCDR2, vhCDR3, vlCDR1, vlCDR2, and vlCDR3 from SEQ ID NOs: 283 and 284.

In some embodiments of the bicistronic iCAR/aCAR construct or monocistronic aCAR and iCAR constructs for co-transduction, the iCAR scFv is SN66E3.3 of SEQ ID NO:286.

In some embodiments of the bicistronic iCAR/aCAR construct or monocistronic aCAR and iCAR constructs for co-transduction, the iCAR scFv component is W6/32.

In some embodiments of the bicistronic iCAR/aCAR construct or monocistronic aCAR and iCAR constructs for co-transduction, the iCAR scFv comprises the Vh and Vl from W6/32 (SEQ ID NOs: 45 and 46) or vhCDR1, vhCDR2, vhCDR3, vlCDR1, vlCDR2, and vlCDR3 from SEQ ID NOs: 45 and 46.

In some embodiments of the bicistronic iCAR/aCAR construct or monocistronic aCAR and iCAR constructs for co-transduction, the iCAR scFv component is BBM.1.

In some embodiments of the bicistronic iCAR/aCAR construct or monocistronic aCAR and iCAR constructs for co-transduction, the iCAR scFv comprises the Vh and Vl from BBM.1 (SEQ ID NOs: 47 and 48) or vhCDR1, vhCDR2, vhCDR3, vlCDR1, vlCDR2, and vlCDR3 from SEQ ID NOs: 47 and 48.

In some embodiments of the bicistronic iCAR/aCAR construct or monocistronic aCAR and iCAR constructs for co-transduction, the iCAR scFv component is Ha5C2.A2.

In some embodiments of the bicistronic iCAR/aCAR construct or monocistronic aCAR and iCAR constructs for co-transduction, the iCAR scFv comprises the Vh and Vl from Ha5C2.A2 (SEQ ID NOs: 51 and 52) or vhCDR1, vhCDR2, vhCDR3, vlCDR1, vlCDR2, and vlCDR3 from SEQ ID NOs: 51 and 52.

T In some embodiments of the bicistronic iCAR/aCAR construct or monocistronic aCAR and iCAR constructs for co-transduction, the iCAR scFv component is MWB1.

In some embodiments of the bicistronic iCAR/aCAR construct or monocistronic aCAR and iCAR constructs for co-transduction, the iCAR scFv comprises the Vh and Vl from MWB1 (SEQ ID NOs: 53 and 54) or vhCDR1, vhCDR2, vhCDR3, vlCDR1, vlCDR2, and vlCDR3 from SEQ ID NOs: 53 and 54.

In some embodiments of the bicistronic iCAR/aCAR construct or monocistronic aCAR and iCAR constructs for co-transduction, the iCAR scFv comprises the Vh and Vl from MWB1-mod (MWB1.1) (SEQ ID NOs: 55 and 56) or vhCDR1, vhCDR2, vhCDR3, vlCDR1, vlCDR2, and vlCDR3 from SEQ ID NOs: 55 and 56.

In some embodiments of the bicistronic iCAR/aCAR construct or monocistronic aCAR and iCAR constructs for co-transduction, the iCAR scFv comprises the Vh and Vl from MWB1.2 (SEQ ID NOs: 163 and 164).

In some embodiments of the bicistronic iCAR/aCAR construct or monocistronic aCAR and iCAR constructs for co-transduction, the iCAR scFv is MWB1.1 scFvVH_VL (SEQ ID NO:273).

In some embodiments of the bicistronic iCAR/aCAR construct or monocistronic aCAR and iCAR constructs for co-transduction, the iCAR scFv is MWB1.2 scFvVH_VL (SEQ ID NO:274).

In some embodiments of the bicistronic iCAR/aCAR construct or monocistronic aCAR and iCAR constructs for co-transduction, the iCAR hinge domain component is selected from a PD-1 hinge, a CD28 hinge, and a CD8 hinge (including a CD8a hinge), a LIR1 Ig3-4 hinge, a LIR1 Ig-4 hinge, a LIR1 52 aa hinge, a LIR1 36 aa hinge, a LIR1 30 aa hinge, a LIR1 26 aa hinge, a LIR1 8 aa hinge, a CD33 hinge, a KIR2DL1 hinge, a PD-1 (47) hinge, a PD-1 (42) hinge, a PD-1 (36) hinge, a PD-1 (30) hinge, a PD-1 (26) hinge, and a PD-1 (20) hinge.

In some embodiments of the bicistronic iCAR/aCAR construct or monocistronic aCAR and iCAR constructs for co-transduction, the iCAR hinge domain component is a PD-1 hinge (SEQ ID NO: 86).

In some embodiments of the bicistronic iCAR/aCAR construct or monocistronic aCAR and iCAR constructs for co-transduction, the iCAR hinge domain component is a CD28 hinge (SEQ ID NO: 85).

In some embodiments of the bicistronic iCAR/aCAR construct or monocistronic aCAR and iCAR constructs for co-transduction, the iCAR hinge domain component is a CD8 alpha hinge (SEQ ID NO: 84).

In some embodiments of the bicistronic iCAR/aCAR construct or monocistronic aCAR and iCAR constructs for co-transduction, the iCAR hinge domain component is a LIR1 Ig3-4 hinge (SEQ ID NO: 87).

In some embodiments of the bicistronic iCAR/aCAR construct or monocistronic aCAR and iCAR constructs for co-transduction, the iCAR hinge domain component is a LIR1 Ig-4 hinge (SEQ ID NO: 88).

In some embodiments of the bicistronic iCAR/aCAR construct or monocistronic aCAR and iCAR constructs for co-transduction, the iCAR hinge domain component is a LIR1 52 aa hinge (SEQ ID NO: 89).

In some embodiments of the bicistronic iCAR/aCAR construct or monocistronic aCAR and iCAR constructs for co-transduction, the iCAR hinge domain component is a LIR1 36 aa hinge (SEQ ID NO: 90).

In some embodiments of the bicistronic iCAR/aCAR construct or monocistronic aCAR and iCAR constructs for co-transduction, the iCAR hinge domain component is a LIR1 30 aa hinge (SEQ ID NO: 91).

In some embodiments of the bicistronic iCAR/aCAR construct or monocistronic aCAR and iCAR constructs for co-transduction, the iCAR hinge domain component is a LIR1 26 aa hinge (SEQ ID NO: 289).

In some embodiments of the bicistronic iCAR/aCAR construct or monocistronic aCAR and iCAR constructs for co-transduction, the iCAR hinge domain component is a LIR1 8 aa hinge (SEQ ID NO:92).

In some embodiments of the bicistronic iCAR/aCAR construct or monocistronic aCAR and iCAR constructs for co-transduction, the iCAR hinge domain component is a CD33 hinge (SEQ ID NO: 93).

In some embodiments of the bicistronic iCAR/aCAR construct or monocistronic aCAR and iCAR constructs for co-transduction, the iCAR hinge domain component is a KIR2DL1 hinge (SEQ ID NO: 94).

In some embodiments of the bicistronic iCAR/aCAR construct or monocistronic aCAR and iCAR constructs for co-transduction, the iCAR hinge domain component is a PD-1 (47) hinge (SEQ ID NO: 290).

In some embodiments of the bicistronic iCAR/aCAR construct or monocistronic aCAR and iCAR constructs for co-transduction, the iCAR hinge domain component is a PD-1 (42) hinge (SEQ ID NO: 291).

In some embodiments of the bicistronic iCAR/aCAR construct or monocistronic aCAR and iCAR constructs for co-transduction, the iCAR hinge domain component is a PD-1 (36) hinge (SEQ ID NO: 292).

In some embodiments of the bicistronic iCAR/aCAR construct or monocistronic aCAR and iCAR constructs for co-transduction, the iCAR hinge domain component is a PD-1 (30) hinge (SEQ ID NO: 293).

In some embodiments of the bicistronic iCAR/aCAR construct or monocistronic aCAR and iCAR constructs for co-transduction, the iCAR hinge domain component is a PD-1 (26) hinge (SEQ ID NO: 294).

In some embodiments of the bicistronic iCAR/aCAR construct or monocistronic aCAR and iCAR constructs for co-transduction, the iCAR hinge domain component is a PD-1 (20) hinge (SEQ ID NO: 295).

In some embodiments of the bicistronic iCAR/aCAR construct or monocistronic aCAR and iCAR constructs for co-transduction, the iCAR TM domain component is selected from a PD-1 TM domain, a CD28 TM domain, a CD8 TM domain (including a CD8a TM domain), a LIR1 TM domain, a CD33 TM domain, and a KIR2DL1 TM domain.

In some embodiments of the bicistronic iCAR/aCAR construct or monocistronic aCAR and iCAR constructs for co-transduction, the iCAR TM domain component is a PD-1 TM domain (SEQ ID NO:97).

In some embodiments of the bicistronic iCAR/aCAR construct or monocistronic aCAR and iCAR constructs for co-transduction, the iCAR TM domain component is a CD28 TM domain (SEQ ID NO:96).

In some embodiments of the bicistronic iCAR/aCAR construct or monocistronic aCAR and iCAR constructs for co-transduction, the iCAR TM domain component is a CD8 alpha TM domain (SEQ ID NO:95).

In some embodiments of the bicistronic iCAR/aCAR construct or monocistronic aCAR and iCAR constructs for co-transduction, the iCAR TM domain component is a LIR1 TM domain (SEQ ID NO:98).

In some embodiments of the bicistronic iCAR/aCAR construct or monocistronic aCAR and iCAR constructs for co-transduction, the iCAR TM domain component is a CD33 TM domain (SEQ ID NO:99).

In some embodiments of the bicistronic iCAR/aCAR construct or monocistronic aCAR and iCAR constructs for co-transduction, the iCAR TM domain component is a KIR2DL1 TM domain (SEQ ID NO:100).

In some embodiments of the bicistronic iCAR/aCAR construct or monocistronic aCAR and iCAR constructs for co-transduction, the iCAR inhibitory domain component is an inhibitory domain from a protein selected from the group consisting of PD-1, KIR2DL1, KIR2DL2, KIR2DL3, KIR2DL4, KIR2DL5A, KIR3DL1, KIR3DL2, KIR3DL3, LAIR1, CD22, CD33, SIGLEC5, SIGLEC6, SIGLEC7, SIGLEC8, SIGLEC9, SIGLEC10, SIGLEC11, SIGLEC12, PECAM1/CD31, CD200R1, FCRL1, FCRL2, FCRL3, FCRL4, FCRL5, SLAMF1, SLAMF5, BTLA, LAG3, 2B4, CD160, CEACAM1, TIM3, VISTA, TIGIT, SIRPalpha, FcγRIIB, CD5, CD300a, CD300f, LIR1, LIR2, LIR3, LIR5, LIRE, Ly9, 2xPD1(G4S), 2xPD1(PD1), PVRIg, and AA2AR.

In some embodiments of the bicistronic iCAR/aCAR construct or monocistronic aCAR and iCAR constructs for co-transduction, the iCAR inhibitory domain component is a PD-1 inhibitory domain (SEQ ID NO:101).

In some embodiments of the bicistronic iCAR/aCAR construct or monocistronic aCAR and iCAR constructs for co-transduction, the iCAR component is a KIR2DL1 inhibitory domain (SEQ ID NO:102).

In some embodiments of the bicistronic iCAR/aCAR construct or monocistronic aCAR and iCAR constructs for co-transduction, the iCAR component is a KIR2DL2 inhibitory domain (SEQ ID NO:103).

In some embodiments of the bicistronic iCAR/aCAR construct or monocistronic aCAR and iCAR constructs for co-transduction, the iCAR component is a KIR2DL3 inhibitory domain (SEQ ID NO:104).

In some embodiments of the bicistronic iCAR/aCAR construct or monocistronic aCAR and iCAR constructs for co-transduction, the iCAR inhibitory domain component is a KIR2DL4 inhibitory domain (SEQ ID NO:105).

In some embodiments of the bicistronic iCAR/aCAR construct or monocistronic aCAR and iCAR constructs for co-transduction, the iCAR inhibitory domain component is a KIR2DL5A inhibitory domain (SEQ ID NO:106).

In some embodiments of the bicistronic iCAR/aCAR construct or monocistronic aCAR and iCAR constructs for co-transduction, the iCAR inhibitory domain component is a KIR3DL1 inhibitory domain (SEQ ID NO:107).

In some embodiments of the bicistronic iCAR/aCAR construct or monocistronic aCAR and iCAR constructs for co-transduction, the iCAR inhibitory domain component is a KIR3DL2 inhibitory domain (SEQ ID NO:108).

In some embodiments of the bicistronic iCAR/aCAR construct or monocistronic aCAR and iCAR constructs for co-transduction, the iCAR inhibitory domain component is a KIR3DL3 inhibitory domain (SEQ ID NO:109).

In some embodiments of the bicistronic iCAR/aCAR construct or monocistronic aCAR and iCAR constructs for co-transduction, the iCAR inhibitory domain component is a LAIR1 inhibitory domain (SEQ ID NO:110).

In some embodiments of the bicistronic iCAR/aCAR construct or monocistronic aCAR and iCAR constructs for co-transduction, the iCAR inhibitory domain component is a CD22 inhibitory domain (SEQ ID NO:111).

In some embodiments of the bicistronic iCAR/aCAR construct or monocistronic aCAR and iCAR constructs for co-transduction, the iCAR inhibitory domain component is a CD33 inhibitory domain (SEQ ID NO:112).

In some embodiments of the bicistronic iCAR/aCAR construct or monocistronic aCAR and iCAR constructs for co-transduction, the iCAR inhibitory domain component is a SIGLEC5 inhibitory domain (SEQ ID NO:113).

In some embodiments of the bicistronic iCAR/aCAR construct or monocistronic aCAR and iCAR constructs for co-transduction, the iCAR inhibitory domain component is a SIGLEC6 inhibitory domain (SEQ ID NO:114).

In some embodiments of the bicistronic iCAR/aCAR construct or monocistronic aCAR and iCAR constructs for co-transduction, the iCAR inhibitory domain component is a SIGLEC7 inhibitory domain (SEQ ID NO:115).

In some embodiments of the bicistronic iCAR/aCAR construct or monocistronic aCAR and iCAR constructs for co-transduction, the iCAR inhibitory domain component is a SIGLEC8 inhibitory domain (SEQ ID NO:116).

In some embodiments of the bicistronic iCAR/aCAR construct or monocistronic aCAR and iCAR constructs for co-transduction, the iCAR inhibitory domain component is a SIGLEC9 inhibitory domain (SEQ ID NO:117).

In some embodiments of the bicistronic iCAR/aCAR construct or monocistronic aCAR and iCAR constructs for co-transduction, the iCAR inhibitory domain component is a SIGLEC10inhibitory domain (SEQ ID NO:118).

In some embodiments of the bicistronic iCAR/aCAR construct or monocistronic aCAR and iCAR constructs for co-transduction, the iCAR inhibitory domain component is a SIGLEC11inhibitory domain (SEQ ID NO:119).

In some embodiments of the bicistronic iCAR/aCAR construct or monocistronic aCAR and iCAR constructs for co-transduction, the iCAR inhibitory domain component is a SIGLEC12inhibitory domain (SEQ ID NO:120).

In some embodiments of the bicistronic iCAR/aCAR construct or monocistronic aCAR and iCAR constructs for co-transduction, the iCAR inhibitory domain component is a PECAM1/CD31 inhibitory domain (SEQ ID NO:121).

In some embodiments of the bicistronic iCAR/aCAR construct or monocistronic aCAR and iCAR constructs for co-transduction, the iCAR inhibitory domain component is a CD200R1inhibitory domain (SEQ ID NO:122).

In some embodiments of the bicistronic iCAR/aCAR construct or monocistronic aCAR and iCAR constructs for co-transduction, the iCAR inhibitory domain component is a FCRL1inhibitory domain (SEQ ID NO:123).

In some embodiments of the bicistronic iCAR/aCAR construct or monocistronic aCAR and iCAR constructs for co-transduction, the iCAR inhibitory domain component is a FCRL2inhibitory domain (SEQ ID NO:124).

In some embodiments of the bicistronic iCAR/aCAR construct or monocistronic aCAR and iCAR constructs for co-transduction, the iCAR inhibitory domain component is a FCRL3inhibitory domain (SEQ ID NO:125).

In some embodiments of the bicistronic iCAR/aCAR construct or monocistronic aCAR and iCAR constructs for co-transduction, the iCAR inhibitory domain component is a FCRL4 inhibitory domain (SEQ ID NO:126).

In some embodiments of the bicistronic iCAR/aCAR construct or monocistronic aCAR and iCAR constructs for co-transduction, the iCAR inhibitory domain component is a FCRL5 inhibitory domain (SEQ ID NO:127).

In some embodiments of the bicistronic iCAR/aCAR construct or monocistronic aCAR and iCAR constructs for co-transduction, the iCAR inhibitory domain component is a SLAMF1 inhibitory domain (SEQ ID NO:128).

In some embodiments of the bicistronic iCAR/aCAR construct or monocistronic aCAR and iCAR constructs for co-transduction, the iCAR inhibitory domain component is a SLAMF5 inhibitory domain (SEQ ID NO:129).

In some embodiments of the bicistronic iCAR/aCAR construct or monocistronic aCAR and iCAR constructs for co-transduction, the iCAR inhibitory domain component is a BTLA inhibitory domain (SEQ ID NO:130).

In some embodiments of the bicistronic iCAR/aCAR construct or monocistronic aCAR and iCAR constructs for co-transduction, the iCAR inhibitory domain component is a LAG3 inhibitory domain (SEQ ID NO:131).

In some embodiments of the bicistronic iCAR/aCAR construct or monocistronic aCAR and iCAR constructs for co-transduction, the iCAR inhibitory domain component is a 2B4 inhibitory domain (SEQ ID NO:132).

In some embodiments of the bicistronic iCAR/aCAR construct or monocistronic aCAR and iCAR constructs for co-transduction, the iCAR inhibitory domain component is a CD160 inhibitory domain (SEQ ID NO:133).

In some embodiments of the bicistronic iCAR/aCAR construct or monocistronic aCAR and iCAR constructs for co-transduction, the iCAR inhibitory domain component is a CEACAM1 inhibitory domain (SEQ ID NO:134).

In some embodiments of the bicistronic iCAR/aCAR construct or monocistronic aCAR and iCAR constructs for co-transduction, the iCAR inhibitory domain component is a TIM3 inhibitory domain (SEQ ID NO:135).

In some embodiments of the bicistronic iCAR/aCAR construct or monocistronic aCAR and iCAR constructs for co-transduction, the iCAR inhibitory domain component is a VISTA inhibitory domain (SEQ ID NO:136).

In some embodiments of the bicistronic iCAR/aCAR construct or monocistronic aCAR and iCAR constructs for co-transduction, the iCAR inhibitory domain component is a TIGIT inhibitory domain (SEQ ID NO:137).

In some embodiments of the bicistronic iCAR/aCAR construct or monocistronic aCAR and iCAR constructs for co-transduction, the iCAR inhibitory domain component is a SIRPalpha inhibitory domain (SEQ ID NO:138).

In some embodiments of the bicistronic iCAR/aCAR construct or monocistronic aCAR and iCAR constructs for co-transduction, the iCAR inhibitory domain component is a FcγRIIB inhibitory domain (SEQ ID NO:139).

In some embodiments of the bicistronic iCAR/aCAR construct or monocistronic aCAR and iCAR constructs for co-transduction, the iCAR inhibitory domain component is a CD5 inhibitory domain (SEQ ID NO:140).

In some embodiments of the bicistronic iCAR/aCAR construct or monocistronic aCAR and iCAR constructs for co-transduction, the iCAR inhibitory domain component is a CD300a inhibitory domain (SEQ ID NO:141).

In some embodiments of the bicistronic iCAR/aCAR construct or monocistronic aCAR and iCAR constructs for co-transduction, the iCAR inhibitory domain component is a CD300f inhibitory domain (SEQ ID NO:142).

In some embodiments of the bicistronic iCAR/aCAR construct or monocistronic aCAR and iCAR constructs for co-transduction, the iCAR inhibitory domain component is a LIR1 inhibitory domain (SEQ ID NO:143).

In some embodiments of the bicistronic iCAR/aCAR construct or monocistronic aCAR and iCAR constructs for co-transduction, the iCAR inhibitory domain component is a LIR2 inhibitory domain (SEQ ID NO:144).

In some embodiments of the bicistronic iCAR/aCAR construct or monocistronic aCAR and iCAR constructs for co-transduction, the iCAR inhibitory domain component is a LIR3 inhibitory domain (SEQ ID NO:145).

In some embodiments of the bicistronic iCAR/aCAR construct or monocistronic aCAR and iCAR constructs for co-transduction, the iCAR inhibitory domain component is a LIR5 inhibitory domain (SEQ ID NO:146).

In some embodiments of the bicistronic iCAR/aCAR construct or monocistronic aCAR and iCAR constructs for co-transduction, the iCAR inhibitory domain component is a LIR8 inhibitory domain (SEQ ID NO:147).

In some embodiments of the bicistronic iCAR/aCAR construct or monocistronic aCAR and iCAR constructs for co-transduction, the iCAR inhibitory domain component is a Ly9 inhibitory domain (SEQ ID NO:148).

In some embodiments of the bicistronic iCAR/aCAR construct or monocistronic aCAR and iCAR constructs for co-transduction, the iCAR inhibitory domain component is a 2xPD1(G4S) inhibitory domain (SEQ ID NO:149).

In some embodiments of the bicistronic iCAR/aCAR construct or monocistronic aCAR and iCAR constructs for co-transduction, the iCAR inhibitory domain component is a 2xPD1(PD1) inhibitory domain (SEQ ID NO:150).

In some embodiments of the bicistronic iCAR/aCAR construct or monocistronic aCAR and iCAR constructs for co-transduction, the iCAR inhibitory domain component is a PVRIg inhibitory domain (SEQ ID NO:151).

In some embodiments of the bicistronic iCAR/aCAR construct or monocistronic aCAR and iCAR constructs for co-transduction, the iCAR inhibitory domain component is a AA2AR inhibitory domain (SEQ ID NO:152).

In some embodiments of the bicistronic iCAR/aCAR construct or monocistronic aCAR and iCAR constructs for co-transduction, the aCAR single chain variable fragment (scFv) component targets Her2.

In some embodiments of the bicistronic iCAR/aCAR construct or monocistronic aCAR and iCAR constructs for co-transduction, the aCAR scFv comprises the Vh and Vl from trastuzumab (SEQ ID NOs:170 and 171, respectively).

In some embodiments of the bicistronic iCAR/aCAR construct or monocistronic aCAR and iCAR constructs for co-transduction, the aCAR scFv is SEQ ID NO:172.

In some embodiments of the bicistronic iCAR/aCAR construct or monocistronic aCAR and iCAR constructs for co-transduction, the aCAR scFv comprises the Vh and Vl from trastuzumab F9G (SEQ ID NOs: 307 and 308).

In some embodiments of the bicistronic iCAR/aCAR construct or monocistronic aCAR and iCAR constructs for co-transduction, the aCAR scFv comprises the Vh and Vl from pertuzumab (SEQ ID NOs:173 and 174, respectively).

In some embodiments of the bicistronic iCAR/aCAR construct or monocistronic aCAR and iCAR constructs for co-transduction, the aCAR scFv is SEQ ID NO:175.

In some embodiments of the bicistronic iCAR/aCAR construct or monocistronic aCAR and iCAR constructs for co-transduction, the aCAR scFv comprises the Vh and Vl from FRP5 (SEQ ID NOs:176 and 177, respectively).

In some embodiments of the bicistronic iCAR/aCAR construct or monocistronic aCAR and iCAR constructs for co-transduction, the aCAR scFv comprises the Vh and Vl from A21 (SEQ ID NOs:178 and 179, respectively).

In some embodiments of the bicistronic iCAR/aCAR construct or monocistronic aCAR and iCAR constructs for co-transduction, the aCAR scFv comprises the Vh and Vl from XMT1517 (SEQ ID NOs:180 and 181, respectively).

In some embodiments of the bicistronic iCAR/aCAR construct or monocistronic aCAR and iCAR constructs for co-transduction, the aCAR scFv comprises the Vh and Vl from XMT1518 (SEQ ID NOs:182 and 183, respectively).

In some embodiments of the bicistronic iCAR/aCAR construct or monocistronic aCAR and iCAR constructs for co-transduction, the aCAR scFv comprises the Vh and Vl from XMT1519 (SEQ ID NOs:184 and 185, respectively).

In some embodiments of the bicistronic iCAR/aCAR construct or monocistronic aCAR and iCAR constructs for co-transduction, the aCAR scFv comprises the Vh and Vl from FWP51 (SEQ ID NOs:186 and 187, respectively).

In some embodiments of the bicistronic iCAR/aCAR construct or monocistronic aCAR and iCAR constructs for co-transduction, the aCAR scFv comprises SEQ ID NOs: 188.

In some embodiments of the bicistronic iCAR/aCAR construct or monocistronic aCAR and iCAR constructs for co-transduction, the aCAR single chain variable fragment (scFv) component targets EGFR.

In some embodiments of the bicistronic iCAR/aCAR construct or monocistronic aCAR and iCAR constructs for co-transduction, the aCAR scFv comprises the Vh and Vl from cetuximab (SEQ ID NOs:189 and 190, respectively).

In some embodiments of the bicistronic iCAR/aCAR construct or monocistronic aCAR and iCAR constructs for co-transduction, the aCAR scFv is SEQ ID NO:191.

In some embodiments of the bicistronic iCAR/aCAR construct or monocistronic aCAR and iCAR constructs for co-transduction, the aCAR scFv comprises the Vh and Vl from panitumumab (SEQ ID NOs:192 and 193, respectively).

In some embodiments of the bicistronic iCAR/aCAR construct or monocistronic aCAR and iCAR constructs for co-transduction, the aCAR scFv is SEQ ID NO:194.

In some embodiments of the bicistronic iCAR/aCAR construct or monocistronic aCAR and iCAR constructs for co-transduction, the aCAR scFv comprises the Vh and Vl from Imgatuzumab (SEQ ID NOs:195 and 196, respectively).

In some embodiments of the bicistronic iCAR/aCAR construct or monocistronic aCAR and iCAR constructs for co-transduction, the aCAR scFv comprises the Vh and Vl from Nimotuzumab (SEQ ID NOs:197 and 198, respectively).

In some embodiments of the bicistronic iCAR/aCAR construct or monocistronic aCAR and iCAR constructs for co-transduction, the aCAR scFv comprises the Vh and Vl from Nimotuzumab (K5) (SEQ ID NOs:310 and 311, respectively).

In some embodiments of the bicistronic iCAR/aCAR construct or monocistronic aCAR and iCAR constructs for co-transduction, the aCAR scFv comprises the Vh and Vl from Necitumumab (SEQ ID NOs:199 and 200, respectively).

In some embodiments of the bicistronic iCAR/aCAR construct or monocistronic aCAR and iCAR constructs for co-transduction, the aCAR scFv comprises the Vh and Vl from ICR62 (SEQ ID NOs:201 and 202, respectively).

In some embodiments of the bicistronic iCAR/aCAR construct or monocistronic aCAR and iCAR constructs for co-transduction, the aCAR scFv comprises the Vh and Vl from Matuzumab (SEQ ID NOs:204 and 205, respectively).

In some embodiments of the bicistronic iCAR/aCAR construct or monocistronic aCAR and iCAR constructs for co-transduction, the aCAR scFv comprises the Vh and Vl from C10 (SEQ ID NOs:206 and 207, respectively).

In some embodiments of the bicistronic iCAR/aCAR construct or monocistronic aCAR and iCAR constructs for co-transduction, the aCAR scFv comprises the Vh and Vl from Zalutumumab (SEQ ID NOs:208 and 209, respectively).

In some embodiments of the bicistronic iCAR/aCAR construct or monocistronic aCAR and iCAR constructs for co-transduction, the aCAR scFv comprises the Vh and Vl from P1X (SEQ ID NOs:210 and 211, respectively).

In some embodiments of the bicistronic iCAR/aCAR construct or monocistronic aCAR and iCAR constructs for co-transduction, the aCAR scFv comprises the Vh and Vl from P2X (SEQ ID NOs:212 and 213, respectively).

In some embodiments of the bicistronic iCAR/aCAR construct or monocistronic aCAR and iCAR constructs for co-transduction, the aCAR scFv comprises the Vh and Vl from P3X (SEQ ID NOs:214 and 215, respectively).

In some embodiments of the bicistronic iCAR/aCAR construct or monocistronic aCAR and iCAR constructs for co-transduction, the aCAR scFv comprises the VH from EGFR-1a1-VHH (SEQ ID NO:216).

In some embodiments of the bicistronic iCAR/aCAR construct or monocistronic aCAR and iCAR constructs for co-transduction, the aCAR scFv comprises the VH from EGFR-VHH (SEQ ID NO:312).

In some embodiments of the bicistronic iCAR/aCAR construct or monocistronic aCAR and iCAR constructs for co-transduction, the aCAR single chain variable fragment (scFv) component targets Mesothelin.

In some embodiments of the bicistronic iCAR/aCAR construct or monocistronic aCAR and iCAR constructs for co-transduction, the aCAR scFv comprise the Vh and Vl from Amatuximab (SEQ ID NOs:217 and 218, respectively).

In some embodiments of the bicistronic iCAR/aCAR construct or monocistronic aCAR and iCAR constructs for co-transduction, the aCAR scFv comprise the Vh and Vl from P4 (SEQ ID NOs:219 and 220, respectively).

In some embodiments of the bicistronic iCAR/aCAR construct or monocistronic aCAR and iCAR constructs for co-transduction, the aCAR scFv comprise the Vh and Vl from SS1 (SEQ ID NOs:222 and 223, respectively).

In some embodiments of the bicistronic iCAR/aCAR construct or monocistronic aCAR and iCAR constructs for co-transduction, the aCAR scFv comprise the VHH from SD1 (SEQ ID NO:225).

In some embodiments of the bicistronic iCAR/aCAR construct or monocistronic aCAR and iCAR constructs for co-transduction, the aCAR scFv comprise the VHH from SD2 (SEQ ID NO:226).

In some embodiments of the bicistronic iCAR/aCAR construct or monocistronic aCAR and iCAR constructs for co-transduction, the aCAR scFv comprise the Vh and Vl from 1H7 (SEQ ID NOs:227 and 228, respectively).

In some embodiments of the bicistronic iCAR/aCAR construct or monocistronic aCAR and iCAR constructs for co-transduction, the aCAR scFv comprise the Vh and Vl from 3C02 (SEQ ID NOs:230 and 231, respectively).

In some embodiments of the bicistronic iCAR/aCAR construct or monocistronic aCAR and iCAR constructs for co-transduction, the hinge TM domain component is selected from the group consisting of a CD28 hinge and a CD8 hinge (including a CD8a hinge domain).

In some embodiments of the bicistronic iCAR/aCAR construct or monocistronic aCAR and iCAR constructs for co-transduction, the hinge TM domain component is a CD28 hinge domain (SEQ ID NO: 85).

In some embodiments of the bicistronic iCAR/aCAR construct or monocistronic aCAR and iCAR constructs for co-transduction, the hinge TM domain component is a CD8 alpha hinge domain (SEQ ID NO: 84).

In some embodiments of the bicistronic iCAR/aCAR construct or monocistronic aCAR and iCAR constructs for co-transduction, the co-stimulatory domain component is selected from the group consisting of a CD137 (4-1BB) co-stimulatory domain, a CD28 co-stimulatory domain, a 28BB co-stimulatory domain, and a CD3z co-stimulatory domain.

In some embodiments of the bicistronic iCAR/aCAR construct or monocistronic aCAR and iCAR constructs for co-transduction, the co-stimulatory domain component is a CD137 (4-1BB) co-stimulatory domain (SEQ ID NO:233).

In some embodiments of the bicistronic iCAR/aCAR construct or monocistronic aCAR and iCAR constructs for co-transduction, the co-stimulatory domain component is a CD28 co-stimulatory domain (SEQ ID NO:234).

In some embodiments of the bicistronic iCAR/aCAR construct or monocistronic aCAR and iCAR constructs for co-transduction, the co-stimulatory domain component a CD3z activation signaling domain (SEQ ID NO:235).

In some embodiments of the bicistronic iCAR/aCAR construct or monocistronic aCAR and iCAR constructs for co-transduction, the ITAM is a CD3 zeta domain.

In some embodiments of the bicistronic iCAR/aCAR construct or monocistronic aCAR and iCAR constructs for co-transduction, the ITAM is a CD3 zeta domain (SEQ ID NO:236).

In some embodiments of the bicistronic iCAR/aCAR construct or monocistronic aCAR and iCAR constructs for co-transduction, the ITAM is a CD3 zeta 3F domain (SEQ ID NO:237).

In some embodiments of the bicistronic iCAR/aCAR construct or monocistronic aCAR and iCAR constructs for co-transduction, the ITAM is a CD3 zeta 4F domain (SEQ ID NO:238).

In some embodiments of the bicistronic iCAR/aCAR construct or monocistronic aCAR and iCAR constructs for co-transduction, the ITAM is a CD3 zeta 4OF domain (SEQ ID NO:239).

In some embodiments of the bicistronic iCAR/aCAR construct or monocistronic aCAR and iCAR constructs for co-transduction, the linker connecting the iCAR portion and the aCAR portion comprises one or more linker selected from the group consisting of T2A (SEQ ID NO:155), F2A (SEQ ID NO:156), P2A (SEQ ID NO:157), E2A (SEQ ID NO:158), and an IRES sequence (SEQ ID NO:159 or 160).

In some embodiments of the bicistronic iCAR/aCAR construct or monocistronic aCAR and iCAR constructs for co-transduction, the linker connecting the iCAR portion and the aCAR portion is GSG T2A (SEQ ID NO:155).

In some embodiments of the bicistronic iCAR/aCAR construct or monocistronic aCAR and iCAR constructs for co-transduction, the bicistronic iCAR/aCAR construct comprises an amino acid sequence encoded by a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments of the bicistronic iCAR/aCAR construct or monocistronic aCAR and iCAR constructs for co-transduction, the bicistronic iCAR/aCAR construct comprises an amino acid sequence encoded by a nucleic acid sequence selected from the group consisting of SEQ ID NO:275, SEQ ID NO:277, SEQ ID NO:279, SEQ ID NO:281, SEQ ID NO:321, SEQ ID NO:323, and SEQ ID NO:325.

In some embodiments of the bicistronic iCAR/aCAR construct or monocistronic aCAR and iCAR constructs for co-transduction, the bicistronic iCAR/aCAR construct comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments of the bicistronic iCAR/aCAR construct or monocistronic aCAR and iCAR constructs for co-transduction, the bicistronic iCAR/aCAR construct comprises an amino acid sequence selected from the group consisting of SEQ ID NO:276, SEQ ID NO:278, SEQ ID NO:280, SEQ ID NO:282, SEQ ID NO:322, SEQ ID NO:324, and SEQ ID NO:326.

In some embodiments of the bicistronic iCAR/aCAR construct or monocistronic aCAR and iCAR constructs for co-transduction, the bicistronic iCAR/aCAR construct further comprises a short hairpin RNA (shRNA).

In some embodiments of the bicistronic iCAR/aCAR construct or monocistronic aCAR and iCAR constructs for co-transduction as described herein comprise an iCAR that comprises a synthetic PD-1 or LIR1 sequence as shown in Table 8, including one selected from the group consisting of SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:296, SEQ ID NO:297, SEQ ID NO:298, SEQ ID NO:299, SEQ ID NO:300, SEQ ID NO:301, SEQ ID NO:302, and SEQ ID NO:304.

In some embodiments of the bicistronic iCAR/aCAR construct or monocistronic aCAR and iCAR constructs for co-transduction as described herein comprise an iCAR comprising an amino acid sequence selected from the group consisting of SEQ ID NO: 255, SEQ ID NO:256, SEQ ID NO:257, SEQ ID NO:258, SEQ ID NO:305, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:327, SEQ ID NO:328, SEQ ID NO:329, SEQ ID NO:330, SEQ ID NO:331, SEQ ID NO:332, SEQ ID NO:333, and SEQ ID NO:334.

In some embodiments of the bicistronic iCAR/aCAR construct or monocistronic aCAR and iCAR constructs for co-transduction as described herein comprise a construct as described in Table 1, Table 11 and/or Table 12.

In some embodiments of the bicistronic iCAR/aCAR construct or monocistronic aCAR and iCAR constructs for co-transduction as described herein comprise a construct or portion thereof as described in any one of Tables 1 to 22.

In some embodiments of the bicistronic iCAR/aCAR construct or monocistronic aCAR and iCAR constructs for co-transduction as described herein comprise a construct as described in any one of Tables 15, 16, 17, and/or 21.

In some embodiments of the bicistronic iCAR/aCAR construct or monocistronic aCAR and iCAR constructs for co-transduction as described herein comprise a construct as described in any one of Tables 1, 2, 4, 9, 10, 11 and/or 12.

The present invention also provides for a nucleic acid composition comprising a nucleic acid that encodes a bicistronic iCAR/aCAR construct or monocistronic aCAR and iCAR constructs for co-transduction according to any one of the preceding claims.

The present invention also provides for a vector comprising a nucleic acid sequence encoding for a bicistronic iCAR/aCAR construct or monocistronic aCAR and iCAR constructs for co-transduction according to any one of the preceding claims.

The present invention also provides for a vector composition comprising the vector according to paragraphs [00192].

In some embodiments, the iCAR/aCAR construct or monocistronic aCAR and iCAR constructs for co-transduction comprises a signal peptide upstream of the iCAR and/or aCAR portions. In some embodiments, the signal peptide is a CD8 alpha signal peptide (SEQ ID NO: 161), a GM-CSF signal peptide (SEQ ID NO: 162), or a mIgK signal peptide (SEQ ID NO: 306).

The present invention also provides for a safe effector cell comprising a nucleic acid or nucleic acid sequence composition as described herein.

The present invention also provides for a safe effector cell comprising a vector or vector composition o as described herein.

A safe effector immune cell expressing a bicistronic iCAR/aCAR construct or monocistronic aCAR and iCAR constructs for co-transduction as described herein.

A method for treating cancer in a patient having a tumor characterized by LOH, comprising administering to the patient a safe effector immune cell as described herein.

A method for treating cancer in a patient having a tumor characterized by a genetic mutation resulting in a complete loss of expression of a target gene or target extracellular polymorphic epitope gene, comprising administering to the patient a safe effector immune cell as described herein.

A method for treating cancer in a patient having a tumor characterized by loss of heterozygosity (LOH), or other genetic loss or allelic imbalance phenotypes including, without limitation, loss of function or expression, resulting from mutations affecting one or more nucleotides, comprising administering to the patient a safe effector immune cell as described herein.

In some embodiments, the cancer is selected from the group consisting of Acute Myeloid Leukemia [LAML], Adrenocortical carcinoma [ACC], Bladder Urothelial Carcinoma [BLCA], Brain Lower Grade Glioma [LGG], Breast invasive carcinoma [BRCA], Cervical squamous cell carcinoma and endocervical adenocarcinoma [CESC], Cholangiocarcinoma [CHOL], Colon adenocarcinoma [COAD], Esophageal carcinoma [ESCA], Glioblastoma multiforme [GBM], Head and Neck squamous cell carcinoma [HNSC], Kidney Chromophobe [KICH], Kidney renal clear cell carcinoma [KIRC], Kidney renal papillary cell carcinoma [KIRP], Liver hepatocellular carcinoma [LIHC], Lung adenocarcinoma [LUAD], Lung squamous cell carcinoma [LUSC], Lymphoid Neoplasm Diffuse Large B-cell Lymphoma [DLBC], Mesothelioma [MESO], Ovarian serous cystadenocarcinoma [OV], Pancreatic adenocarcinoma [PAAD], Pheochromocytoma and Paraganglioma [PCPG], Prostate adenocarcinoma [PRAD], Rectum adenocarcinoma [READ], Sarcoma [SARC], Skin Cutaneous Melanoma [SKCM], Stomach adenocarcinoma [STAD], Testicular Germ Cell Tumors [TGCT], Thymoma [THYM], Thyroid carcinoma [THCA], Uterine Carcinosarcoma [UCS], Uterine Corpus Endometrial Carcinoma [UCEC], Uveal Melanoma [UVM], Non-small cell lung carcinoma [NSCLC], and Small cell lung cancer [SCLC].

### BRIEF DESCRIPTION OF THE DRAWINGS

FIG. 1 shows bicistronic construct design overview and component table.

FIGS. 2A-2H show bicistronic survey—constructs MC0280-MC0300, MC0428, MC0447, MC0449, HLA-A2 shRNA.

FIG. 3 shows BTLA & KIR2DL2 as new iCAR leads.

FIG. 4 shows identification of fully human scFv constructs with higher HLA-A binding avidity.

FIG. 5 shows 3PF12 & SN66E3 PD-1 iCAR exhibit are more stably expressed.

FIG. 6 shows a schematic for luciferase-based cytotoxicity assays.

FIGS. 7A-7B. A) Expression of HER2 Bicistronics Day 9—Donor 466. B) Expression of HER2 Bicistronics Day 9-Donor 149.

FIG. 8 shows luciferase killing results for LIR1 & KIR2DL1 dual CAR. LIR1 inhibits efficiently the aCAR, enabling high protection for MCF7. KIR2DL1 inhibits the aCAR, enabling moderate protection for MCF7.

FIG. 9 shows IFNg ELISA assays showing LIR1 and KIR2DL1 inhibition. LIR1 and KIR2DL1 very efficiently inhibit IFNg secretion against MCF7.

FIG. 10 shows KIR2DL1/2 and LIR1 confirmed as hits in Jurkat assay.

FIGS. 11A-11B shows low dual CAR lentiviral transduction efficiency and variable expression.

FIGS. 12A-12B shows experimental set-up and data regarding target cell killing and CAR-T activation correlate with E/T ratio.

FIG. 13 shows target cell killing and CAR-T activation correlate with E/T ratio.

FIG. 14 shows a quantum bead assay to determine CAR cell surface level.

FIG. 15 shows exceptional differential PD-1 iCAR expression relative to HER2 aCAR.

FIG. 16 shows target antigen quantifications in screen cell-line panel.

FIG. 17 shows PD-1 iCAR directs HLA-A2 specific EGFR a CAR killing (E/T=2).

FIG. 18 shows HLA-A2 POS cancer cells specifically inhibit dual CAR T-cells

FIG. 19 shows iCAR inhibits T-cell degranulation across a wide range of HLA-A2 level.

FIG. 20 shows a PD-1 iCAR directs HLA-A2 specific HER2 aCAR killing.

FIG. 21 shows dual CAR lentiviral expression is highly variable (HER2 aCAR).

FIG. 22 shows cetuximab scFv lentiviral expression is relatively low.

FIG. 23 shows bicistronic constructs express well on Day 8.

FIG. 24 shows bicistronic expression is lower on Day 12

FIG. 25 shows anti-HLA-A2 iCAR screen—construct design

FIGS. 26A-26B shows alternative scFvs with higher HLA binding than BB7.2 identified.

FIG. 27 shows iCAR single chain options.

FIG. 28 shows BB7.2 (two versions), 3PF12, and SN66E3 PD-1 iCAR exhibit are more stably expressed.

FIG. 29 shows KIR2DL1 iCAR identified as hit in FaDu/U87-LUC immune cell killing assay.

FIGS. 30A-30B shows a schematic for IMPT001: A dual CART system designed to kill based on tumor specific loss-of-HLA-A2 gene expression.

FIGS. 31A-31G. shows donor 149 Expression: HER2 Bicistronics Day 12.

FIGS. 32A-32G shows donor 466 Expression: HER2 Bicistronics Day 12.

FIG. 33 shows D149 Luciferase Kill Assay Results Day 12. LIR1 inhibits efficiently the aCAR, enabling high protection for H1703, H1650 and MDA-MB231. KIR2DL1 and CD33 inhibit the aCAR, enabling moderate protection for H1703, H1650 and MDA-MB231.

FIG. 34 shows D466 Luciferase Kill Assay Results Day 12. LIR1 and CD33 inhibits efficiently the aCAR, enabling protection for H1703, H1650. LIR1 and CD33 inhibit very efficiently IFNγ secretion against H1703, H1650 and MCF7.

FIG. 35 shows HER2 Bicistronic Expression Day 8 from an exemplary experiment.

FIG. 36 shows VR51 (LIR1 iDomain) protect HLA-A2POS targets. LIR1 inhibits efficiently the aCAR, allowing high protection for H1650 and moderate protection for MDA-MB-231 cells from an exemplary experiment.

FIG. 37 provides CAR expression on the cell surface. Note: VR52 had very low aCAR expression (excluded from analysis). VR55,56 had no iCAR expression (data not shown) (excluded from analysis). The MFI is of the positive CAR fraction only. To clarify, the aCAR+ fraction of the untransduced cells (3%) has an MFI of 766.

FIGS. 38A-38C showcell staining of transduced PBMCs (raw data).

FIG. 39 show bicistronic iCAR/aCAR constructs show efficacy against A2NEG cell lines.

FIG. 40 show iCAR RNA expression is transient.

FIGS. 41A-41F show in vitro analysis of bicistronic iCAR-aCAR constructs described herein. VR354 was identified as a superior LIR bicistronic construct for protection against HER2 aCAR killing.

FIG. 42 show screen of HLA-A2 scFv as aCAR. All humanized BB7.2 versions expressed well and showed both binding and efficacy against an A2 POS target. The top hit seemed to be VR375 due to even lower EC50 compared to VR370.

FIG. 43 show HLA-A2 enrichment. Anti-PE beads and Miltenyi LS columns were used to achieve successful enrichment of VR51 bicistronic construct in the bound fraction.

FIGS. 44A-44K show screen of synthetic PD1 constructs. Enriched synthetic PD1 constructs screened using the luciferase assay on H1703 isogenic cell lines showed that synthetic constructs containing 1-5 PD1 ITSM repeats showed superior protection compared to 1-5 PD1 ITIM repeats.

FIG. 45 showscreen of 1×vs 2×PD1 constructs. Enriched PD1 constructs screened using luciferase assay and isogenic H1703 cell lines showed that 2×PD1 construct showed better protection than the naturally occurring 1×PD1 construct, with the G4S linker (VR68) providing superior protection over the PD1 linker (VR69).

FIG. 46 show iCAR Engagement Regulates CAR-T Activation. Singular aCAR engagement by iTarget NEG cells induces T-cell activation. Dual aCAR+iCAR engagement inhibits CAR-T activation with iTarget POS cells.

FIG. 47 show iCAR target POS cancer cells inhibit dual CAR T cells.

FIG. 48 show iCAR targeted killing of cancer cell lines.

FIGS. 49A-49B show screen of SN66E3 iCAR scFv constructs. Enriched bicistronic constructs screened using the luciferase assay on H1703 isogenic cell lines showed that constructs containing SN66E3 iCAR scFv showed superior protection.

FIG. 50 show functional Luc results-Screen of Camel VHH EGFR scFv cotransduced with mBB7.2 scFv with LIR1 or PD1x2 iDomains.

FIG. 51 show scheme of the in-vivo study design.

FIG. 52 show scheme of the in-vivo process.

FIGS. 53A-53D show tumor growth kinetics of a representative in-vivo study with main constructs. Both protection and efficacy are observed for the VR354 and VR51.

constructs.

FIGS. 54A-54F show series F in-vivo screen Tumor growth kinetics. The model is the H1703 WT where protection are observed. Top hit for both CAR-T doses is VR428.

FIGS. 55A-55F show series F in-vivo screen Tumor growth kinetics. The model is the H1703 KO where efficacy are observed. Top hit for both CAR-T doses is VR428.

FIG. 56 show in vitro screen for synthetic iDomains comprising variations in the LIR1 ITIM and PD-1 ITSM motifs of the iCAR.

FIG. 57 show series G in vitro screen for humanized and fully human iCAR scFv specific against the HLA-A2 target.

FIG. 58 show in vitro screen for synthetic LIR1 iDomain comprising variations in the ITIM and ITSM motifs of the iCAR.

FIG. 59 show series F in vitro screen for humanized iCAR scFv specific against the HLA-A2 target. Validation of HzBB7.2 iCAR scFv in-vitro.

### DETAILED DESCRIPTION OF THE INVENTION

### I. Introduction

The present invention provides bicistronic and co-administered monocistronic constructs specifically targeting tumor cells while keeping the normal cells protected. The constructs provided herein provide iCAR/aCAR constructs that target single allelic variants of polymorphic cell surface epitopes, which are lost from tumor cells due to loss of heterozygosity (LOH) of the chromosomal region they reside in, while remaining expressed on normal tissue. Because of the polymorphic variation, the iCAR/aCAR pair is able to distinguish the two alleles and target only the tumor cells missing the target allele due to LOH.

### II. Select Definitions

The term “nucleic acid molecule” as used herein refers to a DNA or RNA molecule.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “endogenous” refers to any material from or produced inside an organism, cell, tissue or system.

The term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “genomic variant” as used herein refers to a change of at least one nucleotide at the genomic level in a sequenced sample compared to the reference or consensus sequence at the same genomic position.

The term “corresponding reference allele” as used herein with reference to a variant means the reference or consensus sequence or nucleotide at the same genomic position as the variant.

The term “extracellular domain” as used herein with reference to a protein means a region of the protein which is outside of the cell membrane.

The term “loss of heterozygosity” or “LOH” as used herein means the loss of chromosomal materials such as a complete chromosome or a part thereof, in one copy of the two chromosomes in a somatic cell.

The term “sequence region” as used herein with reference to a variant or a reference allele means a sequence starting upstream and ending downstream from the position of the variant, which can be translated into an “epitope peptide” that can be recognized by an antibody.

The term “CAR”, as that term is used herein, refers to a chimeric polypeptide that shares structural and functional properties with a cell immune-function receptor or adaptor molecule, from e.g., a T cell or a NK cell. CARs include TCARs and NKR-CARs. Upon binding to cognate antigen, a CAR can activate or inactivate the cytotoxic cell in which it is disposed, or modulate the cell's antitumor activity or otherwise modulate the cells immune response.

The term “specific binding” as used herein in the context of an extracellular domain, such as an scFv, that specifically binds to a single allelic variant of a polymorphic cell surface epitope, refers to the relative binding of the scFv to one allelic variant and its failure to bind to the corresponding different allelic variant of the same polymorphic cell surface epitope. Since this depends on the avidity (number of CAR copies on the T cell, number of antigen molecules on the surface of target cells (or cells to be protected) and the affinity of the specific CARs used, a functional definition would be that the specific scFv would provide a significant signal in an ELISA against the single allelic variant of a polymorphic cell surface epitope to which it is specific or cells transfected with a CAR displaying the scFv would be clearly labeled with the single allelic variant of a polymorphic cell surface epitope in a FACS assay, while the same assays using the corresponding different allelic variant of the same polymorphic cell surface epitope would not give any detectable signal.

The term “treating” as used herein refers to means of obtaining a desired physiological effect. The effect may be therapeutic in terms of partially or completely curing a disease and/or symptoms attributed to the disease. The term refers to inhibiting the disease, e.g., arresting its development; or ameliorating the disease, e.g., causing regression of the disease.

As used herein, the terms “subject” or “individual” or “animal” or “patient” or “mammal,” refers to any subject, particularly a mammalian subject, for whom diagnosis, prognosis, or therapy is desired, for example, a human.

The phrase “safe effector immune cell” or “safe effector cell” includes those cells described by the invention that express at least one bicistronic iCAR/aCAR construct, or portion thereof, as described herein, or exhibit co-expression of monocistronic aCAR and iCAR constructs. In some embodiments, the “safe effector immune cell” or “safe effector cell” is capable of administration to a subject. In some embodiments, the “safe effector immune cell” or “safe effector cell” further expresses at least one bicistronic iCAR/aCAR construct, or portion thereof, or exhibit co-expression of monocistronic aCAR and iCAR constructs, as described herein.

Pharmaceutical compositions for use in accordance with the present invention may be formulated in conventional manner using one or more physiologically acceptable carriers or excipients. The carrier(s) must be “acceptable” in the sense of being compatible with the other ingredients of the composition and not deleterious to the recipient thereof.

The phrase “effective amount” or “therapeutically effective amount” are used interchangeably herein, and refer to an amount of a compound, formulation, material, or composition, as described herein effective to achieve a particular biological result.

The term “peripheral blood mononuclear cell (PBMC)” as used herein refers to any blood cell having a round nucleus, such as a lymphocyte, or a monocyte. Methods for isolating PBMCs from blood are readily apparent to those skilled in the art. A non-limiting example is the extraction of these cells from whole blood using ficoll, a hydrophilic polysaccharide that separates layers of blood, with monocytes and lymphocytes forming a buffy coat under a layer of plasma or by leukapheresis, the preparation of leukocyte concentrates with the return of red cells and leukocyte-poor plasma to the donor.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, glioma, and the like.

### III. CAR-T SYSTEM: iCARs and aCARs

LOH, being a genomic event, results in a total loss of a specific variant from the tumor with a very rare probability of gaining back the lost allele. If the LOH event occurs very early in the development of tumors, it ensures a uniform target signature in all tumor cells derived from the initial pre-malignant tissue including metastatic tumors. Additionally, LOH occurs in almost all types of cancer and this concept can therefore be relied upon as a universal tool for developing markers relevant to all these cancer types. Since the LOH events are to some extent random, the present invention further provides for selection of personalized tumor markers for each individual cancer patient, based on the specific LOH events which took place in that patient. The tools relied upon to execute this concept, the aCARs and the iCARs, are well-known and can be easily prepared using methods well-known in the art as taught for example, in WO 2015/142314 and in U.S. Pat. No. 9,745,368, both incorporated by reference as if fully disclosed herein.

According to one strategy, the two CARs in every given pair specifically recognize the product of a different allelic variant of the same target gene for which the patient is heterozygous. The basic principle is as follows: the aCAR targets an allelic variant of a selected cell surface protein that is expressed by the given tumor cells and is not affected by LOH while the iCAR targets the product encoded by the allelic variant of the same gene that has been lost from these tumor cells due to LOH. In other normal tissues of that individual patient that express the said gene, both alleles are present and are known to be equally functional, that is, expression is biallelic in all tissues (in contrast to other genes which may exhibit random monoallelic expression (Chess, 2012; Savova et al., 2016). In one scenario, the two CARs target two related epitopes residing at the same location on the protein product, which differ by one, or only few amino acids. In another scenario, the aCAR targets a non-polymorphic epitope on the same protein while the iCAR is allele-specific. In these embodiments, the density of the aCAR epitope on normal cells would generally be two-fold higher than that of the iCAR one. In some embodiments, a single nucleic acid vector encodes both the aCAR and iCAR, as exemplified with the bicistronic constructs described herein. In some embodiments, the aCAR and iCAR are encoded by separate nucleic acid vectors and co-expressed.

Care must be taken to ensure that the inhibitory signal transmitted by the iCAR is dominant over the aCAR signal and that cross-recognition between the iCAR and the aCAR is limited and/or negligible. Dominance of the iCAR guarantees that activation of the killer cell upon encounter with normal cells expressing both alleles would be prevented. This default brake would not operate upon engagement with tumor cells: in the absence of its target antigen the iCAR would not deliver inhibitory signals, thus unleashing the anticipated aCAR-mediated cellular activation and subsequent tumor cell lysis. Dominance of the iCARs over their aCARs counterparts is a significant portion of how the system functions. The present invention provides novel bicistronic iCAR/aCAR constructs that function in this manner, as well as methods for co-transduction of monocistronic aCAR and iCAR constructs.

The bicistronic constructs of the present invention comprise the following components: an iCAR and aCAR connected via a linker domain. In some embodiments, the iCAR (protective) portion comprises an iCAR scFv, a hinge transmembrane (TM) domain, and inhibitory domain. In some embodiments, the aCAR (efficacy) portion comprises an aCAR scFv, a hinge transmembrane (TM) domain, a co-stimulatory domain, and a CD3 zeta domain.

I. Bicistronic Sequences

In some embodiments, the bicistronic iCAR/aCAR comprises an amino acid sequence encoded by a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325, as provided in Table 1 below. In some embodiments, the bicistronic iCAR/aCAR comprise an amino acid sequence encoded by a nucleic acid sequence comprising SEQ ID NO:1. In some embodiments, the bicistronic iCAR/aCAR comprise an amino acid sequence encoded by a nucleic acid sequence comprising SEQ ID NO:3. In some embodiments, the bicistronic iCAR/aCAR comprise an amino acid sequence encoded by a nucleic acid sequence comprising SEQ ID NO:5. In some embodiments, the bicistronic iCAR/aCAR comprise an amino acid sequence encoded by a nucleic acid sequence comprising SEQ ID NO:7. In some embodiments, the bicistronic iCAR/aCAR comprise an amino acid sequence encoded by a nucleic acid sequence comprising SEQ ID NO:9. In some embodiments, the bicistronic iCAR/aCAR comprise an amino acid sequence encoded by a nucleic acid sequence comprising SEQ ID NO:11. In some embodiments, the bicistronic iCAR/aCAR comprise an amino acid sequence encoded by a nucleic acid sequence comprising SEQ ID NO:13. In some embodiments, the bicistronic iCAR/aCAR comprise an amino acid sequence encoded by a nucleic acid sequence comprising SEQ ID NO:15. In some embodiments, the bicistronic iCAR/aCAR comprise an amino acid sequence encoded by a nucleic acid sequence comprising SEQ ID NO:17. In some embodiments, the bicistronic iCAR/aCAR comprise an amino acid sequence encoded by a nucleic acid sequence comprising SEQ ID NO:19. In some embodiments, the bicistronic iCAR/aCAR comprise an amino acid sequence encoded by a nucleic acid sequence comprising SEQ ID NO:21. In some embodiments, the bicistronic iCAR/aCAR comprise an amino acid sequence encoded by a nucleic acid sequence comprising SEQ ID NO:23. In some embodiments, the bicistronic iCAR/aCAR comprise an amino acid sequence encoded by a nucleic acid sequence comprising SEQ ID NO:25. In some embodiments, the bicistronic iCAR/aCAR comprise an amino acid sequence encoded by a nucleic acid sequence comprising SEQ ID NO:27. In some embodiments, the bicistronic iCAR/aCAR comprise an amino acid sequence encoded by a nucleic acid sequence comprising SEQ ID NO:29. In some embodiments, the bicistronic iCAR/aCAR comprise an amino acid sequence encoded by a nucleic acid sequence comprising SEQ ID NO:31. In some embodiments, the bicistronic iCAR/aCAR comprise an amino acid sequence encoded by a nucleic acid sequence comprising SEQ ID NO:33. In some embodiments, the bicistronic iCAR/aCAR comprise an amino acid sequence encoded by a nucleic acid sequence comprising SEQ ID NO:35. In some embodiments, the bicistronic iCAR/aCAR comprise an amino acid sequence encoded by a nucleic acid sequence comprising SEQ ID NO:275. In some embodiments, the bicistronic iCAR/aCAR comprise an amino acid sequence encoded by a nucleic acid sequence comprising SEQ ID NO:277. In some embodiments, the bicistronic iCAR/aCAR comprise an amino acid sequence encoded by a nucleic acid sequence comprising SEQ ID NO:279. In some embodiments, the bicistronic iCAR/aCAR comprise an amino acid sequence encoded by a nucleic acid sequence comprising SEQ ID NO:281. In some embodiments, the bicistronic iCAR/aCAR comprise an amino acid sequence encoded by a nucleic acid sequence comprising SEQ ID NO:321. In some embodiments, the bicistronic iCAR/aCAR comprise an amino acid sequence encoded by a nucleic acid sequence comprising SEQ ID NO:323. In some embodiments, the bicistronic iCAR/aCAR comprise an amino acid sequence encoded by a nucleic acid sequence comprising SEQ ID NO:325.

In some embodiments, the bicistronic iCAR/aCAR comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326 as provided in Table 1.

below. In some embodiments, the bicistronic iCAR/aCAR comprises SEQ ID NO:2. In some embodiments, the bicistronic iCAR/aCAR comprises SEQ ID NO:4. In some embodiments, the bicistronic iCAR/aCAR comprises SEQ ID NO:6. In some embodiments, the bicistronic iCAR/aCAR comprises SEQ ID NO:8. In some embodiments, the bicistronic iCAR/aCAR comprises SEQ ID NO:10. In some embodiments, the bicistronic iCAR/aCAR comprises SEQ ID NO:12. In some embodiments, the bicistronic iCAR/aCAR comprises SEQ ID NO:14. In some embodiments, the bicistronic iCAR/aCAR comprises SEQ ID NO:16. In some embodiments, the bicistronic iCAR/aCAR comprises SEQ ID NO:18. In some embodiments, the bicistronic iCAR/aCAR comprises SEQ ID NO:20. In some embodiments, the bicistronic iCAR/aCAR comprises SEQ ID NO:22. In some embodiments, the bicistronic iCAR/aCAR comprises SEQ ID NO:24. In some embodiments, the bicistronic iCAR/aCAR comprises SEQ ID NO:26. In some embodiments, the bicistronic iCAR/aCAR comprises SEQ ID NO:28. In some embodiments, the bicistronic iCAR/aCAR comprises SEQ ID NO:30. In some embodiments, the bicistronic iCAR/aCAR comprises SEQ ID NO:32, SEQ ID NO:34. In some embodiments, the bicistronic iCAR/aCAR comprises SEQ ID NO:36. In some embodiments, the bicistronic iCAR/aCAR comprises SEQ ID NO:276. In some embodiments, the bicistronic iCAR/aCAR comprises SEQ ID NO:278. In some embodiments, the bicistronic iCAR/aCAR comprises SEQ ID NO:280. In some embodiments, the bicistronic iCAR/aCAR comprises SEQ ID NO:282. In some embodiments, the bicistronic iCAR/aCAR comprises SEQ ID NO:322. In some embodiments, the bicistronic iCAR/aCAR comprises SEQ ID NO:324. In some embodiments, the bicistronic iCAR/aCAR comprises SEQ ID NO:326.

TABLE 1
Bicistonic iCAR/aCARs: nucleic acid and amino acid sequences

| Sequence | SEQ ID | |
|---|---|---|
| name | NO: | Polynucleotide or polypeptide sequences |
| MC0280- | SEQ ID | ATGGCACTGCCAGTGACCGCCCTGCTGCTGCCTCTGGCCCT |
| BB7.2_28_ | NO: 1 | GCTGCTGCACGCAGCCAGACCCCAGGTGCAGCTGCAGCAGT |
| PD1_HER2 | | CTGGACCTGAGCTGGTGAAGCCAGGAGCCTCCGTGAAGATG |
| Nucleotide | | TCTTGCAAGGCCAGCGGCTACACCTTCACATCTTATCACATC |
| sequence | | CAGTGGGTGAAGCAGCGGCCCGGACAGGGCCTGGAGTGGA |
| (VR280) | | TCGGATGGATCTACCCAGGCGACGGCTCCACACAGTATAAC |
| | | GAGAAGTTCAAGGGCAAGACCACACTGACCGCCGATAAGA |
| | | GCAGCAGCACCGCCTACATGCTGCTGAGCAGCCTGACCAGC |
| | | GAGGACAGCGCCATCTACTTTTGCGCCAGGGAGGGCACATA |
| | | CTATGCTATGGACTATTGGGGCCAGGGCACCAGCGTGACAG |
| | | TGTCTAGCGGAGGAGGAGGCTCCGGAGGAGGAGGCTCTGG |
| | | CGGCGGCGGCAGCGACGTGCTGATGACCCAGACACCACTG |
| | | AGCCTGCCCGTGAGCCTGGGCGATCAGGTGAGCATCTCCTG |
| | | TAGATCCTCTCAGAGCATCGTGCACTCCAACGGCAATACCT |
| | | ACCTGGAGTGGTATCTGCAGAAGCCAGGCCAGTCCCCCAAG |
| | | CTGCTGATCTATAAGGTGTCTAATCGGTTCAGCGGCGTGCCT |
| | | GACAGATTTTCTGGCAGCGGCTCCGGCACCGACTTCACCCT |
| | | GAAGATCAGCCGGGTGGAGGCAGAGGATCTGGGCGTGTAC |
| | | TATTGTTTCCAGGGCTCCCACGTGCCACGCACCTTTGGCGGC |
| | | GGTACCAAGCTGGAGATCAAGATTGAAGTTATGTATCCTCC |
| | | TCCTTACCTAGACAATGAGAAGAGCAATGGAACCATTATCC |
| | | ATGTGAAAGGGAAACACCTTTGTCCAAGTCCCCTATTTCCC |
| | | GGGCCTTCGAAGCCCTTTTGGGTGCTGGTGGTGGTTGGTGG |
| | | AGTCCTGGCTTGCTATAGCTTGCTAGTAACAGTAGCGTTTAT |
| | | TATTTTCTGGGTGTGCAGCAGGGCCGCCCGCGGCACCATCG |
| | | GCGCCAGGCGCACAGGCCAGCCTCTGAAGGAGGACCCTTCC |
| | | GCCGTGCCAGTGTTCTCTGTGGACTACGGCGAGCTGGATTTT |
| | | CAGTGGCGGGAGAAAACCCCAGAGCCACCTGTGCCCTGCGT |
| | | GCCTGAGCAGACCGAGTATGCCACAATCGTGTTTCCATCCG |
| | | GAATGGGCACAAGCTCCCCTGCAAGGAGAGGCAGCGCCGA |
| | | CGGACCACGGTCCGCCCAGCCACTGCGGCCCGAGGATGGCC |
| | | ACTGTTCTTGGCCCCTGCGGAGAAAGCGTGGATCCGGGGAA |
| | | GGCCGAGGCTCCCTTCTAACATGTGGAGATGTCGAGGAAAA |
| | | CCCTGGCCCTATGGCGCTGCCAGTCACTGCATTGTTATTGCC |
| | | TCTGGCCCTGCTTCTCCATGCGGCGCGCCCAGAAGTGCAGC |
| | | TGGTCGAGAGCGGAGGCGGACTGGTTCAACCCGGAGGCAG |
| | | CTTGAGACTGTCCTGCGCGGCCAGCGGCTTCAACATCAAGG |
| | | ATACCTATATCCACTGGGTGAGGCAGGCTCCAGGAAAGGGC |
| | | CTGGAGTGGGTGGCAAGGATTTACCCTACTAATGGATATAC |
| | | ACGCTACGCTGATTCCGTGAAGGGACGCTTTACAATCTCAG |
| | | CAGATACATCCAAAAACACGGCCTATTTACAGATGAATAGT |
| | | TTGCGGGCCGAAGACACGGCTGTATACTATTGTTCTCGGTG |
| | | GGGGGGCGATGGATTTTATGCGATGGATTACTGGGGCCAGG |
| | | GCACCCTGGTAACCGTGTCAAGCGGCTCAACATCCGGGTCC |
| | | GGTAAGCCGGGCTCCGGCGAGGGGTCTACAAAGGGAGATA |
| | | TACAGATGACACAGTCCCCCAGTTCCCTGTCCGCCTCAGTG |
| | | GGAGACCGAGTGACGATTACCTGTCGTGCCAGCCAGGACGT |
| | | CAATACCGCCGTCGCTTGGTATCAGCAAAAACCAGGCAAGG |
| | | CCCCGAAACTATTGATCTACAGTGCCTCTTTTCTGTACTCCG |
| | | GGGTGCCGAGCAGATTTAGTGGCTCCAGGAGCGGAACCGAT |
| | | TTCACCCTAACCATTTCCAGTTTGCAGCCAGAGGATTTCGCG |
| | | ACCTATTACTGCCAGCAACACTACACCACACCGCCAACTTT |
| | | CGGACAAGGAACCAAGGTTGAAATCAAAACTACGACCCCA |
| | | GCACCTAGACCTCCCACCCCAGCTCCAACTATAGCTTCCCA |
| | | GCCATTGTCTCTCCGGCCAGAGGCGTGTCGACCAGCCGCTG |
| | | GAGGGGCCGTTCATACAAGAGGACTCGATTTCGCTTGCGAT |
| | | ATCTACATATGGGCCCCTCTTGCCGGGACATGCGGTGTCCT |
| | | GCTTCTAAGCTTGGTTATTACCCTCTATTGCAAACGCGGCCG |
| | | CAAGAAACTGCTCTACATCTTTAAACAGCCGTTCATGAGGC |
| | | CTGTGCAGACAACGCAGGAAGAGGATGGCTGTAGTTGTCGG |
| | | TTTCCGGAAGAGGAAGAGGGGGGCTGCGAGTTGCGTGTCA |
| | | AATTTTCTCGGTCTGCCGACGCCCCCGCGTACCAGCAAGGG |
| | | CAGAACCAGCTTTATAATGAGCTGAATCTTGGACGACGGGA |
| | | GGAATATGACGTGCTTGACAAGAGGCGAGGTAGGGACCCT |
| | | GAGATGGGGGGAAAACCTCGGAGGAAAAACCCACAGGAAG |
| | | GCCTGTATAACGAACTGCAGAAGGACAAGATGGCTGAAGC |
| | | CTACTCTGAGATTGGAATGAAAGGGGAACGCAGACGCGGC |
| | | AAGGGCCATGATGGCCTCTACCAAGGTCTAAGCACTGCCAC |
| | | CAAGGACACCTATGACGCACTCCACATGCAAGCTCTACCTC |
| | | CCCGTTGATAA |
| MC0280- | SEQ ID | MALPVTALLLPLALLLHAARPQVQLQQSGPELVKPGASVKMS |
| BB7.2_28_ | NO: 2 | CKASGYTFTSYHIQWVKQRPGQGLEWIGWIYPGDGSTQYNEK |
| PD1_HER2 | | FKGKTTLTADKSSSTAYMLLSSLTSEDSAIYFCAREGTYYAMD |
| Protein | | YWGQGTSVTVSSGGGGSGGGGSGGGGSDVLMTQTPLSLPVSL |
| sequence | | GDQVSISCRSSQSIVHSNGNTYLEWYLQKPGQSPKLLIYKVSNR |
| (VR280) | | FSGVPDRESGSGSGTDFTLKISRVEAEDLGVYYCFQGSHVPRTF |
| | | GGGTKLEIKIEVMYPPPYLDNEKSNGTIIHVKGKHLCPSPLFPG |
| | | PSKPFWVLVVVGGVLACYSLLVTVAFIIFWVCSRAARGTIGAR |
| | | RTGQPLKEDPSAVPVFSVDYGELDFQWREKTPEPPVPCVPEQT |
| | | EYATIVFPSGMGTSSPARRGSADGPRSAQPLRPEDGHCSWPLR |
| | | RKRGSGEGRGSLLTCGDVEENPGPMALPVTALLLPLALLLHAA |
| | | RPEVQLVESGGGLVQPGGSLRLSCAASGFNIKDTYIHWVRQAP |
| | | GKGLEWVARIYPTNGYTRYADSVKGRFTISADTSKNTAYLQM |
| | | NSLRAEDTAVYYCSRWGGDGFYAMDYWGQGTLVTVSSGSTS |
| | | GSGKPGSGEGSTKGDIQMTQSPSSLSASVGDRVTITCRASQDV |
| | | NTAVAWYQQKPGKAPKLLIYSASFLYSGVPSRFSGSRSGTDFT |
| | | LTISSLQPEDFATYYCQQHYTTPPTFGQGTKVEIKTTTPAPRPPT |
| | | PAPTIASQPLSLRPEACRPAAGGAVHTRGLDFACDIYIWAPLAG |
| | | TCGVLLLSLVITLYCKRGRKKLLYIFKQPFMRPVQTTQEEDGC |
| | | SCRFPEEEEGGCELRVKESRSADAPAYQQGQNQLYNELNLGR |
| | | REEYDVLDKRRGRDPEMGGKPRRKNPQEGLYNELQKDKMAE |
| | | AYSEIGMKGERRRGKGHDGLYQGLSTATKDTYDALHMQALP |
| | | PR |
| MC0281- | SEQ ID | ATGGCACTGCCAGTGACCGCCCTGCTGCTGCCTCTGGCCCT |
| BB7.2_28_ | NO: 3 | GCTGCTGCACGCAGCCAGACCCCAGGTGCAGCTGCAGCAGT |
| PD1_EGFR | | CTGGACCTGAGCTGGTGAAGCCAGGAGCCTCCGTGAAGATG |
| nucleotide | | TCTTGCAAGGCCAGCGGCTACACCTTCACATCTTATCACATC |
| Sequence | | CAGTGGGTGAAGCAGCGGCCCGGACAGGGCCTGGAGTGGA |
| (VR281) | | TCGGATGGATCTACCCAGGCGACGGCTCCACACAGTATAAC |
| | | GAGAAGTTCAAGGGCAAGACCACACTGACCGCCGATAAGA |
| | | GCAGCAGCACCGCCTACATGCTGCTGAGCAGCCTGACCAGC |
| | | GAGGACAGCGCCATCTACTTTTGCGCCAGGGAGGGCACATA |
| | | CTATGCTATGGACTATTGGGGCCAGGGCACCAGCGTGACAG |
| | | TGTCTAGCGGAGGAGGAGGCTCCGGAGGAGGAGGCTCTGG |
| | | CGGCGGCGGCAGCGACGTGCTGATGACCCAGACACCACTG |
| | | AGCCTGCCCGTGAGCCTGGGCGATCAGGTGAGCATCTCCTG |
| | | TAGATCCTCTCAGAGCATCGTGCACTCCAACGGCAATACCT |
| | | ACCTGGAGTGGTATCTGCAGAAGCCAGGCCAGTCCCCCAAG |
| | | CTGCTGATCTATAAGGTGTCTAATCGGTTCAGCGGCGTGCCT |
| | | GACAGATTTTCTGGCAGCGGCTCCGGCACCGACTTCACCCT |
| | | GAAGATCAGCCGGGTGGAGGCAGAGGATCTGGGCGTGTAC |
| | | TATTGTTTCCAGGGCTCCCACGTGCCACGCACCTTTGGCGGC |
| | | GGTACCAAGCTGGAGATCAAGATTGAAGTTATGTATCCTCC |
| | | TCCTTACCTAGACAATGAGAAGAGCAATGGAACCATTATCC |
| | | ATGTGAAAGGGAAACACCTTTGTCCAAGTCCCCTATTTCCC |
| | | GGGCCTTCGAAGCCCTTTTGGGTGCTGGTGGTGGTTGGTGG |
| | | AGTCCTGGCTTGCTATAGCTTGCTAGTAACAGTAGCGTTTAT |
| | | TATTTTCTGGGTGTGCAGCAGGGCCGCCCGCGGCACCATCG |
| | | GCGCCAGGCGCACAGGCCAGCCTCTGAAGGAGGACCCTTCC |
| | | GCCGTGCCAGTGTTCTCTGTGGACTACGGCGAGCTGGATTTT |
| | | CAGTGGCGGGAGAAAACCCCAGAGCCACCTGTGCCCTGCGT |
| | | GCCTGAGCAGACCGAGTATGCCACAATCGTGTTTCCATCCG |
| | | GAATGGGCACAAGCTCCCCTGCAAGGAGAGGCAGCGCCGA |
| | | CGGACCACGGTCCGCCCAGCCACTGCGGCCCGAGGATGGCC |
| | | ACTGTTCTTGGCCCCTGCGGAGAAAGCGTGGATCCGGGGAA |
| | | GGCCGAGGCTCCCTTCTAACATGTGGAGATGTCGAGGAAAA |
| | | CCCTGGCCCTATGGCGCTGCCAGTCACTGCATTGTTATTGCC |
| | | TCTGGCCCTGCTTCTCCATGCGGCGCGCCCACAAGTGCAGC |
| | | TGAAACAGAGCGGACCAGGACTGGTTCAACCCAGCCAGAG |
| | | CTTGAGCATCACGTGCACGGTTAGCGGCTTCAGTCTGACCA |
| | | ATTATGGTGTGCACTGGGTGAGGCAGTCTCCAGGAAAGGGC |
| | | CTGGAGTGGCTTGGAGTCATTTGGAGCGGTGGGAATACAGA |
| | | TTACAATACACCTTTTACGTCACGTCTCTCCATTAACAAGGA |
| | | CAACTCCAAATCCCAAGTATTTTTCAAAATGAATAGCCTGC |
| | | AGAGTAATGATACCGCCATCTATTACTGTGCACGAGCTTTG |
| | | ACATATTACGACTATGAATTTGCCTATTGGGGTCAAGGCAC |
| | | GCTGGTGACCGTATCAGGCTCAACATCCGGGTCCGGTAAGC |
| | | CGGGCTCCGGCGAGGGGTCTACAAAGGGAGACATCCTTCTG |
| | | ACACAGAGCCCCGTGATCCTGTCCGTGTCCCCCGGCGAGAG |
| | | AGTATCATTTTCCTGTAGGGCTTCTCAGAGCATCGGAACAA |
| | | ATATCCACTGGTATCAGCAACGGACTAACGGATCACCTCGC |
| | | CTGCTCATAAAGTACGCCAGTGAATCTATTAGTGGCATACC |
| | | GAGCCGCTTCAGCGGGAGTGGCTCCGGCACAGACTTTACTC |
| | | TGAGTATAAATTCCGTGGAATCTGAGGACATCGCGGACTAT |
| | | TACTGCCAGCAAAACAATAACTGGCCCACCACGTTCGGCGC |
| | | GGGAACTAAACTAGAACTAAAGACTACGACCCCAGCACCT |
| | | AGACCTCCCACCCCAGCTCCAACTATAGCTTCCCAGCCATT |
| | | GTCTCTCCGGCCAGAGGCGTGTCGACCAGCCGCTGGAGGGG |
| | | CCGTTCATACAAGAGGACTCGATTTCGCTTGCGATATCTAC |
| | | ATATGGGCCCCTCTTGCCGGGACATGCGGTGTCCTGCTTCTA |
| | | AGCTTGGTTATTACCCTCTATTGCAAACGCGGCCGCAAGAA |
| | | ACTGCTCTACATCTTTAAACAGCCGTTCATGAGGCCTGTGCA |
| | | GACAACGCAGGAAGAGGATGGCTGTAGTTGTCGGTTTCCGG |
| | | AAGAGGAAGAGGGGGGCTGCGAGTTGCGTGTCAAATTTTCT |
| | | CGGTCTGCCGACGCCCCCGCGTACCAGCAAGGGCAGAACCA |
| | | GCTTTATAATGAGCTGAATCTTGGACGACGGGAGGAATATG |
| | | ACGTGCTTGACAAGAGGCGAGGTAGGGACCCTGAGATGGG |
| | | GGGAAAACCTCGGAGGAAAAACCCACAGGAAGGCCTGTAT |
| | | AACGAACTGCAGAAGGACAAGATGGCTGAAGCCTACTCTG |
| | | AGATTGGAATGAAAGGGGAACGCAGACGCGGCAAGGGCCA |
| | | TGATGGCCTCTACCAAGGTCTAAGCACTGCCACCAAGGACA |
| | | CCTATGACGCACTCCACATGCAAGCTCTACCTCCCCGTTGAT |
| | | AA |
| MC0281- | SEQ ID | MALPVTALLLPLALLLHAARPQVQLQQSGPELVKPGASVKMS |
| BB7.2_28_ | NO: 4 | CKASGYTFTSYHIQWVKQRPGQGLEWIGWIYPGDGSTQYNEK |
| PD1_EGFR | | FKGKTTLTADKSSSTAYMLLSSLTSEDSAIYFCAREGTYYAMD |
| Protein | | YWGQGTSVTVSSGGGGSGGGGSGGGGSDVLMTQTPLSLPVSL |
| Sequence | | GDQVSISCRSSQSIVHSNGNTYLEWYLQKPGQSPKLLIYKVSNR |
| (VR281) | | FSGVPDRFSGSGSGTDFTLKISRVEAEDLGVYYCFQGSHVPRTF |
| | | GGGTKLEIKIEVMYPPPYLDNEKSNGTIIHVKGKHLCPSPLFPG |
| | | PSKPFWVLVVVGGVLACYSLLVTVAFIIFWVCSRAARGTIGAR |
| | | RTGQPLKEDPSAVPVFSVDYGELDFQWREKTPEPPVPCVPEQT |
| | | EYATIVFPSGMGTSSPARRGSADGPRSAQPLRPEDGHCSWPLR |
| | | RKRGSGEGRGSLLTCGDVEENPGPMALPVTALLLPLALLLHAA |
| | | RPQVQLKQSGPGLVQPSQSLSITCTVSGFSLTNYGVHWVRQSP |
| | | GKGLEWLGVIWSGGNTDYNTPFTSRLSINKDNSKSQVFFKMN |
| | | SLQSNDTAIYYCARALTYYDYEFAYWGQGTLVTVSGSTSGSG |
| | | KPGSGEGSTKGDILLTQSPVILSVSPGERVSFSCRASQSIGTNIH |
| | | WYQQRTNGSPRLLIKYASESISGIPSRFSGSGSGTDFTLSINSVES |
| | | EDIADYYCQQNNNWPTTFGAGTKLELKTTTPAPRPPTPAPTIAS |
| | | QPLSLRPEACRPAAGGAVHTRGLDFACDIYIWAPLAGTCGVLL |
| | | LSLVITLYCKRGRKKLLYIFKQPFMRPVQTTQEEDGCSCRFPEE |
| | | EEGGCELRVKFSRSADAPAYQQGQNQLYNELNLGRREEYDVL |
| | | DKRRGRDPEMGGKPRRKNPQEGLYNELQKDKMAEAYSEIGM |
| | | KGERRRGKGHDGLYQGLSTATKDTYDALHMQALPPR |
| MC0282- | SEQ ID | ATGGCACTGCCAGTGACCGCCCTGCTGCTGCCTCTGGCCCT |
| 3PF12_28_ | NO: 5 | GCTGCTGCACGCAGCCAGACCCCAAGTGCAACTAGTCCAAT |
| PD1_HER2 | | CAGGTGGAGGCGTCGTGCAACCTGGAGGGTCCCTCCGCGTT |
| Nucleotide | | AGCTGCGCCGCATCAGGCGTTACCTTGTCAGACTACGGCAT |
| Sequence | | GCATTGGGTTAGGCAAGCCCCCGGCAAGGGGCTCGAATGG |
| (VR282) | | ATGGCTTTCATTCGGAATGACGGGAGCGATAAATATTACGC |
| | | GGATTCAGTTAAAGGGCGGTTCACCATCAGCCGCGACAATA |
| | | GCAAAAAGACGGTCTCCTTACAGATGTCCAGCTTGCGGGCC |
| | | GAAGACACGGCTGTATACTATTGTGCTAAAAATGGCGAGAG |
| | | CGGCCCCCTGGATTACTGGTACTTTGACCTGTGGGGCAGAG |
| | | GCACCCTGGTCACGGTGTCCTCTGGAGGAGGAGGCTCCGGA |
| | | GGAGGAGGCTCTGGCGGCGGCGGCAGCGACATTGTAATGA |
| | | CCCAGTCACCCTCCTTCCTTAGTGCCTCAGTCGGAGACCGCG |
| | | TGACTATCACTTGTCGTGCCTCACACGGAATTAATAACTACC |
| | | TCGCTTGGTATCAGCAAAAACCAGGCAAGGCCCCGAAACTA |
| | | TTGATCTACGCCGCATCTACTCTGCAGAGCGGAGTACCGAG |
| | | CAGATTTAGTGGTTCCGGCAGCGGAACCGAGTTCACCCTAA |
| | | CCATTTCCAGTTTGCAGCCAGAGGATTTCGCGACCTATTACT |
| | | GCCAGCAATACGATTCATACCCGCCAACTTTCGGAAGAGGT |
| | | ACCAAGGTTGAAATCAAGATTGAAGTTATGTATCCTCCTCC |
| | | TTACCTAGACAATGAGAAGAGCAATGGAACCATTATCCATG |
| | | TGAAAGGGAAACACCTTTGTCCAAGTCCCCTATTTCCCGGG |
| | | CCTTCGAAGCCCTTTTGGGTGCTGGTGGTGGTTGGTGGAGTC |
| | | CTGGCTTGCTATAGCTTGCTAGTAACAGTAGCGTTTATTATT |
| | | TTCTGGGTGTGCAGCAGGGCCGCCCGCGGCACCATCGGCGC |
| | | CAGGCGCACAGGCCAGCCTCTGAAGGAGGACCCTTCCGCCG |
| | | TGCCAGTGTTCTCTGTGGACTACGGCGAGCTGGATTTTCAGT |
| | | GGCGGGAGAAAACCCCAGAGCCACCTGTGCCCTGCGTGCCT |
| | | GAGCAGACCGAGTATGCCACAATCGTGTTTCCATCCGGAAT |
| | | GGGCACAAGCTCCCCTGCAAGGAGAGGCAGCGCCGACGGA |
| | | CCACGGTCCGCCCAGCCACTGCGGCCCGAGGATGGCCACTG |
| | | TTCTTGGCCCCTGCGGAGAAAGCGTGGATCCGGGGAAGGCC |
| | | GAGGCTCCCTTCTAACATGTGGAGATGTCGAGGAAAACCCT |
| | | GGCCCTATGGCGCTGCCAGTCACTGCATTGTTATTGCCTCTG |
| | | GCCCTGCTTCTCCATGCGGCGCGCCCAGAAGTGCAGCTGGT |
| | | CGAGAGCGGAGGCGGACTGGTTCAACCCGGAGGCAGCTTG |
| | | AGACTGTCCTGCGCGGCCAGCGGCTTCAACATCAAGGATAC |
| | | CTATATCCACTGGGTGAGGCAGGCTCCAGGAAAGGGCCTGG |
| | | AGTGGGTGGCAAGGATTTACCCTACTAATGGATATACACGC |
| | | TACGCTGATTCCGTGAAGGGACGCTTTACAATCTCAGCAGA |
| | | TACATCCAAAAACACGGCCTATTTACAGATGAATAGTTTGC |
| | | GGGCCGAAGACACGGCTGTATACTATTGTTCTCGGTGGGGG |
| | | GGCGATGGATTTTATGCGATGGATTACTGGGGCCAGGGCAC |
| | | CCTGGTAACCGTGTCAAGCGGCTCAACATCCGGGTCCGGTA |
| | | AGCCGGGCTCCGGCGAGGGGTCTACAAAGGGAGATATACA |
| | | GATGACACAGTCCCCCAGTTCCCTGTCCGCCTCAGTGGGAG |
| | | ACCGAGTGACGATTACCTGTCGTGCCAGCCAGGACGTCAAT |
| | | ACCGCCGTCGCTTGGTATCAGCAAAAACCAGGCAAGGCCCC |
| | | GAAACTATTGATCTACAGTGCCTCTTTTCTGTACTCCGGGGT |
| | | GCCGAGCAGATTTAGTGGCTCCAGGAGCGGAACCGATTTCA |
| | | CCCTAACCATTTCCAGTTTGCAGCCAGAGGATTTCGCGACCT |
| | | ATTACTGCCAGCAACACTACACCACACCGCCAACTTTCGGA |
| | | CAAGGAACCAAGGTTGAAATCAAAACTACGACCCCAGCAC |
| | | CTAGACCTCCCACCCCAGCTCCAACTATAGCTTCCCAGCCAT |
| | | TGTCTCTCCGGCCAGAGGCGTGTCGACCAGCCGCTGGAGGG |
| | | GCCGTTCATACAAGAGGACTCGATTTCGCTTGCGATATCTA |
| | | CATATGGGCCCCTCTTGCCGGGACATGCGGTGTCCTGCTTCT |
| | | AAGCTTGGTTATTACCCTCTATTGCAAACGCGGCCGCAAGA |
| | | AACTGCTCTACATCTTTAAACAGCCGTTCATGAGGCCTGTGC |
| | | AGACAACGCAGGAAGAGGATGGCTGTAGTTGTCGGTTTCCG |
| | | GAAGAGGAAGAGGGGGGCTGCGAGTTGCGTGTCAAATTTTC |
| | | TCGGTCTGCCGACGCCCCCGCGTACCAGCAAGGGCAGAACC |
| | | AGCTTTATAATGAGCTGAATCTTGGACGACGGGAGGAATAT |
| | | GACGTGCTTGACAAGAGGCGAGGTAGGGACCCTGAGATGG |
| | | GGGGAAAACCTCGGAGGAAAAACCCACAGGAAGGCCTGTA |
| | | TAACGAACTGCAGAAGGACAAGATGGCTGAAGCCTACTCTG |
| | | AGATTGGAATGAAAGGGGAACGCAGACGCGGCAAGGGCCA |
| | | TGATGGCCTCTACCAAGGTCTAAGCACTGCCACCAAGGACA |
| | | CCTATGACGCACTCCACATGCAAGCTCTACCTCCCCGTTGAT |
| | | AA |
| MC0282- | SEQ ID | MALPVTALLLPLALLLHAARPQVQLVQSGGGVVQPGGSLRVS |
| 3PF12_28_ | NO: 6 | CAASGVTLSDYGMHWVRQAPGKGLEWMAFIRNDGSDKYYA |
| PD1_HER2 | | DSVKGRFTISRDNSKKTVSLQMSSLRAEDTAVYYCAKNGESGP |
| Protein | | LDYWYFDLWGRGTLVTVSSGGGGSGGGGSGGGGSDIVMTQS |
| Sequence | | PSFLSASVGDRVTITCRASHGINNYLAWYQQKPGKAPKLLIYA |
| (VR282) | | ASTLQSGVPSRESGSGSGTEFTLTISSLQPEDFATYYCQQYDSY |
| | | PPTFGRGTKVEIKIEVMYPPPYLDNEKSNGTIIHVKGKHLCPSPL |
| | | FPGPSKPFWVLVVVGGVLACYSLLVTVAFIIFWVCSRAARGTI |
| | | GARRTGQPLKEDPSAVPVFSVDYGELDFQWREKTPEPPVPCVP |
| | | EQTEYATIVFPSGMGTSSPARRGSADGPRSAQPLRPEDGHCSW |
| | | PLRRKRGSGEGRGSLLTCGDVEENPGPMALPVTALLLPLALLL |
| | | HAARPEVQLVESGGGLVQPGGSLRLSCAASGFNIKDTYIHWVR |
| | | QAPGKGLEWVARIYPTNGYTRYADSVKGRFTISADTSKNTAY |
| | | LQMNSLRAEDTAVYYCSRWGGDGFYAMDYWGQGTLVTVSS |
| | | GSTSGSGKPGSGEGSTKGDIQMTQSPSSLSASVGDRVTITCRAS |
| | | QDVNTAVAWYQQKPGKAPKLLIYSASFLYSGVPSRFSGSRSGT |
| | | DFTLTISSLQPEDFATYYCQQHYTTPPTFGQGTKVEIKTTTPAP |
| | | RPPTPAPTIASQPLSLRPEACRPAAGGAVHTRGLDFACDIYIWA |
| | | PLAGTCGVLLLSLVITLYCKRGRKKLLYIFKQPFMRPVQTTQEE |
| | | DGCSCRFPEEEEGGCELRVKFSRSADAPAYQQGQNQLYNELN |
| | | LGRREEYDVLDKRRGRDPEMGGKPRRKNPQEGLYNELQKDK |
| | | MAEAYSEIGMKGERRRGKGHDGLYQGLSTATKDTYDALHMQ |
| | | ALPPR |
| MC0283- | SEQ ID | ATGGCACTGCCAGTGACCGCCCTGCTGCTGCCTCTGGCCCT |
| 3PF12_28_ | NO: 7 | GCTGCTGCACGCAGCCAGACCCCAAGTGCAACTAGTCCAAT |
| PD1_EGFR | | CAGGTGGAGGCGTCGTGCAACCTGGAGGGTCCCTCCGCGTT |
| Nucleotide | | AGCTGCGCCGCATCAGGCGTTACCTTGTCAGACTACGGCAT |
| Sequence | | GCATTGGGTTAGGCAAGCCCCCGGCAAGGGGCTCGAATGG |
| (VR283) | | ATGGCTTTCATTCGGAATGACGGGAGCGATAAATATTACGC |
| | | GGATTCAGTTAAAGGGCGGTTCACCATCAGCCGCGACAATA |
| | | GCAAAAAGACGGTCTCCTTACAGATGTCCAGCTTGCGGGCC |
| | | GAAGACACGGCTGTATACTATTGTGCTAAAAATGGCGAGAG |
| | | CGGCCCCCTGGATTACTGGTACTTTGACCTGTGGGGCAGAG |
| | | GCACCCTGGTCACGGTGTCCTCTGGAGGAGGAGGCTCCGGA |
| | | GGAGGAGGCTCTGGCGGCGGCGGCAGCGACATTGTAATGA |
| | | CCCAGTCACCCTCCTTCCTTAGTGCCTCAGTCGGAGACCGCG |
| | | TGACTATCACTTGTCGTGCCTCACACGGAATTAATAACTACC |
| | | TCGCTTGGTATCAGCAAAAACCAGGCAAGGCCCCGAAACTA |
| | | TTGATCTACGCCGCATCTACTCTGCAGAGCGGAGTACCGAG |
| | | CAGATTTAGTGGTTCCGGCAGCGGAACCGAGTTCACCCTAA |
| | | CCATTTCCAGTTTGCAGCCAGAGGATTTCGCGACCTATTACT |
| | | GCCAGCAATACGATTCATACCCGCCAACTTTCGGAAGAGGT |
| | | ACCAAGGTTGAAATCAAGATTGAAGTTATGTATCCTCCTCC |
| | | TTACCTAGACAATGAGAAGAGCAATGGAACCATTATCCATG |
| | | TGAAAGGGAAACACCTTTGTCCAAGTCCCCTATTTCCCGGG |
| | | CCTTCGAAGCCCTTTTGGGTGCTGGTGGTGGTTGGTGGAGTC |
| | | CTGGCTTGCTATAGCTTGCTAGTAACAGTAGCGTTTATTATT |
| | | TTCTGGGTGTGCAGCAGGGCCGCCCGCGGCACCATCGGCGC |
| | | CAGGCGCACAGGCCAGCCTCTGAAGGAGGACCCTTCCGCCG |
| | | TGCCAGTGTTCTCTGTGGACTACGGCGAGCTGGATTTTCAGT |
| | | GGCGGGAGAAAACCCCAGAGCCACCTGTGCCCTGCGTGCCT |
| | | GAGCAGACCGAGTATGCCACAATCGTGTTTCCATCCGGAAT |
| | | GGGCACAAGCTCCCCTGCAAGGAGAGGCAGCGCCGACGGA |
| | | CCACGGTCCGCCCAGCCACTGCGGCCCGAGGATGGCCACTG |
| | | TTCTTGGCCCCTGCGGAGAAAGCGTGGATCCGGGGAAGGCC |
| | | GAGGCTCCCTTCTAACATGTGGAGATGTCGAGGAAAACCCT |
| | | GGCCCTATGGCGCTGCCAGTCACTGCATTGTTATTGCCTCTG |
| | | GCCCTGCTTCTCCATGCGGCGCGCCCACAAGTGCAGCTGAA |
| | | ACAGAGCGGACCAGGACTGGTTCAACCCAGCCAGAGCTTG |
| | | AGCATCACGTGCACGGTTAGCGGCTTCAGTCTGACCAATTA |
| | | TGGTGTGCACTGGGTGAGGCAGTCTCCAGGAAAGGGCCTGG |
| | | AGTGGCTTGGAGTCATTTGGAGCGGTGGGAATACAGATTAC |
| | | AATACACCTTTTACGTCACGTCTCTCCATTAACAAGGACAA |
| | | CTCCAAATCCCAAGTATTTTTCAAAATGAATAGCCTGCAGA |
| | | GTAATGATACCGCCATCTATTACTGTGCACGAGCTTTGACAT |
| | | ATTACGACTATGAATTTGCCTATTGGGGTCAAGGCACGCTG |
| | | GTGACCGTATCAGGCTCAACATCCGGGTCCGGTAAGCCGGG |
| | | CTCCGGCGAGGGGTCTACAAAGGGAGACATCCTTCTGACAC |
| | | AGAGCCCCGTGATCCTGTCCGTGTCCCCCGGCGAGAGAGTA |
| | | TCATTTTCCTGTAGGGCTTCTCAGAGCATCGGAACAAATATC |
| | | CACTGGTATCAGCAACGGACTAACGGATCACCTCGCCTGCT |
| | | CATAAAGTACGCCAGTGAATCTATTAGTGGCATACCGAGCC |
| | | GCTTCAGCGGGAGTGGCTCCGGCACAGACTTTACTCTGAGT |
| | | ATAAATTCCGTGGAATCTGAGGACATCGCGGACTATTACTG |
| | | CCAGCAAAACAATAACTGGCCCACCACGTTCGGCGCGGGA |
| | | ACTAAACTAGAACTAAAGACTACGACCCCAGCACCTAGACC |
| | | TCCCACCCCAGCTCCAACTATAGCTTCCCAGCCATTGTCTCT |
| | | CCGGCCAGAGGCGTGTCGACCAGCCGCTGGAGGGGCCGTTC |
| | | ATACAAGAGGACTCGATTTCGCTTGCGATATCTACATATGG |
| | | GCCCCTCTTGCCGGGACATGCGGTGTCCTGCTTCTAAGCTTG |
| | | GTTATTACCCTCTATTGCAAACGCGGCCGCAAGAAACTGCT |
| | | CTACATCTTTAAACAGCCGTTCATGAGGCCTGTGCAGACAA |
| | | CGCAGGAAGAGGATGGCTGTAGTTGTCGGTTTCCGGAAGAG |
| | | GAAGAGGGGGGCTGCGAGTTGCGTGTCAAATTTTCTCGGTC |
| | | TGCCGACGCCCCCGCGTACCAGCAAGGGCAGAACCAGCTTT |
| | | ATAATGAGCTGAATCTTGGACGACGGGAGGAATATGACGTG |
| | | CTTGACAAGAGGCGAGGTAGGGACCCTGAGATGGGGGGAA |
| | | AACCTCGGAGGAAAAACCCACAGGAAGGCCTGTATAACGA |
| | | ACTGCAGAAGGACAAGATGGCTGAAGCCTACTCTGAGATTG |
| | | GAATGAAAGGGGAACGCAGACGCGGCAAGGGCCATGATGG |
| | | CCTCTACCAAGGTCTAAGCACTGCCACCAAGGACACCTATG |
| | | ACGCACTCCACATGCAAGCTCTACCTCCCCGTTGATAA |
| MC0283- | SEQ ID | MALPVTALLLPLALLLHAARPQVQLVQSGGGVVQPGGSLRVS |
| 3PF12_28_ | NO: 8 | CAASGVTLSDYGMHWVRQAPGKGLEWMAFIRNDGSDKYYA |
| PD1_EGFR | | DSVKGRFTISRDNSKKTVSLQMSSLRAEDTAVYYCAKNGESGP |
| Protein | | LDYWYFDLWGRGTLVTVSSGGGGSGGGGSGGGGSDIVMTQS |
| Sequence | | PSFLSASVGDRVTITCRASHGINNYLAWYQQKPGKAPKLLIYA |
| (VR283) | | ASTLQSGVPSRESGSGSGTEFTLTISSLQPEDFATYYCQQYDSY |
| | | PPTFGRGTKVEIKIEVMYPPPYLDNEKSNGTIIHVKGKHLCPSPL |
| | | FPGPSKPFWVLVVVGGVLACYSLLVTVAFIIFWVCSRAARGTI |
| | | GARRTGQPLKEDPSAVPVFSVDYGELDFQWREKTPEPPVPCVP |
| | | EQTEYATIVFPSGMGTSSPARRGSADGPRSAQPLRPEDGHCSW |
| | | PLRRKRGSGEGRGSLLTCGDVEENPGPMALPVTALLLPLALLL |
| | | HAARPQVQLKQSGPGLVQPSQSLSITCTVSGFSLTNYGVHWVR |
| | | QSPGKGLEWLGVIWSGGNTDYNTPFTSRLSINKDNSKSQVFFK |
| | | MNSLQSNDTAIYYCARALTYYDYEFAYWGQGTLVTVSGSTSG |
| | | SGKPGSGEGSTKGDILLTQSPVILSVSPGERVSFSCRASQSIGTNI |
| | | HWYQQRTNGSPRLLIKYASESISGIPSRFSGSGSGTDFTLSINSV |
| | | ESEDIADYYCQQNNNWPTTFGAGTKLELKTTTPAPRPPTPAPTI |
| | | ASQPLSLRPEACRPAAGGAVHTRGLDFACDIYIWAPLAGTCGV |
| | | LLLSLVITLYCKRGRKKLLYIFKQPFMRPVQTTQEEDGCSCRFP |
| | | EEEEGGCELRVKFSRSADAPAYQQGQNQLYNELNLGRREEYD |
| | | VLDKRRGRDPEMGGKPRRKNPQEGLYNELQKDKMAEAYSEI |
| | | GMKGERRRGKGHDGLYQGLSTATKDTYDALHMQALPPR |
| MC0284- | SEQ ID | ATGGCACTGCCAGTGACCGCCCTGCTGCTGCCTCTGGCCCT |
| BB7.2_8_ | NO: 9 | GCTGCTGCACGCAGCCAGACCCCAGGTGCAGCTGCAGCAGT |
| PD1_HER2 | | CTGGACCTGAGCTGGTGAAGCCAGGAGCCTCCGTGAAGATG |
| Nucleotide | | TCTTGCAAGGCCAGCGGCTACACCTTCACATCTTATCACATC |
| Sequence | | CAGTGGGTGAAGCAGCGGCCCGGACAGGGCCTGGAGTGGA |
| (VR284) | | TCGGATGGATCTACCCAGGCGACGGCTCCACACAGTATAAC |
| | | GAGAAGTTCAAGGGCAAGACCACACTGACCGCCGATAAGA |
| | | GCAGCAGCACCGCCTACATGCTGCTGAGCAGCCTGACCAGC |
| | | GAGGACAGCGCCATCTACTTTTGCGCCAGGGAGGGCACATA |
| | | CTATGCTATGGACTATTGGGGCCAGGGCACCAGCGTGACAG |
| | | TGTCTAGCGGAGGAGGAGGCTCCGGAGGAGGAGGCTCTGG |
| | | CGGCGGCGGCAGCGACGTGCTGATGACCCAGACACCACTG |
| | | AGCCTGCCCGTGAGCCTGGGCGATCAGGTGAGCATCTCCTG |
| | | TAGATCCTCTCAGAGCATCGTGCACTCCAACGGCAATACCT |
| | | ACCTGGAGTGGTATCTGCAGAAGCCAGGCCAGTCCCCCAAG |
| | | CTGCTGATCTATAAGGTGTCTAATCGGTTCAGCGGCGTGCCT |
| | | GACAGATTTTCTGGCAGCGGCTCCGGCACCGACTTCACCCT |
| | | GAAGATCAGCCGGGTGGAGGCAGAGGATCTGGGCGTGTAC |
| | | TATTGTTTCCAGGGCTCCCACGTGCCACGCACCTTTGGCGGC |
| | | GGTACCAAGCTGGAGATCAAGACTACGACCCCAGCACCTAG |
| | | ACCTCCCACCCCAGCTCCAACTATAGCTTCCCAGCCATTGTC |
| | | TCTCCGGCCAGAGGCGTGTCGACCAGCCGCTGGAGGGGCCG |
| | | TTCATACAAGAGGACTCGATTTCGCTTGCGATATCTACATAT |
| | | GGGCCCCTCTTGCCGGGACATGCGGTGTCCTGCTTCTAAGCT |
| | | TGGTTATTACCCTCTATTGCTGCAGCAGGGCCGCCCGCGGC |
| | | ACCATCGGCGCCAGGCGCACAGGCCAGCCTCTGAAGGAGG |
| | | ACCCTTCCGCCGTGCCAGTGTTCTCTGTGGACTACGGCGAG |
| | | CTGGATTTTCAGTGGCGGGAGAAAACCCCAGAGCCACCTGT |
| | | GCCCTGCGTGCCTGAGCAGACCGAGTATGCCACAATCGTGT |
| | | TTCCATCCGGAATGGGCACAAGCTCCCCTGCAAGGAGAGGC |
| | | AGCGCCGACGGACCACGGTCCGCCCAGCCACTGCGGCCCGA |
| | | GGATGGCCACTGTTCTTGGCCCCTGCGGAGAAAGCGTGGAT |
| | | CCGGGGAAGGCCGAGGCTCCCTTCTAACATGTGGAGATGTC |
| | | GAGGAAAACCCTGGCCCTATGGCGCTGCCAGTCACTGCATT |
| | | GTTATTGCCTCTGGCCCTGCTTCTCCATGCGGCGCGCCCAGA |
| | | AGTGCAGCTGGTCGAGAGCGGAGGCGGACTGGTTCAACCC |
| | | GGAGGCAGCTTGAGACTGTCCTGCGCGGCCAGCGGCTTCAA |
| | | CATCAAGGATACCTATATCCACTGGGTGAGGCAGGCTCCAG |
| | | GAAAGGGCCTGGAGTGGGTGGCAAGGATTTACCCTACTAAT |
| | | GGATATACACGCTACGCTGATTCCGTGAAGGGACGCTTTAC |
| | | AATCTCAGCAGATACATCCAAAAACACGGCCTATTTACAGA |
| | | TGAATAGTTTGCGGGCCGAAGACACGGCTGTATACTATTGT |
| | | TCTCGGTGGGGGGGCGATGGATTTTATGCGATGGATTACTG |
| | | GGGCCAGGGCACCCTGGTAACCGTGTCAAGCGGCTCAACAT |
| | | CCGGGTCCGGTAAGCCGGGCTCCGGCGAGGGGTCTACAAA |
| | | GGGAGATATACAGATGACACAGTCCCCCAGTTCCCTGTCCG |
| | | CCTCAGTGGGAGACCGAGTGACGATTACCTGTCGTGCCAGC |
| | | CAGGACGTCAATACCGCCGTCGCTTGGTATCAGCAAAAACC |
| | | AGGCAAGGCCCCGAAACTATTGATCTACAGTGCCTCTTTTCT |
| | | GTACTCCGGGGTGCCGAGCAGATTTAGTGGCTCCAGGAGCG |
| | | GAACCGATTTCACCCTAACCATTTCCAGTTTGCAGCCAGAG |
| | | GATTTCGCGACCTATTACTGCCAGCAACACTACACCACACC |
| | | GCCAACTTTCGGACAAGGAACCAAGGTTGAAATCAAAATTG |
| | | AAGTTATGTATCCTCCTCCTTACCTAGACAATGAGAAGAGC |
| | | AATGGAACCATTATCCATGTGAAAGGGAAACACCTTTGTCC |
| | | AAGTCCCCTATTTCCCGGGCCTTCGAAGCCCTTTTGGGTGCT |
| | | GGTGGTGGTTGGTGGAGTCCTGGCTTGCTATAGCTTGCTAGT |
| | | AACAGTAGCGTTTATTATTTTCTGGGTGAAACGCGGCCGCA |
| | | AGAAACTGCTCTACATCTTTAAACAGCCGTTCATGAGGCCT |
| | | GTGCAGACAACGCAGGAAGAGGATGGCTGTAGTTGTCGGTT |
| | | TCCGGAAGAGGAAGAGGGGGGCTGCGAGTTGCGTGTCAAA |
| | | TTTTCTCGGTCTGCCGACGCCCCCGCGTACCAGCAAGGGCA |
| | | GAACCAGCTTTATAATGAGCTGAATCTTGGACGACGGGAGG |
| | | AATATGACGTGCTTGACAAGAGGCGAGGTAGGGACCCTGA |
| | | GATGGGGGGAAAACCTCGGAGGAAAAACCCACAGGAAGGC |
| | | CTGTATAACGAACTGCAGAAGGACAAGATGGCTGAAGCCT |
| | | ACTCTGAGATTGGAATGAAAGGGGAACGCAGACGCGGCAA |
| | | GGGCCATGATGGCCTCTACCAAGGTCTAAGCACTGCCACCA |
| | | AGGACACCTATGACGCACTCCACATGCAAGCTCTACCTCCC |
| | | CGTTGATAA |
| MC0284- | SEQ ID | MALPVTALLLPLALLLHAARPQVQLQQSGPELVKPGASVKMS |
| BB7.2_8_ | NO: 10 | CKASGYTFTSYHIQWVKQRPGQGLEWIGWIYPGDGSTQYNEK |
| PD1_HER2 | | FKGKTTLTADKSSSTAYMLLSSLTSEDSAIYFCAREGTYYAMD |
| Protein | | YWGQGTSVTVSSGGGGSGGGGSGGGGSDVLMTQTPLSLPVSL |
| Sequence | | GDQVSISCRSSQSIVHSNGNTYLEWYLQKPGQSPKLLIYKVSNR |
| (VR284) | | FSGVPDRESGSGSGTDFTLKISRVEAEDLGVYYCFQGSHVPRTF |
| | | GGGTKLEIKTTTPAPRPPTPAPTIASQPLSLRPEACRPAAGGAV |
| | | HTRGLDFACDIYIWAPLAGTCGVLLLSLVITLYCCSRAARGTIG |
| | | ARRTGQPLKEDPSAVPVFSVDYGELDFQWREKTPEPPVPCVPE |
| | | QTEYATIVFPSGMGTSSPARRGSADGPRSAQPLRPEDGHCSWP |
| | | LRRKRGSGEGRGSLLTCGDVEENPGPMALPVTALLLPLALLLH |
| | | AARPEVQLVESGGGLVQPGGSLRLSCAASGFNIKDTYIHWVRQ |
| | | APGKGLEWVARIYPTNGYTRYADSVKGRFTISADTSKNTAYL |
| | | QMNSLRAEDTAVYYCSRWGGDGFYAMDYWGQGTLVTVSSG |
| | | STSGSGKPGSGEGSTKGDIQMTQSPSSLSASVGDRVTITCRASQ |
| | | DVNTAVAWYQQKPGKAPKLLIYSASFLYSGVPSRESGSRSGTD |
| | | FTLTISSLQPEDFATYYCQQHYTTPPTFGQGTKVEIKIEVMYPPP |
| | | YLDNEKSNGTIIHVKGKHLCPSPLFPGPSKPFWVLVVVGGVLA |
| | | CYSLLVTVAFIIFWVKRGRKKLLYIFKQPFMRPVQTTQEEDGC |
| | | SCRFPEEEEGGCELRVKFSRSADAPAYQQGQNQLYNELNLGR |
| | | REEYDVLDKRRGRDPEMGGKPRRKNPQEGLYNELQKDKMAE |
| | | AYSEIGMKGERRRGKGHDGLYQGLSTATKDTYDALHMQALP |
| | | PR |
| MC0285- | SEQ ID | ATGGCACTGCCAGTGACCGCCCTGCTGCTGCCTCTGGCCCT |
| 3PF12_8_ | NO: 11 | GCTGCTGCACGCAGCCAGACCCCAAGTGCAACTAGTCCAAT |
| PD1_HER2 | | CAGGTGGAGGCGTCGTGCAACCTGGAGGGTCCCTCCGCGTT |
| nucleotide | | AGCTGCGCCGCATCAGGCGTTACCTTGTCAGACTACGGCAT |
| Sequence | | GCATTGGGTTAGGCAAGCCCCCGGCAAGGGGCTCGAATGG |
| (VR285) | | ATGGCTTTCATTCGGAATGACGGGAGCGATAAATATTACGC |
| | | GGATTCAGTTAAAGGGCGGTTCACCATCAGCCGCGACAATA |
| | | GCAAAAAGACGGTCTCCTTACAGATGTCCAGCTTGCGGGCC |
| | | GAAGACACGGCTGTATACTATTGTGCTAAAAATGGCGAGAG |
| | | CGGCCCCCTGGATTACTGGTACTTTGACCTGTGGGGCAGAG |
| | | GCACCCTGGTCACGGTGTCCTCTGGAGGAGGAGGCTCCGGA |
| | | GGAGGAGGCTCTGGCGGCGGCGGCAGCGACATTGTAATGA |
| | | CCCAGTCACCCTCCTTCCTTAGTGCCTCAGTCGGAGACCGCG |
| | | TGACTATCACTTGTCGTGCCTCACACGGAATTAATAACTACC |
| | | TCGCTTGGTATCAGCAAAAACCAGGCAAGGCCCCGAAACTA |
| | | TTGATCTACGCCGCATCTACTCTGCAGAGCGGAGTACCGAG |
| | | CAGATTTAGTGGTTCCGGCAGCGGAACCGAGTTCACCCTAA |
| | | CCATTTCCAGTTTGCAGCCAGAGGATTTCGCGACCTATTACT |
| | | GCCAGCAATACGATTCATACCCGCCAACTTTCGGAAGAGGT |
| | | ACCAAGGTTGAAATCAAGACTACGACCCCAGCACCTAGACC |
| | | TCCCACCCCAGCTCCAACTATAGCTTCCCAGCCATTGTCTCT |
| | | CCGGCCAGAGGCGTGTCGACCAGCCGCTGGAGGGGCCGTTC |
| | | ATACAAGAGGACTCGATTTCGCTTGCGATATCTACATATGG |
| | | GCCCCTCTTGCCGGGACATGCGGTGTCCTGCTTCTAAGCTTG |
| | | GTTATTACCCTCTATTGCTGCAGCAGGGCCGCCCGCGGCAC |
| | | CATCGGCGCCAGGCGCACAGGCCAGCCTCTGAAGGAGGAC |
| | | CCTTCCGCCGTGCCAGTGTTCTCTGTGGACTACGGCGAGCTG |
| | | GATTTTCAGTGGCGGGAGAAAACCCCAGAGCCACCTGTGCC |
| | | CTGCGTGCCTGAGCAGACCGAGTATGCCACAATCGTGTTTC |
| | | CATCCGGAATGGGCACAAGCTCCCCTGCAAGGAGAGGCAG |
| | | CGCCGACGGACCACGGTCCGCCCAGCCACTGCGGCCCGAGG |
| | | ATGGCCACTGTTCTTGGCCCCTGCGGAGAAAGCGTGGATCC |
| | | GGGGAAGGCCGAGGCTCCCTTCTAACATGTGGAGATGTCGA |
| | | GGAAAACCCTGGCCCTATGGCGCTGCCAGTCACTGCATTGT |
| | | TATTGCCTCTGGCCCTGCTTCTCCATGCGGCGCGCCCAGAAG |
| | | TGCAGCTGGTCGAGAGCGGAGGCGGACTGGTTCAACCCGG |
| | | AGGCAGCTTGAGACTGTCCTGCGCGGCCAGCGGCTTCAACA |
| | | TCAAGGATACCTATATCCACTGGGTGAGGCAGGCTCCAGGA |
| | | AAGGGCCTGGAGTGGGTGGCAAGGATTTACCCTACTAATGG |
| | | ATATACACGCTACGCTGATTCCGTGAAGGGACGCTTTACAA |
| | | TCTCAGCAGATACATCCAAAAACACGGCCTATTTACAGATG |
| | | AATAGTTTGCGGGCCGAAGACACGGCTGTATACTATTGTTC |
| | | TCGGTGGGGGGGCGATGGATTTTATGCGATGGATTACTGGG |
| | | GCCAGGGCACCCTGGTAACCGTGTCAAGCGGCTCAACATCC |
| | | GGGTCCGGTAAGCCGGGCTCCGGCGAGGGGTCTACAAAGG |
| | | GAGATATACAGATGACACAGTCCCCCAGTTCCCTGTCCGCC |
| | | TCAGTGGGAGACCGAGTGACGATTACCTGTCGTGCCAGCCA |
| | | GGACGTCAATACCGCCGTCGCTTGGTATCAGCAAAAACCAG |
| | | GCAAGGCCCCGAAACTATTGATCTACAGTGCCTCTTTTCTGT |
| | | ACTCCGGGGTGCCGAGCAGATTTAGTGGCTCCAGGAGCGGA |
| | | ACCGATTTCACCCTAACCATTTCCAGTTTGCAGCCAGAGGA |
| | | TTTCGCGACCTATTACTGCCAGCAACACTACACCACACCGC |
| | | CAACTTTCGGACAAGGAACCAAGGTTGAAATCAAAATTGAA |
| | | GTTATGTATCCTCCTCCTTACCTAGACAATGAGAAGAGCAA |
| | | TGGAACCATTATCCATGTGAAAGGGAAACACCTTTGTCCAA |
| | | GTCCCCTATTTCCCGGGCCTTCGAAGCCCTTTTGGGTGCTGG |
| | | TGGTGGTTGGTGGAGTCCTGGCTTGCTATAGCTTGCTAGTAA |
| | | CAGTAGCGTTTATTATTTTCTGGGTGAAACGCGGCCGCAAG |
| | | AAACTGCTCTACATCTTTAAACAGCCGTTCATGAGGCCTGT |
| | | GCAGACAACGCAGGAAGAGGATGGCTGTAGTTGTCGGTTTC |
| | | CGGAAGAGGAAGAGGGGGGCTGCGAGTTGCGTGTCAAATT |
| | | TTCTCGGTCTGCCGACGCCCCCGCGTACCAGCAAGGGCAGA |
| | | ACCAGCTTTATAATGAGCTGAATCTTGGACGACGGGAGGAA |
| | | TATGACGTGCTTGACAAGAGGCGAGGTAGGGACCCTGAGAT |
| | | GGGGGGAAAACCTCGGAGGAAAAACCCACAGGAAGGCCTG |
| | | TATAACGAACTGCAGAAGGACAAGATGGCTGAAGCCTACTC |
| | | TGAGATTGGAATGAAAGGGGAACGCAGACGCGGCAAGGGC |
| | | CATGATGGCCTCTACCAAGGTCTAAGCACTGCCACCAAGGA |
| | | CACCTATGACGCACTCCACATGCAAGCTCTACCTCCCCGTTG |
| | | ATAAMC |
| MC0285- | SEQ ID | MALPVTALLLPLALLLHAARPQVQLVQSGGGVVQPGGSLRVS |
| 3PF12_8_ | NO: 12 | CAASGVTLSDYGMHWVRQAPGKGLEWMAFIRNDGSDKYYA |
| PD1_HER2 | | DSVKGRFTISRDNSKKTVSLQMSSLRAEDTAVYYCAKNGESGP |
| Protein | | LDYWYFDLWGRGTLVTVSSGGGGSGGGGSGGGGSDIVMTQS |
| Sequence | | PSFLSASVGDRVTITCRASHGINNYLAWYQQKPGKAPKLLIYA |
| (VR285) | | ASTLQSGVPSRFSGSGSGTEFTLTISSLQPEDFATYYCQQYDSY |
| | | PPTFGRGTKVEIKTTTPAPRPPTPAPTIASQPLSLRPEACRPAAG |
| | | GAVHTRGLDFACDIYIWAPLAGTCGVLLLSLVITLYCCSRAAR |
| | | GTIGARRTGQPLKEDPSAVPVFSVDYGELDFQWREKTPEPPVP |
| | | CVPEQTEYATIVFPSGMGTSSPARRGSADGPRSAQPLRPEDGH |
| | | CSWPLRRKRGSGEGRGSLLTCGDVEENPGPMALPVTALLLPLA |
| | | LLLHAARPEVQLVESGGGLVQPGGSLRLSCAASGFNIKDTYIH |
| | | WVRQAPGKGLEWVARIYPTNGYTRYADSVKGRFTISADTSKN |
| | | TAYLQMNSLRAEDTAVYYCSRWGGDGFYAMDYWGQGTLVT |
| | | VSSGSTSGSGKPGSGEGSTKGDIQMTQSPSSLSASVGDRVTITC |
| | | RASQDVNTAVAWYQQKPGKAPKLLIYSASFLYSGVPSRFSGSR |
| | | SGTDFTLTISSLQPEDFATYYCQQHYTTPPTFGQGTKVEIKIEV |
| | | MYPPPYLDNEKSNGTIIHVKGKHLCPSPLFPGPSKPFWVLVVV |
| | | GGVLACYSLLVTVAFIIFWVKRGRKKLLYIFKQPFMRPVQTTQ |
| | | EEDGCSCRFPEEEEGGCELRVKESRSADAPAYQQGQNQLYNEL |
| | | NLGRREEYDVLDKRRGRDPEMGGKPRRKNPQEGLYNELQKD |
| | | KMAEAYSEIGMKGERRRGKGHDGLYQGLSTATKDTYDALHM |
| | | QALPPR |
| MC0286- | SEQ ID | ATGGCACTGCCAGTGACCGCCCTGCTGCTGCCTCTGGCCCT |
| BB7.2_8_ | NO: 13 | GCTGCTGCACGCAGCCAGACCCCAGGTGCAGCTGCAGCAGT |
| PD1_EGFR | | CTGGACCTGAGCTGGTGAAGCCAGGAGCCTCCGTGAAGATG |
| nucleotide | | TCTTGCAAGGCCAGCGGCTACACCTTCACATCTTATCACATC |
| Sequence | | CAGTGGGTGAAGCAGCGGCCCGGACAGGGCCTGGAGTGGA |
| (VR286) | | TCGGATGGATCTACCCAGGCGACGGCTCCACACAGTATAAC |
| | | GAGAAGTTCAAGGGCAAGACCACACTGACCGCCGATAAGA |
| | | GCAGCAGCACCGCCTACATGCTGCTGAGCAGCCTGACCAGC |
| | | GAGGACAGCGCCATCTACTTTTGCGCCAGGGAGGGCACATA |
| | | CTATGCTATGGACTATTGGGGCCAGGGCACCAGCGTGACAG |
| | | TGTCTAGCGGAGGAGGAGGCTCCGGAGGAGGAGGCTCTGG |
| | | CGGCGGCGGCAGCGACGTGCTGATGACCCAGACACCACTG |
| | | AGCCTGCCCGTGAGCCTGGGCGATCAGGTGAGCATCTCCTG |
| | | TAGATCCTCTCAGAGCATCGTGCACTCCAACGGCAATACCT |
| | | ACCTGGAGTGGTATCTGCAGAAGCCAGGCCAGTCCCCCAAG |
| | | CTGCTGATCTATAAGGTGTCTAATCGGTTCAGCGGCGTGCCT |
| | | GACAGATTTTCTGGCAGCGGCTCCGGCACCGACTTCACCCT |
| | | GAAGATCAGCCGGGTGGAGGCAGAGGATCTGGGCGTGTAC |
| | | TATTGTTTCCAGGGCTCCCACGTGCCACGCACCTTTGGCGGC |
| | | GGTACCAAGCTGGAGATCAAGACTACGACCCCAGCACCTAG |
| | | ACCTCCCACCCCAGCTCCAACTATAGCTTCCCAGCCATTGTC |
| | | TCTCCGGCCAGAGGCGTGTCGACCAGCCGCTGGAGGGGCCG |
| | | TTCATACAAGAGGACTCGATTTCGCTTGCGATATCTACATAT |
| | | GGGCCCCTCTTGCCGGGACATGCGGTGTCCTGCTTCTAAGCT |
| | | TGGTTATTACCCTCTATTGCTGCAGCAGGGCCGCCCGCGGC |
| | | ACCATCGGCGCCAGGCGCACAGGCCAGCCTCTGAAGGAGG |
| | | ACCCTTCCGCCGTGCCAGTGTTCTCTGTGGACTACGGCGAG |
| | | CTGGATTTTCAGTGGCGGGAGAAAACCCCAGAGCCACCTGT |
| | | GCCCTGCGTGCCTGAGCAGACCGAGTATGCCACAATCGTGT |
| | | TTCCATCCGGAATGGGCACAAGCTCCCCTGCAAGGAGAGGC |
| | | AGCGCCGACGGACCACGGTCCGCCCAGCCACTGCGGCCCGA |
| | | GGATGGCCACTGTTCTTGGCCCCTGCGGAGAAAGCGTGGAT |
| | | CCGGGGAAGGCCGAGGCTCCCTTCTAACATGTGGAGATGTC |
| | | GAGGAAAACCCTGGCCCTATGGCGCTGCCAGTCACTGCATT |
| | | GTTATTGCCTCTGGCCCTGCTTCTCCATGCGGCGCGCCCACA |
| | | AGTGCAGCTGAAACAGAGCGGACCAGGACTGGTTCAACCC |
| | | AGCCAGAGCTTGAGCATCACGTGCACGGTTAGCGGCTTCAG |
| | | TCTGACCAATTATGGTGTGCACTGGGTGAGGCAGTCTCCAG |
| | | GAAAGGGCCTGGAGTGGCTTGGAGTCATTTGGAGCGGTGGG |
| | | AATACAGATTACAATACACCTTTTACGTCACGTCTCTCCATT |
| | | AACAAGGACAACTCCAAATCCCAAGTATTTTTCAAAATGAA |
| | | TAGCCTGCAGAGTAATGATACCGCCATCTATTACTGTGCAC |
| | | GAGCTTTGACATATTACGACTATGAATTTGCCTATTGGGGTC |
| | | AAGGCACGCTGGTGACCGTATCAGGCTCAACATCCGGGTCC |
| | | GGTAAGCCGGGCTCCGGCGAGGGGTCTACAAAGGGAGACA |
| | | TCCTTCTGACACAGAGCCCCGTGATCCTGTCCGTGTCCCCCG |
| | | GCGAGAGAGTATCATTTTCCTGTAGGGCTTCTCAGAGCATC |
| | | GGAACAAATATCCACTGGTATCAGCAACGGACTAACGGATC |
| | | ACCTCGCCTGCTCATAAAGTACGCCAGTGAATCTATTAGTG |
| | | GCATACCGAGCCGCTTCAGCGGGAGTGGCTCCGGCACAGAC |
| | | TTTACTCTGAGTATAAATTCCGTGGAATCTGAGGACATCGC |
| | | GGACTATTACTGCCAGCAAAACAATAACTGGCCCACCACGT |
| | | TCGGCGCGGGAACTAAACTAGAACTAAAGATTGAAGTTATG |
| | | TATCCTCCTCCTTACCTAGACAATGAGAAGAGCAATGGAAC |
| | | CATTATCCATGTGAAAGGGAAACACCTTTGTCCAAGTCCCC |
| | | TATTTCCCGGGCCTTCGAAGCCCTTTTGGGTGCTGGTGGTGG |
| | | TTGGTGGAGTCCTGGCTTGCTATAGCTTGCTAGTAACAGTA |
| | | GCGTTTATTATTTTCTGGGTGAAACGCGGCCGCAAGAAACT |
| | | GCTCTACATCTTTAAACAGCCGTTCATGAGGCCTGTGCAGA |
| | | CAACGCAGGAAGAGGATGGCTGTAGTTGTCGGTTTCCGGAA |
| | | GAGGAAGAGGGGGGCTGCGAGTTGCGTGTCAAATTTTCTCG |
| | | GTCTGCCGACGCCCCCGCGTACCAGCAAGGGCAGAACCAGC |
| | | TTTATAATGAGCTGAATCTTGGACGACGGGAGGAATATGAC |
| | | GTGCTTGACAAGAGGCGAGGTAGGGACCCTGAGATGGGGG |
| | | GAAAACCTCGGAGGAAAAACCCACAGGAAGGCCTGTATAA |
| | | CGAACTGCAGAAGGACAAGATGGCTGAAGCCTACTCTGAG |
| | | ATTGGAATGAAAGGGGAACGCAGACGCGGCAAGGGCCATG |
| | | ATGGCCTCTACCAAGGTCTAAGCACTGCCACCAAGGACACC |
| | | TATGACGCACTCCACATGCAAGCTCTACCTCCCCGTTGATA |
| | | A |
| MC0286- | SEQ ID | MALPVTALLLPLALLLHAARPQVQLQQSGPELVKPGASVKMS |
| BB7.2_8_ | NO: 14 | CKASGYTFTSYHIQWVKQRPGQGLEWIGWIYPGDGSTQYNEK |
| PD1_EGFR | | FKGKTTLTADKSSSTAYMLLSSLTSEDSAIYFCAREGTYYAMD |
| Protein | | YWGQGTSVTVSSGGGGSGGGGSGGGGSDVLMTQTPLSLPVSL |
| Sequence | | GDQVSISCRSSQSIVHSNGNTYLEWYLQKPGQSPKLLIYKVSNR |
| (VR286) | | FSGVPDRFSGSGSGTDFTLKISRVEAEDLGVYYCFQGSHVPRTF |
| | | GGGTKLEIKTTTPAPRPPTPAPTIASQPLSLRPEACRPAAGGAV |
| | | HTRGLDFACDIYIWAPLAGTCGVLLLSLVITLYCCSRAARGTIG |
| | | ARRTGQPLKEDPSAVPVFSVDYGELDFQWREKTPEPPVPCVPE |
| | | QTEYATIVFPSGMGTSSPARRGSADGPRSAQPLRPEDGHCSWP |
| | | LRRKRGSGEGRGSLLTCGDVEENPGPMALPVTALLLPLALLLH |
| | | AARPQVQLKQSGPGLVQPSQSLSITCTVSGFSLTNYGVHWVRQ |
| | | SPGKGLEWLGVIWSGGNTDYNTPFTSRLSINKDNSKSQVFFKM |
| | | NSLQSNDTAIYYCARALTYYDYEFAYWGQGTLVTVSGSTSGS |
| | | GKPGSGEGSTKGDILLTQSPVILSVSPGERVSFSCRASQSIGTNI |
| | | HWYQQRTNGSPRLLIKYASESISGIPSRFSGSGSGTDFTLSINSV |
| | | ESEDIADYYCQQNNNWPTTFGAGTKLELKIEVMYPPPYLDNE |
| | | KSNGTIIHVKGKHLCPSPLFPGPSKPFWVLVVVGGVLACYSLL |
| | | VTVAFIIFWVKRGRKKLLYIFKQPFMRPVQTTQEEDGCSCRFPE |
| | | EEEGGCELRVKFSRSADAPAYQQGQNQLYNELNLGRREEYDV |
| | | LDKRRGRDPEMGGKPRRKNPQEGLYNELQKDKMAEAYSEIG |
| | | MKGERRRGKGHDGLYQGLSTATKDTYDALHMQALPPR |
| MC0287- | SEQ ID | ATGGCACTGCCAGTGACCGCCCTGCTGCTGCCTCTGGCCCT |
| 3PF12_8_P | NO: 15 | GCTGCTGCACGCAGCCAGACCCCAAGTGCAACTAGTCCAAT |
| D1_EGFR | | CAGGTGGAGGCGTCGTGCAACCTGGAGGGTCCCTCCGCGTT |
| Nucleotide | | AGCTGCGCCGCATCAGGCGTTACCTTGTCAGACTACGGCAT |
| Sequence | | GCATTGGGTTAGGCAAGCCCCCGGCAAGGGGCTCGAATGG |
| (VR287) | | ATGGCTTTCATTCGGAATGACGGGAGCGATAAATATTACGC |
| | | GGATTCAGTTAAAGGGCGGTTCACCATCAGCCGCGACAATA |
| | | GCAAAAAGACGGTCTCCTTACAGATGTCCAGCTTGCGGGCC |
| | | GAAGACACGGCTGTATACTATTGTGCTAAAAATGGCGAGAG |
| | | CGGCCCCCTGGATTACTGGTACTTTGACCTGTGGGGCAGAG |
| | | GCACCCTGGTCACGGTGTCCTCTGGAGGAGGAGGCTCCGGA |
| | | GGAGGAGGCTCTGGCGGCGGCGGCAGCGACATTGTAATGA |
| | | CCCAGTCACCCTCCTTCCTTAGTGCCTCAGTCGGAGACCGCG |
| | | TGACTATCACTTGTCGTGCCTCACACGGAATTAATAACTACC |
| | | TCGCTTGGTATCAGCAAAAACCAGGCAAGGCCCCGAAACTA |
| | | TTGATCTACGCCGCATCTACTCTGCAGAGCGGAGTACCGAG |
| | | CAGATTTAGTGGTTCCGGCAGCGGAACCGAGTTCACCCTAA |
| | | CCATTTCCAGTTTGCAGCCAGAGGATTTCGCGACCTATTACT |
| | | GCCAGCAATACGATTCATACCCGCCAACTTTCGGAAGAGGT |
| | | ACCAAGGTTGAAATCAAGACTACGACCCCAGCACCTAGACC |
| | | TCCCACCCCAGCTCCAACTATAGCTTCCCAGCCATTGTCTCT |
| | | CCGGCCAGAGGCGTGTCGACCAGCCGCTGGAGGGGCCGTTC |
| | | ATACAAGAGGACTCGATTTCGCTTGCGATATCTACATATGG |
| | | GCCCCTCTTGCCGGGACATGCGGTGTCCTGCTTCTAAGCTTG |
| | | GTTATTACCCTCTATTGCTGCAGCAGGGCCGCCCGCGGCAC |
| | | CATCGGCGCCAGGCGCACAGGCCAGCCTCTGAAGGAGGAC |
| | | CCTTCCGCCGTGCCAGTGTTCTCTGTGGACTACGGCGAGCTG |
| | | GATTTTCAGTGGCGGGAGAAAACCCCAGAGCCACCTGTGCC |
| | | CTGCGTGCCTGAGCAGACCGAGTATGCCACAATCGTGTTTC |
| | | CATCCGGAATGGGCACAAGCTCCCCTGCAAGGAGAGGCAG |
| | | CGCCGACGGACCACGGTCCGCCCAGCCACTGCGGCCCGAGG |
| | | ATGGCCACTGTTCTTGGCCCCTGCGGAGAAAGCGTGGATCC |
| | | GGGGAAGGCCGAGGCTCCCTTCTAACATGTGGAGATGTCGA |
| | | GGAAAACCCTGGCCCTATGGCGCTGCCAGTCACTGCATTGT |
| | | TATTGCCTCTGGCCCTGCTTCTCCATGCGGCGCGCCCACAAG |
| | | TGCAGCTGAAACAGAGCGGACCAGGACTGGTTCAACCCAG |
| | | CCAGAGCTTGAGCATCACGTGCACGGTTAGCGGCTTCAGTC |
| | | TGACCAATTATGGTGTGCACTGGGTGAGGCAGTCTCCAGGA |
| | | AAGGGCCTGGAGTGGCTTGGAGTCATTTGGAGCGGTGGGAA |
| | | TACAGATTACAATACACCTTTTACGTCACGTCTCTCCATTAA |
| | | CAAGGACAACTCCAAATCCCAAGTATTTTTCAAAATGAATA |
| | | GCCTGCAGAGTAATGATACCGCCATCTATTACTGTGCACGA |
| | | GCTTTGACATATTACGACTATGAATTTGCCTATTGGGGTCAA |
| | | GGCACGCTGGTGACCGTATCAGGCTCAACATCCGGGTCCGG |
| | | TAAGCCGGGCTCCGGCGAGGGGTCTACAAAGGGAGACATC |
| | | CTTCTGACACAGAGCCCCGTGATCCTGTCCGTGTCCCCCGGC |
| | | GAGAGAGTATCATTTTCCTGTAGGGCTTCTCAGAGCATCGG |
| | | AACAAATATCCACTGGTATCAGCAACGGACTAACGGATCAC |
| | | CTCGCCTGCTCATAAAGTACGCCAGTGAATCTATTAGTGGC |
| | | ATACCGAGCCGCTTCAGCGGGAGTGGCTCCGGCACAGACTT |
| | | TACTCTGAGTATAAATTCCGTGGAATCTGAGGACATCGCGG |
| | | ACTATTACTGCCAGCAAAACAATAACTGGCCCACCACGTTC |
| | | GGCGCGGGAACTAAACTAGAACTAAAGATTGAAGTTATGTA |
| | | TCCTCCTCCTTACCTAGACAATGAGAAGAGCAATGGAACCA |
| | | TTATCCATGTGAAAGGGAAACACCTTTGTCCAAGTCCCCTA |
| | | TTTCCCGGGCCTTCGAAGCCCTTTTGGGTGCTGGTGGTGGTT |
| | | GGTGGAGTCCTGGCTTGCTATAGCTTGCTAGTAACAGTAGC |
| | | GTTTATTATTTTCTGGGTGAAACGCGGCCGCAAGAAACTGC |
| | | TCTACATCTTTAAACAGCCGTTCATGAGGCCTGTGCAGACA |
| | | ACGCAGGAAGAGGATGGCTGTAGTTGTCGGTTTCCGGAAGA |
| | | GGAAGAGGGGGGCTGCGAGTTGCGTGTCAAATTTTCTCGGT |
| | | CTGCCGACGCCCCCGCGTACCAGCAAGGGCAGAACCAGCTT |
| | | TATAATGAGCTGAATCTTGGACGACGGGAGGAATATGACGT |
| | | GCTTGACAAGAGGCGAGGTAGGGACCCTGAGATGGGGGGA |
| | | AAACCTCGGAGGAAAAACCCACAGGAAGGCCTGTATAACG |
| | | AACTGCAGAAGGACAAGATGGCTGAAGCCTACTCTGAGATT |
| | | GGAATGAAAGGGGAACGCAGACGCGGCAAGGGCCATGATG |
| | | GCCTCTACCAAGGTCTAAGCACTGCCACCAAGGACACCTAT |
| | | GACGCACTCCACATGCAAGCTCTACCTCCCCGTTGATAA |
| MC0287- | SEQ ID | MALPVTALLLPLALLLHAARPQVQLVQSGGGVVQPGGSLRVS |
| 3PF12_8_ | NO: 16 | CAASGVTLSDYGMHWVRQAPGKGLEWMAFIRNDGSDKYYA |
| PD1_EGFR | | DSVKGRFTISRDNSKKTVSLQMSSLRAEDTAVYYCAKNGESGP |
| Protein | | LDYWYFDLWGRGTLVTVSSGGGGSGGGGSGGGGSDIVMTQS |
| Sequence | | PSFLSASVGDRVTITCRASHGINNYLAWYQQKPGKAPKLLIYA |
| (VR287) | | ASTLQSGVPSRFSGSGSGTEFTLTISSLQPEDFATYYCQQYDSY |
| | | PPTFGRGTKVEIKTTTPAPRPPTPAPTIASQPLSLRPEACRPAAG |
| | | GAVHTRGLDFACDIYIWAPLAGTCGVLLLSLVITLYCCSRAAR |
| | | GTIGARRTGQPLKEDPSAVPVFSVDYGELDFQWREKTPEPPVP |
| | | CVPEQTEYATIVFPSGMGTSSPARRGSADGPRSAQPLRPEDGH |
| | | CSWPLRRKRGSGEGRGSLLTCGDVEENPGPMALPVTALLLPLA |
| | | LLLHAARPQVQLKQSGPGLVQPSQSLSITCTVSGFSLTNYGVH |
| | | WVRQSPGKGLEWLGVIWSGGNTDYNTPFTSRLSINKDNSKSQ |
| | | VFFKMNSLQSNDTAIYYCARALTYYDYEFAYWGQGTLVTVSG |
| | | STSGSGKPGSGEGSTKGDILLTQSPVILSVSPGERVSFSCRASQSI |
| | | GTNIHWYQQRTNGSPRLLIKYASESISGIPSRFSGSGSGTDFTLSI |
| | | NSVESEDIADYYCQQNNNWPTTFGAGTKLELKIEVMYPPPYLD |
| | | NEKSNGTIIHVKGKHLCPSPLFPGPSKPFWVLVVVGGVLACYS |
| | | LLVTVAFIIFWVKRGRKKLLYIFKQPFMRPVQTTQEEDGCSCRF |
| | | PEEEEGGCELRVKFSRSADAPAYQQGQNQLYNELNLGRREEY |
| | | DVLDKRRGRDPEMGGKPRRKNPQEGLYNELQKDKMAEAYSE |
| | | IGMKGERRRGKGHDGLYQGLSTATKDTYDALHMQALPPR |
| MC0288- | SEQ ID | ATGGCACTGCCAGTGACCGCCCTGCTGCTGCCTCTGGCCCT |
| BB7.2_28_ | NO: 17 | GCTGCTGCACGCAGCCAGACCCCAGGTGCAGCTGCAGCAGT |
| Pdel_HER2 | | CTGGACCTGAGCTGGTGAAGCCAGGAGCCTCCGTGAAGATG |
| Nucleotide | | TCTTGCAAGGCCAGCGGCTACACCTTCACATCTTATCACATC |
| Sequence | | CAGTGGGTGAAGCAGCGGCCCGGACAGGGCCTGGAGTGGA |
| (VR288) | | TCGGATGGATCTACCCAGGCGACGGCTCCACACAGTATAAC |
| | | GAGAAGTTCAAGGGCAAGACCACACTGACCGCCGATAAGA |
| | | GCAGCAGCACCGCCTACATGCTGCTGAGCAGCCTGACCAGC |
| | | GAGGACAGCGCCATCTACTTTTGCGCCAGGGAGGGCACATA |
| | | CTATGCTATGGACTATTGGGGCCAGGGCACCAGCGTGACAG |
| | | TGTCTAGCGGAGGAGGAGGCTCCGGAGGAGGAGGCTCTGG |
| | | CGGCGGCGGCAGCGACGTGCTGATGACCCAGACACCACTG |
| | | AGCCTGCCCGTGAGCCTGGGCGATCAGGTGAGCATCTCCTG |
| | | TAGATCCTCTCAGAGCATCGTGCACTCCAACGGCAATACCT |
| | | ACCTGGAGTGGTATCTGCAGAAGCCAGGCCAGTCCCCCAAG |
| | | CTGCTGATCTATAAGGTGTCTAATCGGTTCAGCGGCGTGCCT |
| | | GACAGATTTTCTGGCAGCGGCTCCGGCACCGACTTCACCCT |
| | | GAAGATCAGCCGGGTGGAGGCAGAGGATCTGGGCGTGTAC |
| | | TATTGTTTCCAGGGCTCCCACGTGCCACGCACCTTTGGCGGC |
| | | GGTACCAAGCTGGAGATCAAGATTGAAGTTATGTATCCTCC |
| | | TCCTTACCTAGACAATGAGAAGAGCAATGGAACCATTATCC |
| | | ATGTGAAAGGGAAACACCTTTGTCCAAGTCCCCTATTTCCC |
| | | GGGCCTTCGAAGCCCTTTTGGGTGCTGGTGGTGGTTGGTGG |
| | | AGTCCTGGCTTGCTATAGCTTGCTAGTAACAGTAGCGTTTAT |
| | | TATTTTCTGGGTGCGGAGAAAGCGTGGATCCGGGGAAGGCC |
| | | GAGGCTCCCTTCTAACATGTGGAGATGTCGAGGAAAACCCT |
| | | GGCCCTATGGCGCTGCCAGTCACTGCATTGTTATTGCCTCTG |
| | | GCCCTGCTTCTCCATGCGGCGCGCCCAGAAGTGCAGCTGGT |
| | | CGAGAGCGGAGGCGGACTGGTTCAACCCGGAGGCAGCTTG |
| | | AGACTGTCCTGCGCGGCCAGCGGCTTCAACATCAAGGATAC |
| | | CTATATCCACTGGGTGAGGCAGGCTCCAGGAAAGGGCCTGG |
| | | AGTGGGTGGCAAGGATTTACCCTACTAATGGATATACACGC |
| | | TACGCTGATTCCGTGAAGGGACGCTTTACAATCTCAGCAGA |
| | | TACATCCAAAAACACGGCCTATTTACAGATGAATAGTTTGC |
| | | GGGCCGAAGACACGGCTGTATACTATTGTTCTCGGTGGGGG |
| | | GGCGATGGATTTTATGCGATGGATTACTGGGGCCAGGGCAC |
| | | CCTGGTAACCGTGTCAAGCGGCTCAACATCCGGGTCCGGTA |
| | | AGCCGGGCTCCGGCGAGGGGTCTACAAAGGGAGATATACA |
| | | GATGACACAGTCCCCCAGTTCCCTGTCCGCCTCAGTGGGAG |
| | | ACCGAGTGACGATTACCTGTCGTGCCAGCCAGGACGTCAAT |
| | | ACCGCCGTCGCTTGGTATCAGCAAAAACCAGGCAAGGCCCC |
| | | GAAACTATTGATCTACAGTGCCTCTTTTCTGTACTCCGGGGT |
| | | GCCGAGCAGATTTAGTGGCTCCAGGAGCGGAACCGATTTCA |
| | | CCCTAACCATTTCCAGTTTGCAGCCAGAGGATTTCGCGACCT |
| | | ATTACTGCCAGCAACACTACACCACACCGCCAACTTTCGGA |
| | | CAAGGAACCAAGGTTGAAATCAAAACTACGACCCCAGCAC |
| | | CTAGACCTCCCACCCCAGCTCCAACTATAGCTTCCCAGCCAT |
| | | TGTCTCTCCGGCCAGAGGCGTGTCGACCAGCCGCTGGAGGG |
| | | GCCGTTCATACAAGAGGACTCGATTTCGCTTGCGATATCTA |
| | | CATATGGGCCCCTCTTGCCGGGACATGCGGTGTCCTGCTTCT |
| | | AAGCTTGGTTATTACCCTCTATTGCAAACGCGGCCGCAAGA |
| | | AACTGCTCTACATCTTTAAACAGCCGTTCATGAGGCCTGTGC |
| | | AGACAACGCAGGAAGAGGATGGCTGTAGTTGTCGGTTTCCG |
| | | GAAGAGGAAGAGGGGGGCTGCGAGTTGCGTGTCAAATTTTC |
| | | TCGGTCTGCCGACGCCCCCGCGTACCAGCAAGGGCAGAACC |
| | | AGCTTTATAATGAGCTGAATCTTGGACGACGGGAGGAATAT |
| | | GACGTGCTTGACAAGAGGCGAGGTAGGGACCCTGAGATGG |
| | | GGGGAAAACCTCGGAGGAAAAACCCACAGGAAGGCCTGTA |
| | | TAACGAACTGCAGAAGGACAAGATGGCTGAAGCCTACTCTG |
| | | AGATTGGAATGAAAGGGGAACGCAGACGCGGCAAGGGCCA |
| | | TGATGGCCTCTACCAAGGTCTAAGCACTGCCACCAAGGACA |
| | | CCTATGACGCACTCCACATGCAAGCTCTACCTCCCCGTTGAT |
| | | AA |
| MC0288- | SEQ ID | MALPVTALLLPLALLLHAARPQVQLQQSGPELVKPGASVKMS |
| BB7.2_28_ | NO: 18 | CKASGYTFTSYHIQWVKQRPGQGLEWIGWIYPGDGSTQYNEK |
| Pdel_HER2 | | FKGKTTLTADKSSSTAYMLLSSLTSEDSAIYFCAREGTYYAMD |
| Protein | | YWGQGTSVTVSSGGGGSGGGGSGGGGSDVLMTQTPLSLPVSL |
| Sequence | | GDQVSISCRSSQSIVHSNGNTYLEWYLQKPGQSPKLLIYKVSNR |
| (VR288) | | FSGVPDRFSGSGSGTDFTLKISRVEAEDLGVYYCFQGSHVPRTF |
| | | GGGTKLEIKIEVMYPPPYLDNEKSNGTIIHVKGKHLCPSPLFPG |
| | | PSKPFWVLVVVGGVLACYSLLVTVAFIIFWVRRKRGSGEGRGS |
| | | LLTCGDVEENPGPMALPVTALLLPLALLLHAARPEVQLVESGG |
| | | GLVQPGGSLRLSCAASGFNIKDTYIHWVRQAPGKGLEWVARI |
| | | YPTNGYTRYADSVKGRFTISADTSKNTAYLQMNSLRAEDTAV |
| | | YYCSRWGGDGFYAMDYWGQGTLVTVSSGSTSGSGKPGSGEG |
| | | STKGDIQMTQSPSSLSASVGDRVTITCRASQDVNTAVAWYQQ |
| | | KPGKAPKLLIYSASFLYSGVPSRFSGSRSGTDFTLTISSLQPEDF |
| | | ATYYCQQHYTTPPTFGQGTKVEIKTTTPAPRPPTPAPTIASQPLS |
| | | LRPEACRPAAGGAVHTRGLDFACDIYIWAPLAGTCGVLLLSLV |
| | | ITLYCKRGRKKLLYIFKQPFMRPVQTTQEEDGCSCRFPEEEEGG |
| | | CELRVKFSRSADAPAYQQGQNQLYNELNLGRREEYDVLDKRR |
| | | GRDPEMGGKPRRKNPQEGLYNELQKDKMAEAYSEIGMKGER |
| | | RRGKGHDGLYQGLSTATKDTYDALHMQALPPR |
| MC0289- | SEQ ID | ATGGCACTGCCAGTGACCGCCCTGCTGCTGCCTCTGGCCCT |
| 3PF12_28 | NO: 19 | GCTGCTGCACGCAGCCAGACCCCAAGTGCAACTAGTCCAAT |
| Pdel_HER2 | | CAGGTGGAGGCGTCGTGCAACCTGGAGGGTCCCTCCGCGTT |
| Nucleotide | | AGCTGCGCCGCATCAGGCGTTACCTTGTCAGACTACGGCAT |
| Sequence | | GCATTGGGTTAGGCAAGCCCCCGGCAAGGGGCTCGAATGG |
| (VR289) | | ATGGCTTTCATTCGGAATGACGGGAGCGATAAATATTACGC |
| | | GGATTCAGTTAAAGGGCGGTTCACCATCAGCCGCGACAATA |
| | | GCAAAAAGACGGTCTCCTTACAGATGTCCAGCTTGCGGGCC |
| | | GAAGACACGGCTGTATACTATTGTGCTAAAAATGGCGAGAG |
| | | CGGCCCCCTGGATTACTGGTACTTTGACCTGTGGGGCAGAG |
| | | GCACCCTGGTCACGGTGTCCTCTGGAGGAGGAGGCTCCGGA |
| | | GGAGGAGGCTCTGGCGGCGGCGGCAGCGACATTGTAATGA |
| | | CCCAGTCACCCTCCTTCCTTAGTGCCTCAGTCGGAGACCGCG |
| | | TGACTATCACTTGTCGTGCCTCACACGGAATTAATAACTACC |
| | | TCGCTTGGTATCAGCAAAAACCAGGCAAGGCCCCGAAACTA |
| | | TTGATCTACGCCGCATCTACTCTGCAGAGCGGAGTACCGAG |
| | | CAGATTTAGTGGTTCCGGCAGCGGAACCGAGTTCACCCTAA |
| | | CCATTTCCAGTTTGCAGCCAGAGGATTTCGCGACCTATTACT |
| | | GCCAGCAATACGATTCATACCCGCCAACTTTCGGAAGAGGT |
| | | ACCAAGGTTGAAATCAAGATTGAAGTTATGTATCCTCCTCC |
| | | TTACCTAGACAATGAGAAGAGCAATGGAACCATTATCCATG |
| | | TGAAAGGGAAACACCTTTGTCCAAGTCCCCTATTTCCCGGG |
| | | CCTTCGAAGCCCTTTTGGGTGCTGGTGGTGGTTGGTGGAGTC |
| | | CTGGCTTGCTATAGCTTGCTAGTAACAGTAGCGTTTATTATT |
| | | TTCTGGGTGCGGAGAAAGCGTGGATCCGGGGAAGGCCGAG |
| | | GCTCCCTTCTAACATGTGGAGATGTCGAGGAAAACCCTGGC |
| | | CCTATGGCGCTGCCAGTCACTGCATTGTTATTGCCTCTGGCC |
| | | CTGCTTCTCCATGCGGCGCGCCCAGAAGTGCAGCTGGTCGA |
| | | GAGCGGAGGCGGACTGGTTCAACCCGGAGGCAGCTTGAGA |
| | | CTGTCCTGCGCGGCCAGCGGCTTCAACATCAAGGATACCTA |
| | | TATCCACTGGGTGAGGCAGGCTCCAGGAAAGGGCCTGGAGT |
| | | GGGTGGCAAGGATTTACCCTACTAATGGATATACACGCTAC |
| | | GCTGATTCCGTGAAGGGACGCTTTACAATCTCAGCAGATAC |
| | | ATCCAAAAACACGGCCTATTTACAGATGAATAGTTTGCGGG |
| | | CCGAAGACACGGCTGTATACTATTGTTCTCGGTGGGGGGGC |
| | | GATGGATTTTATGCGATGGATTACTGGGGCCAGGGCACCCT |
| | | GGTAACCGTGTCAAGCGGCTCAACATCCGGGTCCGGTAAGC |
| | | CGGGCTCCGGCGAGGGGTCTACAAAGGGAGATATACAGAT |
| | | GACACAGTCCCCCAGTTCCCTGTCCGCCTCAGTGGGAGACC |
| | | GAGTGACGATTACCTGTCGTGCCAGCCAGGACGTCAATACC |
| | | GCCGTCGCTTGGTATCAGCAAAAACCAGGCAAGGCCCCGAA |
| | | ACTATTGATCTACAGTGCCTCTTTTCTGTACTCCGGGGTGCC |
| | | GAGCAGATTTAGTGGCTCCAGGAGCGGAACCGATTTCACCC |
| | | TAACCATTTCCAGTTTGCAGCCAGAGGATTTCGCGACCTATT |
| | | ACTGCCAGCAACACTACACCACACCGCCAACTTTCGGACAA |
| | | GGAACCAAGGTTGAAATCAAAACTACGACCCCAGCACCTA |
| | | GACCTCCCACCCCAGCTCCAACTATAGCTTCCCAGCCATTGT |
| | | CTCTCCGGCCAGAGGCGTGTCGACCAGCCGCTGGAGGGGCC |
| | | GTTCATACAAGAGGACTCGATTTCGCTTGCGATATCTACAT |
| | | ATGGGCCCCTCTTGCCGGGACATGCGGTGTCCTGCTTCTAA |
| | | GCTTGGTTATTACCCTCTATTGCAAACGCGGCCGCAAGAAA |
| | | CTGCTCTACATCTTTAAACAGCCGTTCATGAGGCCTGTGCAG |
| | | ACAACGCAGGAAGAGGATGGCTGTAGTTGTCGGTTTCCGGA |
| | | AGAGGAAGAGGGGGGCTGCGAGTTGCGTGTCAAATTTTCTC |
| | | GGTCTGCCGACGCCCCCGCGTACCAGCAAGGGCAGAACCA |
| | | GCTTTATAATGAGCTGAATCTTGGACGACGGGAGGAATATG |
| | | ACGTGCTTGACAAGAGGCGAGGTAGGGACCCTGAGATGGG |
| | | GGGAAAACCTCGGAGGAAAAACCCACAGGAAGGCCTGTAT |
| | | AACGAACTGCAGAAGGACAAGATGGCTGAAGCCTACTCTG |
| | | AGATTGGAATGAAAGGGGAACGCAGACGCGGCAAGGGCCA |
| | | TGATGGCCTCTACCAAGGTCTAAGCACTGCCACCAAGGACA |
| | | CCTATGACGCACTCCACATGCAAGCTCTACCTCCCCGTTGAT |
| | | AA |
| MC0289- | SEQ ID | MALPVTALLLPLALLLHAARPQVQLVQSGGGVVQPGGSLRVS |
| 3PF12_28 | NO: 20 | CAASGVTLSDYGMHWVRQAPGKGLEWMAFIRNDGSDKYYA |
| Pdel_HER2 | | DSVKGRFTISRDNSKKTVSLQMSSLRAEDTAVYYCAKNGESGP |
| Protein | | LDYWYFDLWGRGTLVTVSSGGGGSGGGGSGGGGSDIVMTQS |
| Sequence | | PSFLSASVGDRVTITCRASHGINNYLAWYQQKPGKAPKLLIYA |
| (VR289) | | ASTLQSGVPSRFSGSGSGTEFTLTISSLQPEDFATYYCQQYDSY |
| | | PPTFGRGTKVEIKIEVMYPPPYLDNEKSNGTIIHVKGKHLCPSPL |
| | | FPGPSKPFWVLVVVGGVLACYSLLVTVAFIIFWVRRKRGSGEG |
| | | RGSLLTCGDVEENPGPMALPVTALLLPLALLLHAARPEVQLVE |
| | | SGGGLVQPGGSLRLSCAASGFNIKDTYIHWVRQAPGKGLEWV |
| | | ARIYPTNGYTRYADSVKGRFTISADTSKNTAYLQMNSLRAEDT |
| | | AVYYCSRWGGDGFYAMDYWGQGTLVTVSSGSTSGSGKPGSG |
| | | EGSTKGDIQMTQSPSSLSASVGDRVTITCRASQDVNTAVAWY |
| | | QQKPGKAPKLLIYSASFLYSGVPSRESGSRSGTDFTLTISSLQPE |
| | | DFATYYCQQHYTTPPTFGQGTKVEIKTTTPAPRPPTPAPTIASQ |
| | | PLSLRPEACRPAAGGAVHTRGLDFACDIYIWAPLAGTCGVLLL |
| | | SLVITLYCKRGRKKLLYIFKQPFMRPVQTTQEEDGCSCRFPEEE |
| | | EGGCELRVKFSRSADAPAYQQGQNQLYNELNLGRREEYDVLD |
| | | KRRGRDPEMGGKPRRKNPQEGLYNELQKDKMAEAYSEIGMK |
| | | GERRRGKGHDGLYQGLSTATKDTYDALHMQALPPR |
| MC0290- | SEQ ID | ATGGCACTGCCAGTGACCGCCCTGCTGCTGCCTCTGGCCCT |
| 3PF12_28 | NO: 21 | GCTGCTGCACGCAGCCAGACCCCAAGTGCAACTAGTCCAAT |
| LIR1_HER2 | | CAGGTGGAGGCGTCGTGCAACCTGGAGGGTCCCTCCGCGTT |
| Nucleic | | AGCTGCGCCGCATCAGGCGTTACCTTGTCAGACTACGGCAT |
| acid | | GCATTGGGTTAGGCAAGCCCCCGGCAAGGGGCTCGAATGG |
| sequence | | ATGGCTTTCATTCGGAATGACGGGAGCGATAAATATTACGC |
| (VR290) | | GGATTCAGTTAAAGGGCGGTTCACCATCAGCCGCGACAATA |
| | | GCAAAAAGACGGTCTCCTTACAGATGTCCAGCTTGCGGGCC |
| | | GAAGACACGGCTGTATACTATTGTGCTAAAAATGGCGAGAG |
| | | CGGCCCCCTGGATTACTGGTACTTTGACCTGTGGGGCAGAG |
| | | GCACCCTGGTCACGGTGTCCTCTGGAGGAGGAGGCTCCGGA |
| | | GGAGGAGGCTCTGGCGGCGGCGGCAGCGACATTGTAATGA |
| | | CCCAGTCACCCTCCTTCCTTAGTGCCTCAGTCGGAGACCGCG |
| | | TGACTATCACTTGTCGTGCCTCACACGGAATTAATAACTACC |
| | | TCGCTTGGTATCAGCAAAAACCAGGCAAGGCCCCGAAACTA |
| | | TTGATCTACGCCGCATCTACTCTGCAGAGCGGAGTACCGAG |
| | | CAGATTTAGTGGTTCCGGCAGCGGAACCGAGTTCACCCTAA |
| | | CCATTTCCAGTTTGCAGCCAGAGGATTTCGCGACCTATTACT |
| | | GCCAGCAATACGATTCATACCCGCCAACTTTCGGAAGAGGT |
| | | ACCAAGGTTGAAATCAAGATTGAAGTTATGTATCCTCCTCC |
| | | TTACCTAGACAATGAGAAGAGCAATGGAACCATTATCCATG |
| | | TGAAAGGGAAACACCTTTGTCCAAGTCCCCTATTTCCCGGG |
| | | CCTTCGAAGCCCTTTTGGGTGCTGGTGGTGGTTGGTGGAGTC |
| | | CTGGCTTGCTATAGCTTGCTAGTAACAGTAGCGTTTATTATT |
| | | TTCTGGGTGCTGCGCCACAGGAGACAGGGCAAGCACTGGAC |
| | | CAGCACCCAGCGGAAGGCCGACTTTCAGCACCCTGCCGGCG |
| | | CCGTGGGCCCTGAGCCTACCGACAGGGGCCTGCAGTGGAGG |
| | | AGCTCCCCAGCCGCCGATGCCCAGGAGGAGAATCTGTACGC |
| | | CGCCGTGAAGCACACCCAGCCAGAGGACGGCGTGGAGATG |
| | | GACACCCGCTCCCCACACGACGAGGATCCACAGGCCGTGAC |
| | | CTACGCCGAGGTGAAGCACAGCCGCCCCAGACGCGAGATG |
| | | GCCAGCCCACCCAGCCCCCTGTCCGGCGAGTTCCTGGACAC |
| | | CAAGGACAGGCAGGCCGAGGAGGACCGGCAGATGGACACC |
| | | GAGGCCGCCGCCTCCGAGGCCCCCCAGGACGTGACCTACGC |
| | | CCAGCTGCACTCCCTGACCCTGCGGAGAGAGGCCACCGAGC |
| | | CCCCACCCAGCCAGGAGGGCCCCTCCCCCGCCGTGCCTAGC |
| | | ATCTACGCCACCCTGGCCATCCACCGGAGAAAGCGTGGATC |
| | | CGGGGAAGGCCGAGGCTCCCTTCTAACATGTGGAGATGTCG |
| | | AGGAAAACCCTGGCCCTATGGCGCTGCCAGTCACTGCATTG |
| | | TTATTGCCTCTGGCCCTGCTTCTCCATGCGGCGCGCCCAGAA |
| | | GTGCAGCTGGTCGAGAGCGGAGGCGGACTGGTTCAACCCG |
| | | GAGGCAGCTTGAGACTGTCCTGCGCGGCCAGCGGCTTCAAC |
| | | ATCAAGGATACCTATATCCACTGGGTGAGGCAGGCTCCAGG |
| | | AAAGGGCCTGGAGTGGGTGGCAAGGATTTACCCTACTAATG |
| | | GATATACACGCTACGCTGATTCCGTGAAGGGACGCTTTACA |
| | | ATCTCAGCAGATACATCCAAAAACACGGCCTATTTACAGAT |
| | | GAATAGTTTGCGGGCCGAAGACACGGCTGTATACTATTGTT |
| | | CTCGGTGGGGGGGCGATGGATTTTATGCGATGGATTACTGG |
| | | GGCCAGGGCACCCTGGTAACCGTGTCAAGCGGCTCAACATC |
| | | CGGGTCCGGTAAGCCGGGCTCCGGCGAGGGGTCTACAAAG |
| | | GGAGATATACAGATGACACAGTCCCCCAGTTCCCTGTCCGC |
| | | CTCAGTGGGAGACCGAGTGACGATTACCTGTCGTGCCAGCC |
| | | AGGACGTCAATACCGCCGTCGCTTGGTATCAGCAAAAACCA |
| | | GGCAAGGCCCCGAAACTATTGATCTACAGTGCCTCTTTTCTG |
| | | TACTCCGGGGTGCCGAGCAGATTTAGTGGCTCCAGGAGCGG |
| | | AACCGATTTCACCCTAACCATTTCCAGTTTGCAGCCAGAGG |
| | | ATTTCGCGACCTATTACTGCCAGCAACACTACACCACACCG |
| | | CCAACTTTCGGACAAGGAACCAAGGTTGAAATCAAAACTAC |
| | | GACCCCAGCACCTAGACCTCCCACCCCAGCTCCAACTATAG |
| | | CTTCCCAGCCATTGTCTCTCCGGCCAGAGGCGTGTCGACCA |
| | | GCCGCTGGAGGGGCCGTTCATACAAGAGGACTCGATTTCGC |
| | | TTGCGATATCTACATATGGGCCCCTCTTGCCGGGACATGCG |
| | | GTGTCCTGCTTCTAAGCTTGGTTATTACCCTCTATTGCAAAC |
| | | GCGGCCGCAAGAAACTGCTCTACATCTTTAAACAGCCGTTC |
| | | ATGAGGCCTGTGCAGACAACGCAGGAAGAGGATGGCTGTA |
| | | GTTGTCGGTTTCCGGAAGAGGAAGAGGGGGGCTGCGAGTTG |
| | | CGTGTCAAATTTTCTCGGTCTGCCGACGCCCCCGCGTACCAG |
| | | CAAGGGCAGAACCAGCTTTATAATGAGCTGAATCTTGGACG |
| | | ACGGGAGGAATATGACGTGCTTGACAAGAGGCGAGGTAGG |
| | | GACCCTGAGATGGGGGGAAAACCTCGGAGGAAAAACCCAC |
| | | AGGAAGGCCTGTATAACGAACTGCAGAAGGACAAGATGGC |
| | | TGAAGCCTACTCTGAGATTGGAATGAAAGGGGAACGCAGA |
| | | CGCGGCAAGGGCCATGATGGCCTCTACCAAGGTCTAAGCAC |
| | | TGCCACCAAGGACACCTATGACGCACTCCACATGCAAGCTC |
| | | TACCTCCCCGTTGATAA |
| MC0290- | SEQ ID | MALPVTALLLPLALLLHAARPQVQLVQSGGGVVQPGGSLRVS |
| 3PF12_28 | NO: 22 | CAASGVTLSDYGMHWVRQAPGKGLEWMAFIRNDGSDKYYA |
| LIR1_HER2 | | DSVKGRFTISRDNSKKTVSLQMSSLRAEDTAVYYCAKNGESGP |
| Protein | | LDYWYFDLWGRGTLVTVSSGGGGSGGGGSGGGGSDIVMTQS |
| sequence | | PSFLSASVGDRVTITCRASHGINNYLAWYQQKPGKAPKLLIYA |
| (VR290) | | ASTLQSGVPSRESGSGSGTEFTLTISSLQPEDFATYYCQQYDSY |
| | | PPTFGRGTKVEIKIEVMYPPPYLDNEKSNGTIIHVKGKHLCPSPL |
| | | FPGPSKPFWVLVVVGGVLACYSLLVTVAFIIFWVLRHRRQGKH |
| | | WTSTQRKADFQHPAGAVGPEPTDRGLQWRSSPAADAQEENL |
| | | YAAVKHTQPEDGVEMDTRSPHDEDPQAVTYAEVKHSRPRRE |
| | | MASPPSPLSGEFLDTKDRQAEEDRQMDTEAAASEAPQDVTYA |
| | | QLHSLTLRREATEPPPSQEGPSPAVPSIYATLAIHRRKRGSGEGR |
| | | GSLLTCGDVEENPGPMALPVTALLLPLALLLHAARPEVQLVES |
| | | GGGLVQPGGSLRLSCAASGFNIKDTYIHWVRQAPGKGLEWVA |
| | | RIYPTNGYTRYADSVKGRFTISADTSKNTAYLQMNSLRAEDTA |
| | | VYYCSRWGGDGFYAMDYWGQGTLVTVSSGSTSGSGKPGSGE |
| | | GSTKGDIQMTQSPSSLSASVGDRVTITCRASQDVNTAVAWYQ |
| | | QKPGKAPKLLIYSASFLYSGVPSRFSGSRSGTDFTLTISSLQPED |
| | | FATYYCQQHYTTPPTFGQGTKVEIKTTTPAPRPPTPAPTIASQPL |
| | | SLRPEACRPAAGGAVHTRGLDFACDIYIWAPLAGTCGVLLLSL |
| | | VITLYCKRGRKKLLYIFKQPFMRPVQTTQEEDGCSCRFPEEEEG |
| | | GCELRVKFSRSADAPAYQQGQNQLYNELNLGRREEYDVLDKR |
| | | RGRDPEMGGKPRRKNPQEGLYNELQKDKMAEAYSEIGMKGE |
| | | RRRGKGHDGLYQGLSTATKDTYDALHMQALPPR |
| MC0291- | SEQ ID | ATGGCACTGCCAGTGACCGCCCTGCTGCTGCCTCTGGCCCT |
| 3PF12_28_ | NO: 23 | GCTGCTGCACGCAGCCAGACCCCAAGTGCAACTAGTCCAAT |
| KIR2DL1_ | | CAGGTGGAGGCGTCGTGCAACCTGGAGGGTCCCTCCGCGTT |
| HER2 | | AGCTGCGCCGCATCAGGCGTTACCTTGTCAGACTACGGCAT |
| nucleotide | | GCATTGGGTTAGGCAAGCCCCCGGCAAGGGGCTCGAATGG |
| Sequence | | ATGGCTTTCATTCGGAATGACGGGAGCGATAAATATTACGC |
| (VR291) | | GGATTCAGTTAAAGGGCGGTTCACCATCAGCCGCGACAATA |
| | | GCAAAAAGACGGTCTCCTTACAGATGTCCAGCTTGCGGGCC |
| | | GAAGACACGGCTGTATACTATTGTGCTAAAAATGGCGAGAG |
| | | CGGCCCCCTGGATTACTGGTACTTTGACCTGTGGGGCAGAG |
| | | GCACCCTGGTCACGGTGTCCTCTGGAGGAGGAGGCTCCGGA |
| | | GGAGGAGGCTCTGGCGGCGGCGGCAGCGACATTGTAATGA |
| | | CCCAGTCACCCTCCTTCCTTAGTGCCTCAGTCGGAGACCGCG |
| | | TGACTATCACTTGTCGTGCCTCACACGGAATTAATAACTACC |
| | | TCGCTTGGTATCAGCAAAAACCAGGCAAGGCCCCGAAACTA |
| | | TTGATCTACGCCGCATCTACTCTGCAGAGCGGAGTACCGAG |
| | | CAGATTTAGTGGTTCCGGCAGCGGAACCGAGTTCACCCTAA |
| | | CCATTTCCAGTTTGCAGCCAGAGGATTTCGCGACCTATTACT |
| | | GCCAGCAATACGATTCATACCCGCCAACTTTCGGAAGAGGT |
| | | ACCAAGGTTGAAATCAAGATTGAAGTTATGTATCCTCCTCC |
| | | TTACCTAGACAATGAGAAGAGCAATGGAACCATTATCCATG |
| | | TGAAAGGGAAACACCTTTGTCCAAGTCCCCTATTTCCCGGG |
| | | CCTTCGAAGCCCTTTTGGGTGCTGGTGGTGGTTGGTGGAGTC |
| | | CTGGCTTGCTATAGCTTGCTAGTAACAGTAGCGTTTATTATT |
| | | TTCTGGGTGCATAGGTGGTGCTCAAACAAAAAGAATGCTGC |
| | | CGTCATGGACCAGGAGAGCGCGGGCAATCGGACCGCAAAC |
| | | TCAGAGGACTCAGATGAACAAGATCCACAGGAAGTGACCT |
| | | ACACTCAGCTGAACCATTGTGTGTTTACACAGCGCAAGATT |
| | | ACTCGTCCAAGCCAGCGTCCTAAGACCCCCCCGACCGATAT |
| | | CATTGTGTATACCGAGCTTCCTAATGCCGAATCCCGCAGCA |
| | | AGGTGGTCTCCTGCCCGCGGAGAAAGCGTGGATCCGGGGA |
| | | AGGCCGAGGCTCCCTTCTAACATGTGGAGATGTCGAGGAAA |
| | | ACCCTGGCCCTATGGCGCTGCCAGTCACTGCATTGTTATTGC |
| | | CTCTGGCCCTGCTTCTCCATGCGGCGCGCCCAGAAGTGCAG |
| | | CTGGTCGAGAGCGGAGGCGGACTGGTTCAACCCGGAGGCA |
| | | GCTTGAGACTGTCCTGCGCGGCCAGCGGCTTCAACATCAAG |
| | | GATACCTATATCCACTGGGTGAGGCAGGCTCCAGGAAAGGG |
| | | CCTGGAGTGGGTGGCAAGGATTTACCCTACTAATGGATATA |
| | | CACGCTACGCTGATTCCGTGAAGGGACGCTTTACAATCTCA |
| | | GCAGATACATCCAAAAACACGGCCTATTTACAGATGAATAG |
| | | TTTGCGGGCCGAAGACACGGCTGTATACTATTGTTCTCGGT |
| | | GGGGGGGCGATGGATTTTATGCGATGGATTACTGGGGCCAG |
| | | GGCACCCTGGTAACCGTGTCAAGCGGCTCAACATCCGGGTC |
| | | CGGTAAGCCGGGCTCCGGCGAGGGGTCTACAAAGGGAGAT |
| | | ATACAGATGACACAGTCCCCCAGTTCCCTGTCCGCCTCAGT |
| | | GGGAGACCGAGTGACGATTACCTGTCGTGCCAGCCAGGACG |
| | | TCAATACCGCCGTCGCTTGGTATCAGCAAAAACCAGGCAAG |
| | | GCCCCGAAACTATTGATCTACAGTGCCTCTTTTCTGTACTCC |
| | | GGGGTGCCGAGCAGATTTAGTGGCTCCAGGAGCGGAACCG |
| | | ATTTCACCCTAACCATTTCCAGTTTGCAGCCAGAGGATTTCG |
| | | CGACCTATTACTGCCAGCAACACTACACCACACCGCCAACT |
| | | TTCGGACAAGGAACCAAGGTTGAAATCAAAACTACGACCCC |
| | | AGCACCTAGACCTCCCACCCCAGCTCCAACTATAGCTTCCC |
| | | AGCCATTGTCTCTCCGGCCAGAGGCGTGTCGACCAGCCGCT |
| | | GGAGGGGCCGTTCATACAAGAGGACTCGATTTCGCTTGCGA |
| | | TATCTACATATGGGCCCCTCTTGCCGGGACATGCGGTGTCCT |
| | | GCTTCTAAGCTTGGTTATTACCCTCTATTGCAAACGCGGCCG |
| | | CAAGAAACTGCTCTACATCTTTAAACAGCCGTTCATGAGGC |
| | | CTGTGCAGACAACGCAGGAAGAGGATGGCTGTAGTTGTCGG |
| | | TTTCCGGAAGAGGAAGAGGGGGGCTGCGAGTTGCGTGTCA |
| | | AATTTTCTCGGTCTGCCGACGCCCCCGCGTACCAGCAAGGG |
| | | CAGAACCAGCTTTATAATGAGCTGAATCTTGGACGACGGGA |
| | | GGAATATGACGTGCTTGACAAGAGGCGAGGTAGGGACCCT |
| | | GAGATGGGGGGAAAACCTCGGAGGAAAAACCCACAGGAAG |
| | | GCCTGTATAACGAACTGCAGAAGGACAAGATGGCTGAAGC |
| | | CTACTCTGAGATTGGAATGAAAGGGGAACGCAGACGCGGC |
| | | AAGGGCCATGATGGCCTCTACCAAGGTCTAAGCACTGCCAC |
| | | CAAGGACACCTATGACGCACTCCACATGCAAGCTCTACCTC |
| | | CCCGTTGATAA |
| MC0291- | SEQ ID | MALPVTALLLPLALLLHAARPQVQLVQSGGGVVQPGGSLRVS |
| 3PF12_28 | NO: 24 | CAASGVTLSDYGMHWVRQAPGKGLEWMAFIRNDGSDKYYA |
| KIR2DL1_ | | DSVKGRFTISRDNSKKTVSLQMSSLRAEDTAVYYCAKNGESGP |
| HER2 | | LDYWYFDLWGRGTLVTVSSGGGGSGGGGSGGGGSDIVMTQS |
| Protein | | PSFLSASVGDRVTITCRASHGINNYLAWYQQKPGKAPKLLIYA |
| Sequence | | ASTLQSGVPSRFSGSGSGTEFTLTISSLQPEDFATYYCQQYDSY |
| (VR291) | | PPTFGRGTKVEIKIEVMYPPPYLDNEKSNGTIIHVKGKHLCPSPL |
| | | FPGPSKPFWVLVVVGGVLACYSLLVTVAFIIFWVHRWCSNKK |
| | | NAAVMDQESAGNRTANSEDSDEQDPQEVTYTQLNHCVFTQR |
| | | KITRPSQRPKTPPTDIIVYTELPNAESRSKVVSCPRRKRGSGEGR |
| | | GSLLTCGDVEENPGPMALPVTALLLPLALLLHAARPEVQLVES |
| | | GGGLVQPGGSLRLSCAASGFNIKDTYIHWVRQAPGKGLEWVA |
| | | RIYPTNGYTRYADSVKGRFTISADTSKNTAYLQMNSLRAEDTA |
| | | VYYCSRWGGDGFYAMDYWGQGTLVTVSSGSTSGSGKPGSGE |
| | | GSTKGDIQMTQSPSSLSASVGDRVTITCRASQDVNTAVAWYQ |
| | | QKPGKAPKLLIYSASFLYSGVPSRESGSRSGTDFTLTISSLQPED |
| | | FATYYCQQHYTTPPTFGQGTKVEIKTTTPAPRPPTPAPTIASQPL |
| | | SLRPEACRPAAGGAVHTRGLDFACDIYIWAPLAGTCGVLLLSL |
| | | VITLYCKRGRKKLLYIFKQPFMRPVQTTQEEDGCSCRFPEEEEG |
| | | GCELRVKFSRSADAPAYQQGQNQLYNELNLGRREEYDVLDKR |
| | | RGRDPEMGGKPRRKNPQEGLYNELQKDKMAEAYSEIGMKGE |
| | | RRRGKGHDGLYQGLSTATKDTYDALHMQALPPR |
| MC0292- | SEQ ID | ATGGCACTGCCAGTGACCGCCCTGCTGCTGCCTCTGGCCCT |
| BB7.2_28_ | NO: 25 | GCTGCTGCACGCAGCCAGACCCCAGGTGCAGCTGCAGCAGT |
| LIR1_HER2 | | CTGGACCTGAGCTGGTGAAGCCAGGAGCCTCCGTGAAGATG |
| nucleotide | | TCTTGCAAGGCCAGCGGCTACACCTTCACATCTTATCACATC |
| Sequence | | CAGTGGGTGAAGCAGCGGCCCGGACAGGGCCTGGAGTGGA |
| (VR292) | | TCGGATGGATCTACCCAGGCGACGGCTCCACACAGTATAAC |
| | | GAGAAGTTCAAGGGCAAGACCACACTGACCGCCGATAAGA |
| | | GCAGCAGCACCGCCTACATGCTGCTGAGCAGCCTGACCAGC |
| | | GAGGACAGCGCCATCTACTTTTGCGCCAGGGAGGGCACATA |
| | | CTATGCTATGGACTATTGGGGCCAGGGCACCAGCGTGACAG |
| | | TGTCTAGCGGAGGAGGAGGCTCCGGAGGAGGAGGCTCTGG |
| | | CGGCGGCGGCAGCGACGTGCTGATGACCCAGACACCACTG |
| | | AGCCTGCCCGTGAGCCTGGGCGATCAGGTGAGCATCTCCTG |
| | | TAGATCCTCTCAGAGCATCGTGCACTCCAACGGCAATACCT |
| | | ACCTGGAGTGGTATCTGCAGAAGCCAGGCCAGTCCCCCAAG |
| | | CTGCTGATCTATAAGGTGTCTAATCGGTTCAGCGGCGTGCCT |
| | | GACAGATTTTCTGGCAGCGGCTCCGGCACCGACTTCACCCT |
| | | GAAGATCAGCCGGGTGGAGGCAGAGGATCTGGGCGTGTAC |
| | | TATTGTTTCCAGGGCTCCCACGTGCCACGCACCTTTGGCGGC |
| | | GGTACCAAGCTGGAGATCAAGATTGAAGTTATGTATCCTCC |
| | | TCCTTACCTAGACAATGAGAAGAGCAATGGAACCATTATCC |
| | | ATGTGAAAGGGAAACACCTTTGTCCAAGTCCCCTATTTCCC |
| | | GGGCCTTCGAAGCCCTTTTGGGTGCTGGTGGTGGTTGGTGG |
| | | AGTCCTGGCTTGCTATAGCTTGCTAGTAACAGTAGCGTTTAT |
| | | TATTTTCTGGGTGCTGCGCCACAGGAGACAGGGCAAGCACT |
| | | GGACCAGCACCCAGCGGAAGGCCGACTTTCAGCACCCTGCC |
| | | GGCGCCGTGGGCCCTGAGCCTACCGACAGGGGCCTGCAGTG |
| | | GAGGAGCTCCCCAGCCGCCGATGCCCAGGAGGAGAATCTGT |
| | | ACGCCGCCGTGAAGCACACCCAGCCAGAGGACGGCGTGGA |
| | | GATGGACACCCGCTCCCCACACGACGAGGATCCACAGGCCG |
| | | TGACCTACGCCGAGGTGAAGCACAGCCGCCCCAGACGCGA |
| | | GATGGCCAGCCCACCCAGCCCCCTGTCCGGCGAGTTCCTGG |
| | | ACACCAAGGACAGGCAGGCCGAGGAGGACCGGCAGATGGA |
| | | CACCGAGGCCGCCGCCTCCGAGGCCCCCCAGGACGTGACCT |
| | | ACGCCCAGCTGCACTCCCTGACCCTGCGGAGAGAGGCCACC |
| | | GAGCCCCCACCCAGCCAGGAGGGCCCCTCCCCCGCCGTGCC |
| | | TAGCATCTACGCCACCCTGGCCATCCACCGGAGAAAGCGTG |
| | | GATCCGGGGAAGGCCGAGGCTCCCTTCTAACATGTGGAGAT |
| | | GTCGAGGAAAACCCTGGCCCTATGGCGCTGCCAGTCACTGC |
| | | ATTGTTATTGCCTCTGGCCCTGCTTCTCCATGCGGCGCGCCC |
| | | AGAAGTGCAGCTGGTCGAGAGCGGAGGCGGACTGGTTCAA |
| | | CCCGGAGGCAGCTTGAGACTGTCCTGCGCGGCCAGCGGCTT |
| | | CAACATCAAGGATACCTATATCCACTGGGTGAGGCAGGCTC |
| | | CAGGAAAGGGCCTGGAGTGGGTGGCAAGGATTTACCCTACT |
| | | AATGGATATACACGCTACGCTGATTCCGTGAAGGGACGCTT |
| | | TACAATCTCAGCAGATACATCCAAAAACACGGCCTATTTAC |
| | | AGATGAATAGTTTGCGGGCCGAAGACACGGCTGTATACTAT |
| | | TGTTCTCGGTGGGGGGGCGATGGATTTTATGCGATGGATTA |
| | | CTGGGGCCAGGGCACCCTGGTAACCGTGTCAAGCGGCTCAA |
| | | CATCCGGGTCCGGTAAGCCGGGCTCCGGCGAGGGGTCTACA |
| | | AAGGGAGATATACAGATGACACAGTCCCCCAGTTCCCTGTC |
| | | CGCCTCAGTGGGAGACCGAGTGACGATTACCTGTCGTGCCA |
| | | GCCAGGACGTCAATACCGCCGTCGCTTGGTATCAGCAAAAA |
| | | CCAGGCAAGGCCCCGAAACTATTGATCTACAGTGCCTCTTT |
| | | TCTGTACTCCGGGGTGCCGAGCAGATTTAGTGGCTCCAGGA |
| | | GCGGAACCGATTTCACCCTAACCATTTCCAGTTTGCAGCCA |
| | | GAGGATTTCGCGACCTATTACTGCCAGCAACACTACACCAC |
| | | ACCGCCAACTTTCGGACAAGGAACCAAGGTTGAAATCAAA |
| | | ACTACGACCCCAGCACCTAGACCTCCCACCCCAGCTCCAAC |
| | | TATAGCTTCCCAGCCATTGTCTCTCCGGCCAGAGGCGTGTCG |
| | | ACCAGCCGCTGGAGGGGCCGTTCATACAAGAGGACTCGATT |
| | | TCGCTTGCGATATCTACATATGGGCCCCTCTTGCCGGGACAT |
| | | GCGGTGTCCTGCTTCTAAGCTTGGTTATTACCCTCTATTGCA |
| | | AACGCGGCCGCAAGAAACTGCTCTACATCTTTAAACAGCCG |
| | | TTCATGAGGCCTGTGCAGACAACGCAGGAAGAGGATGGCT |
| | | GTAGTTGTCGGTTTCCGGAAGAGGAAGAGGGGGGCTGCGA |
| | | GTTGCGTGTCAAATTTTCTCGGTCTGCCGACGCCCCCGCGTA |
| | | CCAGCAAGGGCAGAACCAGCTTTATAATGAGCTGAATCTTG |
| | | GACGACGGGAGGAATATGACGTGCTTGACAAGAGGCGAGG |
| | | TAGGGACCCTGAGATGGGGGGAAAACCTCGGAGGAAAAAC |
| | | CCACAGGAAGGCCTGTATAACGAACTGCAGAAGGACAAGA |
| | | TGGCTGAAGCCTACTCTGAGATTGGAATGAAAGGGGAACGC |
| | | AGACGCGGCAAGGGCCATGATGGCCTCTACCAAGGTCTAAG |
| | | CACTGCCACCAAGGACACCTATGACGCACTCCACATGCAAG |
| | | CTCTACCTCCCCGTTGATAA |
| MC0292- | SEQ ID | MALPVTALLLPLALLLHAARPQVQLQQSGPELVKPGASVKMS |
| BB7.2_28_ | NO: 26 | CKASGYTFTSYHIQWVKQRPGQGLEWIGWIYPGDGSTQYNEK |
| LIR1_HER2 | | FKGKTTLTADKSSSTAYMLLSSLTSEDSAIYFCAREGTYYAMD |
| Protein | | YWGQGTSVTVSSGGGGSGGGGSGGGGSDVLMTQTPLSLPVSL |
| Sequence | | GDQVSISCRSSQSIVHSNGNTYLEWYLQKPGQSPKLLIYKVSNR |
| (VR292) | | FSGVPDRFSGSGSGTDFTLKISRVEAEDLGVYYCFQGSHVPRTF |
| | | GGGTKLEIKIEVMYPPPYLDNEKSNGTIIHVKGKHLCPSPLFPG |
| | | PSKPFWVLVVVGGVLACYSLLVTVAFIIFWVLRHRRQGKHWT |
| | | STQRKADFQHPAGAVGPEPTDRGLQWRSSPAADAQEENLYAA |
| | | VKHTQPEDGVEMDTRSPHDEDPQAVTYAEVKHSRPRREMASP |
| | | PSPLSGEFLDTKDRQAEEDRQMDTEAAASEAPQDVTYAQLHS |
| | | LTLRREATEPPPSQEGPSPAVPSIYATLAIHRRKRGSGEGRGSLL |
| | | TCGDVEENPGPMALPVTALLLPLALLLHAARPEVQLVESGGGL |
| | | VQPGGSLRLSCAASGFNIKDTYIHWVRQAPGKGLEWVARIYPT |
| | | NGYTRYADSVKGRFTISADTSKNTAYLQMNSLRAEDTAVYYC |
| | | SRWGGDGFYAMDYWGQGTLVTVSSGSTSGSGKPGSGEGSTK |
| | | GDIQMTQSPSSLSASVGDRVTITCRASQDVNTAVAWYQQKPG |
| | | KAPKLLIYSASFLYSGVPSRFSGSRSGTDFTLTISSLQPEDFATY |
| | | YCQQHYTTPPTFGQGTKVEIKTTTPAPRPPTPAPTIASQPLSLRP |
| | | EACRPAAGGAVHTRGLDFACDIYIWAPLAGTCGVLLLSLVITL |
| | | YCKRGRKKLLYIFKQPFMRPVQTTQEEDGCSCRFPEEEEGGCE |
| | | LRVKFSRSADAPAYQQGQNQLYNELNLGRREEYDVLDKRRG |
| | | RDPEMGGKPRRKNPQEGLYNELQKDKMAEAYSEIGMKGERR |
| | | RGKGHDGLYQGLSTATKDTYDALHMQALPPR |
| MC0293- | SEQ ID | ATGGCACTGCCAGTGACCGCCCTGCTGCTGCCTCTGGCCCT |
| BB7.2_28_ | NO: 27 | GCTGCTGCACGCAGCCAGACCCCAGGTGCAGCTGCAGCAGT |
| KIR2DL1 | | CTGGACCTGAGCTGGTGAAGCCAGGAGCCTCCGTGAAGATG |
| HER2 | | TCTTGCAAGGCCAGCGGCTACACCTTCACATCTTATCACATC |
| nucleotide | | CAGTGGGTGAAGCAGCGGCCCGGACAGGGCCTGGAGTGGA |
| sequence | | TCGGATGGATCTACCCAGGCGACGGCTCCACACAGTATAAC |
| (VR293) | | GAGAAGTTCAAGGGCAAGACCACACTGACCGCCGATAAGA |
| | | GCAGCAGCACCGCCTACATGCTGCTGAGCAGCCTGACCAGC |
| | | GAGGACAGCGCCATCTACTTTTGCGCCAGGGAGGGCACATA |
| | | CTATGCTATGGACTATTGGGGCCAGGGCACCAGCGTGACAG |
| | | TGTCTAGCGGAGGAGGAGGCTCCGGAGGAGGAGGCTCTGG |
| | | CGGCGGCGGCAGCGACGTGCTGATGACCCAGACACCACTG |
| | | AGCCTGCCCGTGAGCCTGGGCGATCAGGTGAGCATCTCCTG |
| | | TAGATCCTCTCAGAGCATCGTGCACTCCAACGGCAATACCT |
| | | ACCTGGAGTGGTATCTGCAGAAGCCAGGCCAGTCCCCCAAG |
| | | CTGCTGATCTATAAGGTGTCTAATCGGTTCAGCGGCGTGCCT |
| | | GACAGATTTTCTGGCAGCGGCTCCGGCACCGACTTCACCCT |
| | | GAAGATCAGCCGGGTGGAGGCAGAGGATCTGGGCGTGTAC |
| | | TATTGTTTCCAGGGCTCCCACGTGCCACGCACCTTTGGCGGC |
| | | GGTACCAAGCTGGAGATCAAGATTGAAGTTATGTATCCTCC |
| | | TCCTTACCTAGACAATGAGAAGAGCAATGGAACCATTATCC |
| | | ATGTGAAAGGGAAACACCTTTGTCCAAGTCCCCTATTTCCC |
| | | GGGCCTTCGAAGCCCTTTTGGGTGCTGGTGGTGGTTGGTGG |
| | | AGTCCTGGCTTGCTATAGCTTGCTAGTAACAGTAGCGTTTAT |
| | | TATTTTCTGGGTGCATAGGTGGTGCTCAAACAAAAAGAATG |
| | | CTGCCGTCATGGACCAGGAGAGCGCGGGCAATCGGACCGC |
| | | AAACTCAGAGGACTCAGATGAACAAGATCCACAGGAAGTG |
| | | ACCTACACTCAGCTGAACCATTGTGTGTTTACACAGCGCAA |
| | | GATTACTCGTCCAAGCCAGCGTCCTAAGACCCCCCCGACCG |
| | | ATATCATTGTGTATACCGAGCTTCCTAATGCCGAATCCCGCA |
| | | GCAAGGTGGTCTCCTGCCCGCGGAGAAAGCGTGGATCCGGG |
| | | GAAGGCCGAGGCTCCCTTCTAACATGTGGAGATGTCGAGGA |
| | | AAACCCTGGCCCTATGGCGCTGCCAGTCACTGCATTGTTATT |
| | | GCCTCTGGCCCTGCTTCTCCATGCGGCGCGCCCAGAAGTGC |
| | | AGCTGGTCGAGAGCGGAGGCGGACTGGTTCAACCCGGAGG |
| | | CAGCTTGAGACTGTCCTGCGCGGCCAGCGGCTTCAACATCA |
| | | AGGATACCTATATCCACTGGGTGAGGCAGGCTCCAGGAAAG |
| | | GGCCTGGAGTGGGTGGCAAGGATTTACCCTACTAATGGATA |
| | | TACACGCTACGCTGATTCCGTGAAGGGACGCTTTACAATCT |
| | | CAGCAGATACATCCAAAAACACGGCCTATTTACAGATGAAT |
| | | AGTTTGCGGGCCGAAGACACGGCTGTATACTATTGTTCTCG |
| | | GTGGGGGGGCGATGGATTTTATGCGATGGATTACTGGGGCC |
| | | AGGGCACCCTGGTAACCGTGTCAAGCGGCTCAACATCCGGG |
| | | TCCGGTAAGCCGGGCTCCGGCGAGGGGTCTACAAAGGGAG |
| | | ATATACAGATGACACAGTCCCCCAGTTCCCTGTCCGCCTCA |
| | | GTGGGAGACCGAGTGACGATTACCTGTCGTGCCAGCCAGGA |
| | | CGTCAATACCGCCGTCGCTTGGTATCAGCAAAAACCAGGCA |
| | | AGGCCCCGAAACTATTGATCTACAGTGCCTCTTTTCTGTACT |
| | | CCGGGGTGCCGAGCAGATTTAGTGGCTCCAGGAGCGGAACC |
| | | GATTTCACCCTAACCATTTCCAGTTTGCAGCCAGAGGATTTC |
| | | GCGACCTATTACTGCCAGCAACACTACACCACACCGCCAAC |
| | | TTTCGGACAAGGAACCAAGGTTGAAATCAAAACTACGACCC |
| | | CAGCACCTAGACCTCCCACCCCAGCTCCAACTATAGCTTCC |
| | | CAGCCATTGTCTCTCCGGCCAGAGGCGTGTCGACCAGCCGC |
| | | TGGAGGGGCCGTTCATACAAGAGGACTCGATTTCGCTTGCG |
| | | ATATCTACATATGGGCCCCTCTTGCCGGGACATGCGGTGTC |
| | | CTGCTTCTAAGCTTGGTTATTACCCTCTATTGCAAACGCGGC |
| | | CGCAAGAAACTGCTCTACATCTTTAAACAGCCGTTCATGAG |
| | | GCCTGTGCAGACAACGCAGGAAGAGGATGGCTGTAGTTGTC |
| | | GGTTTCCGGAAGAGGAAGAGGGGGGCTGCGAGTTGCGTGT |
| | | CAAATTTTCTCGGTCTGCCGACGCCCCCGCGTACCAGCAAG |
| | | GGCAGAACCAGCTTTATAATGAGCTGAATCTTGGACGACGG |
| | | GAGGAATATGACGTGCTTGACAAGAGGCGAGGTAGGGACC |
| | | CTGAGATGGGGGGAAAACCTCGGAGGAAAAACCCACAGGA |
| | | AGGCCTGTATAACGAACTGCAGAAGGACAAGATGGCTGAA |
| | | GCCTACTCTGAGATTGGAATGAAAGGGGAACGCAGACGCG |
| | | GCAAGGGCCATGATGGCCTCTACCAAGGTCTAAGCACTGCC |
| | | ACCAAGGACACCTATGACGCACTCCACATGCAAGCTCTACC |
| | | TCCCCGTTGATAA |
| MC0293- | SEQ ID | MALPVTALLLPLALLLHAARPQVQLQQSGPELVKPGASVKMS |
| BB7.2_28_ | NO: 28 | CKASGYTFTSYHIQWVKQRPGQGLEWIGWIYPGDGSTQYNEK |
| KIR2DL1_ | | FKGKTTLTADKSSSTAYMLLSSLTSEDSAIYFCAREGTYYAMD |
| HER2 | | YWGQGTSVTVSSGGGGSGGGGSGGGGSDVLMTQTPLSLPVSL |
| Protein | | GDQVSISCRSSQSIVHSNGNTYLEWYLQKPGQSPKLLIYKVSNR |
| Sequence | | FSGVPDRFSGSGSGTDFTLKISRVEAEDLGVYYCFQGSHVPRTF |
| (VR293) | | GGGTKLEIKIEVMYPPPYLDNEKSNGTIIHVKGKHLCPSPLFPG |
| | | PSKPFWVLVVVGGVLACYSLLVTVAFIIFWVHRWCSNKKNAA |
| | | VMDQESAGNRTANSEDSDEQDPQEVTYTQLNHCVFTQRKITR |
| | | PSQRPKTPPTDIIVYTELPNAESRSKVVSCPRRKRGSGEGRGSLL |
| | | TCGDVEENPGPMALPVTALLLPLALLLHAARPEVQLVESGGGL |
| | | VQPGGSLRLSCAASGFNIKDTYIHWVRQAPGKGLEWVARIYPT |
| | | NGYTRYADSVKGRFTISADTSKNTAYLQMNSLRAEDTAVYYC |
| | | SRWGGDGFYAMDYWGQGTLVTVSSGSTSGSGKPGSGEGSTK |
| | | GDIQMTQSPSSLSASVGDRVTITCRASQDVNTAVAWYQQKPG |
| | | KAPKLLIYSASFLYSGVPSRESGSRSGTDFTLTISSLQPEDFATY |
| | | YCQQHYTTPPTFGQGTKVEIKTTTPAPRPPTPAPTIASQPLSLRP |
| | | EACRPAAGGAVHTRGLDFACDIYIWAPLAGTCGVLLLSLVITL |
| | | YCKRGRKKLLYIFKQPFMRPVQTTQEEDGCSCRFPEEEEGGCE |
| | | LRVKFSRSADAPAYQQGQNQLYNELNLGRREEYDVLDKRRG |
| | | RDPEMGGKPRRKNPQEGLYNELQKDKMAEAYSEIGMKGERR |
| | | RGKGHDGLYQGLSTATKDTYDALHMQALPPR |
| MC0294- | SEQ ID | ATGGCACTGCCAGTGACCGCCCTGCTGCTGCCTCTGGCCCT |
| 3PF12_CD8_ | NO: 29 | GCTGCTGCACGCAGCCAGACCCCAAGTGCAACTAGTCCAAT |
| LIR1_HER2 | | CAGGTGGAGGCGTCGTGCAACCTGGAGGGTCCCTCCGCGTT |
| Nucleotide | | AGCTGCGCCGCATCAGGCGTTACCTTGTCAGACTACGGCAT |
| sequence | | GCATTGGGTTAGGCAAGCCCCCGGCAAGGGGCTCGAATGG |
| (VR294) | | ATGGCTTTCATTCGGAATGACGGGAGCGATAAATATTACGC |
| | | GGATTCAGTTAAAGGGCGGTTCACCATCAGCCGCGACAATA |
| | | GCAAAAAGACGGTCTCCTTACAGATGTCCAGCTTGCGGGCC |
| | | GAAGACACGGCTGTATACTATTGTGCTAAAAATGGCGAGAG |
| | | CGGCCCCCTGGATTACTGGTACTTTGACCTGTGGGGCAGAG |
| | | GCACCCTGGTCACGGTGTCCTCTGGAGGAGGAGGCTCCGGA |
| | | GGAGGAGGCTCTGGCGGCGGCGGCAGCGACATTGTAATGA |
| | | CCCAGTCACCCTCCTTCCTTAGTGCCTCAGTCGGAGACCGCG |
| | | TGACTATCACTTGTCGTGCCTCACACGGAATTAATAACTACC |
| | | TCGCTTGGTATCAGCAAAAACCAGGCAAGGCCCCGAAACTA |
| | | TTGATCTACGCCGCATCTACTCTGCAGAGCGGAGTACCGAG |
| | | CAGATTTAGTGGTTCCGGCAGCGGAACCGAGTTCACCCTAA |
| | | CCATTTCCAGTTTGCAGCCAGAGGATTTCGCGACCTATTACT |
| | | GCCAGCAATACGATTCATACCCGCCAACTTTCGGAAGAGGT |
| | | ACCAAGGTTGAAATCAAGACTACGACCCCAGCACCTAGACC |
| | | TCCCACCCCAGCTCCAACTATAGCTTCCCAGCCATTGTCTCT |
| | | CCGGCCAGAGGCGTGTCGACCAGCCGCTGGAGGGGCCGTTC |
| | | ATACAAGAGGACTCGATTTCGCTTGCGATATCTACATATGG |
| | | GCCCCTCTTGCCGGGACATGCGGTGTCCTGCTTCTAAGCTTG |
| | | GTTATTACCCTCTATTGCCTGCGCCACAGGAGACAGGGCAA |
| | | GCACTGGACCAGCACCCAGCGGAAGGCCGACTTTCAGCACC |
| | | CTGCCGGCGCCGTGGGCCCTGAGCCTACCGACAGGGGCCTG |
| | | CAGTGGAGGAGCTCCCCAGCCGCCGATGCCCAGGAGGAGA |
| | | ATCTGTACGCCGCCGTGAAGCACACCCAGCCAGAGGACGGC |
| | | GTGGAGATGGACACCCGCTCCCCACACGACGAGGATCCACA |
| | | GGCCGTGACCTACGCCGAGGTGAAGCACAGCCGCCCCAGA |
| | | CGCGAGATGGCCAGCCCACCCAGCCCCCTGTCCGGCGAGTT |
| | | CCTGGACACCAAGGACAGGCAGGCCGAGGAGGACCGGCAG |
| | | ATGGACACCGAGGCCGCCGCCTCCGAGGCCCCCCAGGACGT |
| | | GACCTACGCCCAGCTGCACTCCCTGACCCTGCGGAGAGAGG |
| | | CCACCGAGCCCCCACCCAGCCAGGAGGGCCCCTCCCCCGCC |
| | | GTGCCTAGCATCTACGCCACCCTGGCCATCCACCGGAGAAA |
| | | GCGTGGATCCGGGGAAGGCCGAGGCTCCCTTCTAACATGTG |
| | | GAGATGTCGAGGAAAACCCTGGCCCTATGGCGCTGCCAGTC |
| | | ACTGCATTGTTATTGCCTCTGGCCCTGCTTCTCCATGCGGCG |
| | | CGCCCAGAAGTGCAGCTGGTCGAGAGCGGAGGCGGACTGG |
| | | TTCAACCCGGAGGCAGCTTGAGACTGTCCTGCGCGGCCAGC |
| | | GGCTTCAACATCAAGGATACCTATATCCACTGGGTGAGGCA |
| | | GGCTCCAGGAAAGGGCCTGGAGTGGGTGGCAAGGATTTAC |
| | | CCTACTAATGGATATACACGCTACGCTGATTCCGTGAAGGG |
| | | ACGCTTTACAATCTCAGCAGATACATCCAAAAACACGGCCT |
| | | ATTTACAGATGAATAGTTTGCGGGCCGAAGACACGGCTGTA |
| | | TACTATTGTTCTCGGTGGGGGGGCGATGGATTTTATGCGAT |
| | | GGATTACTGGGGCCAGGGCACCCTGGTAACCGTGTCAAGCG |
| | | GCTCAACATCCGGGTCCGGTAAGCCGGGCTCCGGCGAGGGG |
| | | TCTACAAAGGGAGATATACAGATGACACAGTCCCCCAGTTC |
| | | CCTGTCCGCCTCAGTGGGAGACCGAGTGACGATTACCTGTC |
| | | GTGCCAGCCAGGACGTCAATACCGCCGTCGCTTGGTATCAG |
| | | CAAAAACCAGGCAAGGCCCCGAAACTATTGATCTACAGTGC |
| | | CTCTTTTCTGTACTCCGGGGTGCCGAGCAGATTTAGTGGCTC |
| | | CAGGAGCGGAACCGATTTCACCCTAACCATTTCCAGTTTGC |
| | | AGCCAGAGGATTTCGCGACCTATTACTGCCAGCAACACTAC |
| | | ACCACACCGCCAACTTTCGGACAAGGAACCAAGGTTGAAAT |
| | | CAAAATTGAAGTTATGTATCCTCCTCCTTACCTAGACAATGA |
| | | GAAGAGCAATGGAACCATTATCCATGTGAAAGGGAAACAC |
| | | CTTTGTCCAAGTCCCCTATTTCCCGGGCCTTCGAAGCCCTTT |
| | | TGGGTGCTGGTGGTGGTTGGTGGAGTCCTGGCTTGCTATAG |
| | | CTTGCTAGTAACAGTAGCGTTTATTATTTTCTGGGTGAAACG |
| | | CGGCCGCAAGAAACTGCTCTACATCTTTAAACAGCCGTTCA |
| | | TGAGGCCTGTGCAGACAACGCAGGAAGAGGATGGCTGTAG |
| | | TTGTCGGTTTCCGGAAGAGGAAGAGGGGGGCTGCGAGTTGC |
| | | GTGTCAAATTTTCTCGGTCTGCCGACGCCCCCGCGTACCAGC |
| | | AAGGGCAGAACCAGCTTTATAATGAGCTGAATCTTGGACGA |
| | | CGGGAGGAATATGACGTGCTTGACAAGAGGCGAGGTAGGG |
| | | ACCCTGAGATGGGGGGAAAACCTCGGAGGAAAAACCCACA |
| | | GGAAGGCCTGTATAACGAACTGCAGAAGGACAAGATGGCT |
| | | GAAGCCTACTCTGAGATTGGAATGAAAGGGGAACGCAGAC |
| | | GCGGCAAGGGCCATGATGGCCTCTACCAAGGTCTAAGCACT |
| | | GCCACCAAGGACACCTATGACGCACTCCACATGCAAGCTCT |
| | | ACCTCCCCGTTGATAA |
| MC0294- | SEQ ID | MALPVTALLLPLALLLHAARPQVQLVQSGGGVVQPGGSLRVS |
| 3PF12_CD8_ | NO: 30 | CAASGVTLSDYGMHWVRQAPGKGLEWMAFIRNDGSDKYYA |
| LIR1_HER2 | | DSVKGRFTISRDNSKKTVSLQMSSLRAEDTAVYYCAKNGESGP |
| Protein | | LDYWYFDLWGRGTLVTVSSGGGGSGGGGSGGGGSDIVMTQS |
| Sequence | | PSFLSASVGDRVTITCRASHGINNYLAWYQQKPGKAPKLLIYA |
| (VR294) | | ASTLQSGVPSRFSGSGSGTEFTLTISSLQPEDFATYYCQQYDSY |
| | | PPTFGRGTKVEIKTTTPAPRPPTPAPTIASQPLSLRPEACRPAAG |
| | | GAVHTRGLDFACDIYIWAPLAGTCGVLLLSLVITLYCLRHRRQ |
| | | GKHWTSTQRKADFQHPAGAVGPEPTDRGLQWRSSPAADAQE |
| | | ENLYAAVKHTQPEDGVEMDTRSPHDEDPQAVTYAEVKHSRPR |
| | | REMASPPSPLSGEFLDTKDRQAEEDRQMDTEAAASEAPQDVT |
| | | YAQLHSLTLRREATEPPPSQEGPSPAVPSIYATLAIHRRKRGSG |
| | | EGRGSLLTCGDVEENPGPMALPVTALLLPLALLLHAARPEVQL |
| | | VESGGGLVQPGGSLRLSCAASGFNIKDTYIHWVRQAPGKGLE |
| | | WVARIYPTNGYTRYADSVKGRFTISADTSKNTAYLQMNSLRA |
| | | EDTAVYYCSRWGGDGFYAMDYWGQGTLVTVSSGSTSGSGKP |
| | | GSGEGSTKGDIQMTQSPSSLSASVGDRVTITCRASQDVNTAVA |
| | | WYQQKPGKAPKLLIYSASFLYSGVPSRFSGSRSGTDFTLTISSL |
| | | QPEDFATYYCQQHYTTPPTFGQGTKVEIKIEVMYPPPYLDNEK |
| | | SNGTIIHVKGKHLCPSPLFPGPSKPFWVLVVVGGVLACYSLLV |
| | | TVAFIIFWVKRGRKKLLYIFKQPFMRPVQTTQEEDGCSCRFPEE |
| | | EEGGCELRVKFSRSADAPAYQQGQNQLYNELNLGRREEYDVL |
| | | DKRRGRDPEMGGKPRRKNPQEGLYNELQKDKMAEAYSEIGM |
| | | KGERRRGKGHDGLYQGLSTATKDTYDALHMQALPPR |
| MC0295- | SEQ ID | ATGGCACTGCCAGTGACCGCCCTGCTGCTGCCTCTGGCCCT |
| 3PF12_CD8_ | NO: 31 | GCTGCTGCACGCAGCCAGACCCCAAGTGCAACTAGTCCAAT |
| KIR2DL1_ | | CAGGTGGAGGCGTCGTGCAACCTGGAGGGTCCCTCCGCGTT |
| HER2 | | AGCTGCGCCGCATCAGGCGTTACCTTGTCAGACTACGGCAT |
| nucleotide | | GCATTGGGTTAGGCAAGCCCCCGGCAAGGGGCTCGAATGG |
| Sequence | | ATGGCTTTCATTCGGAATGACGGGAGCGATAAATATTACGC |
| (VR295) | | GGATTCAGTTAAAGGGCGGTTCACCATCAGCCGCGACAATA |
| | | GCAAAAAGACGGTCTCCTTACAGATGTCCAGCTTGCGGGCC |
| | | GAAGACACGGCTGTATACTATTGTGCTAAAAATGGCGAGAG |
| | | CGGCCCCCTGGATTACTGGTACTTTGACCTGTGGGGCAGAG |
| | | GCACCCTGGTCACGGTGTCCTCTGGAGGAGGAGGCTCCGGA |
| | | GGAGGAGGCTCTGGCGGCGGCGGCAGCGACATTGTAATGA |
| | | CCCAGTCACCCTCCTTCCTTAGTGCCTCAGTCGGAGACCGCG |
| | | TGACTATCACTTGTCGTGCCTCACACGGAATTAATAACTACC |
| | | TCGCTTGGTATCAGCAAAAACCAGGCAAGGCCCCGAAACTA |
| | | TTGATCTACGCCGCATCTACTCTGCAGAGCGGAGTACCGAG |
| | | CAGATTTAGTGGTTCCGGCAGCGGAACCGAGTTCACCCTAA |
| | | CCATTTCCAGTTTGCAGCCAGAGGATTTCGCGACCTATTACT |
| | | GCCAGCAATACGATTCATACCCGCCAACTTTCGGAAGAGGT |
| | | ACCAAGGTTGAAATCAAGACTACGACCCCAGCACCTAGACC |
| | | TCCCACCCCAGCTCCAACTATAGCTTCCCAGCCATTGTCTCT |
| | | CCGGCCAGAGGCGTGTCGACCAGCCGCTGGAGGGGCCGTTC |
| | | ATACAAGAGGACTCGATTTCGCTTGCGATATCTACATATGG |
| | | GCCCCTCTTGCCGGGACATGCGGTGTCCTGCTTCTAAGCTTG |
| | | GTTATTACCCTCTATTGCCATAGGTGGTGCTCAAACAAAAA |
| | | GAATGCTGCCGTCATGGACCAGGAGAGCGCGGGCAATCGG |
| | | ACCGCAAACTCAGAGGACTCAGATGAACAAGATCCACAGG |
| | | AAGTGACCTACACTCAGCTGAACCATTGTGTGTTTACACAG |
| | | CGCAAGATTACTCGTCCAAGCCAGCGTCCTAAGACCCCCCC |
| | | GACCGATATCATTGTGTATACCGAGCTTCCTAATGCCGAAT |
| | | CCCGCAGCAAGGTGGTCTCCTGCCCGCGGAGAAAGCGTGGA |
| | | TCCGGGGAAGGCCGAGGCTCCCTTCTAACATGTGGAGATGT |
| | | CGAGGAAAACCCTGGCCCTATGGCGCTGCCAGTCACTGCAT |
| | | TGTTATTGCCTCTGGCCCTGCTTCTCCATGCGGCGCGCCCAG |
| | | AAGTGCAGCTGGTCGAGAGCGGAGGCGGACTGGTTCAACC |
| | | CGGAGGCAGCTTGAGACTGTCCTGCGCGGCCAGCGGCTTCA |
| | | ACATCAAGGATACCTATATCCACTGGGTGAGGCAGGCTCCA |
| | | GGAAAGGGCCTGGAGTGGGTGGCAAGGATTTACCCTACTAA |
| | | TGGATATACACGCTACGCTGATTCCGTGAAGGGACGCTTTA |
| | | CAATCTCAGCAGATACATCCAAAAACACGGCCTATTTACAG |
| | | ATGAATAGTTTGCGGGCCGAAGACACGGCTGTATACTATTG |
| | | TTCTCGGTGGGGGGGCGATGGATTTTATGCGATGGATTACT |
| | | GGGGCCAGGGCACCCTGGTAACCGTGTCAAGCGGCTCAACA |
| | | TCCGGGTCCGGTAAGCCGGGCTCCGGCGAGGGGTCTACAAA |
| | | GGGAGATATACAGATGACACAGTCCCCCAGTTCCCTGTCCG |
| | | CCTCAGTGGGAGACCGAGTGACGATTACCTGTCGTGCCAGC |
| | | CAGGACGTCAATACCGCCGTCGCTTGGTATCAGCAAAAACC |
| | | AGGCAAGGCCCCGAAACTATTGATCTACAGTGCCTCTTTTCT |
| | | GTACTCCGGGGTGCCGAGCAGATTTAGTGGCTCCAGGAGCG |
| | | GAACCGATTTCACCCTAACCATTTCCAGTTTGCAGCCAGAG |
| | | GATTTCGCGACCTATTACTGCCAGCAACACTACACCACACC |
| | | GCCAACTTTCGGACAAGGAACCAAGGTTGAAATCAAAATTG |
| | | AAGTTATGTATCCTCCTCCTTACCTAGACAATGAGAAGAGC |
| | | AATGGAACCATTATCCATGTGAAAGGGAAACACCTTTGTCC |
| | | AAGTCCCCTATTTCCCGGGCCTTCGAAGCCCTTTTGGGTGCT |
| | | GGTGGTGGTTGGTGGAGTCCTGGCTTGCTATAGCTTGCTAGT |
| | | AACAGTAGCGTTTATTATTTTCTGGGTGAAACGCGGCCGCA |
| | | AGAAACTGCTCTACATCTTTAAACAGCCGTTCATGAGGCCT |
| | | GTGCAGACAACGCAGGAAGAGGATGGCTGTAGTTGTCGGTT |
| | | TCCGGAAGAGGAAGAGGGGGGCTGCGAGTTGCGTGTCAAA |
| | | TTTTCTCGGTCTGCCGACGCCCCCGCGTACCAGCAAGGGCA |
| | | GAACCAGCTTTATAATGAGCTGAATCTTGGACGACGGGAGG |
| | | AATATGACGTGCTTGACAAGAGGCGAGGTAGGGACCCTGA |
| | | GATGGGGGGAAAACCTCGGAGGAAAAACCCACAGGAAGGC |
| | | CTGTATAACGAACTGCAGAAGGACAAGATGGCTGAAGCCT |
| | | ACTCTGAGATTGGAATGAAAGGGGAACGCAGACGCGGCAA |
| | | GGGCCATGATGGCCTCTACCAAGGTCTAAGCACTGCCACCA |
| | | AGGACACCTATGACGCACTCCACATGCAAGCTCTACCTCCC |
| | | CGTTGATAA |
| MC0295- | SEQ ID | MALPVTALLLPLALLLHAARPQVQLVQSGGGVVQPGGSLRVS |
| 3PF12_CD8 | NO: 32 | CAASGVTLSDYGMHWVRQAPGKGLEWMAFIRNDGSDKYYA |
| KIR2DL1_ | | DSVKGRFTISRDNSKKTVSLQMSSLRAEDTAVYYCAKNGESGP |
| HER2 | | LDYWYFDLWGRGTLVTVSSGGGGSGGGGSGGGGSDIVMTQS |
| Protein | | PSFLSASVGDRVTITCRASHGINNYLAWYQQKPGKAPKLLIYA |
| Sequence | | ASTLQSGVPSRFSGSGSGTEFTLTISSLQPEDFATYYCQQYDSY |
| (VR295) | | PPTFGRGTKVEIKTTTPAPRPPTPAPTIASQPLSLRPEACRPAAG |
| | | GAVHTRGLDFACDIYIWAPLAGTCGVLLLSLVITLYCHRWCSN |
| | | KKNAAVMDQESAGNRTANSEDSDEQDPQEVTYTQLNHCVFT |
| | | QRKITRPSQRPKTPPTDIIVYTELPNAESRSKVVSCPRRKRGSGE |
| | | GRGSLLTCGDVEENPGPMALPVTALLLPLALLLHAARPEVQLV |
| | | ESGGGLVQPGGSLRLSCAASGFNIKDTYIHWVRQAPGKGLEW |
| | | VARIYPTNGYTRYADSVKGRFTISADTSKNTAYLQMNSLRAED |
| | | TAVYYCSRWGGDGFYAMDYWGQGTLVTVSSGSTSGSGKPGS |
| | | GEGSTKGDIQMTQSPSSLSASVGDRVTITCRASQDVNTAVAW |
| | | YQQKPGKAPKLLIYSASFLYSGVPSRFSGSRSGTDFTLTISSLQP |
| | | EDFATYYCQQHYTTPPTFGQGTKVEIKIEVMYPPPYLDNEKSN |
| | | GTIIHVKGKHLCPSPLFPGPSKPFWVLVVVGGVLACYSLLVTV |
| | | AFIIFWVKRGRKKLLYIFKQPFMRPVQTTQEEDGCSCRFPEEEE |
| | | GGCELRVKFSRSADAPAYQQGQNQLYNELNLGRREEYDVLD |
| | | KRRGRDPEMGGKPRRKNPQEGLYNELQKDKMAEAYSEIGMK |
| | | GERRRGKGHDGLYQGLSTATKDTYDALHMQALPPR |
| | | ATGGCACTGCCAGTGACCGCCCTGCTGCTGCCTCTGGCCCT |
| | | GCTGCTGCACGCAGCCAGACCCCAGGTGCAGCTGCAGCAGT |
| | | CTGGACCTGAGCTGGTGAAGCCAGGAGCCTCCGTGAAGATG |
| | | TCTTGCAAGGCCAGCGGCTACACCTTCACATCTTATCACATC |
| | | CAGTGGGTGAAGCAGCGGCCCGGACAGGGCCTGGAGTGGA |
| | | TCGGATGGATCTACCCAGGCGACGGCTCCACACAGTATAAC |
| | | GAGAAGTTCAAGGGCAAGACCACACTGACCGCCGATAAGA |
| | | GCAGCAGCACCGCCTACATGCTGCTGAGCAGCCTGACCAGC |
| | | GAGGACAGCGCCATCTACTTTTGCGCCAGGGAGGGCACATA |
| | | CTATGCTATGGACTATTGGGGCCAGGGCACCAGCGTGACAG |
| MC00296- | SEQ ID | TGTCTAGCGGAGGAGGAGGCTCCGGAGGAGGAGGCTCTGG |
| BB7.2_CD8_ | NO: 33 | CGGCGGCGGCAGCGACGTGCTGATGACCCAGACACCACTG |
| LIR1_HER2 | | AGCCTGCCCGTGAGCCTGGGCGATCAGGTGAGCATCTCCTG |
| (VR296) | | TAGATCCTCTCAGAGCATCGTGCACTCCAACGGCAATACCT |
| | | ACCTGGAGTGGTATCTGCAGAAGCCAGGCCAGTCCCCCAAG |
| | | CTGCTGATCTATAAGGTGTCTAATCGGTTCAGCGGCGTGCCT |
| | | GACAGATTTTCTGGCAGCGGCTCCGGCACCGACTTCACCCT |
| | | GAAGATCAGCCGGGTGGAGGCAGAGGATCTGGGCGTGTAC |
| | | TATTGTTTCCAGGGCTCCCACGTGCCACGCACCTTTGGCGGC |
| | | GGTACCAAGCTGGAGATCAAGACTACGACCCCAGCACCTAG |
| | | ACCTCCCACCCCAGCTCCAACTATAGCTTCCCAGCCATTGTC |
| | | TCTCCGGCCAGAGGCGTGTCGACCAGCCGCTGGAGGGGCCG |
| | | TTCATACAAGAGGACTCGATTTCGCTTGCGATATCTACATAT |
| | | GGGCCCCTCTTGCCGGGACATGCGGTGTCCTGCTTCTAAGCT |
| | | TGGTTATTACCCTCTATTGCCTGCGCCACAGGAGACAGGGC |
| | | AAGCACTGGACCAGCACCCAGCGGAAGGCCGACTTTCAGC |
| | | ACCCTGCCGGCGCCGTGGGCCCTGAGCCTACCGACAGGGGC |
| | | CTGCAGTGGAGGAGCTCCCCAGCCGCCGATGCCCAGGAGG |
| | | AGAATCTGTACGCCGCCGTGAAGCACACCCAGCCAGAGGA |
| | | CGGCGTGGAGATGGACACCCGCTCCCCACACGACGAGGATC |
| | | CACAGGCCGTGACCTACGCCGAGGTGAAGCACAGCCGCCCC |
| | | AGACGCGAGATGGCCAGCCCACCCAGCCCCCTGTCCGGCGA |
| | | GTTCCTGGACACCAAGGACAGGCAGGCCGAGGAGGACCGG |
| | | CAGATGGACACCGAGGCCGCCGCCTCCGAGGCCCCCCAGG |
| | | ACGTGACCTACGCCCAGCTGCACTCCCTGACCCTGCGGAGA |
| | | GAGGCCACCGAGCCCCCACCCAGCCAGGAGGGCCCCTCCCC |
| | | CGCCGTGCCTAGCATCTACGCCACCCTGGCCATCCACCGGA |
| | | GAAAGCGTGGATCCGGGGAAGGCCGAGGCTCCCTTCTAACA |
| | | TGTGGAGATGTCGAGGAAAACCCTGGCCCTATGGCGCTGCC |
| | | AGTCACTGCATTGTTATTGCCTCTGGCCCTGCTTCTCCATGC |
| | | GGCGCGCCCAGAAGTGCAGCTGGTCGAGAGCGGAGGCGGA |
| | | CTGGTTCAACCCGGAGGCAGCTTGAGACTGTCCTGCGCGGC |
| | | CAGCGGCTTCAACATCAAGGATACCTATATCCACTGGGTGA |
| | | GGCAGGCTCCAGGAAAGGGCCTGGAGTGGGTGGCAAGGAT |
| | | TTACCCTACTAATGGATATACACGCTACGCTGATTCCGTGA |
| | | AGGGACGCTTTACAATCTCAGCAGATACATCCAAAAACACG |
| | | GCCTATTTACAGATGAATAGTTTGCGGGCCGAAGACACGGC |
| | | TGTATACTATTGTTCTCGGTGGGGGGGCGATGGATTTTATGC |
| | | GATGGATTACTGGGGCCAGGGCACCCTGGTAACCGTGTCAA |
| | | GCGGCTCAACATCCGGGTCCGGTAAGCCGGGCTCCGGCGAG |
| | | GGGTCTACAAAGGGAGATATACAGATGACACAGTCCCCCA |
| | | GTTCCCTGTCCGCCTCAGTGGGAGACCGAGTGACGATTACC |
| | | TGTCGTGCCAGCCAGGACGTCAATACCGCCGTCGCTTGGTA |
| | | TCAGCAAAAACCAGGCAAGGCCCCGAAACTATTGATCTACA |
| | | GTGCCTCTTTTCTGTACTCCGGGGTGCCGAGCAGATTTAGTG |
| | | GCTCCAGGAGCGGAACCGATTTCACCCTAACCATTTCCAGT |
| | | TTGCAGCCAGAGGATTTCGCGACCTATTACTGCCAGCAACA |
| | | CTACACCACACCGCCAACTTTCGGACAAGGAACCAAGGTTG |
| | | AAATCAAAATTGAAGTTATGTATCCTCCTCCTTACCTAGACA |
| | | ATGAGAAGAGCAATGGAACCATTATCCATGTGAAAGGGAA |
| | | ACACCTTTGTCCAAGTCCCCTATTTCCCGGGCCTTCGAAGCC |
| | | CTTTTGGGTGCTGGTGGTGGTTGGTGGAGTCCTGGCTTGCTA |
| | | TAGCTTGCTAGTAACAGTAGCGTTTATTATTTTCTGGGTGAA |
| | | ACGCGGCCGCAAGAAACTGCTCTACATCTTTAAACAGCCGT |
| | | TCATGAGGCCTGTGCAGACAACGCAGGAAGAGGATGGCTG |
| | | TAGTTGTCGGTTTCCGGAAGAGGAAGAGGGGGGCTGCGAGT |
| | | TGCGTGTCAAATTTTCTCGGTCTGCCGACGCCCCCGCGTACC |
| | | AGCAAGGGCAGAACCAGCTTTATAATGAGCTGAATCTTGGA |
| | | CGACGGGAGGAATATGACGTGCTTGACAAGAGGCGAGGTA |
| | | GGGACCCTGAGATGGGGGGAAAACCTCGGAGGAAAAACCC |
| | | ACAGGAAGGCCTGTATAACGAACTGCAGAAGGACAAGATG |
| | | GCTGAAGCCTACTCTGAGATTGGAATGAAAGGGGAACGCA |
| | | GACGCGGCAAGGGCCATGATGGCCTCTACCAAGGTCTAAGC |
| | | ACTGCCACCAAGGACACCTATGACGCACTCCACATGCAAGC |
| | | TCTACCTCCCCGTTGATAA |
| MC00296- | SEQ ID | MALPVTALLLPLALLLHAARPQVQLQQSGPELVKPGASVKMS |
| BB7.2_CD8_ | NO: 34 | CKASGYTFTSYHIQWVKQRPGQGLEWIGWIYPGDGSTQYNEK |
| LIR1_HER2 | | FKGKTTLTADKSSSTAYMLLSSLTSEDSAIYFCAREGTYYAMD |
| protein | | YWGQGTSVTVSSGGGGSGGGGSGGGGSDVLMTQTPLSLPVSL |
| Sequence | | GDQVSISCRSSQSIVHSNGNTYLEWYLQKPGQSPKLLIYKVSNR |
| (VR296) | | FSGVPDRFSGSGSGTDFTLKISRVEAEDLGVYYCFQGSHVPRTF |
| | | GGGTKLEIKTTTPAPRPPTPAPTIASQPLSLRPEACRPAAGGAV |
| | | HTRGLDFACDIYIWAPLAGTCGVLLLSLVITLYCLRHRRQGKH |
| | | WTSTQRKADFQHPAGAVGPEPTDRGLQWRSSPAADAQEENL |
| | | YAAVKHTQPEDGVEMDTRSPHDEDPQAVTYAEVKHSRPRRE |
| | | MASPPSPLSGEFLDTKDRQAEEDRQMDTEAAASEAPQDVTYA |
| | | QLHSLTLRREATEPPPSQEGPSPAVPSIYATLAIHRRKRGSGEGR |
| | | GSLLTCGDVEENPGPMALPVTALLLPLALLLHAARPEVQLVES |
| | | GGGLVQPGGSLRLSCAASGFNIKDTYIHWVRQAPGKGLEWVA |
| | | RIYPTNGYTRYADSVKGRFTISADTSKNTAYLQMNSLRAEDTA |
| | | VYYCSRWGGDGFYAMDYWGQGTLVTVSSGSTSGSGKPGSGE |
| | | GSTKGDIQMTQSPSSLSASVGDRVTITCRASQDVNTAVAWYQ |
| | | QKPGKAPKLLIYSASFLYSGVPSRFSGSRSGTDFTLTISSLQPED |
| | | FATYYCQQHYTTPPTFGQGTKVEIKIEVMYPPPYLDNEKSNGTI |
| | | IHVKGKHLCPSPLFPGPSKPFWVLVVVGGVLACYSLLVTVAFII |
| | | FWVKRGRKKLLYIFKQPFMRPVQTTQEEDGCSCRFPEEEEGGC |
| | | ELRVKFSRSADAPAYQQGQNQLYNELNLGRREEYDVLDKRRG |
| | | RDPEMGGKPRRKNPQEGLYNELQKDKMAEAYSEIGMKGERR |
| | | RGKGHDGLYQGLSTATKDTYDALHMQALPPR |
| MC00297- | SEQ ID | ATGGCACTGCCAGTGACCGCCCTGCTGCTGCCTCTGGCCCT |
| BB7.2_CD8_ | NO: 35 | GCTGCTGCACGCAGCCAGACCCCAGGTGCAGCTGCAGCAGT |
| KIR2DL1_ | | CTGGACCTGAGCTGGTGAAGCCAGGAGCCTCCGTGAAGATG |
| HER2- | | TCTTGCAAGGCCAGCGGCTACACCTTCACATCTTATCACATC |
| nucleic acid | | CAGTGGGTGAAGCAGCGGCCCGGACAGGGCCTGGAGTGGA |
| (VR297) | | TCGGATGGATCTACCCAGGCGACGGCTCCACACAGTATAAC |
| | | GAGAAGTTCAAGGGCAAGACCACACTGACCGCCGATAAGA |
| | | GCAGCAGCACCGCCTACATGCTGCTGAGCAGCCTGACCAGC |
| | | GAGGACAGCGCCATCTACTTTTGCGCCAGGGAGGGCACATA |
| | | CTATGCTATGGACTATTGGGGCCAGGGCACCAGCGTGACAG |
| | | TGTCTAGCGGAGGAGGAGGCTCCGGAGGAGGAGGCTCTGG |
| | | CGGCGGCGGCAGCGACGTGCTGATGACCCAGACACCACTG |
| | | AGCCTGCCCGTGAGCCTGGGCGATCAGGTGAGCATCTCCTG |
| | | TAGATCCTCTCAGAGCATCGTGCACTCCAACGGCAATACCT |
| | | ACCTGGAGTGGTATCTGCAGAAGCCAGGCCAGTCCCCCAAG |
| | | CTGCTGATCTATAAGGTGTCTAATCGGTTCAGCGGCGTGCCT |
| | | GACAGATTTTCTGGCAGCGGCTCCGGCACCGACTTCACCCT |
| | | GAAGATCAGCCGGGTGGAGGCAGAGGATCTGGGCGTGTAC |
| | | TATTGTTTCCAGGGCTCCCACGTGCCACGCACCTTTGGCGGC |
| | | GGTACCAAGCTGGAGATCAAGACTACGACCCCAGCACCTAG |
| | | ACCTCCCACCCCAGCTCCAACTATAGCTTCCCAGCCATTGTC |
| | | TCTCCGGCCAGAGGCGTGTCGACCAGCCGCTGGAGGGGCCG |
| | | TTCATACAAGAGGACTCGATTTCGCTTGCGATATCTACATAT |
| | | GGGCCCCTCTTGCCGGGACATGCGGTGTCCTGCTTCTAAGCT |
| | | TGGTTATTACCCTCTATTGCCATAGGTGGTGCTCAAACAAA |
| | | AAGAATGCTGCCGTCATGGACCAGGAGAGCGCGGGCAATC |
| | | GGACCGCAAACTCAGAGGACTCAGATGAACAAGATCCACA |
| | | GGAAGTGACCTACACTCAGCTGAACCATTGTGTGTTTACAC |
| | | AGCGCAAGATTACTCGTCCAAGCCAGCGTCCTAAGACCCCC |
| | | CCGACCGATATCATTGTGTATACCGAGCTTCCTAATGCCGA |
| | | ATCCCGCAGCAAGGTGGTCTCCTGCCCGCGGAGAAAGCGTG |
| | | GATCCGGGGAAGGCCGAGGCTCCCTTCTAACATGTGGAGAT |
| | | GTCGAGGAAAACCCTGGCCCTATGGCGCTGCCAGTCACTGC |
| | | ATTGTTATTGCCTCTGGCCCTGCTTCTCCATGCGGCGCGCCC |
| | | AGAAGTGCAGCTGGTCGAGAGCGGAGGCGGACTGGTTCAA |
| | | CCCGGAGGCAGCTTGAGACTGTCCTGCGCGGCCAGCGGCTT |
| | | CAACATCAAGGATACCTATATCCACTGGGTGAGGCAGGCTC |
| | | CAGGAAAGGGCCTGGAGTGGGTGGCAAGGATTTACCCTACT |
| | | AATGGATATACACGCTACGCTGATTCCGTGAAGGGACGCTT |
| | | TACAATCTCAGCAGATACATCCAAAAACACGGCCTATTTAC |
| | | AGATGAATAGTTTGCGGGCCGAAGACACGGCTGTATACTAT |
| | | TGTTCTCGGTGGGGGGGCGATGGATTTTATGCGATGGATTA |
| | | CTGGGGCCAGGGCACCCTGGTAACCGTGTCAAGCGGCTCAA |
| | | CATCCGGGTCCGGTAAGCCGGGCTCCGGCGAGGGGTCTACA |
| | | AAGGGAGATATACAGATGACACAGTCCCCCAGTTCCCTGTC |
| | | CGCCTCAGTGGGAGACCGAGTGACGATTACCTGTCGTGCCA |
| | | GCCAGGACGTCAATACCGCCGTCGCTTGGTATCAGCAAAAA |
| | | CCAGGCAAGGCCCCGAAACTATTGATCTACAGTGCCTCTTT |
| | | TCTGTACTCCGGGGTGCCGAGCAGATTTAGTGGCTCCAGGA |
| | | GCGGAACCGATTTCACCCTAACCATTTCCAGTTTGCAGCCA |
| | | GAGGATTTCGCGACCTATTACTGCCAGCAACACTACACCAC |
| | | ACCGCCAACTTTCGGACAAGGAACCAAGGTTGAAATCAAA |
| | | ATTGAAGTTATGTATCCTCCTCCTTACCTAGACAATGAGAA |
| | | GAGCAATGGAACCATTATCCATGTGAAAGGGAAACACCTTT |
| | | GTCCAAGTCCCCTATTTCCCGGGCCTTCGAAGCCCTTTTGGG |
| | | TGCTGGTGGTGGTTGGTGGAGTCCTGGCTTGCTATAGCTTGC |
| | | TAGTAACAGTAGCGTTTATTATTTTCTGGGTGAAACGCGGC |
| | | CGCAAGAAACTGCTCTACATCTTTAAACAGCCGTTCATGAG |
| | | GCCTGTGCAGACAACGCAGGAAGAGGATGGCTGTAGTTGTC |
| | | GGTTTCCGGAAGAGGAAGAGGGGGGCTGCGAGTTGCGTGT |
| | | CAAATTTTCTCGGTCTGCCGACGCCCCCGCGTACCAGCAAG |
| | | GGCAGAACCAGCTTTATAATGAGCTGAATCTTGGACGACGG |
| | | GAGGAATATGACGTGCTTGACAAGAGGCGAGGTAGGGACC |
| | | CTGAGATGGGGGGAAAACCTCGGAGGAAAAACCCACAGGA |
| | | AGGCCTGTATAACGAACTGCAGAAGGACAAGATGGCTGAA |
| | | GCCTACTCTGAGATTGGAATGAAAGGGGAACGCAGACGCG |
| | | GCAAGGGCCATGATGGCCTCTACCAAGGTCTAAGCACTGCC |
| | | ACCAAGGACACCTATGACGCACTCCACATGCAAGCTCTACC |
| | | TCCCCGTTGATAA |
| MC00297: | SEQ ID | MALPVTALLLPLALLLHAARPQVQLQQSGPELVKPGASVKMS |
| BB7.2_CD8_ | NO: 36 | CKASGYTFTSYHIQWVKQRPGQGLEWIGWIYPGDGSTQYNEK |
| KIR2DL1_ | | FKGKTTLTADKSSSTAYMLLSSLTSEDSAIYFCAREGTYYAMD |
| HER2 | | YWGQGTSVTVSSGGGGSGGGGSGGGGSDVLMTQTPLSLPVSL |
| protein | | GDQVSISCRSSQSIVHSNGNTYLEWYLQKPGQSPKLLIYKVSNR |
| Sequence | | FSGVPDRFSGSGSGTDFTLKISRVEAEDLGVYYCFQGSHVPRTF |
| (VR297) | | GGGTKLEIKTTTPAPRPPTPAPTIASQPLSLRPEACRPAAGGAV |
| | | HTRGLDFACDIYIWAPLAGTCGVLLLSLVITLYCHRWCSNKKN |
| | | AAVMDQESAGNRTANSEDSDEQDPQEVTYTQLNHCVFTQRKI |
| | | TRPSQRPKTPPTDIIVYTELPNAESRSKVVSCPRRKRGSGEGRG |
| | | SLLTCGDVEENPGPMALPVTALLLPLALLLHAARPEVQLVESG |
| | | GGLLVQPGGSLRLSCAASGFNIKDTYIHWVRQAPGKGLEWVA |
| | | RIYPTNGYTRYADSVKGRFTISADTSKNTAYLQMNSLRAEDTA |
| | | VYYCSRWGGDGFYAMDYWGQGTLVTVSSGSTSGSGKPGSGE |
| | | GSTKGDIQMTQSPSSLSASVGDRVTITCRASQDVNTAVAWYQ |
| | | QKPGKAPKLLIYSASFLYSGVPSRFSGSRSGTDFTLTISSLQPED |
| | | FATYYCQQHYTTPPTFGQGTKVEIKIEVMYPPPYLDNEKSNGTI |
| | | IHVKGKHLCPSPLFPGPSKPFWVLVVVGGVLACYSLLVTVAFII |
| | | FWVKRGRKKLLYIFKQPFMRPVQTTQEEDGCSCRFPEEEEGGC |
| | | ELRVKFSRSADAPAYQQGQNQLYNELNLGRREEYDVLDKRRG |
| | | RDPEMGGKPRRKNPQEGLYNELQKDKMAEAYSEIGMKGERR |
| | | RGKGHDGLYQGLSTATKDTYDALHMQALPPR |
| MC0421 | SEQ ID | ATGGCGCTGCCAGTCACTGCATTGTTATTGCCTCTGGCCCTG |
| HzBB7.2.2 | NO: 275 | CTTCTCCATGCGGCAAGGCCACAGGTGCAACTGGTTCAATC |
| LIR1(52)_ | | TGGTGCTGAGGTGAAAAAGCCCGGCGCATCCGTGAAAGTG |
| 2A_HER2 | | AGCTGTAAGGCATCAGGGTACACCTTCACCAGCTATCACAT |
| nucleotide | | ACAATGGGTCCGCCAGGCCCCCGGACAGAGGTTGGAATGG |
| sequence | | ATTGGGTGGATTTACCCGGGTGACGGCTCAACCCAGTACAA |
| (VR421) | | TGAGAAGTTCAAGGGCAGGGTGACTATCACACGCGATACCT |
| | | CCGCGAGCACAGCTTACATGGAGTTATCTAGCCTGAGATCC |
| | | GAAGATACGGCGGTGTATTACTGCGCGCGGGAAGGGACCT |
| | | ACTATGCCATGGACTATTGGGGACAAGGGACCCTGGTTACC |
| | | GTGAGTTCTGGGGGCGGGGGTTCCGGGGGAGGGGGATCTG |
| | | GGGGTGGAGGGAGCGATGTGGTAATGACCCAGACACCTTTG |
| | | TCTTTGAGTGTCACCCCCGGACAGCCGGCAAGTATATCCTG |
| | | TAGATCATCCCAATCAATCGTGCACTCCAACGGAAACACAT |
| | | ACTTGGAATGGTATCTCCAGAAACCTGGACAGTCCCCACAG |
| | | TTGCTCATCTACAAAGTGTCAAATCGCTTTTCAGGCGTGCCC |
| | | GATCGTTTCAGCGGCTCAGGCTCCGGGACAGACTTTACATT |
| | | GAAGATTAGCCGCGTAGAGGCAGAGGATGTGGGCGTTTACT |
| | | ATTGTTTTCAAGGGTCACACGTGCCACGCACATTCGGCGGC |
| | | GGTACCAAGGTGGAAATTAAGCACCCCAGCGACCCGCTGG |
| | | AGCTCGTTGTGTCCGGACCATCAGGGGGCCCGAGTAGCCCT |
| | | ACAACCGGCCCCACTTCTACCAGTGGACCGGAAGATCAACC |
| | | ACTTACACCAACGGGCAGCGACCCCCAGTCAGGCCTAGGGC |
| | | GCCACCTGGGTGTGGTCATCGGGATACTGGTCGCTGTCATC |
| | | CTGCTTCTGCTCCTTCTCTTGCTCCTATTCCTAATCCTGCGCC |
| | | ACAGGAGACAGGGCAAGCACTGGACCAGCACCCAGCGGAA |
| | | GGCCGACTTTCAGCACCCTGCCGGCGCCGTGGGCCCTGAGC |
| | | CTACCGACAGGGGCCTGCAGTGGAGGAGCTCCCCAGCCGCC |
| | | GATGCCCAGGAGGAGAATCTGTACGCCGCCGTGAAGCACA |
| | | CCCAGCCAGAGGACGGCGTGGAGATGGACACCCGCTCCCC |
| | | ACACGACGAGGACCCACAGGCCGTGACCTACGCCGAGGTG |
| | | AAGCACAGCCGCCCCAGACGCGAGATGGCCAGCCCACCCA |
| | | GCCCCCTGTCCGGCGAGTTCCTGGACACCAAGGACAGGCAG |
| | | GCCGAGGAGGACCGGCAGATGGACACCGAGGCCGCCGCCT |
| | | CCGAGGCCCCCCAGGACGTGACCTACGCCCAGCTGCACTCC |
| | | CTGACCCTGCGGAGAGAGGCCACCGAGCCCCCACCCAGCCA |
| | | GGAGGGCCCCTCCCCCGCCGTGCCTAGCATCTACGCCACCC |
| | | TGGCCATCCACCGGAGAAAGCGTGGATCCGGGGAAGGCCG |
| | | AGGCTCCCTTCTAACATGTGGAGATGTCGAGGAAAACCCTG |
| | | GCCCTATGGCGCTGCCAGTCACTGCATTGTTATTGCCTCTGG |
| | | CCCTGCTTCTCCATGCGGCGCGCCCAGACATCCAGATGACC |
| | | CAATCCCCAAGCAGTCTCTCAGCCAGCGTGGGAGACAGGGT |
| | | TACAATCACGTGCCGCGCCAGCCAGGACGTCAACACCGCTG |
| | | TGGCTTGGTATCAGCAAAAGCCCGGGAAGGCACCAAAGCT |
| | | GCTTATTTATAGCGCCTCCTTCTTGTATTCTGGAGTGCCATC |
| | | CAGGTTTTCCGGGTCACGTAGCGGGACTGACTTTACCCTCA |
| | | CCATATCCAGCCTCCAGCCCGAGGATTTCGCCACCTATTACT |
| | | GTCAGCAACACTACACGACTCCACCGACTTTTGGACAGGGC |
| | | ACTAAAGTGGAGATTAAGGGCAGCACGAGTGGGAGTGGAA |
| | | AGCCCGGCAGCGGGGAGGGGTCTACCAAGGGAGAGGTCCA |
| | | GCTGGTTGAATCCGGAGGCGGGCTTGTGCAACCTGGAGGCT |
| | | CCCTGAGGCTTAGTTGTGCCGCGTCAGGATTCAACATTAAG |
| | | GATACCTATATTCATTGGGTCCGACAAGCCCCGGGCAAGGG |
| | | CTTGGAGTGGGTGGCCAGAATCTATCCGACCAACGGATATA |
| | | CAAGGTACGCCGATTCTGTGAAAGGACGCTTCACCATCAGC |
| | | GCGGACACATCCAAAAACACAGCCTATCTGCAGATGAACTC |
| | | CCTTCGCGCCGAGGATACAGCCGTGTACTATTGTAGTCGGT |
| | | GGGGAGGCGACGGCTTCTACGCGATGGACTATTGGGGACA |
| | | AGGAACACTGGTGACTGTCAGTAGCACTACGACCCCAGCAC |
| | | CTAGACCTCCCACCCCAGCTCCAACTATAGCTTCCCAGCCAT |
| | | TGTCTCTCCGGCCAGAGGCGTGTCGACCAGCCGCTGGAGGG |
| | | GCCGTTCATACAAGAGGACTCGATTTCGCTTGCGATATCTA |
| | | CATATGGGCCCCTCTTGCCGGGACATGCGGTGTCCTGCTTCT |
| | | AAGCTTGGTTATTACCCTCTATTGCAAAAGAGGACGAAAGA |
| | | AACTGCTTTATATATTCAAGCAACCTTTCATGCGCCCCGTAC |
| | | AGACCACGCAGGAGGAAGATGGGTGTAGCTGTCGCTTCCCT |
| | | GAGGAAGAGGAAGGTGGATGCGAGTTGCGGGTGAAGTTCA |
| | | GTCGATCCGCCGATGCGCCTGCCTATCAGCAAGGGCAGAAC |
| | | CAGCTTTATAACGAGTTAAACCTTGGCCGCCGGGAAGAGTA |
| | | TGACGTGTTGGACAAGCGTCGCGGGAGAGACCCTGAGATG |
| | | GGCGGAAAACCAAGGAGAAAAAATCCACAGGAAGGCTTAT |
| | | ATAACGAGTTGCAGAAAGACAAGATGGCCGAGGCATACTC |
| | | CGAAATCGGAATGAAGGGCGAGCGACGGCGCGGCAAAGGC |
| | | CACGATGGACTCTATCAGGGCTTAAGCACCGCCACCAAAGA |
| | | CACCTACGATGCACTTCATATGCAGGCACTCCCACCTAGAT |
| | | GATAA |
| MC0421 | SEQ ID | MALPVTALLLPLALLLHAARPQVQLVQSGAEVKKPGASVKVS |
| HzBB7.2.2_ | NO: 276 | CKASGYTFTSYHIQWVRQAPGQRLEWIGWIYPGDGSTQYNEK |
| LIR1(52)_ | | FKGRVTITRDTSASTAYMELSSLRSEDTAVYYCAREGTYYAM |
| 2A_HER2 | | DYWGQGTLVTVSSGGGGSGGGGSGGGGSDVVMTQTPLSLSV |
| Protein | | TPGQPASISCRSSQSIVHSNGNTYLEWYLQKPGQSPQLLIYKVS |
| Sequence | | NRFSGVPDRFSGSGSGTDFTLKISRVEAEDVGVYYCFQGSHVP |
| (VR421) | | RTFGGGTKVEIKHPSDPLELVVSGPSGGPSSPTTGPTSTSGPEDQ |
| | | PLTPTGSDPQSGLGRHLGVVIGILVAVILLLLLLLLLFLILRHRR |
| | | QGKHWTSTQRKADFQHPAGAVGPEPTDRGLQWRSSPAADAQ |
| | | EENLYAAVKHTQPEDGVEMDTRSPHDEDPQAVTYAEVKHSRP |
| | | RREMASPPSPLSGEFLDTKDRQAEEDRQMDTEAAASEAPQDV |
| | | TYAQLHSLTLRREATEPPPSQEGPSPAVPSIYATLAIHRRKRGS |
| | | GEGRGSLLTCGDVEENPGPMALPVTALLLPLALLLHAARPDIQ |
| | | MTQSPSSLSASVGDRVTITCRASQDVNTAVAWYQQKPGKAPK |
| | | LLIYSASFLYSGVPSRFSGSRSGTDFTLTISSLQPEDFATYYCQQ |
| | | HYTTPPTFGQGTKVEIKGSTSGSGKPGSGEGSTKGEVQLVESG |
| | | GGLVQPGGSLRLSCAASGFNIKDTYIHWVRQAPGKGLEWVAR |
| | | IYPTNGYTRYADSVKGRFTISADTSKNTAYLQMNSLRAEDTAV |
| | | YYCSRWGGDGFYAMDYWGQGTLVTVSSTTTPAPRPPTPAPTI |
| | | ASQPLSLRPEACRPAAGGAVHTRGLDFACDIYIWAPLAGTCGV |
| | | LLLSLVITLYCKRGRKKLLYIFKQPFMRPVQTTQEEDGCSCRFP |
| | | EEEEGGCELRVKFSRSADAPAYQQGQNQLYNELNLGRREEYD |
| | | VLDKRRGRDPEMGGKPRRKNPQEGLYNELQKDKMAEAYSEI |
| | | GMKGERRRGKGHDGLYQGLSTATKDTYDALHMQALPPR |
| MC00428 | SEQ ID | ATGGCGCTGCCAGTCACTGCATTGTTATTGCCTCTGGCCCTG |
| HzBB7.2.1_ | NO: 277 | CTTCTCCATGCGGCAAGGCCACAGGTGCAACTGGTTCAATC |
| LIR1(52)_ | | TGGTGCTGAGGTGAAAAAGCCCGGCAGCTCTGTGAAAGTGA |
| (IRESL)_HER2 | | GCTGTAAGGCATCAGGGTATACCTTCACCAGCTATCACATA |
| nucleotide | | CAATGGGTCCGCCAGGCCCCCGGACAGGGATTGGAATGGAT |
| sequence | | GGGGTGGATTTACCCGGGTGACGGCTCAACCCAGTACAATG |
| (VR428) | | AGAAGTTCAAGGGCAGGACAACTATCACAGCCGATAAGTC |
| | | CACGAGCACAGCTTACATGGAGTTATCTAGCCTGAGATCCG |
| | | AAGATACGGCGGTGTATTACTGCGCGCGGGAAGGGACCTAC |
| | | TATGCCATGGACTATTGGGGACAAGGGACCCTGGTTACCGT |
| | | GAGTTCTGGGGGCGGGGGTTCCGGGGGAGGGGGATCTGGG |
| | | GGTGGAGGGAGCGATGTGGTAATGACCCAGACACCTTTGTC |
| | | TTTGAGTGTCACCCCCGGACAGCCGGCAAGTATATCCTGTA |
| | | GATCATCCCAATCAATCGTGCACTCCAACGGAAACACATAC |
| | | TTGGAATGGTATCTCCAGAAACCTGGACAGTCCCCACAGTT |
| | | GCTCATCTACAAAGTGTCAAATCGCTTTTCAGGCGTGCCCG |
| | | ATCGTTTCAGCGGCTCAGGCTCCGGGACAGACTTTACATTG |
| | | AAGATTAGCCGCGTAGAGGCAGAGGATGTGGGCGTTTACTA |
| | | TTGTTTTCAAGGGTCACACGTGCCACGCACATTCGGCGGCG |
| | | GTACCAAGGTGGAAATTAAGCACCCCAGCGACCCGCTGGA |
| | | GCTCGTTGTGTCCGGACCATCAGGGGGCCCGAGTAGCCCTA |
| | | CAACCGGCCCCACTTCTACCAGTGGACCGGAAGATCAACCA |
| | | CTTACACCAACGGGCAGCGACCCCCAGTCAGGCCTAGGGCG |
| | | CCACCTGGGTGTGGTCATCGGGATACTGGTCGCTGTCATCCT |
| | | GCTTCTGCTCCTTCTCTTGCTCCTATTCCTAATCCTGCGCCAC |
| | | AGGAGACAGGGCAAGCACTGGACCAGCACCCAGCGGAAGG |
| | | CCGACTTTCAGCACCCTGCCGGCGCCGTGGGCCCTGAGCCT |
| | | ACCGACAGGGGCCTGCAGTGGAGGAGCTCCCCAGCCGCCG |
| | | ATGCCCAGGAGGAGAATCTGTACGCCGCCGTGAAGCACACC |
| | | CAGCCAGAGGACGGCGTGGAGATGGACACCCGCTCCCCAC |
| | | ACGACGAGGACCCACAGGCCGTGACCTACGCCGAGGTGAA |
| | | GCACAGCCGCCCCAGACGCGAGATGGCCAGCCCACCCAGC |
| | | CCCCTGTCCGGCGAGTTCCTGGACACCAAGGACAGGCAGGC |
| | | CGAGGAGGACCGGCAGATGGACACCGAGGCCGCCGCCTCC |
| | | GAGGCCCCCCAGGACGTGACCTACGCCCAGCTGCACTCCCT |
| | | GACCCTGCGGAGAGAGGCCACCGAGCCCCCACCCAGCCAG |
| | | GAGGGCCCCTCCCCCGCCGTGCCTAGCATCTACGCCACCCT |
| | | GGCCATCCACTGATAACCCCCCCCCCTAACGTTACTGGCCG |
| | | AAGCCGCTTGGAATAAGGCCGGTGTGCGTTTGTCTATATGT |
| | | TATTTTCCACCATATTGCCGTCTTTTGGCAATGTGAGGGCCC |
| | | GGAAACCTGGCCCTGTCTTCTTGACGAGCATTCCTAGGGGT |
| | | CTTTCCCCTCTCGCCAAAGGAATGCAAGGTCTGTTGAATGTC |
| | | GTGAAGGAAGCAGTTCCTCTGGAAGCTTCTTGAAGACAAAC |
| | | AACGTCTGTAGCGACCCTTTGCAGGCAGCGGAACCCCCCAC |
| | | CTGGCGACAGGTGCCTCTGCGGCCAAAAGCCACGTGTATAA |
| | | GATACACCTGCAAAGGCGGCACAACCCCAGTGCCACGTTGT |
| | | GAGTTGGATAGTTGTGGAAAGAGTCAAATGGCTCTCCTCAA |
| | | GCGTATTCAACAAGGGGCTGAAGGATGCCCAGAAGGTACC |
| | | CCATTGTATGGGATCTGATCTGGGGCCTCGGTGCACATGCTT |
| | | TACATGTGTTTAGTCGAGGTTAAAAAAACGTCTAGGCCCCC |
| | | CGAACCACGGGGACGTGGTTTTCCTTTGAAAAACACGATGA |
| | | TAATATGATGGCGCTGCCAGTCACTGCATTGTTATTGCCTCT |
| | | GGCCCTGCTTCTCCATGCGGCGCGCCCAGACATCCAGATGA |
| | | CCCAATCCCCAAGCAGTCTCTCAGCCAGCGTGGGAGACAGG |
| | | GTTACAATCACGTGCCGCGCCAGCCAGGACGTCAACACCGC |
| | | TGTGGCTTGGTATCAGCAAAAGCCCGGGAAGGCACCAAAG |
| | | CTGCTTATTTATAGCGCCTCCTTCTTGTATTCTGGAGTGCCA |
| | | TCCAGGTTTTCCGGGTCACGTAGCGGGACTGACTTTACCCTC |
| | | ACCATATCCAGCCTCCAGCCCGAGGATTTCGCCACCTATTA |
| | | CTGTCAGCAACACTACACGACTCCACCGACTTTTGGACAGG |
| | | GCACTAAAGTGGAGATTAAGGGCAGCACGAGTGGGAGTGG |
| | | AAAGCCCGGCAGCGGGGAGGGGTCTACCAAGGGAGAGGTC |
| | | CAGCTGGTTGAATCCGGAGGCGGGCTTGTGCAACCTGGAGG |
| | | CTCCCTGAGGCTTAGTTGTGCCGCGTCAGGATTCAACATTA |
| | | AGGATACCTATATTCATTGGGTCCGACAAGCCCCGGGCAAG |
| | | GGCTTGGAGTGGGTGGCCAGAATCTATCCGACCAACGGATA |
| | | TACAAGGTACGCCGATTCTGTGAAAGGACGCTTCACCATCA |
| | | GCGCGGACACATCCAAAAACACAGCCTATCTGCAGATGAAC |
| | | TCCCTTCGCGCCGAGGATACAGCCGTGTACTATTGTAGTCG |
| | | GTGGGGAGGCGACGGCTTCTACGCGATGGACTATTGGGGAC |
| | | AAGGAACACTGGTGACTGTCAGTAGCACTACGACCCCAGCA |
| | | CCTAGACCTCCCACCCCAGCTCCAACTATAGCTTCCCAGCC |
| | | ATTGTCTCTCCGGCCAGAGGCGTGTCGACCAGCCGCTGGAG |
| | | GGGCCGTTCATACAAGAGGACTCGATTTCGCTTGCGATATC |
| | | TACATATGGGCCCCTCTTGCCGGGACATGCGGTGTCCTGCTT |
| | | CTAAGCTTGGTTATTACCCTCTATTGCAAAAGAGGACGAAA |
| | | GAAACTGCTTTATATATTCAAGCAACCTTTCATGCGCCCCGT |
| | | ACAGACCACGCAGGAGGAAGATGGGTGTAGCTGTCGCTTCC |
| | | CTGAGGAAGAGGAAGGTGGATGCGAGTTGCGGGTGAAGTT |
| | | CAGTCGATCCGCCGATGCGCCTGCCTATCAGCAAGGGCAGA |
| | | ACCAGCTTTATAACGAGTTAAACCTTGGCCGCCGGGAAGAG |
| | | TATGACGTGTTGGACAAGCGTCGCGGGAGAGACCCTGAGAT |
| | | GGGCGGAAAACCAAGGAGAAAAAATCCACAGGAAGGCTTA |
| | | TATAACGAGTTGCAGAAAGACAAGATGGCCGAGGCATACT |
| | | CCGAAATCGGAATGAAGGGCGAGCGACGGCGCGGCAAAGG |
| | | CCACGATGGACTCTATCAGGGCTTAAGCACCGCCACCAAAG |
| | | ACACCTACGATGCACTTCATATGCAGGCACTCCCACCTAGA |
| | | TGATAA |
| MC0428 | SEQ ID | MALPVTALLLPLALLLHAARPQVQLVQSGAEVKKPGSSVKVS |
| HzBB7.2.1_ | NO: 278 | CKASGYTFTSYHIQWVRQAPGQGLEWMGWIYPGDGSTQYNE |
| LIR1(52)_ | | KFKGRTTITADKSTSTAYMELSSLRSEDTAVYYCAREGTYYA |
| (IRESL)_ | | MDYWGQGTLVTVSSGGGGSGGGGSGGGGSDVVMTQTPLSLS |
| HER2 Protein | | VTPGQPASISCRSSQSIVHSNGNTYLEWYLQKPGQSPQLLIYKV |
| sequence | | SNRFSGVPDRFSGSGSGTDFTLKISRVEAEDVGVYYCFQGSHV |
| (VR428) | | PRTFGGGTKVEIKHPSDPLELVVSGPSGGPSSPTTGPTSTSGPED |
| | | QPLTPTGSDPQSGLGRHLGVVIGILVAVILLLLLLLLLFLILRHR |
| | | RQGKHWTSTQRKADFQHPAGAVGPEPTDRGLQWRSSPAADA |
| | | QEENLYAAVKHTQPEDGVEMDTRSPHDEDPQAVTYAEVKHS |
| | | RPRREMASPPSPLSGEFLDTKDRQAEEDRQMDTEAAASEAPQD |
| | | VTYAQLHSLTLRREATEPPPSQEGPSPAVPSIYATLAIH* |
| | | MALPVTALLLPLALLLHAARPDIQMTQSPSSLSASVGDRVTITC |
| | | RASQDVNTAVAWYQQKPGKAPKLLIYSASFLYSGVPSRFSGSR |
| | | SGTDFTLTISSLQPEDFATYYCQQHYTTPPTFGQGTKVEIKGST |
| | | SGSGKPGSGEGSTKGEVQLVESGGGLVQPGGSLRLSCAASGEN |
| | | IKDTYIHWVRQAPGKGLEWVARIYPTNGYTRYADSVKGRFTIS |
| | | ADTSKNTAYLQMNSLRAEDTAVYYCSRWGGDGFYAMDYWG |
| | | QGTLVTVSSTTTPAPRPPTPAPTIASQPLSLRPEACRPAAGGAV |
| | | HTRGLDFACDIYIWAPLAGTCGVLLLSLVITLYCKRGRKKLLYI |
| | | FKQPFMRPVQTTQEEDGCSCRFPEEEEGGCELRVKFSRSADAP |
| | | AYQQGQNQLYNELNLGRREEYDVLDKRRGRDPEMGGKPRRK |
| | | NPQEGLYNELQKDKMAEAYSEIGMKGERRRGKGHDGLYQGL |
| | | STATKDTYDALHMQALPPR* |
| MC0447 | SEQ ID | ATGGCGCTGCCAGTCACTGCATTGTTATTGCCTCTGGCCCTG |
| SN66E3.2 | NO: 279 | CTTCTCCATGCGGCAAGGCCAGATATAGTGATGACACAGTC |
| (LH)_LIR1 | | CCCCGACTCCCTGGCTGTCTCACTGGGAGAACGAGCGACGA |
| (30)_ | | TTAGTTGTAAGTCTAGCCAGAGCGTCCTGTATTCAAGCAAT |
| (IRESL)_HER2 | | AACAAGAATTACCTCGCCTGGTATCAGCAAAAGCCGGGACA |
| nucleotide | | GCCACCCAAACTGTTGATTTACTGGGCCAGCACGAGAGAGA |
| Sequence | | GCGGAGTGCCCGACCGCTTCAGCGGATCCGGGTCAGGCACA |
| (VR447) | | GATTTTACCCTGACTATTAGCTCCCTTCAAGCGGAAGATGTC |
| | | GCCGTCTACTATTGCCAGCAATATTACGGAACTCCATTCAC |
| | | ATTCGGCGGTGGGACCAAAGTAGAGATAAAGGGTGGCGGG |
| | | GGATCCGGCGGTGGCGGTAGCGGGGGAGGCGGGTCCCAAG |
| | | TGCAACTAGTCCAATCAGGTGCCGAAGTCAAGAAACCAGGT |
| | | GCATCCGTGAAAGTGTCTTGCAAAGCCAGTGGCTACACTTT |
| | | TACTGACTACTATCTGCACTGGGTGCGTCAAGCACCCGGCC |
| | | AGGGGCTTGAATGGATGGGCTGGATTAACCCTTATACTGGA |
| | | GGGACAAATTACGCTCAGAAGTTCCAGGGACGCGTTACAAT |
| | | GACCCGAGACACCAGCATCAGCACAGCGTACATGGAGTTA |
| | | AGTGGGCTGACTTCCGACGATACCGCCGTGTATTACTGCGC |
| | | TCGGGCAGGGGCCTCTTACTATGATTTTTGGTCCGGTTGGGT |
| | | CTTCGATTACTGGGGGCAGGGAACCCTGGTGACAGTGTCCT |
| | | CAGGCCCCACTTCTACCAGTGGACCGGAAGATCAACCACTT |
| | | ACACCAACGGGCAGCGACCCCCAGTCAGGCCTAGGGCGCC |
| | | ACCTGGGTGTGGTCATCGGGATACTGGTCGCTGTCATCCTG |
| | | CTTCTGCTCCTTCTCTTGCTCCTATTCCTAATCCTGCGCCACA |
| | | GGAGACAGGGCAAGCACTGGACCAGCACCCAGCGGAAGGC |
| | | CGACTTTCAGCACCCTGCCGGCGCCGTGGGCCCTGAGCCTA |
| | | CCGACAGGGGCCTGCAGTGGAGGAGCTCCCCAGCCGCCGAT |
| | | GCCCAGGAGGAGAATCTGTACGCCGCCGTGAAGCACACCC |
| | | AGCCAGAGGACGGCGTGGAGATGGACACCCGCTCCCCACA |
| | | CGACGAGGACCCACAGGCCGTGACCTACGCCGAGGTGAAG |
| | | CACAGCCGCCCCAGACGCGAGATGGCCAGCCCACCCAGCCC |
| | | CCTGTCCGGCGAGTTCCTGGACACCAAGGACAGGCAGGCCG |
| | | AGGAGGACCGGCAGATGGACACCGAGGCCGCCGCCTCCGA |
| | | GGCCCCCCAGGACGTGACCTACGCCCAGCTGCACTCCCTGA |
| | | CCCTGCGGAGAGAGGCCACCGAGCCCCCACCCAGCCAGGA |
| | | GGGCCCCTCCCCCGCCGTGCCTAGCATCTACGCCACCCTGG |
| | | CCATCCACTGATAACCCCCCCCCCTAACGTTACTGGCCGAA |
| | | GCCGCTTGGAATAAGGCCGGTGTGCGTTTGTCTATATGTTAT |
| | | TTTCCACCATATTGCCGTCTTTTGGCAATGTGAGGGCCCGGA |
| | | AACCTGGCCCTGTCTTCTTGACGAGCATTCCTAGGGGTCTTT |
| | | CCCCTCTCGCCAAAGGAATGCAAGGTCTGTTGAATGTCGTG |
| | | AAGGAAGCAGTTCCTCTGGAAGCTTCTTGAAGACAAACAAC |
| | | GTCTGTAGCGACCCTTTGCAGGCAGCGGAACCCCCCACCTG |
| | | GCGACAGGTGCCTCTGCGGCCAAAAGCCACGTGTATAAGAT |
| | | ACACCTGCAAAGGCGGCACAACCCCAGTGCCACGTTGTGAG |
| | | TTGGATAGTTGTGGAAAGAGTCAAATGGCTCTCCTCAAGCG |
| | | TATTCAACAAGGGGCTGAAGGATGCCCAGAAGGTACCCCAT |
| | | TGTATGGGATCTGATCTGGGGCCTCGGTGCACATGCTTTAC |
| | | ATGTGTTTAGTCGAGGTTAAAAAAACGTCTAGGCCCCCCGA |
| | | ACCACGGGGACGTGGTTTTCCTTTGAAAAACACGATGATAA |
| | | TATGATGGCGCTGCCAGTCACTGCATTGTTATTGCCTCTGGC |
| | | CCTGCTTCTCCATGCGGCGCGCCCAGACATCCAGATGACCC |
| | | AATCCCCAAGCAGTCTCTCAGCCAGCGTGGGAGACAGGGTT |
| | | ACAATCACGTGCCGCGCCAGCCAGGACGTCAACACCGCTGT |
| | | GGCTTGGTATCAGCAAAAGCCCGGGAAGGCACCAAAGCTG |
| | | CTTATTTATAGCGCCTCCTTCTTGTATTCTGGAGTGCCATCC |
| | | AGGTTTTCCGGGTCACGTAGCGGGACTGACTTTACCCTCAC |
| | | CATATCCAGCCTCCAGCCCGAGGATTTCGCCACCTATTACTG |
| | | TCAGCAACACTACACGACTCCACCGACTTTTGGACAGGGCA |
| | | CTAAAGTGGAGATTAAGGGCAGCACGAGTGGGAGTGGAAA |
| | | GCCCGGCAGCGGGGAGGGGTCTACCAAGGGAGAGGTCCAG |
| | | CTGGTTGAATCCGGAGGCGGGCTTGTGCAACCTGGAGGCTC |
| | | CCTGAGGCTTAGTTGTGCCGCGTCAGGATTCAACATTAAGG |
| | | ATACCTATATTCATTGGGTCCGACAAGCCCCGGGCAAGGGC |
| | | TTGGAGTGGGTGGCCAGAATCTATCCGACCAACGGATATAC |
| | | AAGGTACGCCGATTCTGTGAAAGGACGCTTCACCATCAGCG |
| | | CGGACACATCCAAAAACACAGCCTATCTGCAGATGAACTCC |
| | | CTTCGCGCCGAGGATACAGCCGTGTACTATTGTAGTCGGTG |
| | | GGGAGGCGACGGCTTCTACGCGATGGACTATTGGGGACAA |
| | | GGAACACTGGTGACTGTCAGTAGCACTACGACCCCAGCACC |
| | | TAGACCTCCCACCCCAGCTCCAACTATAGCTTCCCAGCCATT |
| | | GTCTCTCCGGCCAGAGGCGTGTCGACCAGCCGCTGGAGGGG |
| | | CCGTTCATACAAGAGGACTCGATTTCGCTTGCGATATCTAC |
| | | ATATGGGCCCCTCTTGCCGGGACATGCGGTGTCCTGCTTCTA |
| | | AGCTTGGTTATTACCCTCTATTGCAAAAGAGGACGAAAGAA |
| | | ACTGCTTTATATATTCAAGCAACCTTTCATGCGCCCCGTACA |
| | | GACCACGCAGGAGGAAGATGGGTGTAGCTGTCGCTTCCCTG |
| | | AGGAAGAGGAAGGTGGATGCGAGTTGCGGGTGAAGTTCAG |
| | | TCGATCCGCCGATGCGCCTGCCTATCAGCAAGGGCAGAACC |
| | | AGCTTTATAACGAGTTAAACCTTGGCCGCCGGGAAGAGTAT |
| | | GACGTGTTGGACAAGCGTCGCGGGAGAGACCCTGAGATGG |
| | | GCGGAAAACCAAGGAGAAAAAATCCACAGGAAGGCTTATA |
| | | TAACGAGTTGCAGAAAGACAAGATGGCCGAGGCATACTCC |
| | | GAAATCGGAATGAAGGGCGAGCGACGGCGCGGCAAAGGCC |
| | | ACGATGGACTCTATCAGGGCTTAAGCACCGCCACCAAAGAC |
| | | ACCTACGATGCACTTCATATGCAGGCACTCCCACCTAGATG |
| | | ATAA |
| MC0447 | SEQ ID | MALPVTALLLPLALLLHAARPDIVMTQSPDSLAVSLGERATISC |
| SN66E3.2 | NO: 280 | KSSQSVLYSSNNKNYLAWYQQKPGQPPKLLIYWASTRESGVP |
| (LH)_LIR1 | | DRESGSGSGTDFTLTISSLQAEDVAVYYCQQYYGTPFTFGGGT |
| (30)_ | | KVEIKGGGGSGGGGSGGGGSQVQLVQSGAEVKKPGASVKVS |
| (IRESL)_HER2 | | CKASGYTFTDYYLHWVRQAPGQGLEWMGWINPYTGGTNYA |
| Protein | | QKFQGRVTMTRDTSISTAYMELSGLTSDDTAVYYCARAGASY |
| Sequence | | YDFWSGWVFDYWGQGTLVTVSSGPTSTSGPEDQPLTPTGSDP |
| (VR447) | | QSGLGRHLGVVIGILVAVILLLLLLLLLFLILRHRRQGKHWTST |
| | | QRKADFQHPAGAVGPEPTDRGLQWRSSPAADAQEENLYAAV |
| | | KHTQPEDGVEMDTRSPHDEDPQAVTYAEVKHSRPRREMASPP |
| | | SPLSGEFLDTKDRQAEEDRQMDTEAAASEAPQDVTYAQLHSL |
| | | TLRREATEPPPSQEGPSPAVPSIYATLAIH* |
| | | MALPVTALLLPLALLLHAARPDIQMTQSPSSLSASVGDRVTITC |
| | | RASQDVNTAVAWYQQKPGKAPKLLIYSASFLYSGVPSRFSGSR |
| | | SGTDFTLTISSLQPEDFATYYCQQHYTTPPTFGQGTKVEIKGST |
| | | SGSGKPGSGEGSTKGEVQLVESGGGLVQPGGSLRLSCAASGEN |
| | | IKDTYIHWVRQAPGKGLEWVARIYPTNGYTRYADSVKGRFTIS |
| | | ADTSKNTAYLQMNSLRAEDTAVYYCSRWGGDGFYAMDYWG |
| | | QGTLVTVSSTTTPAPRPPTPAPTIASQPLSLRPEACRPAAGGAV |
| | | HTRGLDFACDIYIWAPLAGTCGVLLLSLVITLYCKRGRKKLLYI |
| | | FKQPFMRPVQTTQEEDGCSCRFPEEEEGGCELRVKFSRSADAP |
| | | AYQQGQNQLYNELNLGRREEYDVLDKRRGRDPEMGGKPRRK |
| | | NPQEGLYNELQKDKMAEAYSEIGMKGERRRGKGHDGLYQGL |
| | | STATKDTYDALHMQALPPR* |
| MC0449 | SEQ ID | ATGGCGCTGCCAGTCACTGCATTGTTATTGCCTCTGGCCCTG |
| SN66E3.3 | NO: 281 | CTTCTCCATGCGGCAAGGCCAGATATAGTGATGACACAGTC |
| (LH)_LIR1 | | CCCCGACTCCCTGGCTGTCTCACTGGGAGAACGAGCGACGA |
| (26)_ | | TTAGTTGTAAGTCTAGCCAGAGCGTCCTGTATTCAAGCAAT |
| (IRESL)_HER2 | | AACAAGAATTACCTCGCCTGGTATCAGCAAAAGCCGGGACA |
| Nucleotide | | GCCACCCAAACTGTTGATTTACTGGGCCAGCACGAGAGAGA |
| Sequence | | GCGGAGTGCCCGACCGCTTCAGCGGATCCGGGTCAGGCACA |
| (VR449) | | GATTTTACCCTGACTATTAGCTCCCTTCAAGCGGAAGATGTC |
| | | GCCGTCTACTATTGCCAGCAATATTACGGAACTCCATTCAC |
| | | ATTCGGCGGTGGGACCAAAGTAGAGATAAAGGGTGGCGGG |
| | | GGATCCGGCGGTGGCGGTAGCGGGGGAGGCGGGTCCCAAG |
| | | TGCAACTAGTCCAATCAGGTGCCGAAGTCAAGAAACCAGGT |
| | | GCATCCGTGAAAGTGTCTTGCAAAGCCAGTGGCTACACTTT |
| | | TACTGACTACTATCTGCACTGGGTGCGTCAAGCACCCGGCC |
| | | AGGGGCTTGAATGGATGGGCTGGATTAACCCTTATACTGGA |
| | | GGGACAAATTACGCTCAGAAGTTCCAGGGACGCGTTACAAT |
| | | GACCCGAGACACCAGCATCAGCACAGCGTACATGGAGTTA |
| | | AGTAGGCTGAGGTCCGAAGATACCGCCGTGTATTACTGCGC |
| | | TCGGGCAGGGGCCTCTTACTATGATTTTTGGTCCGGTTGGGT |
| | | CTTCGATTACTGGGGGCAGGGAACCCTGGTGACAGTGTCCT |
| | | CAACCAGTGGACCGGAAGATCAACCACTTACACCAACGGG |
| | | CAGCGACCCCCAGTCAGGCCTAGGGCGCCACCTGGGTGTGG |
| | | TCATCGGGATACTGGTCGCTGTCATCCTGCTTCTGCTCCTTC |
| | | TCTTGCTCCTATTCCTAATCCTGCGCCACAGGAGACAGGGC |
| | | AAGCACTGGACCAGCACCCAGCGGAAGGCCGACTTTCAGC |
| | | ACCCTGCCGGCGCCGTGGGCCCTGAGCCTACCGACAGGGGC |
| | | CTGCAGTGGAGGAGCTCCCCAGCCGCCGATGCCCAGGAGG |
| | | AGAATCTGTACGCCGCCGTGAAGCACACCCAGCCAGAGGA |
| | | CGGCGTGGAGATGGACACCCGCTCCCCACACGACGAGGAC |
| | | CCACAGGCCGTGACCTACGCCGAGGTGAAGCACAGCCGCCC |
| | | CAGACGCGAGATGGCCAGCCCACCCAGCCCCCTGTCCGGCG |
| | | AGTTCCTGGACACCAAGGACAGGCAGGCCGAGGAGGACCG |
| | | GCAGATGGACACCGAGGCCGCCGCCTCCGAGGCCCCCCAG |
| | | GACGTGACCTACGCCCAGCTGCACTCCCTGACCCTGCGGAG |
| | | AGAGGCCACCGAGCCCCCACCCAGCCAGGAGGGCCCCTCCC |
| | | CCGCCGTGCCTAGCATCTACGCCACCCTGGCCATCCACTGA |
| | | TAACCCCCCCCCCTAACGTTACTGGCCGAAGCCGCTTGGAA |
| | | TAAGGCCGGTGTGCGTTTGTCTATATGTTATTTTCCACCATA |
| | | TTGCCGTCTTTTGGCAATGTGAGGGCCCGGAAACCTGGCCC |
| | | TGTCTTCTTGACGAGCATTCCTAGGGGTCTTTCCCCTCTCGC |
| | | CAAAGGAATGCAAGGTCTGTTGAATGTCGTGAAGGAAGCA |
| | | GTTCCTCTGGAAGCTTCTTGAAGACAAACAACGTCTGTAGC |
| | | GACCCTTTGCAGGCAGCGGAACCCCCCACCTGGCGACAGGT |
| | | GCCTCTGCGGCCAAAAGCCACGTGTATAAGATACACCTGCA |
| | | AAGGCGGCACAACCCCAGTGCCACGTTGTGAGTTGGATAGT |
| | | TGTGGAAAGAGTCAAATGGCTCTCCTCAAGCGTATTCAACA |
| | | AGGGGCTGAAGGATGCCCAGAAGGTACCCCATTGTATGGG |
| | | ATCTGATCTGGGGCCTCGGTGCACATGCTTTACATGTGTTTA |
| | | GTCGAGGTTAAAAAAACGTCTAGGCCCCCCGAACCACGGG |
| | | GACGTGGTTTTCCTTTGAAAAACACGATGATAATATGATGG |
| | | CGCTGCCAGTCACTGCATTGTTATTGCCTCTGGCCCTGCTTC |
| | | TCCATGCGGCGCGCCCAGACATCCAGATGACCCAATCCCCA |
| | | AGCAGTCTCTCAGCCAGCGTGGGAGACAGGGTTACAATCAC |
| | | GTGCCGCGCCAGCCAGGACGTCAACACCGCTGTGGCTTGGT |
| | | ATCAGCAAAAGCCCGGGAAGGCACCAAAGCTGCTTATTTAT |
| | | AGCGCCTCCTTCTTGTATTCTGGAGTGCCATCCAGGTTTTCC |
| | | GGGTCACGTAGCGGGACTGACTTTACCCTCACCATATCCAG |
| | | CCTCCAGCCCGAGGATTTCGCCACCTATTACTGTCAGCAAC |
| | | ACTACACGACTCCACCGACTTTTGGACAGGGCACTAAAGTG |
| | | GAGATTAAGGGCAGCACGAGTGGGAGTGGAAAGCCCGGCA |
| | | GCGGGGAGGGGTCTACCAAGGGAGAGGTCCAGCTGGTTGA |
| | | ATCCGGAGGCGGGCTTGTGCAACCTGGAGGCTCCCTGAGGC |
| | | TTAGTTGTGCCGCGTCAGGATTCAACATTAAGGATACCTAT |
| | | ATTCATTGGGTCCGACAAGCCCCGGGCAAGGGCTTGGAGTG |
| | | GGTGGCCAGAATCTATCCGACCAACGGATATACAAGGTACG |
| | | CCGATTCTGTGAAAGGACGCTTCACCATCAGCGCGGACACA |
| | | TCCAAAAACACAGCCTATCTGCAGATGAACTCCCTTCGCGC |
| | | CGAGGATACAGCCGTGTACTATTGTAGTCGGTGGGGAGGCG |
| | | ACGGCTTCTACGCGATGGACTATTGGGGACAAGGAACACTG |
| | | GTGACTGTCAGTAGCACTACGACCCCAGCACCTAGACCTCC |
| | | CACCCCAGCTCCAACTATAGCTTCCCAGCCATTGTCTCTCCG |
| | | GCCAGAGGCGTGTCGACCAGCCGCTGGAGGGGCCGTTCATA |
| | | CAAGAGGACTCGATTTCGCTTGCGATATCTACATATGGGCC |
| | | CCTCTTGCCGGGACATGCGGTGTCCTGCTTCTAAGCTTGGTT |
| | | ATTACCCTCTATTGCAAAAGAGGACGAAAGAAACTGCTTTA |
| | | TATATTCAAGCAACCTTTCATGCGCCCCGTACAGACCACGC |
| | | AGGAGGAAGATGGGTGTAGCTGTCGCTTCCCTGAGGAAGA |
| | | GGAAGGTGGATGCGAGTTGCGGGTGAAGTTCAGTCGATCCG |
| | | CCGATGCGCCTGCCTATCAGCAAGGGCAGAACCAGCTTTAT |
| | | AACGAGTTAAACCTTGGCCGCCGGGAAGAGTATGACGTGTT |
| | | GGACAAGCGTCGCGGGAGAGACCCTGAGATGGGCGGAAAA |
| | | CCAAGGAGAAAAAATCCACAGGAAGGCTTATATAACGAGT |
| | | TGCAGAAAGACAAGATGGCCGAGGCATACTCCGAAATCGG |
| | | AATGAAGGGCGAGCGACGGCGCGGCAAAGGCCACGATGGA |
| | | CTCTATCAGGGCTTAAGCACCGCCACCAAAGACACCTACGA |
| | | TGCACTTCATATGCAGGCACTCCCACCTAGATGATAA |
| MC0449 | SEQ ID | MALPVTALLLPLALLLHAARPDIVMTQSPDSLAVSLGERATISC |
| SN66E3.3 | NO: 282 | KSSQSVLYSSNNKNYLAWYQQKPGQPPKLLIYWASTRESGVP |
| (LH)_LIR1 | | DRFSGSGSGTDFTLTISSLQAEDVAVYYCQQYYGTPFTFGGGT |
| (26)_ | | KVEIKGGGGSGGGGSGGGGSQVQLVQSGAEVKKPGASVKVS |
| (IRESL)_HER2 | | CKASGYTFTDYYLHWVRQAPGQGLEWMGWINPYTGGTNYA |
| Protein | | QKFQGRVTMTRDTSISTAYMELSRLRSEDTAVYYCARAGASY |
| Sequence | | YDFWSGWVFDYWGQGTLVTVSSTSGPEDQPLTPTGSDPQSGL |
| (VR449) | | GRHLGVVIGILVAVILLLLLLLLLFLILRHRRQGKHWTSTQRKA |
| | | DFQHPAGAVGPEPTDRGLQWRSSPAADAQEENLYAAVKHTQP |
| | | EDGVEMDTRSPHDEDPQAVTYAEVKHSRPRREMASPPSPLSGE |
| | | FLDTKDRQAEEDRQMDTEAAASEAPQDVTYAQLHSLTLRREA |
| | | TEPPPSQEGPSPAVPSIYATLAIH* |
| | | MALPVTALLLPLALLLHAARPDIQMTQSPSSLSASVGDRVTITC |
| | | RASQDVNTAVAWYQQKPGKAPKLLIYSASFLYSGVPSRESGSR |
| | | SGTDFTLTISSLQPEDFATYYCQQHYTTPPTFGQGTKVEIKGST |
| | | SGSGKPGSGEGSTKGEVQLVESGGGLVQPGGSLRLSCAASGEN |
| | | IKDTYIHWVRQAPGKGLEWVARIYPTNGYTRYADSVKGRFTIS |
| | | ADTSKNTAYLQMNSLRAEDTAVYYCSRWGGDGFYAMDYWG |
| | | QGTLVTVSSTTTPAPRPPTPAPTIASQPLSLRPEACRPAAGGAV |
| | | HTRGLDFACDIYIWAPLAGTCGVLLLSLVITLYCKRGRKKLLYI |
| | | FKQPFMRPVQTTQEEDGCSCRFPEEEEGGCELRVKFSRSADAP |
| | | AYQQGQNQLYNELNLGRREEYDVLDKRRGRDPEMGGKPRRK |
| | | NPQEGLYNELQKDKMAEAYSEIGMKGERRRGKGHDGLYQGL |
| | | STATKDTYDALHMQALPPR* |
| MC0515- | SEQ ID | ATGGCGCTGCCAGTCACTGCATTGTTATTGCCTCTGGCCCTG |
| HzBB7.2(2)_ | NO: 321 | TGGTGCTGAGGTGAAAAAGCCCGGCGCATCCGTGAAAGTG |
| LIR1(30)_ | | CTTCTCCATGCGGCAAGGCCACAGGTGCAACTGGTTCAATC |
| 2A_HER2 | | AGCTGTAAGGCATCAGGGTACACCTTCACCAGCTATCACAT |
| Nucleotide | | ACAATGGGTCCGCCAGGCCCCCGGACAGAGGTTGGAATGG |
| Sequence | | TGAGAAGTTCAAGGGCAGGGTGACTATCACACGCGATACCT |
| (VR515) | | ATTGGGTGGATTTACCCGGGTGACGGCTCAACCCAGTACAA |
| | | CCGCGAGCACAGCTTACATGGAGTTATCTAGCCTGAGATCC |
| | | GAAGATACGGCGGTGTATTACTGCGCGCGGGAAGGGACCT |
| | | ACTATGCCATGGACTATTGGGGACAAGGGACCCTGGTTACC |
| | | GTGAGTTCTGGGGGCGGGGGTTCCGGGGGAGGGGGATCTG |
| | | GGGGTGGAGGGAGCGATGTGGTAATGACCCAGACACCTTTG |
| | | TCTTTGAGTGTCACCCCCGGACAGCCGGCAAGTATATCCTG |
| | | TAGATCATCCCAATCAATCGTGCACTCCAACGGAAACACAT |
| | | ACTTGGAATGGTATCTCCAGAAACCTGGACAGTCCCCACAG |
| | | TTGCTCATCTACAAAGTGTCAAATCGCTTTTCAGGCGTGCCC |
| | | GATCGTTTCAGCGGCTCAGGCTCCGGGACAGACTTTACATT |
| | | GAAGATTAGCCGCGTAGAGGCAGAGGATGTGGGCGTTTACT |
| | | ATTGTTTTCAAGGGTCACACGTGCCACGCACATTCGGCGGC |
| | | GGTACCAAGGTGGAAATTAAGGGCCCCACTTCTACCAGTGG |
| | | ACCGGAAGATCAACCACTTACACCAACGGGCAGCGACCCCC |
| | | AGTCAGGCCTAGGGCGCCACCTGGGTGTGGTCATCGGGATA |
| | | CTGGTCGCTGTCATCCTGCTTCTGCTCCTTCTCTTGCTCCTAT |
| | | TCCTAATCCTGCGCCACAGGAGACAGGGCAAGCACTGGACC |
| | | AGCACCCAGCGGAAGGCCGACTTTCAGCACCCTGCCGGCGC |
| | | CGTGGGCCCTGAGCCTACCGACAGGGGCCTGCAGTGGAGG |
| | | AGCTCCCCAGCCGCCGATGCCCAGGAGGAGAATCTGTACGC |
| | | CGCCGTGAAGCACACCCAGCCAGAGGACGGCGTGGAGATG |
| | | GACACCCGCTCCCCACACGACGAGGACCCACAGGCCGTGAC |
| | | CTACGCCGAGGTGAAGCACAGCCGCCCCAGACGCGAGATG |
| | | GCCAGCCCACCCAGCCCCCTGTCCGGCGAGTTCCTGGACAC |
| | | CAAGGACAGGCAGGCCGAGGAGGACCGGCAGATGGACACC |
| | | GAGGCCGCCGCCTCCGAGGCCCCCCAGGACGTGACCTACGC |
| | | CCAGCTGCACTCCCTGACCCTGCGGAGAGAGGCCACCGAGC |
| | | CCCCACCCAGCCAGGAGGGCCCCTCCCCCGCCGTGCCTAGC |
| | | ATCTACGCCACCCTGGCCATCCACGGATCCGGGGAAGGCCG |
| | | AGGCTCCCTTCTAACATGTGGAGATGTCGAGGAAAACCCTG |
| | | GCCCTATGGCGCTGCCAGTCACTGCATTGTTATTGCCTCTGG |
| | | CCCTGCTTCTCCATGCGGCGCGCCCAGACATCCAGATGACC |
| | | CAATCCCCAAGCAGTCTCTCAGCCAGCGTGGGAGACAGGGT |
| | | TACAATCACGTGCCGCGCCAGCCAGGACGTCAACACCGCTG |
| | | TGGCTTGGTATCAGCAAAAGCCCGGGAAGGCACCAAAGCT |
| | | GCTTATTTATAGCGCCTCCTTCTTGTATTCTGGAGTGCCATC |
| | | CAGGTTTTCCGGGTCACGTAGCGGGACTGACTTTACCCTCA |
| | | CCATATCCAGCCTCCAGCCCGAGGATTTCGCCACCTATTACT |
| | | GTCAGCAACACTACACGACTCCACCGACTTTTGGACAGGGC |
| | | ACTAAAGTGGAGATTAAGGGCAGCACGAGTGGGAGTGGAA |
| | | AGCCCGGCAGCGGGGAGGGGTCTACCAAGGGAGAGGTCCA |
| | | GCTGGTTGAATCCGGAGGCGGGCTTGTGCAACCTGGAGGCT |
| | | CCCTGAGGCTTAGTTGTGCCGCGTCAGGATTCAACATTAAG |
| | | GATACCTATATTCATTGGGTCCGACAAGCCCCGGGCAAGGG |
| | | CTTGGAGTGGGTGGCCAGAATCTATCCGACCAACGGATATA |
| | | CAAGGTACGCCGATTCTGTGAAAGGACGCTTCACCATCAGC |
| | | GCGGACACATCCAAAAACACAGCCTATCTGCAGATGAACTC |
| | | CCTTCGCGCCGAGGATACAGCCGTGTACTATTGTAGTCGGT |
| | | GGGGAGGCGACGGCTTCTACGCGATGGACTATTGGGGACA |
| | | AGGAACACTGGTGACTGTCAGTAGCACTACGACCCCAGCAC |
| | | CTAGACCTCCCACCCCAGCTCCAACTATAGCTTCCCAGCCAT |
| | | TGTCTCTCCGGCCAGAGGCGTGTCGACCAGCCGCTGGAGGG |
| | | GCCGTTCATACAAGAGGACTCGATTTCGCTTGCGATATCTA |
| | | CATATGGGCCCCTCTTGCCGGGACATGCGGTGTCCTGCTTCT |
| | | AAGCTTGGTTATTACCCTCTATTGCAAAAGAGGACGAAAGA |
| | | AACTGCTTTATATATTCAAGCAACCTTTCATGCGCCCCGTAC |
| | | AGACCACGCAGGAGGAAGATGGGTGTAGCTGTCGCTTCCCT |
| | | GAGGAAGAGGAAGGTGGATGCGAGTTGCGGGTGAAGTTCA |
| | | GTCGATCCGCCGATGCGCCTGCCTATCAGCAAGGGCAGAAC |
| | | CAGCTTTATAACGAGTTAAACCTTGGCCGCCGGGAAGAGTA |
| | | TGACGTGTTGGACAAGCGTCGCGGGAGAGACCCTGAGATG |
| | | GGCGGAAAACCAAGGAGAAAAAATCCACAGGAAGGCTTAT |
| | | ATAACGAGTTGCAGAAAGACAAGATGGCCGAGGCATACTC |
| | | CGAAATCGGAATGAAGGGCGAGCGACGGCGCGGCAAAGGC |
| | | CACGATGGACTCTATCAGGGCTTAAGCACCGCCACCAAAGA |
| | | CACCTACGATGCACTTCATATGCAGGCACTCCCACCTAGAT |
| | | GATAA |
| MC0515 | SEQ ID | MALPVTALLLPLALLLHAARPQVQLVQSGAEVKKPGASVKVS |
| HzBB7.2(2)_ | NO: 322 | CKASGYTFTSYHIQWVRQAPGQRLEWIGWIYPGDGSTQYNEK |
| LIR1(30)_ | | FKGRVTITRDTSASTAYMELSSLRSEDTAVYYCAREGTYYAM |
| 2A_HER2 | | DYWGQGTLVTVSSGGGGSGGGGSGGGGSDVVMTQTPLSLSV |
| Protein | | TPGQPASISCRSSQSIVHSNGNTYLEWYLQKPGQSPQLLIYKVS |
| Sequence | | NRFSGVPDRFSGSGSGTDFTLKISRVEAEDVGVYYCFQGSHVP |
| (VR515) | | RTFGGGTKVEIKGPTSTSGPEDQPLTPTGSDPQSGLGRHLGVVI |
| | | GILVAVILLLLLLLLLFLILRHRRQGKHWTSTQRKADFQHPAG |
| | | AVGPEPTDRGLQWRSSPAADAQEENLYAAVKHTQPEDGVEM |
| | | DTRSPHDEDPQAVTYAEVKHSRPRREMASPPSPLSGEFLDTKD |
| | | RQAEEDRQMDTEAAASEAPQDVTYAQLHSLTLRREATEPPPS |
| | | QEGPSPAVPSIYATLAIHGSGEGRGSLLTCGDVEENPGPMALPV |
| | | TALLLPLALLLHAARPDIQMTQSPSSLSASVGDRVTITCRASQD |
| | | VNTAVAWYQQKPGKAPKLLIYSASFLYSGVPSRFSGSRSGTDF |
| | | TLTISSLQPEDFATYYCQQHYTTPPTFGQGTKVEIKGSTSGSGK |
| | | PGSGEGSTKGEVQLVESGGGLVQPGGSLRLSCAASGFNIKDTY |
| | | IHWVRQAPGKGLEWVARIYPTNGYTRYADSVKGRFTISADTS |
| | | KNTAYLQMNSLRAEDTAVYYCSRWGGDGFYAMDYWGQGTL |
| | | VTVSSTTTPAPRPPTPAPTIASQPLSLRPEACRPAAGGAVHTRG |
| | | LDFACDIYIWAPLAGTCGVLLLSLVITLYCKRGRKKLLYIFKQP |
| | | FMRPVQTTQEEDGCSCRFPEEEEGGCELRVKFSRSADAPAYQQ |
| | | GQNQLYNELNLGRREEYDVLDKRRGRDPEMGGKPRRKNPQE |
| | | GLYNELQKDKMAEAYSEIGMKGERRRGKGHDGLYQGLSTAT |
| | | KDTYDALHMQALPPR |
| MC0516- | SEQ ID | ATGGCGCTGCCAGTCACTGCATTGTTATTGCCTCTGGCCCTG |
| SN66E3.2 | NO: 323 | CTTCTCCATGCGGCAAGGCCAGATATAGTGATGACACAGTC |
| (LH)_LIR1 | | CCCCGACTCCCTGGCTGTCTCACTGGGAGAACGAGCGACGA |
| (30)_2A_HER2 | | TTAGTTGTAAGTCTAGCCAGAGCGTCCTGTATTCAAGCAAT |
| Nucleotide | | AACAAGAATTACCTCGCCTGGTATCAGCAAAAGCCGGGACA |
| Sequence | | GCCACCCAAACTGTTGATTTACTGGGCCAGCACGAGAGAGA |
| (VR516) | | GCGGAGTGCCCGACCGCTTCAGCGGATCCGGGTCAGGCACA |
| | | GATTTTACCCTGACTATTAGCTCCCTTCAAGCGGAAGATGTC |
| | | GCCGTCTACTATTGCCAGCAATATTACGGAACTCCATTCAC |
| | | ATTCGGCGGTGGGACCAAAGTAGAGATAAAGGGTGGCGGG |
| | | GGATCCGGCGGTGGCGGTAGCGGGGGAGGCGGGTCCCAAG |
| | | TGCAACTAGTCCAATCAGGTGCCGAAGTCAAGAAACCAGGT |
| | | GCATCCGTGAAAGTGTCTTGCAAAGCCAGTGGCTACACTTT |
| | | TACTGACTACTATCTGCACTGGGTGCGTCAAGCACCCGGCC |
| | | AGGGGCTTGAATGGATGGGCTGGATTAACCCTTATACTGGA |
| | | GGGACAAATTACGCTCAGAAGTTCCAGGGACGCGTTACAAT |
| | | GACCCGAGACACCAGCATCAGCACAGCGTACATGGAGTTA |
| | | AGTGGGCTGACTTCCGACGATACCGCCGTGTATTACTGCGC |
| | | TCGGGCAGGGGCCTCTTACTATGATTTTTGGTCCGGTTGGGT |
| | | CTTCGATTACTGGGGGCAGGGAACCCTGGTGACAGTGTCCT |
| | | CAGGCCCCACTTCTACCAGTGGACCGGAAGATCAACCACTT |
| | | ACACCAACGGGCAGCGACCCCCAGTCAGGCCTAGGGCGCC |
| | | ACCTGGGTGTGGTCATCGGGATACTGGTCGCTGTCATCCTG |
| | | CTTCTGCTCCTTCTCTTGCTCCTATTCCTAATCCTGCGCCACA |
| | | GGAGACAGGGCAAGCACTGGACCAGCACCCAGCGGAAGGC |
| | | CGACTTTCAGCACCCTGCCGGCGCCGTGGGCCCTGAGCCTA |
| | | CCGACAGGGGCCTGCAGTGGAGGAGCTCCCCAGCCGCCGAT |
| | | GCCCAGGAGGAGAATCTGTACGCCGCCGTGAAGCACACCC |
| | | AGCCAGAGGACGGCGTGGAGATGGACACCCGCTCCCCACA |
| | | CGACGAGGACCCACAGGCCGTGACCTACGCCGAGGTGAAG |
| | | CACAGCCGCCCCAGACGCGAGATGGCCAGCCCACCCAGCCC |
| | | CCTGTCCGGCGAGTTCCTGGACACCAAGGACAGGCAGGCCG |
| | | AGGAGGACCGGCAGATGGACACCGAGGCCGCCGCCTCCGA |
| | | GGCCCCCCAGGACGTGACCTACGCCCAGCTGCACTCCCTGA |
| | | CCCTGCGGAGAGAGGCCACCGAGCCCCCACCCAGCCAGGA |
| | | GGGCCCCTCCCCCGCCGTGCCTAGCATCTACGCCACCCTGG |
| | | CCATCCACGGATCCGGGGAAGGCCGAGGCTCCCTTCTAACA |
| | | TGTGGAGATGTCGAGGAAAACCCTGGCCCTATGGCGCTGCC |
| | | AGTCACTGCATTGTTATTGCCTCTGGCCCTGCTTCTCCATGC |
| | | GGCGCGCCCAGACATCCAGATGACCCAATCCCCAAGCAGTC |
| | | TCTCAGCCAGCGTGGGAGACAGGGTTACAATCACGTGCCGC |
| | | GCCAGCCAGGACGTCAACACCGCTGTGGCTTGGTATCAGCA |
| | | AAAGCCCGGGAAGGCACCAAAGCTGCTTATTTATAGCGCCT |
| | | CCTTCTTGTATTCTGGAGTGCCATCCAGGTTTTCCGGGTCAC |
| | | GTAGCGGGACTGACTTTACCCTCACCATATCCAGCCTCCAG |
| | | CCCGAGGATTTCGCCACCTATTACTGTCAGCAACACTACAC |
| | | GACTCCACCGACTTTTGGACAGGGCACTAAAGTGGAGATTA |
| | | AGGGCAGCACGAGTGGGAGTGGAAAGCCCGGCAGCGGGGA |
| | | GGGGTCTACCAAGGGAGAGGTCCAGCTGGTTGAATCCGGA |
| | | GGCGGGCTTGTGCAACCTGGAGGCTCCCTGAGGCTTAGTTG |
| | | TGCCGCGTCAGGATTCAACATTAAGGATACCTATATTCATT |
| | | GGGTCCGACAAGCCCCGGGCAAGGGCTTGGAGTGGGTGGC |
| | | CAGAATCTATCCGACCAACGGATATACAAGGTACGCCGATT |
| | | CTGTGAAAGGACGCTTCACCATCAGCGCGGACACATCCAAA |
| | | AACACAGCCTATCTGCAGATGAACTCCCTTCGCGCCGAGGA |
| | | TACAGCCGTGTACTATTGTAGTCGGTGGGGAGGCGACGGCT |
| | | TCTACGCGATGGACTATTGGGGACAAGGAACACTGGTGACT |
| | | GTCAGTAGCACTACGACCCCAGCACCTAGACCTCCCACCCC |
| | | AGCTCCAACTATAGCTTCCCAGCCATTGTCTCTCCGGCCAGA |
| | | GGCGTGTCGACCAGCCGCTGGAGGGGCCGTTCATACAAGAG |
| | | GACTCGATTTCGCTTGCGATATCTACATATGGGCCCCTCTTG |
| | | CCGGGACATGCGGTGTCCTGCTTCTAAGCTTGGTTATTACCC |
| | | TCTATTGCAAAAGAGGACGAAAGAAACTGCTTTATATATTC |
| | | AAGCAACCTTTCATGCGCCCCGTACAGACCACGCAGGAGGA |
| | | AGATGGGTGTAGCTGTCGCTTCCCTGAGGAAGAGGAAGGTG |
| | | GATGCGAGTTGCGGGTGAAGTTCAGTCGATCCGCCGATGCG |
| | | CCTGCCTATCAGCAAGGGCAGAACCAGCTTTATAACGAGTT |
| | | AAACCTTGGCCGCCGGGAAGAGTATGACGTGTTGGACAAGC |
| | | GTCGCGGGAGAGACCCTGAGATGGGCGGAAAACCAAGGAG |
| | | AAAAAATCCACAGGAAGGCTTATATAACGAGTTGCAGAAA |
| | | GACAAGATGGCCGAGGCATACTCCGAAATCGGAATGAAGG |
| | | GCGAGCGACGGCGCGGCAAAGGCCACGATGGACTCTATCA |
| | | GGGCTTAAGCACCGCCACCAAAGACACCTACGATGCACTTC |
| | | ATATGCAGGCACTCCCACCTAGATGATAA |
| MC0516- | SEQ ID | MALPVTALLLPLALLLHAARPDIVMTQSPDSLAVSLGERATISC |
| SN66E3.2 | NO: 324 | KSSQSVLYSSNNKNYLAWYQQKPGQPPKLLIYWASTRESGVP |
| (LH)_LIR1 | | DRFSGSGSGTDFTLTISSLQAEDVAVYYCQQYYGTPFTFGGGT |
| (30)_2A_HER2 | | KVEIKGGGGSGGGGSGGGGSQVQLVQSGAEVKKPGASVKVS |
| Protein | | CKASGYTFTDYYLHWVRQAPGQGLEWMGWINPYTGGTNYA |
| Sequence | | QKFQGRVTMTRDTSISTAYMELSGLTSDDTAVYYCARAGASY |
| (VR516) | | YDFWSGWVFDYWGQGTLVTVSSGPTSTSGPEDQPLTPTGSDP |
| | | QSGLGRHLGVVIGILVAVILLLLLLLLLFLILRHRRQGKHWTST |
| | | QRKADFQHPAGAVGPEPTDRGLQWRSSPAADAQEENLYAAV |
| | | KHTQPEDGVEMDTRSPHDEDPQAVTYAEVKHSRPRREMASPP |
| | | SPLSGEFLDTKDRQAEEDRQMDTEAAASEAPQDVTYAQLHSL |
| | | TLRREATEPPPSQEGPSPAVPSIYATLAIHGSGEGRGSLLTCGDV |
| | | EENPGPMALPVTALLLPLALLLHAARPDIQMTQSPSSLSASVG |
| | | DRVTITCRASQDVNTAVAWYQQKPGKAPKLLIYSASFLYSGVP |
| | | SRESGSRSGTDFTLTISSLQPEDFATYYCQQHYTTPPTFGQGTK |
| | | VEIKGSTSGSGKPGSGEGSTKGEVQLVESGGGLVQPGGSLRLS |
| | | CAASGFNIKDTYIHWVRQAPGKGLEWVARIYPTNGYTRYADS |
| | | VKGRFTISADTSKNTAYLQMNSLRAEDTAVYYCSRWGGDGFY |
| | | AMDYWGQGTLVTVSSTTTPAPRPPTPAPTIASQPLSLRPEACRP |
| | | AAGGAVHTRGLDFACDIYIWAPLAGTCGVLLLSLVITLYCKRG |
| | | RKKLLYIFKQPFMRPVQTTQEEDGCSCRFPEEEEGGCELRVKFS |
| | | RSADAPAYQQGQNQLYNELNLGRREEYDVLDKRRGRDPEMG |
| | | GKPRRKNPQEGLYNELQKDKMAEAYSEIGMKGERRRGKGHD |
| | | GLYQGLSTATKDTYDALHMQALPPR |
| MC0517- | SEQ ID | ATGGCGCTGCCAGTCACTGCATTGTTATTGCCTCTGGCCCTG |
| SN66E3.3_ | NO: 325 | CTTCTCCATGCGGCAAGGCCAGATATAGTGATGACACAGTC |
| (LH)_LIRI | | CCCCGACTCCCTGGCTGTCTCACTGGGAGAACGAGCGACGA |
| (26)_2A_HER2 | | TTAGTTGTAAGTCTAGCCAGAGCGTCCTGTATTCAAGCAAT |
| Nuclotide | | AACAAGAATTACCTCGCCTGGTATCAGCAAAAGCCGGGACA |
| Sequence | | GCCACCCAAACTGTTGATTTACTGGGCCAGCACGAGAGAGA |
| (VR517) | | GCGGAGTGCCCGACCGCTTCAGCGGATCCGGGTCAGGCACA |
| | | GATTTTACCCTGACTATTAGCTCCCTTCAAGCGGAAGATGTC |
| | | GCCGTCTACTATTGCCAGCAATATTACGGAACTCCATTCAC |
| | | ATTCGGCGGTGGGACCAAAGTAGAGATAAAGGGTGGCGGG |
| | | GGATCCGGCGGTGGCGGTAGCGGGGGAGGCGGGTCCCAAG |
| | | TGCAACTAGTCCAATCAGGTGCCGAAGTCAAGAAACCAGGT |
| | | GCATCCGTGAAAGTGTCTTGCAAAGCCAGTGGCTACACTTT |
| | | TACTGACTACTATCTGCACTGGGTGCGTCAAGCACCCGGCC |
| | | AGGGGCTTGAATGGATGGGCTGGATTAACCCTTATACTGGA |
| | | GGGACAAATTACGCTCAGAAGTTCCAGGGACGCGTTACAAT |
| | | GACCCGAGACACCAGCATCAGCACAGCGTACATGGAGTTA |
| | | AGTAGGCTGAGGTCCGAAGATACCGCCGTGTATTACTGCGC |
| | | TCGGGCAGGGGCCTCTTACTATGATTTTTGGTCCGGTTGGGT |
| | | CTTCGATTACTGGGGGCAGGGAACCCTGGTGACAGTGTCCT |
| | | CAACCAGTGGACCGGAAGATCAACCACTTACACCAACGGG |
| | | CAGCGACCCCCAGTCAGGCCTAGGGCGCCACCTGGGTGTGG |
| | | TCATCGGGATACTGGTCGCTGTCATCCTGCTTCTGCTCCTTC |
| | | TCTTGCTCCTATTCCTAATCCTGCGCCACAGGAGACAGGGC |
| | | AAGCACTGGACCAGCACCCAGCGGAAGGCCGACTTTCAGC |
| | | ACCCTGCCGGCGCCGTGGGCCCTGAGCCTACCGACAGGGGC |
| | | CTGCAGTGGAGGAGCTCCCCAGCCGCCGATGCCCAGGAGG |
| | | AGAATCTGTACGCCGCCGTGAAGCACACCCAGCCAGAGGA |
| | | CGGCGTGGAGATGGACACCCGCTCCCCACACGACGAGGAC |
| | | CCACAGGCCGTGACCTACGCCGAGGTGAAGCACAGCCGCCC |
| | | CAGACGCGAGATGGCCAGCCCACCCAGCCCCCTGTCCGGCG |
| | | AGTTCCTGGACACCAAGGACAGGCAGGCCGAGGAGGACCG |
| | | GCAGATGGACACCGAGGCCGCCGCCTCCGAGGCCCCCCAG |
| | | GACGTGACCTACGCCCAGCTGCACTCCCTGACCCTGCGGAG |
| | | AGAGGCCACCGAGCCCCCACCCAGCCAGGAGGGCCCCTCCC |
| | | CCGCCGTGCCTAGCATCTACGCCACCCTGGCCATCCACGGA |
| | | TCCGGGGAAGGCCGAGGCTCCCTTCTAACATGTGGAGATGT |
| | | CGAGGAAAACCCTGGCCCTATGGCGCTGCCAGTCACTGCAT |
| | | TGTTATTGCCTCTGGCCCTGCTTCTCCATGCGGCGCGCCCAG |
| | | ACATCCAGATGACCCAATCCCCAAGCAGTCTCTCAGCCAGC |
| | | GTGGGAGACAGGGTTACAATCACGTGCCGCGCCAGCCAGG |
| | | ACGTCAACACCGCTGTGGCTTGGTATCAGCAAAAGCCCGGG |
| | | AAGGCACCAAAGCTGCTTATTTATAGCGCCTCCTTCTTGTAT |
| | | TCTGGAGTGCCATCCAGGTTTTCCGGGTCACGTAGCGGGAC |
| | | TGACTTTACCCTCACCATATCCAGCCTCCAGCCCGAGGATTT |
| | | CGCCACCTATTACTGTCAGCAACACTACACGACTCCACCGA |
| | | CTTTTGGACAGGGCACTAAAGTGGAGATTAAGGGCAGCACG |
| | | AGTGGGAGTGGAAAGCCCGGCAGCGGGGAGGGGTCTACCA |
| | | AGGGAGAGGTCCAGCTGGTTGAATCCGGAGGCGGGCTTGTG |
| | | CAACCTGGAGGCTCCCTGAGGCTTAGTTGTGCCGCGTCAGG |
| | | ATTCAACATTAAGGATACCTATATTCATTGGGTCCGACAAG |
| | | CCCCGGGCAAGGGCTTGGAGTGGGTGGCCAGAATCTATCCG |
| | | ACCAACGGATATACAAGGTACGCCGATTCTGTGAAAGGACG |
| | | CTTCACCATCAGCGCGGACACATCCAAAAACACAGCCTATC |
| | | TGCAGATGAACTCCCTTCGCGCCGAGGATACAGCCGTGTAC |
| | | TATTGTAGTCGGTGGGGAGGCGACGGCTTCTACGCGATGGA |
| | | CTATTGGGGACAAGGAACACTGGTGACTGTCAGTAGCACTA |
| | | CGACCCCAGCACCTAGACCTCCCACCCCAGCTCCAACTATA |
| | | GCTTCCCAGCCATTGTCTCTCCGGCCAGAGGCGTGTCGACC |
| | | AGCCGCTGGAGGGGCCGTTCATACAAGAGGACTCGATTTCG |
| | | CTTGCGATATCTACATATGGGCCCCTCTTGCCGGGACATGC |
| | | GGTGTCCTGCTTCTAAGCTTGGTTATTACCCTCTATTGCAAA |
| | | AGAGGACGAAAGAAACTGCTTTATATATTCAAGCAACCTTT |
| | | CATGCGCCCCGTACAGACCACGCAGGAGGAAGATGGGTGT |
| | | AGCTGTCGCTTCCCTGAGGAAGAGGAAGGTGGATGCGAGTT |
| | | GCGGGTGAAGTTCAGTCGATCCGCCGATGCGCCTGCCTATC |
| | | AGCAAGGGCAGAACCAGCTTTATAACGAGTTAAACCTTGGC |
| | | CGCCGGGAAGAGTATGACGTGTTGGACAAGCGTCGCGGGA |
| | | GAGACCCTGAGATGGGCGGAAAACCAAGGAGAAAAAATCC |
| | | ACAGGAAGGCTTATATAACGAGTTGCAGAAAGACAAGATG |
| | | GCCGAGGCATACTCCGAAATCGGAATGAAGGGCGAGCGAC |
| | | GGCGCGGCAAAGGCCACGATGGACTCTATCAGGGCTTAAGC |
| | | ACCGCCACCAAAGACACCTACGATGCACTTCATATGCAGGC |
| | | ACTCCCACCTAGATGATAA |
| MC0517- | SEQ ID | MALPVTALLLPLALLLHAARPDIVMTQSPDSLAVSLGERATISC |
| SN66E3.3 | NO: 326 | KSSQSVLYSSNNKNYLAWYQQKPGQPPKLLIYWASTRESGVP |
| (LH)_LIR1 | | DRESGSGSGTDFTLTISSLQAEDVAVYYCQQYYGTPFTFGGGT |
| (26)_2A_HER2 | | KVEIKGGGGSGGGGSGGGGSQVQLVQSGAEVKKPGASVKVS |
| Protein | | CKASGYTFTDYYLHWVRQAPGQGLEWMGWINPYTGGTNYA |
| Sequence | | QKFQGRVTMTRDTSISTAYMELSRLRSEDTAVYYCARAGASY |
| (VR517) | | YDFWSGWVFDYWGQGTLVTVSSTSGPEDQPLTPTGSDPQSGL |
| | | GRHLGVVIGILVAVILLLLLLLLLFLILRHRRQGKHWTSTQRKA |
| | | DFQHPAGAVGPEPTDRGLQWRSSPAADAQEENLYAAVKHTQP |
| | | EDGVEMDTRSPHDEDPQAVTYAEVKHSRPRREMASPPSPLSGE |
| | | FLDTKDRQAEEDRQMDTEAAASEAPQDVTYAQLHSLTLRREA |
| | | TEPPPSQEGPSPAVPSIYATLAIHGSGEGRGSLLTCGDVEENPGP |
| | | MALPVTALLLPLALLLHAARPDIQMTQSPSSLSASVGDRVTITC |
| | | RASQDVNTAVAWYQQKPGKAPKLLIYSASFLYSGVPSRFSGSR |
| | | SGTDFTLTISSLQPEDFATYYCQQHYTTPPTFGQGTKVEIKGST |
| | | SGSGKPGSGEGSTKGEVQLVESGGGLVQPGGSLRLSCAASGEN |
| | | IKDTYIHWVRQAPGKGLEWVARIYPTNGYTRYADSVKGRFTIS |
| | | ADTSKNTAYLQMNSLRAEDTAVYYCSRWGGDGFYAMDYWG |
| | | QGTLVTVSSTTTPAPRPPTPAPTIASQPLSLRPEACRPAAGGAV |
| | | HTRGLDFACDIYIWAPLAGTCGVLLLSLVITLYCKRGRKKLLYI |
| | | FKQPFMRPVQTTQEEDGCSCRFPEEEEGGCELRVKFSRSADAP |
| | | AYQQGQNQLYNELNLGRREEYDVLDKRRGRDPEMGGKPRRK |
| | | NPQEGLYNELQKDKMAEAYSEIGMKGERRRGKGHDGLYQGL |
| | | STATKDTYDALHMQALPPR |

ii. Bicistronic iCAR Portion

In some embodiments, the bicistronic iCAR portions described below can be included as part of monocistronic iCAR constructs for use in co-transduction methods along with a described monocistronic aCAR construct.

1. iCAR Portion: scFv Component

In some embodiments, the bicistronic construct comprises an iCAR portion comprising a single chain variable fragment (scFv) component. In some embodiments, the iCAR portion comprises a single chain variable fragment (scFv) component. In some embodiments, the scFv targets an HLA antigen. In some embodiments, the HLA antigen is selected from the group consisting of HLA-A2, HLA-A3, HLA-A, HLA-B, HLA-C, HLA-G, HLA-E, HLA-F, HLA-DPA1, HLA-DQA1, HLA-DQB1, HLA-DQB2, HLA-DRB1, and HLA-DRB5. In some embodiments, the iCAR comprises an scFv. In some embodiments, the scFv is selected from the group consisting of BB7.2, 3PF12, 3PF12/C4, 3PF12/F12, 3PF12/B11, W6/32, BBM.1, SN66E3.1, SN66E3.2, SN66E.3, Ha5C2.A2, MWB1, MWB1-mod, Hz.BB7.2VH1-69_A18VK, Hz.BB7.2VH1-69 (27,30)_A18, HzBB7.2VH1-69 (27,30,48) A18, Hz.BB7.2 VH1-69 (27,30,67)_A18, Hz.BB7.2 VH1-69 (27,30,69)_A18, Hz.BB7.2 VH1-69 (27,30,67,69)_A18, Hz.BB7.2 VH1-3_A18, Hz.BB7.2 VH1-3(48)_A18, Hz.BB7.2 VH1-3(67)_A18, Hz.BB7.2 VH1-3(69)_A18, Hz.BB7.2 VH1-3(71)_A18, Hz.BB7.2 VH1-3(73)_A18, and MWB1.2. In some embodiments, the scFv has the VL and VH sequences of BB7.2 (SEQ ID NOs: 37 and 38). In some embodiments, the scFv has the VL and VH sequences of 3PF12/C4 (SEQ ID NOs: 39 and 40). In some embodiments, the scFv has the VL and VH sequences of 3PF12/F12 (SEQ ID NOs: 41 and 42). In some embodiments, the scFv has the VL and VH sequences of 3PF12/B11 (SEQ ID NOs: 43 and 44). In some embodiments, the scFv has the VL and VH sequences of W6/32 (SEQ ID NOs: 45 and 46). In some embodiments, the scFv has the VL and VH sequences of BBM.1 (SEQ ID NOs: 47 and 48). In some embodiments, the scFv has the VL and VH sequences of SN66E3 (SEQ ID NOs: 49 and 50). In some embodiments, the scFv has the VL and VH sequences of Ha5C2.A2 (SEQ ID NOs: 51 and 52). In some embodiments, the scFv has the VL and VH sequences of MWB1 (SEQ ID NOs: 53 and 54). In some embodiments, the scFv has the VL and VH sequences of MWB1-mod (SEQ ID NOs: 55 and 56). In some embodiments, the scFv has the VL and VH sequences of Hz.BB7.2 VH1-69_A18VK (SEQ ID NOs: 57 and 58). In some embodiments, the scFv has the VL and VH sequences of Hz.BB7.2 VH1-69 (27,30)_A18 (SEQ ID NOs: 59 and 60). In some embodiments, the scFv has the VL and VH sequences of Hz.BB7.2 VH1-69 (27,30,48)_A18 (SEQ ID NOs: 61 and 62). In some embodiments, the scFv has the VL and VH sequences of Hz.BB7.2 VH1-69 (27,30,67)_A18 (SEQ ID NOs: 63 and 64). In some embodiments, the scFv has the VL and VH sequences of Hz.BB7.2 VH1-69 (27,30,69) A18 (SEQ ID NOs: 65 and 66). In some embodiments, the scFv has the VL and VH sequences of Hz.BB7.2 VH1-69 (27,30,67,69)_A18 (SEQ ID NOs: 67 and 68). In some embodiments, the scFv has the VL and VH sequences of Hz.BB7.2 VH1-3_A18 (SEQ ID NOs: 69 and 70). In some embodiments, the scFv has the VL and VH sequences of Hz.BB7.2 VH1-3(48)_A18 (SEQ ID NOs: 71 and 72). In some embodiments, the scFv has the VL and VH sequences of Hz.BB7.2 VH1-3(67)_A18 (SEQ ID NOs: 73 and 74). In some embodiments, the scFv has the VL and VH sequences of Hz.BB7.2 VH1-3(69)_A18 (SEQ ID NOs: 75 and 76). In some embodiments, the scFv has the VL and VH sequences of Hz.BB7.2 VH1-3(71)_A18 (SEQ ID NOs: 77 and 78). In some embodiments, the scFv has the VL and VH sequences of Hz.BB7.2 VH1-3(73)_A18 (SEQ ID NOs: 79 and 80). In some embodiments, the scFv has the VL and VH sequences of MWB1.2 (SEQ ID NOs: 163 and 164). In some embodiments, the scFv has the VL and VH sequences of SN66E3.2 (SEQ ID NOs: 165 and 166). In some embodiments, the scFv has the VL and VH sequences of SN66E3.3 (SEQ ID NOs: 283 and 284) In some embodiments, the scFv is BB7.2 (SEQ ID NO:167). In some embodiments, the scFv is 3PF12 (SEQ ID NO:168). In some embodiments, the scFv is SN66E3.1 (SEQ ID NO:169). In some embodiments, the scFv is SN66E3.2 (SEQ ID NO:285). In some embodiments, the scFv is SN66E3.3 (SEQ ID NO:286). In some embodiments, the scFv is Hz BB7.2.1 (SEQ ID NO:287). In some embodiments, the scFv is HzBB7.2.2 (SEQ ID NO:288). In some embodiments, the scFv is MWB1.1 (SEQ ID NO:273). In some embodiments, the scFv is MWB1.2 (SEQ ID NO:274). In some embodiments, the scFv is 3PF12/C4. In some embodiments, the scFv is 3PF12/F12. In some embodiments, the scFv is 3PF12/B11. In some embodiments, the scFv is W6/32. In some embodiments, the scFv is BBM.1. In some embodiments, the scFv is Ha5C2.A2. In some embodiments, the scFv is MWB1. In some embodiments, the scFv is MWB1-mod. In some embodiments, the scFv is BB7.2. In some embodiments, the scFv is 3PF12. In some embodiments, the scFv is SN66E3.1. In some embodiments, the scFv is SN66E3.2. In some embodiments, the scFv is SN66E3.3. In some embodiments, the scFv is Hz BB7.2.1. In some embodiments, the scFv is HzBB7.2.2. In some embodiments, the scFv is MWB1.1. In some embodiments, the scFv is MWB1.2. In some embodiments, the scFv is Hz.BB7.2 VH1-69_A18VK. In some embodiments, the scFv is Hz.BB7.2 VH1-69 (27,30)_A18. In some embodiments, the scFv is Hz.BB7.2 VH1-69 (27,30,48)_A18. In some embodiments, the scFv is Hz.BB7.2 VH1-69 (27, 30, 67)_A18. In some embodiments, the scFv is Hz.BB7.2 VH1-69 (27, 30, 69)_A18. In some embodiments, the scFv is Hz.BB7.2 VH1-69 (27, 30, 67, 69)_A18. In some embodiments, the scFv is Hz.BB7.2VH1-3_A18. In some embodiments, the scFv is Hz.BB7.2 VH1-3(48)_A18. In some embodiments, the scFv is Hz.BB7.2-3(67)_A18. In some embodiments, the scFv is Hz.BB7.2 VH1-3(69)_A18. In some embodiments, the scFv is Hz.BB7.2 VH1-3(71)_A18. In some embodiments, the scFv is Hz.BB7.2 VH1-3(73)_A18. In some embodiments, the scFv is MWB1.2. In some embodiments, the scFv is SN66E3.2. In some embodiments, the scFv is MWB1.1. In some embodiments, the scFv is MWB1.2. In some embodiments, the scFv comprises Hz.BB7.2 heavy chain Hz.BB7.2VH1-69. In some embodiments, the scFv comprises Hz.BB7.2 Heavy chain Hz.BB7.2VH1-69 (H27Y, H30S. In some embodiments, the scFv comprises Hz.BB7.2 heavy chain HZ.BB7.2VH1-69 (H27Y, H30S, H48I). In some embodiments, the scFv comprises Hz.BB7.2 Heavy chain Hz.BB7.2VH1-69 (H27Y, H30S, H67T). In some embodiments, the scFv comprises Hz. BB7.2 Heavy chain Hz.BB7.2VH1-69 (H27Y, H30S, H69L). In some embodiments, the scFv comprises Hz.BB7.2 Heavy Chain HZ.BB7.2VH1-69 (H27Y, H30S, VH67T, H69L). In some embodiments, the scFv comprises Hz.BB7.2 Heavy Chain Hz.BB7.2 VH1-3. In some embodiments, the scFv comprises Hz.BB7.2 Heavy Chain Hz.BB7.2 VH1-3 (H48I). In some embodiments, the scFv comprises Hz.BB7.2 Heavy Chain VH1-3 (H67T). In some embodiments, the scFv comprises Hz.BB7.2 Heavy Chain Hz.BB7.2 VH1-3 (H69L). In some embodiments, the scFv comprises Hz.BB7.2 Heavy Chain Hz.BB7.2 VH1-3 (H71A). In some embodiments, the scFv comprises Hz.BB7.2 Heavy Chain Hz.BB7.2 VH1-3 (H73A). In some embodiments, the scFv comprises Hz.BB7.2 Light chain VKA18. The 6 CDR sequences for the variable heavy and variable light chains are shown in bold and underline in Table 2 for each sequence, also referred to as vhCDR1, vhCDR2, vhCDR3, vlCDR1, vlCDR2, and vlCDR3. In some embodiments, the iCAR comprises the 6 CDR sequences for the variable heavy and variable light chains as show in bold and underline in Table 2 for each sequence, also referred to as vhCDR1, vhCDR2, vhCDR3, vlCDR1, vlCDR2, and vlCDR3. In some embodiments, the iCAR comprises the 6 CDR sequences for the variable heavy and variable light chains as show in bold and underline in Table 2 for each sequence, also referred to as vhCDR1, vhCDR2, vhCDR3, vlCDR1, vlCDR2, and vlCDR3, wherein each CDR individually optionally comprises one more substitutions. In some embodiments, the iCAR comprises the 6 CDR sequences for the variable heavy and variable light chains as show in bold and underline in Table 2 for each sequence, also referred to as vhCDR1, vhCDR2, vhCDR3, vlCDR1, vlCDR2, and vlCDR3, wherein each CDR individually optionally comprises 1, 2, and/or 3 substitutions. In some embodiments, the iCAR comprises the 6 CDR sequences for the variable heavy and variable light chains as show in bold and underline in Table 2 for each sequence, also referred to as vhCDR1, vhCDR2, vhCDR3, vlCDR1, vlCDR2, and vlCDR3, wherein each CDR individually comprises one more substitutions. In some embodiments, the iCAR comprises the 6 CDR sequences for the variable heavy and variable light chains as show in bold and underline in Table 2 for each sequence, also referred to as vhCDR1, vhCDR2, vhCDR3, vlCDR1, vlCDR2, and vlCDR3, wherein each CDR individually comprises 1, 2, and/or 3 substitutions.

TABLE 2
iCAR vh, vl, and scFv sequences

| Sequence | SEQ | |
|---|---|---|
| Information | ID NO | Amino acid sequence |
| BB7.2 variable | 37 | DVLMTQTPLSLPVSLGDQVSISCRSSQSIVHSNGNTYLEWY |
| light chain | | LQKPGQSPKLLIYKVSNRFSGVPDRFSGSGSGTDFTLKISRV |
| | | EAEDLGVYYCFQGSHVPRTFGGGTKLEIK |
| BB7.2 variable | 38 | QVQLQQSGPELVKPGASVKMSCKASGYTFTSYHIQWVKQR |
| heavy chain | | PGQGLEWIGWIYPGDGSTQYNEKFKGKTTLTADKSSSTAY |
| | | MLLSSLTSEDSAIYFCAREGTYYAMDYWGQGTSVTVSS |
| 3PF12/C4 | 39 | DIVMTQSPSFLSASVGDRVTITCRASHGINNYLAWYQQKPG |
| variable light | | KAPKLLIYAASTLQSGVPSRFSGSGSGTEFTLTISSLQPEDFA |
| chain | | TYYCQQYDSYPPTFGRGTKVEIK |
| 3PF12/C4 | 40 | QVQLVQSGGGVVQPGGSLRVSCAASGVTLSDYGMHWVRQ |
| variable heavy | | APGKGLEWMAFIRNDGSDKYYADSVKGRFTISRDNSKKTV |
| chain | | SLQMSSLRAEDTAVYYCAKNGESGPLDYWYFDLWGRGTL |
| | | VTVSS |
| 3PF12/F12 | 41 | DVVMTQSPSSLSASVGDRVTITCQASQDISNYLNWYQQKP |
| variable light | | GKAPKLLIYDASNLETGVPSRFSGSGSGTDFTFTISSLQPEDF |
| chain | | ATYYCQQYSSFPLTFGGGTKVDIK |
| 3PF12/F12 | 42 | QVQLVQSGGGVVQPGGSLRVSCAASGVTLSDYGMHWVRQ |
| variable heavy | | APGKGLEWMAFIRNDGSDKYYADSVKGRFTISRDNSKKTV |
| chain | | SLQMSSLRAEDTAVYYCAKNGESGPLDYWYFDLWGRGTL |
| | | VTVSS |
| 3PF12/B11 | 43 | DVVMTQSPSSLSASVGDRVTITCQASQDISNYLNWYQQKP |
| variable light | | GKAPKLLIYDASNLETGVPSRFSGSGSGTDFTFTISSLQPEDI |
| chain | | ATYYCQQYDNLPPTFGGGTKLEIV |
| 3PF12/B11 | 44 | QVQLVQSGGGVVQPGGSLRVSCAASGVTLSDYGMHWVRQ |
| variable heavy | | APGKGLEWMAFIRNDGSDKYYADSVKGRFTISRDNSKKTV |
| chain | | SLQMSSLRAEDTAVYYCAKNGESGPLDYWYFDLWGRGTL |
| | | VTVSS |
| W6/32 variable | 45 | SIVMTQTPKFLLVSAGDRVTITCKASQSVSNDVAWYQQKP |
| light chain | | GQSPKLLIYYASNRYTGVPDRFTGSGYGTDFTFTISTVQAED |
| | | LAVYFCQQDYSSPPWTFGGGTKLEIR |
| W6/32 variable | 46 | QVQLKQSGPGLVQPSQSLSLTCTVSGFSLTSYGVHWVRQPP |
| heavy chain | | GKGLEWLGVIWSGGSTDYNAAFISRLSIRKDNSKSQVFFK |
| | | MNSLQADDTAIYYCARTFTTSTSAWFAYWGQGTLVTVSA |
| BBM.1 variable | 47 | DIQMTQSPASQSASLGESVTITCLASQTIGTWLAWYQQKPG |
| light chain | | KSPQLLIYAATSLADGVPSRFSGSGSGTKFSLKIRTLQAEDF |
| | | VSYYCQQLYSKPYTFGGGTKLEIK |
| BBM.1 variable | 48 | EVQLQQSGAELVKPGASVKLSCTPSGFNVKDTYIHWVKQR |
| heavy chain | | PKQGLEWIGRIDPSDGDIKYDPKFQGKATITADTSSNTVSL |
| | | QLSSLTSEDTAVYYCARWFGDYGAMNYWGQGTSVTVSS |
| SN66E3.1 | 49 | DIVMTQSPDSLAVSLGERATISCKSSQSVLYSSNNKNYLAW |
| variable light | | YQQKLGQPPKLLIYWASTRESGVPDRESGSGSGTNFTLTISS |
| chain | | LQAENVAVYYCQQYYGTPFTFGGGTKVEIK |
| SN66E3.1 | 50 | QVQLVQSGAEVKKPGASVKVSCKASGYTFTDYYLHWVRQ |
| variable heavy | | APGQGLEWMGWINPYTGGTNYAQKFQGRVTMTRDASIST |
| chain | | VYMELSGLTSDDTAVHFCARAGASYYDFWSGWVFDYWG |
| | | QGTLVTVSS |
| Ha5C2.A2 | 51 | DIQMTQSPSSLSASVGDRVTITCRASQSISTYLNWYQQKPG |
| variable light | | KAPKLLIYAASSLQSGVPSRFSGSGSGTDFTLTISSLQPEDFA |
| chain | | TYQCQQSYSTPFTFGGGTKVEIK |
| Ha5C2.A2 | 52 | QVQLQESGPGLVKPSETLSLTCTVSGGSISSYYWSWIRQPAG |
| variable heavy | | KGLEWIGRIYISGGTNYNPSLKSRVTMSVDTSKNQVSLKLS |
| chain | | SVTAADTAVYYCARDILGGVSGWSHYGMDVWGQGTTVT |
| | | VSS |
| MWB1 variable | 53 | QSALTQPPSASGSPGQSVTISCTGTSSDVGGYKYVSWYQH |
| light chain | | HPDKAPKLMIYEVNKRPSGVPDRESGSKSDNTASLTVSGLQ |
| | | AEDEADYYCSSYAGSNNWVFGGGTKLTVL |
| MWB1 variable | 54 | QVQLVESGGGVVQPGGSLRLSCAASGFTFSTYGMHWVRQ |
| heavy chain | | APGKGLEWAASVSYDGSNKYYADSGQGRFTISRDTSMNSL |
| | | YLQVNSLRDETAVYYCAIGIYGAYSFDYWGQGTLVTVSS |
| MWB1.1 | 55 | QSALTQPPSASGSPGQSVTISCTGTSSDVGGYKYVSWYQH |
| (MWB1.1) | | HPDKAPKLMIYEVNKRPSGVPDRESGSKSDNTASLTVSGLQ |
| variable light | | AEDEADYYCSSYAGSNNWVFGGGTKLTVL |
| chain | | |
| MWB1.1(MWB1.1) | 56 | QVQLVESGGGVVQPGGSLRLSCAASGFTFSTYGMHWVRQ |
| variable heavy | | APGKGLEWVASISYDGSNKYYADSGQGRFTISRDTSKNSL |
| chain | | YLQMNSLRAEDTAVYYCAIGIYGAYSFDYWGQGTLVTVSS |
| Hz.BB7.2_ | 57 | DVVMTQTPLSLSVTPGQPASISCRSSQSIVHSNGNTYLEWY |
| A18VK variable | | LQKPGQSPQLLIYKVSNRFSGVPDRESGSGSGTDFTLKISRV |
| light chain | | EAEDVGVYYCFQGSHVPRTFGGGTKVEIK |
| Hz.BB7.2 VH1-69 | 58 | QVQLVQSGAEVKKPGSSVKVSCKASGGTFSSYHIQWVRQA |
| variable heavy | | PGQGLEWMGWIYPGDGSTQYNEKFKGRVTITADKSTSTA |
| chain | | YMELSSLRSEDTAVYYCAREGTYYAMDYWGQGTLVTVSS |
| Hz.BB7.2 VH1-69 | 59 | DVVMTQTPLSLSVTPGQPASISCRSSQSIVHSNGNTYLEWY |
| (27, 30) | | LQKPGQSPQLLIYKVSNRFSGVPDRESGSGSGTDFTLKISRV |
| variable light | | EAEDVGVYYCFQGSHVPRTFGGGTKVEIK |
| chain | | |
| Hz.BB7.2 Heavy | 60 | QVQLVQSGAEVKKPGSSVKVSCKASGYTFTSYHIQWVRQA |
| chain VH1-69 | | PGQGLEWMGWIYPGDGSTQYNEKFKGRVTITADKSTSTA |
| (H27Y, H30S) | | YMELSSLRSEDTAVYYCAREGTYYAMDYWGQGTLVTVSS |
| HZ.BB7.2VH1-69 | 61 | DVVMTQTPLSLSVTPGQPASISCRSSQSIVHSNGNTYLEWY |
| (27, 30, 4)_ | | LQKPGQSPQLLIYKVSNRFSGVPDRESGSGSGTDFTLKISRV |
| A18 variable | | EAEDVGVYYCFQGSHVPRTFGGGTKVEIK |
| light chain | | |
| Hz.BB7.2 heavy | 62 | QVQLVQSGAEVKKPGSSVKVSCKASGYTFTSYHIQWVRQA |
| chain VH1-69 | | PGQGLEWIGWIYPGDGSTQYNEKFKGRVTITADKSTSTAY |
| (H27Y, H30S, | | MELSSLRSEDTAVYYCAREGTYYAMDYWGQGTLVTVSS |
| H48I)) | | |
| Hz.BB7.2 VH1-69 | 63 | DVVMTQTPLSLSVTPGQPASISCRSSQSIVHSNGNTYLEWY |
| (27, 30, 67)_A18 | | LQKPGQSPQLLIYKVSNRFSGVPDRESGSGSGTDFTLKISRV |
| variable light | | EAEDVGVYYCFQGSHVPRTFGGGTKVEIK |
| chain | | |
| Hz.BB7.2 Heavy | 64 | QVQLVQSGAEVKKPGSSVKVSCKASGYTFTSYHIQWVRQA |
| chain VH1-69 | | PGQGLEWMGWIYPGDGSTQYNEKFKGRTTITADKSTSTA |
| (H27Y, H30S, | | YMELSSLRSEDTAVYYCAREGTYYAMDYWGQGTLVTVSS |
| H67T)) | | |
| HZ.BB7.2VH1-69 | 65 | DVVMTQTPLSLSVTPGQPASISCRSSQSIVHSNGNTYLEWY |
| (27, 30, 69)_ | | LQKPGQSPQLLIYKVSNRFSGVPDRFSGSGSGTDFTLKISRV |
| A18 variable | | EAEDVGVYYCFQGSHVPRTFGGGTKVEIK |
| light chain | | |
| Hz. BB7.2 Heavy | 66 | QVQLVQSGAEVKKPGSSVKVSCKASGYTFTSYHIQWVRQA |
| chain VH1-69 | | PGQGLEWMGWIYPGDGSTQYNEKFKGRVTLTADKSTSTA |
| (H27Y, H30S, | | YMELSSLRSEDTAVYYCAREGTYYAMDYWGQGTLVTVSS |
| H69L)) | | |
| Hz.BB7.2 VH1-69 | 67 | DVVMTQTPLSLSVTPGQPASISCRSSQSIVHSNGNTYLEWY |
| (27, 30, 67, 69)_ | | LQKPGQSPQLLIYKVSNRFSGVPDRFSGSGSGTDFTLKISRV |
| A18 variable | | EAEDVGVYYCFQGSHVPRTFGGGTKVEIK |
| light chain | | |
| Hz.BB7.2 Heavy | 68 | QVQLVQSGAEVKKPGSSVKVSCKASGYTFTSYHIQWVRQA |
| Chain VH1-69 | | PGQGLEWMGWIYPGDGSTQYNEKFKGRTTLTADKSTSTA |
| (H27Y, H30S, | | YMELSSLRSEDTAVYYCAREGTYYAMDYWGQGTLVTVSS |
| VH67T, H69L)) | | |
| Hz.BB7.2VH1-3_ | 69 | DVVMTQTPLSLSVTPGQPASISCRSSQSIVHSNGNTYLEWY |
| A18 variable | | LQKPGQSPQLLIYKVSNRFSGVPDRESGSGSGTDFTLKISRV |
| light chain | | EAEDVGVYYCFQGSHVPRTFGGGTKVEIK |
| Hz.BB7.2 Heavy | 70 | QVQLVQSGAEVKKPGASVKVSCKASGYTFTSYHIQWVRQ |
| Chain VH1-3) | | APGQRLEWMGWIYPGDGSTQYNEKFKGRVTITRDTSAST |
| | | AYMELSSLRSEDTAVYYCAREGTYYAMDYWGQGTLVTVS |
| | | S |
| Hz.BB7.2VH1-3 | 71 | DVVMTQTPLSLSVTPGQPASISCRSSQSIVHSNGNTYLEWY |
| (48)_A18 | | LQKPGQSPQLLIYKVSNRFSGVPDRESGSGSGTDFTLKISRV |
| variable light | | EAEDVGVYYCFQGSHVPRTFGGGTKVEIK |
| chain | | |
| Hz.BB7.2 Heavy | 72 | QVQLVQSGAEVKKPGASVKVSCKASGYTFTSYHIQWVRQ |
| Chain VH1-3 | | APGQRLEWIGWIYPGDGSTQYNEKFKGRVTITRDTSASTA |
| (H48I)) | | YMELSSLRSEDTAVYYCAREGTYYAMDYWGQGTLVTVSS |
| Hz.BB7.2VH1-3 | 73 | DVVMTQTPLSLSVTPGQPASISCRSSQSIVHSNGNTYLEWY |
| (67)_A18 | | LQKPGQSPQLLIYKVSNRFSGVPDRFSGSGSGTDFTLKISRV |
| variable light | | EAEDVGVYYCFQGSHVPRTFGGGTKVEIK |
| chain | | |
| (Hz.BB7.2 Light | | |
| chain VKA18) | | |
| Hz.BB7.2 Heavy | 74 | QVQLVQSGAEVKKPGASVKVSCKASGYTFTSYHIQWVRQ |
| Chain VH1-3 | | APGQRLEWMGWIYPGDGSTQYNEKFKGRTTITRDTSAST |
| (H67T)) | | AYMELSSLRSEDTAVYYCAREGTYYAMDYWGQGTLVTVS |
| | | S |
| Hz.BB.2VH1-3 | 75 | DVVMTQTPLSLSVTPGQPASISCRSSQSIVHSNGNTYLEWY |
| (69)_A18 | | LQKPGQSPQLLIYKVSNRFSGVPDRFSGSGSGTDFTLKISRV |
| variable light | | EAEDVGVYYCFQGSHVPRTFGGGTKVEIK |
| chain | | |
| Hz.BB7.2 Heavy | 76 | QVQLVQSGAEVKKPGASVKVSCKASGYTFTSYHIQWVRQ |
| Chain VH1-3 | | APGQRLEWMGWIYPGDGSTQYNEKFKGRVTLTRDTSAST |
| (H69L)) | | AYMELSSLRSEDTAVYYCAREGTYYAMDYWGQGTLVTVS |
| | | S |
| Hz.BB7.2VH1-3 | 77 | DVVMTQTPLSLSVTPGQPASISCRSSQSIVHSNGNTYLEWY |
| (71)_A18 | | LQKPGQSPQLLIYKVSNRFSGVPDRFSGSGSGTDFTLKISRV |
| variable light | | EAEDVGVYYCFQGSHVPRTFGGGTKVEIK |
| chain | | |
| Hz.BB7.2 VH1-3 | 78 | QVQLVQSGAEVKKPGASVKVSCKASGYTFTSYHIQWVRQ |
| (71)_variable | | APGQRLEWMGWIYPGDGSTQYNEKFKGRVTITADTSAST |
| heavy chain | | AYMELSSLRSEDTAVYYCAREGTYYAMDYWGQGTLVTVS |
| | | S |
| Hz.BB7.2VH1-3 | 79 | DVVMTQTPLSLSVTPGQPASISCRSSQSIVHSNGNTYLEWY |
| (73)_A18 | | LQKPGQSPQLLIYKVSNRFSGVPDRESGSGSGTDFTLKISRV |
| variable light | | EAEDVGVYYCFQGSHVPRTFGGGTKVEIK |
| chain | | |
| Hz.BB7.2VH1-3 | 80 | QVQLVQSGAEVKKPGASVKVSCKASGYTFTSYHIQWVRQ |
| (73)_A18 | | APGQRLEWMGWIYPGDGSTQYNEKFKGRVTITRDKSAST |
| variable heavy | | AYMELSSLRSEDTAVYYCAREGTYYAMDYWGQGTLVTVS |
| chain | | S |
| MWB1.2 variable | 163 | QSALTQPPSASGSPGQSVTISCTGTSSDVGGYKYVSWYQQ |
| light chain | | HPGKAPKLMIYEVNKRPSGVPDRFSGSKSGNTASLTVSGLQ |
| | | AEDEADYYCSSYAGSNNWVFGGGTKLTVL |
| MWB1.2 variable | 164 | QVQLVESGGGVVQPGGSLRLSCAASGFTFSTYGMHWVRQ |
| heavy chain | | APGKGLEWVASISYDGSNKYYADSGQGRFTISRDTSKNSL |
| | | YLQMNSLRAEDTAVYYCAIGIYGAYSFDYWGQGTLVTVSS |
| SN66E3.2 | 165 | DIVMTQSPDSLAVSLGERATISCKSSQSVLYSSNNKNYLAW |
| variable light | | YQQKPGQPPKLLIYWASTRESGVPDRFSGSGSGTDFTLTISS |
| chain | | LQAEDVAVYYCQQYYGTPFTFGGGTKVEIK |
| SN66E3.2 | 166 | QVQLVQSGAEVKKPGASVKVSCKASGYTFTDYYLHWVRQ |
| variable heavy | | APGQGLEWMGWINPYTGGTNYAQKFQGRVTMTRDTSIST |
| chain | | AYMELSGLTSDDTAVYYCARAGASYYDFWSGWVFDYWG |
| | | QGTLVTVSS |
| MWB1.1 | 273 | QVQLVESGGGVVQPGGSLRLSCAASGFTFSTYGMHWVRQA |
| scFvVH_VL | | PGKGLEWVASISYDGSNKYYADSGQGRFTISRDTSKNSLYL |
| | | QMNSLRAEDTAVYYCAIGIYGAYSFDYWGQGTLVTVSSGG |
| | | GGSGGGGSGGGGSQSALTQPPSASGSPGQSVTISCTGTSSDV |
| | | GGYKYVSWYQHHPDKAPKLMIYEVNKRPSGVPDRESGSKS |
| | | DNTASLTVSGLQAEDEADYYCSSYAGSNNWVFGGGTKLTV |
| | | L |
| MWB1.2scFvVH_ | 274 | QVQLVESGGGVVQPGGSLRLSCAASGFTFSTYGMHWVRQ |
| VL | | APGKGLEWVASISYDGSNKYYADSGQGRFTISRDTSKNSL |
| | | YLQMNSLRAEDTAVYYCAIGIYGAYSFDYWGQGTLVTVSS |
| | | GGGGSGGGGSGGGGSQSALTQPPSASGSPGQSVTISCTGTSS |
| | | DVGGYKYVSWYQQHPGKAPKLMIYEVNKRPSGVPDRFSG |
| | | SKSGNTASLTVSGLQAEDEADYYCSSYAGSNNWVFGGGTK |
| | | LTVL |
| SN66E3.3 | 283 | DIVMTQSPDSLAVSLGERATISCKSSQSVLYSSNNKNYLAW |
| Variable Light | | YQQKPGQPPKLLIYWASTRESGVPDRFSGSGSGTDFTLTISS |
| chain | | LQAEDVAVYYCQQYYGTPFTFGGGTKVEIK |
| SN66E3.3 | 284 | QVQLVQSGAEVKKPGASVKVSCKASGYTFTDYYLHWVRQ |
| variable Heavy | | APGQGLEWMGWINPYTGGTNYAQKFQGRVTMTRDTSIST |
| chain | | AYMELSRLRSEDTAVYYCARAGASYYDFWSGWVFDYWG |
| | | QGTLVTVSS |

In some embodiments, the orientation of the iCAR VH and VL regions is VH-VL. In some embodiments, the orientation of the iCAR VH and VL regions is VL-VH.

In some embodiments, the iCAR scFv comprises a linker that covalently connects the VH and the VL to form the iCAR scFv.

In some embodiments, the heavy and light chains of the scFv are covalently connected via a linker. In some embodiments, the linker is a gly-ser polypeptide linker, i.e., a peptide that consists of glycine and serine residues. Exemplary gly-ser polypeptide linkers comprise the amino acid sequence Ser(Gly₄Ser)ₙ, as well as (Gly₄Ser)ₙ and/or (Gly₄Ser 3)ₙ. In some embodiments, n=1. In some embodiments, n=2. In some embodiments, n=3, i.e., Ser(Gly₄Ser) 3. In some embodiments, n=4, i.e., Ser(Gly₄Ser) 4. In some embodiments, n=5. In some embodiments, n=6. In some embodiments, n=7. In some embodiments, n=8. In some embodiments, n=9. In some embodiments, n=10. Another exemplary gly-ser polypeptide linker comprises the amino acid sequence Ser(Gly₄Ser)ₙ. In some embodiments, n=1. In some embodiments, n=2. In some embodiments, n=3. In another embodiment, n=4. In some embodiments, n=5. In some embodiments, n=6. Another exemplary gly-ser polypeptide linker comprises (Gly₄Ser)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly 3 Ser)ₙ. In some embodiments, n=1. In some embodiments, n=2. In some embodiments, n=3. In some embodiments, n=4. In another embodiment, n=5. In yet another embodiment, n=6. Another exemplary gly-ser polypeptide linker comprises (Gly₄Ser 3)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly 3 Ser)ₙ. In some embodiments, n=1. In some embodiments, n=2. In some embodiments, n=3. In some embodiments, n=4. In another embodiment, n=5. In yet another embodiment, n=6.

In some embodiments, the iCAR comprises a GS based linker sequence, connecting the VH and VL or the VL and VH to form the scFv. In some embodiments, the GS linker comprises GGGGS (SEQ ID NO:153). In some embodiments, the iCAR comprises a Whitlow linker sequence, e.g., GSTSGSGKPGSGEGSTKG (SEQ ID NO:82). In some embodiments, the iCAR comprises the Vh and Vl sequences in the Vh-Vl orientation. In some embodiments, the iCAR comprises the Vh and Vl sequences in the Vl-Vh orientation. In some embodiments, the iCAR comprises a linker between the Vh and Vl sequences. In some embodiments, the iCAR does not comprise a linker between the Vh and Vl sequences.

TABLE 3
iCAR linkers

| Sequence | SEQ ID | |
|---|---|---|
| Information | NO | Amino acid sequence |
| (G4S)X3 linker | 81 | GGGGSGGGGSGGGGS |
| Whitlow linker | 82 | GSTSGSGKPGSGEGSTKG |
| PD1 linker | 83 | DFQWREKTPEPPVPCVPEQ |
| G4S | 153 | GGGGS |

In some embodiments, the iCAR scFv comprises a linker. In some embodiments, the iCAR scFv is selected from the group consisting of BB7.2 scFv (SEQ ID NO: 167), 3PF12 scFv (SEQ ID NO: 168), SN66E3.1 scFv (SEQ ID NO: 169), SN66E3.2 scFv (SEQ ID NO: 285), SN66E3.3 scFv (SEQ ID NO: 286), Hz BB7.2.1 scFv (SEQ ID NO: 287), and Hz BB7.2.2 scFv (SEQ ID NO: 288). In some embodiments, the iCAR scFv is BB7.2 scFv (SEQ ID NO: 167). In some embodiments, the iCAR scFv is 3PF12 scFv (SEQ ID NO: 168). In some embodiments, the iCAR scFv is SN66E3.1 scFv (SEQ ID NO: 169). In some embodiments, the iCAR scFv is SN66E3.2 scFv (SEQ ID NO: 285). In some embodiments, the iCAR scFv is SN66E3.3 scFv (SEQ ID NO: 286). In some embodiments, the iCAR scFv is Hz BB7.2.1 scFv (SEQ ID NO: 287). In some embodiments, the iCAR scFv is Hz BB7.2.2 scFv (SEQ ID NO: 288).

TABLE 4
iCAR scFv sequences with linkers

| Sequence | SEQ | |
|---|---|---|
| Information | ID NO | Amino acid sequence |
| BB7.2 scFv | 167 | QVQLQQSGPELVKPGASVKMSCKASGYTFTSYHIQWVKQ |
| | | RPGQGLEWIGWIYPGDGSTQYNEKFKGKTTLTADKSSSTA |
| | | YMLLSSLTSEDSAIYFCAREGTYYAMDYWGQGTSVTVSS |
| | | GGGGSGGGGSGGGGSDVLMTQTPLSLPVSLGDQVSISCRS |
| | | SQSIVHSNGNTYLEWYLQKPGQSPKLLIYKVSNRFSGVPD |
| | | RFSGSGSGTDFTLKISRVEAEDLGVYYCFQGSHVPRTFGG |
| | | GTKLEIK |
| 3PF12 scFv | 168 | QVQLVQSGGGVVQPGGSLRVSCAASGVTLSDYGMHWVR |
| | | QAPGKGLEWMAFIRNDGSDKYYADSVKGRFTISRDNSKK |
| | | TVSLQMSSLRAEDTAVYYCAKNGESGPLDYWYFDLWGR |
| | | GTLVTVSSGGGGSGGGGSGGGGSDIVMTQSPSFLSASVGD |
| | | RVTITCRASHGINNYLAWYQQKPGKAPKLLIYAASTLQSG |
| | | VPSRFSGSGSGTEFTLTISSLQPEDFATYYCQQYDSYPPTFG |
| | | RGTKVEIK |
| SN66E3.1 scFv | 169 | QVQLVQSGAEVKKPGASVKVSCKASGYTFTDYYLHWVR |
| | | QAPGQGLEWMGWINPYTGGTNYAQKFQGRVTMTRDASIS |
| | | TVYMELSGLTSDDTAVHFCARAGASYYDFWSGWVFDYW |
| | | GQGTLVTVSSGGGGSGGGGSGGGGSDIVMTQSPDSLAVSL |
| | | GERATISCKSSQSVLYSSNNKNYLAWYQQKLGQPPKLLIY |
| | | WASTRESGVPDRFSGSGSGTNFTLTISSLQAENVAVYYCQ |
| | | QYYGTPFTFGGGTKVEIK |
| SN66E3.2 scFv | 285 | DIVMTQSPDSLAVSLGERATISCKSSQSVLYSSNNKNYLA |
| | | WYQQKPGQPPKLLIYWASTRESGVPDRESGSGSGTDFTLTI |
| | | SSLQAEDVAVYYCQQYYGTPFTFGGGTKVEIKGGGGSGG |
| | | GGSGGGGSQVQLVQSGAEVKKPGASVKVSCKASGYTFTD |
| | | YYLHWVRQAPGQGLEWMGWINPYTGGTNYAQKFQGRV |
| | | TMTRDTSISTAYMELSGLTSDDTAVYYCARAGASYYDFW |
| | | SGWVFDYWGQGTLVTVSS |
| SN66E3.3 scFv | 286 | DIVMTQSPDSLAVSLGERATISCKSSQSVLYSSNNKNYLA |
| | | WYQQKPGQPPKLLIYWASTRESGVPDRFSGSGSGTDFTLTI |
| | | SSLQAEDVAVYYCQQYYGTPFTFGGGTKVEIKGGGGSGG |
| | | GGSGGGGSQVQLVQSGAEVKKPGASVKVSCKASGYTFTD |
| | | YYLHWVRQAPGQGLEWMGWINPYTGGTNYAQKFQGRV |
| | | TMTRDTSISTAYMELSRLRSEDTAVYYCARAGASYYDFW |
| | | SGWVFDYWGQGTLVTVSS |
| Hz BB7.2.1 scFv | 287 | QVQLVQSGAEVKKPGSSVKVSCKASGYTFTSYHIQWVRQ |
| | | APGQGLEWMGWIYPGDGSTQYNEKFKGRTTITADKSTST |
| | | AYMELSSLRSEDTAVYYCAREGTYYAMDYWGQGTLVTV |
| | | SSGGGGSGGGGSGGGGSDVVMTQTPLSLSVTPGQPASISC |
| | | RSSQSIVHSNGNTYLEWYLQKPGQSPQLLIYKVSNRFSGVP |
| | | DRFSGSGSGTDFTLKISRVEAEDVGVYYCFQGSHVPRTFG |
| | | GGTKVEIK |
| HzBB7.2.2 scFV | 288 | QVQLVQSGAEVKKPGASVKVSCKASGYTFTSYHIQWVRQ |
| | | APGQRLEWIGWIYPGDGSTQYNEKFKGRVTITRDTSASTA |
| | | YMELSSLRSEDTAVYYCAREGTYYAMDYWGQGTLVTVS |
| | | SGGGGSGGGGSGGGGSDVVMTQTPLSLSVTPGQPASISCR |
| | | SSQSIVHSNGNTYLEWYLQKPGQSPQLLIYKVSNRFSGVP |
| | | DRFSGSGSGTDFTLKISRVEAEDVGVYYCFQGSHVPRTFG |
| | | GGTKVEIK |

In some embodiments, the iCAR scFv linker is a gly-ser polypeptide linker, i.e., a peptide that consists of glycine and serine residues. Exemplary gly-ser polypeptide linkers comprise the amino acid sequence Ser(Gly₄Ser)ₙ, as well as (Gly₄Ser)ₙ and/or (Gly₄Ser 3)ₙ. In some embodiments, n=1. In some embodiments, n=2. In some embodiments, n=3, i.e., Ser(Gly₄Ser) 3. In some embodiments, n=4, i.e., Ser(Gly₄Ser) 4. In some embodiments, n=5. In some embodiments, n=6. In some embodiments, n=7. In some embodiments, n=8. In some embodiments, n=9. In some embodiments, n=10. Another exemplary gly-ser polypeptide linker comprises the amino acid sequence Ser(Gly₄Ser)ₙ. In some embodiments, n=1. In some embodiments, n=2. In some embodiments, n=3. In another embodiment, n=4. In some embodiments, n=5. In some embodiments, n=6. Another exemplary gly-ser polypeptide linker comprises (Gly₄Ser)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly 3 Ser)ₙ. In some embodiments, n=1. In some embodiments, n=2. In some embodiments, n=3. In some embodiments, n=4. In another embodiment, n=5. In yet another embodiment, n=6. Another exemplary gly-ser polypeptide linker comprises (Gly₄Ser 3)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly 3 Ser)ₙ. In some embodiments, n=1. In some embodiments, n=2. In some embodiments, n=3. In some embodiments, n=4. In another embodiment, n=5. In yet another embodiment, n=6.

2. iCAR Portion: Hinge Domain

In some embodiments, the bicistronic construct comprises an iCAR portion comprising a hinge domain component. In some embodiments, the hinge domain comprises a hinge selected from the group consisting of a PD-1 hinge domain, a CD28 hinge domain, and a CD8 hinge domain (including a CD8a hinge domain) a LIR1 Ig3-4 hinge domain, a LIR1 Ig-4 hinge domain, a LIR1 52 aa hinge domain, a LIR1 36 aa hinge domain, a LIR1 30 aa hinge domain, a LIR1 8 aa hinge domain, a CD33 hinge domain, and a KIR2DL1 hinge domain. In some embodiments, the hinge domain is a PD-1 hinge (SEQ ID NO: 86). In some embodiments, the hinge domain is a CD28 hinge domain (SEQ ID NO:85). In some embodiments, the vector comprises a CD8 hinge domain. In some embodiments, the vector comprises a CD8a hinge domain (SEQ ID NO:84). In some embodiments, the vector comprises aLIR1 Ig3-4 hinge domain (SEQ ID NO:87). In some embodiments, the vector comprises a LIR1 Ig-4 hinge domain (SEQ ID NO:88). In some embodiments, the vector comprises a LIR1 52 aa hinge domain (SEQ ID NO:89). In some embodiments, the vector comprises a LIR1 36 aa hinge domain (SEQ ID NO:90). In some embodiments, the vector comprises a LIR1 30 aa hinge domain (SEQ ID NO:91). In some embodiments, the vector comprises a LIR1 8 aa hinge domain (SEQ ID NO:92). In some embodiments, the vector comprises a CD33 hinge domain (SEQ ID NO:93). In some embodiments, the vector comprises aKIR2DL1 hinge domain (SEQ ID NO:94). In some embodiments, the iCAR comprises aLIR1 26 aa (SEQ ID NO: 289). In some embodiments, the iCAR comprises PD-1 (47) (SEQ ID NO: 290). In some embodiments, the iCAR comprises PD-1 (42) (SEQ ID NO: 291). In some embodiments, the iCAR comprises PD-1 (36) (SEQ ID NO: 292). In some embodiments, the iCAR comprises PD-1 (30) (SEQ ID NO: 293). In some embodiments, the iCAR comprises PD-1 (26) (SEQ ID NO: 294). In some embodiments, the iCAR comprises PD-1 (20) (SEQ ID NO: 295).

TABLE 5
iCAR hinge sequences

| Sequence | SEQ ID | |
|---|---|---|
| Information | NO | Amino acid sequence |
| CD8 alpha | 84 | TTTPAPRPPTPAPTIASQPLSLRPEACRPAAGGAVHTR |
| | | GLDFACD |
| CD28 | 85 | IEVMYPPPYLDNEKSNGTIIHVKGKHLCPSPLFPGPSKP |
| PD-1 | 86 | TERRAEVPTAHPSPSPRPAGQFQTLV |
| LIR1 Ig3-4 | 87 | VSKKPSLSVQPGPIVAPEETLTLQCGSDAGYNRFVLYK |
| | | DGERDFLQLAGAQPQAGLSQANFTLGPVSRSYGGQY |
| | | RCYGAHNLSSEWSAPSDPLDILIAGQFYDRVSLSVQPG |
| | | PTVASGENVTLLCQSQGWMQTFLLTKEGAADDPWRL |
| | | RSTYQSQKYQAEFPMGPVTSAHAGTYRCYGSQSSKP |
| | | YLLTHPSDPLELVVSGPSGGPSSPTTGPTSTSGPEDQPL |
| | | TPTGSDPQSGLGRHLGV |
| LIR1 Ig-4 | 88 | PLDILIAGQFYDRVSLSVQPGPTVASGENVTLLCQSQG |
| | | WMQTFLLTKEGAADDPWRLRSTYQSQKYQAEFPMG |
| | | PVTSAHAGTYRCYGSQSSKPYLLTHPSDPLELVVSGPS |
| | | GGPSSPTTGPTSTSGPEDQPLTPTGSDPQSGLGRHLGV |
| LIR1 52 aa | 89 | HPSDPLELVVSGPSGGPSSPTTGPTSTSGPEDQPLTPTG |
| | | SDPQSGLGRHLGV |
| LIR1 36 aa | 90 | PSSPTTGPTSTSGPEDQPLTPTGSDPQSGLGRHLGV |
| LIR1 30 aa | 91 | GPTSTSGPEDQPLTPTGSDPQSGLGRHLGV |
| LIR1 8 aa | 92 | GLGRHLGV |
| CD33 | 93 | LNVTYVPQNPTTGIFPGDGSGKQETRAGVVH |
| KIR2DL1 | 94 | PYEWSKSSDPLLVSVTGNPSNSWPSPTEPSSKTGNPRH |
| | | LH |
| LIR1 26 aa | 289 | TSGPEDQPLTPTGSDPQSGLGRHLGV |
| PD-1 (47) | 290 | GAISLAPKAQIKESLRAELRVTERRAEVPTAHPSPSPRP |
| | | AGQFQTLV |
| PD-1 (42) | 291 | APKAQIKESLRAELRVTERRAEVPTAHPSPSPRPAGQF |
| | | QTLV |
| PD-1 (36) | 292 | KESLRAELRVTERRAEVPTAHPSPSPRPAGQFQTLV |
| PD-1 (30) | 293 | ELRVTERRAEVPTAHPSPSPRPAGQFQTLV |
| PD-1 (26) | 294 | TERRAEVPTAHPSPSPRPAGQFQTLV |
| PD-1 (20) | 295 | VPTAHPSPSPRPAGQFQTLV |

3. iCAR Portion: Transmembrane Domain

In some embodiments, the bicistronic construct comprises an iCAR portion comprising a transmembrane (TM) domain component. In some embodiments, the TM domain comprises a TM domain selected from the group consisting of a PD-1 TM domain, a CD28 TM domain, a CD8 TM domain (including a CD8a TM domain), a LIR1 TM domain, a CD33 TM domain, and a KIR2DL1 TM domain. In some embodiments, the TM domain is a PD-1 TM domain (SEQ ID NO:97). In some embodiments, the TM domain is a CD28 TM domain (SEQ ID NO:96). In some embodiments, the vector comprises a CD8 TM domain. In some embodiments, the vector comprises a CD8a TM domain (SEQ ID NO:95). In some embodiments, the vector comprises a LIR1 TM domain (SEQ ID NO:98). In some embodiments, the vector comprises a CD33 TM domain (SEQ ID NO:99). In some embodiments, the vector comprises a KIR2DL1 TM domain (SEQ ID NO:100).

TABLE 6
iCAR transmembrane sequences

| Sequence | SEQ ID | |
|---|---|---|
| Information | NO | Amino acid sequence |
| CD8 alpha | 95 | IYIWAPLAGTCGVLLLSLVITL |
| | | YC |
| CD28 | 96 | FWVLVVVGGVLACYSLLVTV |
| | | AFIIFWV |
| PD-1 | 97 | VGVVGGLLGSLVLLVWVLA |
| | | VI |
| LIR1 | 98 | VIGILVAVILLLLLLLLLFLI |
| CD33 | 99 | GAIGGAGVTALLALCLCLIFFI |
| | | V |
| KIR2DL1 | 100 | ILIGTSVVIILFILLFFLL |

4. iCAR Portion: Inhibitory Domain

In some embodiments, the bicistronic construct comprises an iCAR portion comprising an inhibitory domain component. In some embodiments, the iCAR portion comprises an inhibitory domain. In some embodiments, the inhibitory domain is selected from the group consisting of PD-1, KIR2DL1, KIR2DL2, KIR2DL3, KIR2DL4, KIR2DL5A, KIR3DL1, KIR3DL2, KIR3DL3, LAIR1, CD22, CD33, SIGLEC5, SIGLEC6, SIGLEC7, SIGLEC8, SIGLEC9, SIGLEC10, SIGLEC11, SIGLEC12, PECAM1/CD31, CD200R1, FCRL1, FCRL2, FCRL3, FCRL4, FCRL5, SLAMF1, SLAMF5, BTLA, LAGS, 2B4, CD160, CEACAM1, TIM3, VISTA, TIGIT, SIRPalpha, FcγRIIB, CD5, CD300a, CD300f, LIR1, LIR2, LIR3, LIR5, LIRE, Ly9, 2xPD1(G4S), 2xPD1(PD1), PVRIg, and AA2ARKIR2DL1, LIR1, and PD-1. In some embodiments, the inhibitory domain is KIR2DL1 (SEQ ID NO:102). In some embodiments, the inhibitory domain is LIR1 (SEQ ID NO:143). In some embodiments, the inhibitory domain is PD-1 (SEQ ID NO:101). In some embodiments, the inhibitory domain is KIR2DL2 (SEQ ID NO:103). In some embodiments, the inhibitory domain is KIR2DL3 (SEQ ID NO:104). In some embodiments, the inhibitory domain is KIR2DL4 (SEQ ID NO:105). In some embodiments, the inhibitory domain is KIR2DL5A (SEQ ID NO:106). In some embodiments, the inhibitory domain is KIR3DL1 (SEQ ID NO:107). In some embodiments, the inhibitory domain is KIR3DL2 (SEQ ID NO:108). In some embodiments, the inhibitory domain is KIR3DL3 (SEQ ID NO:109). In some embodiments, the inhibitory domain is LAIR1 (SEQ ID NO:110). In some embodiments, the inhibitory domain is CD22 (SEQ ID NO:111). In some embodiments, the inhibitory domain is CD33 (SEQ ID NO:112). In some embodiments, the inhibitory domain is SIGLEC5 (SEQ ID NO:113). In some embodiments, the inhibitory domain is SIGLEC6 (SEQ ID NO:114). In some embodiments, the inhibitory domain is SIGLEC7 (SEQ ID NO:115). In some embodiments, the inhibitory domain is SIGLEC8 (SEQ ID NO:116). In some embodiments, the inhibitory domain is SIGLEC9 (SEQ ID NO:117). In some embodiments, the inhibitory domain is SIGLEC10 (SEQ ID NO:118). In some embodiments, the inhibitory domain is SIGLEC11 (SEQ ID NO:119). In some embodiments, the inhibitory domain is SIGLEC12 (SEQ ID NO:120). In some embodiments, the inhibitory domain is PECAM1/CD31 (SEQ ID NO:121). In some embodiments, the inhibitory domain is CD200R1 (SEQ ID NO:122). In some embodiments, the inhibitory domain is FCRL1 (SEQ ID NO:123). In some embodiments, the inhibitory domain is FCRL2 (SEQ ID NO:124). In some embodiments, the inhibitory domain is FCRL3 (SEQ ID NO:125). In some embodiments, the inhibitory domain is FCRL4 (SEQ ID NO:126). In some embodiments, the inhibitory domain is FCRL5 (SEQ ID NO:127). In some embodiments, the inhibitory domain is SLAMF1 (SEQ ID NO:128). In some embodiments, the inhibitory domain is SLAMF5 (SEQ ID NO:129). In some embodiments, the inhibitory domain is BTLA (SEQ ID NO:130). In some embodiments, the inhibitory domain is LAG3 (SEQ ID NO:131). In some embodiments, the inhibitory domain is 2B4 (SEQ ID NO:132). In some embodiments, the inhibitory domain is CD160 (SEQ ID NO:133). In some embodiments, the inhibitory domain is CEACAM1 (SEQ ID NO:134). In some embodiments, the inhibitory domain is TIM3 (SEQ ID NO:135). In some embodiments, the inhibitory domain is VISTA (SEQ ID NO:136). In some embodiments, the inhibitory domain is TIGIT (SEQ ID NO:137). In some embodiments, the inhibitory domain is SIRPalpha (SEQ ID NO:138). In some embodiments, the inhibitory domain is FcγRIIB (SEQ ID NO:139). In some embodiments, the inhibitory domain is CD5 (SEQ ID NO:140). In some embodiments, the inhibitory domain is CD300a (SEQ ID NO:141). In some embodiments, the inhibitory domain is CD300f (SEQ ID NO:142). In some embodiments, the inhibitory domain is LIR2 (SEQ ID NO:144). In some embodiments, the inhibitory domain is LIR3 (SEQ ID NO:145). In some embodiments, the inhibitory domain is LIR5 (SEQ ID NO:146). In some embodiments, the inhibitory domain is LIR8 (SEQ ID NO:147). In some embodiments, the inhibitory domain is Ly9 (SEQ ID NO:148). In some embodiments, the inhibitory domain is 2xPD1(G4S) (SEQ ID NO:149). In some embodiments, the inhibitory domain is 2xPD1(PD1) (SEQ ID NO:150). In some embodiments, the inhibitory domain is PVRIg (SEQ ID NO:151). In some embodiments, the inhibitory domain is AA2AR (SEQ ID NO:152).

TABLE 7
iCAR inhibitory domain sequences

| | SEQ | |
|---|---|---|
| Sequence | ID | |
| Information | NO | Amino acid sequence |
| PD-1 | 101 | CSRAARGTIGARRTGQPLKEDPSAVPVFSVDYGELDFQWRE |
| | | KTPEPPVPCVPEQTEYATIVFPSGMGTSSPARRGSADGPRSA |
| | | QPLRPEDGHCSWPL |
| KIR2DL1 | 102 | HRWCSNKKNAAVMDQESAGNRTANSEDSDEQDPQEVTYT |
| | | QLNHCVFTQRKITRPSQRPKTPPTDIIVYTELPNAESRSKVVS |
| | | CP |
| KIR2DL2 | 103 | HRWCSNKKNAAVMDQESAGNRTANSEDSDEQDPQEVTYT |
| | | QLNHCVFTQRKITRPSQRPKTPPTDIIVYAELPNAESRSKVVS |
| | | CP |
| KIR2DL3 | 104 | HRWCCNKKNAVVMDQEPAGNRTVNREDSDEQDPQEVTYA |
| | | QLNHCVFTQRKITRPSQRPKTPPTDIIVYTELPNAEP |
| KIR2DL4 | 105 | RWCSKKKDAAVMNQEPAGHRTVNREDSDEQDPQEVTYAQ |
| | | LDHCIFTQRKITGPSQRSKRPSTDTSVCIELPNAEPRALSPAH |
| | | EHHSQALMGSSRETTALSQTQLASSNVPAAGI |
| KIR2DL5A | 106 | LHCCCSNKKNAAVMDQEPAGDRTVNREDSDDQDPQEVTY |
| | | AQLDHCVFTQTKITSPSQRPKTPPTDTTMYMELPNAKPRSLS |
| | | PAHKHHSQALRGSSRETTALSQNRVASSHVPAAGI |
| KIR3DL1 | 107 | HLWCSNKKNAAVMDQEPAGNRTANSEDSDEQDPEEVTYA |
| | | QLDHCVFTQRKITRPSQRPKTPPTDTILYTELPNAKPRSKVV |
| | | SCP |
| KIR3DL2 | 108 | YRWCSNKKNAAVMDQEPAGDRTVNRQDSDEQDPQEVTYA |
| | | QLDHCVFIQRKISRPSQRPKTPLTDTSVYTELPNAEPRSKVVS |
| | | CPRAPQSGLEGVF |
| KIR3DL3 | 109 | HRWCANKKNAVVMDQEPAGNRTVNREDSDEQDPQEVTYA |
| | | QLNHCVFTQRKITRPSQRPKTPPTDTSV |
| LAIR1 | 110 | HRQNQIKQGPPRSKDEEQKPQQRPDLAVDVLERTADKATV |
| | | NGLPEKDRETDTSALAAGSSQEVTYAQLDHWALTQRTARA |
| | | VSPQSTKPMAESITYAAVARH |
| CD22 | 111 | KLQRRWKRTQSQQGLQENSSGQSFFVRNKKVRRAPLSEGP |
| | | HSLGCYNPMMEDGISYTTLRFPEMNIPRTGDAESSEMQRPPP |
| | | DCDDTVTYSALHKRQVGDYENVIPDFPEDEGIHYSELIQFGV |
| | | GERPQAQENVDYVILKH |
| CD33 | 112 | KTHRRKAARTAVGRNDTHPTTGSASPKHQKKSKLHGPTETS |
| | | SCSGAAPTVEMDEELHYASLNFHGMNPSKDTSTEYSEVRTQ |
| SIGLEC5 | 113 | KARRKQAAGRPEKMDDEDPIMGTITSGSRKKPWPDSPGDQ |
| | | ASPPGDAPPLEEQKELHYASLSFSEMKSREPKDQEAPSTTEY |
| | | SEIKTSK |
| SIGLEC6 | 114 | RVKTRRKKAAQPVQNTDDVNPVMVSGSRGHQHQFQTGIVS |
| | | DHPAEAGPISEDEQELHYAVLHFHKVQPQEPKVTDTEYSEIK |
| | | IHK |
| SIGLEC7 | 115 | RSCRKKSARPAADVGDIGMKDANTIRGSASQGNLTESWAD |
| | | DNPRHHGLAAHSSGEEREIQYAPLSFHKGEPQDLSGQEATN |
| | | NEYSEIKIPK |
| SIGLEC8 | 116 | RSCRKKSARPAAGVGDTGMEDAKAIRGSASQGPLTESWKD |
| | | GNPLKKPPPAVAPSSGEEGELHYATLSFHKVKPQDPQGQEA |
| | | TDSEYSEIKIHKRETAETQACLRNHNPSSKEVRG |
| SIGLEC9 | 117 | VRSCRKKSARPAAGVGDTGIEDANAVRGSASQGPLTEPWA |
| | | EDSPPDQPPPASARSSVGEGELQYASLSFQMVKPWDSRGQE |
| | | ATDTEYSEIKIHR |
| SIGLEC10 | 118 | KILPKRRTQTETPRPRFSRHSTILDYINVVPTAGPLAQKRNQK |
| | | ATPNSPRTPLPPGAPSPESKKNQKKQYQLPSFPEPKSSTQAPE |
| | | SQESQEELHYATLNFPGVRPRPEARMPKGTQADYAEVKFQ |
| SIGLEC11 | 119 | KICRKEARKRAAAEQDVPSTLGPISQGHQHECSAGSSQDHP |
| | | PPGAATYTPGKGEEQELHYASLSFQGLRLWEPADQEAPSTT |
| | | EYSEIKIHTGQPLRGPGFGLQLEREMSGMVPK |
| SIGLEC12 | 120 | RSCRKKSARPAVGVGDTGMEDANAVRGSASQGPLIESPAD |
| | | DSPPHHAPPALATPSPEEGEIQYASLSFHKARPQYPQEQEAIG |
| | | YEYSEINIPK |
| PECAM1/CD31 | 121 | KCYFLRKAKAKQMPVEMSRPAVPLLNSNNEKMSDPNMEA |
| | | NSHYGHNDDVRNHAMKPINDNKEPLNSDVQYTEVQVSSAE |
| | | SHKDLGKKDTETVYSEVRKAVPDAVESRYSRTEGSLDGT |
| CD200R1 | 122 | KVNGCRKYKLNKTESTPVVEEDEMQPYASYTEKNNPLYDT |
| | | TNKVKASEALQSEVDTDLHTL |
| FCRL1 | 123 | GLKRKIGRRSARDPLRSLPSPLPQEFTYLNSPTPGQLQPIYEN |
| | | VNVVSGDEVYSLAYYNQPEQESVAAETLGTHMEDKVSLDI |
| | | YSRLRKANITDVDYEDAM |
| FCRL2 | 124 | HKISGESSATNEPRGASRPNPQEFTYSSPTPDMEELQPVYVN |
| | | VGSVDVDVVYSQVWSMQQPESSANIRTLLENKDSQVIYSSV |
| | | KKS |
| FCRL3 | 125 | HYARARRKPGGLSATGTSSHSPSECQEPSSSRPSRIDPQEPTH |
| | | SKPLAPMELEPMYSNVNPGDSNPIYSQIWSIQHTKENSANCP |
| | | MMHQEHEELTVLYSELKKTHPDDSAGEASSRGRAHEEDDE |
| | | ENYENVPRVLLASDH |
| FCRL4 | 126 | HCWRRRKSGVGFLGDETRLPPAPGPGESSHSICPAQVELQSL |
| | | YVDVHPKKGDLVYSEIQTTQLGEEEEANTSRTLLEDKDVSV |
| | | VYSEVKTQHPDNSAGKISSKDEES |
| FCRL5 | 127 | LSRKAGRKPASDPARSPSDSDSQEPTYHNVPAWEELQPVYT |
| | | NANPRGENVVYSEVRIIQEKKKHAVASDPRHLRNKGSPIIYS |
| | | EVKVASTPVSGSLFLASSAPHR |
| SLAMF1 | 128 | QLRRRGKTNHYQTTVEKKSLTIYAQVQKPGPLQKKLDSFPA |
| | | QDPCTTIYVAATEPVPESVQETNSITVYASVTLPES |
| SLAMF5 | 129 | RLFKRRQGRIFPEGSCLNTFTKNPYAASKKTIYTYIMASRNT |
| | | QPAESRIYDEILQSKVLPSKEEPVNTVYSEVQFADKMGKAST |
| | | QDSKPPGTSSYEIVI |
| BTLA | 130 | RRHQGKQNELSDTAGREINLVDAHLKSEQTEASTRQNSQVL |
| | | LSETGIYDNDPDLCFRMQEGSEVYSNPCLEENKPGIVYASLN |
| | | HSVIGPNSRLARNVKEAPTEYASICVRS |
| LAG3 | 131 | HLWRRQWRPRRFSALEQGIHPPQAQSKIEELEQEPEPEPEPE |
| | | PEPEPEPEPEQL |
| 2B4 | 132 | WRRKRKEKQSETSPKEFLTIYEDVKDLKTRRNHEQEQTFPG |
| | | GGSTIYSMIQSQSSAPTSQEPAYTLYSLIQPSRKSGSRKRNHS |
| | | PSFNSTIYEVIGKSQPKAQNPARLSRKELENFDVYS |
| CD160 | 133 | GCINITSSASQEGTRLNLICTVWHKKEEAEGFVVFLCKDRSG |
| | | DCSPETSLKQLRLKRDPGIDGVGEISSQLMFTISQVTPLHSGT |
| | | YQCCARSQKSGIRLQGHFFSILFTETGNYTVTGLKQRQHLEF |
| | | SHNEGTLS |
| CEACAM1 | 134 | HFGKTGRASDQRDLTEHKPSVSNHTQDHSNDPPNKMNEVT |
| | | YSTLNFEAQQPTQPTSASPSLTATEIIYSEVKKQ |
| TIM3 | 135 | FKWYSHSKEKIQNLSLISLANLPPSGLANAVAEGIRSEENIYT |
| | | IEENVYEVEEPNEYYCYVSSRQQPSQPLGCRFAMP |
| VISTA | 136 | YKQRQAASNRRAQELVRMDSNIQGIENPGFEASPPAQGIPE |
| | | AKVRHPLSYVAQRQPSESGRHLLSEPSTPLSPPGPGDVFFPSL |
| | | DPVPDSPNFEVI |
| TIGIT | 137 | LTRKKKALRIHSVEGDLRRKSAGQEEWSPSAPSPPGSCVQA |
| | | EAAPAGLCGEQRGEDCAELHDYFNVLSYRSLGNCSFFTETG |
| SIRPalpha | 138 | RIRQKKAQGSTSSTRLHEPEKNAREITQDTNDITYADLNLPK |
| | | GKKPAPQAAEPNNHTEYASIQTSPQPASEDTLTYADLDMVH |
| | | LNRTPKQPAPKPEPSFSEYASVQVPRK |
| FcγRIIB | 139 | VVALIYCRKKRISALPGYPECREMGETLPEKPANPTNPDEAD |
| | | KVGAENTITYSLLMHPDALEEPDDQNRI |
| CD5 | 140 | KKLVKKFRQKKQRQWIGPTGMNQNMSFHRNHTATVRSHA |
| | | ENPTASHVDNEYSQPPRNSHLSAYPALEGALHRSSMQPDNS |
| | | SDSDYDLHGAQRL |
| CD300a | 141 | RMFQKWIKAGDHSELSQNPKQAATQSELHYANLELLMWPL |
| | | QEKPAPPREVEVEYSTVASPREELHYASVVFDSNTNRIAAQ |
| | | RPREEEPDSDYSVIRKT |
| CD300f | 142 | WRMMKYQQKAAGMSPEQVLQPLEGDLCYADLTLQLAGTS |
| | | PQKATTKLSSAQVDQVEVEYVTMASLPKEDISYASLTLGAE |
| | | DQEPTYCNMGHLSSHLPGRGPEEPTEYSTISRP |
| LIR1 | 143 | LRHRRQGKHWTSTQRKADFQHPAGAVGPEPTDRGLQWRSS |
| | | PAADAQEENLYAAVKHTQPEDGVEMDTRSPHDEDPQAVTY |
| | | AEVKHSRPRREMASPPSPLSGEFLDTKDRQAEEDRQMDTEA |
| | | AASEAPQDVTYAQLHSLTLRREATEPPPSQEGPSPAVPSIYA |
| | | TLAIH |
| LIR2 | 144 | LRHRRQGKHWTSTQRKADFQHPAGAVGPEPTDRGLQWRSS |
| | | PAADAQEENLYAAVKDTQPEDGVEMDTRAAASEAPQDVT |
| | | YAQLHSLTLRRKATEPPPSQEREPPAEPSIYATLAIH |
| LIR3 | 145 | RRQRHSKHRTSDQRKTDFQRPAGAAETEPKDRGLLRRSSPA |
| | | ADVQEENLYAAVKDTQSEDRVELDSQSPHDEDPQAVTYAP |
| | | VKHSSPRREMASPPSSLSGEFLDTKDRQVEEDRQMDTEAAA |
| | | SEASQDVTYAQLHSLTLRRKATEPPPSQEGEPPAEPSIYATL |
| | | AIH |
| LIR5 | 146 | QHWRQGKHRTLAQRQADFQRPPGAAEPEPKDGGLQRRSSP |
| | | AADVQGENFCAAVKNTQPEDGVEMDTRQSPHDEDPQAVT |
| | | YAKVKHSRPRREMASPPSPLSGEFLDTKDRQAEEDRQMDTE |
| | | AAASEAPQDVTYAQLHSFTLRQKATEPPPSQEGASPAEPSV |
| | | YATLAIH |
| LIR8 | 147 | RHRHQSKHRTSAHFYRPAGAAGPEPKDQGLQKRASPVADI |
| | | QEEILNAAVKDTQPKDGVEMDARAAASEAPQDVTYAQLHS |
| | | LTLRREATEPPPSQEREPPAEPSIYAPLAIH |
| Ly9 | 148 | KRKGRCSVPAFCSSQAEAPADTPEPTAGHTLYSVLSQGYEK |
| | | LDTPLRPARQQPTPTSDSSSDSNLTTEEDEDRPEVHKPISGRY |
| | | EVFDQVTQEGAGHDPAPEGQADYDPVTPYVTEVESVVGEN |
| | | TMYAQVFNLQGKTPVSQKEESSATIYCSIRKPQVVPPPQQN |
| | | DLEIPESPTYENFT |
| 2xPD1(G4S) | 149 | CSRAARGTIGARRTGQPLKEDPSAVPVFSVDYGELDFQWRE |
| | | KTPEPPVPCVPEQTEYATIVFPSGMGTSSPARRGSADGPRSA |
| | | QPLRPEDGHCSWPLGGGGSGGGGSCSRAARGTIGARRTGQP |
| | | LKEDPSAVPVFSVDYGELDFQWREKTPEPPVPCVPEQTEYA |
| | | TIVFPSGMGTSSPARRGSADGPRSAQPLRPEDGHCSWPL |
| 2xPD1(PD1) | 150 | CSRAARGTIGARRTGQPLKEDPSAVPVFSVDYGELDFQWRE |
| | | KTPEPPVPCVPEQTEYATIDFQWREKTPEPPVPCVPEQVDYG |
| | | ELDFQWREKTPEPPVPCVPEQTEYATIVFPSGMGTSSPARRG |
| | | SADGPRSAQPLRPEDGHCSWPL |
| PVRIg | 151 | LRRHKHRPAPRLQPSRTSPQAPRARAWAPSQASQAALHVPY |
| | | ATINTSCRPATLDTAHPHGGPSWWASLPTHAAHRPQGPAA |
| | | WASTPIPARGSFVSVENGLYAQAGERPPHTGPGLTLFPDPRG |
| | | PRAMEGPLGVR |
| AA2AR | 152 | RIREFRQTFRKIIRSHVLRQQEPFKAAGTSARVLAAHGSDGE |
| | | QVSLRLNGHPPGVWANGSAPHPERRPNGYALGLVSGGSAQ |
| | | ESQGNTGLPDVELLSHELKGVCPEPPGLDDPLAQDGAGVS |

5. Optional Synthetic PD-1

In some embodiments, the iCAR construct comprises an optional synthetic PD-1 sequence. In some embodiments, the iCAR comprises a synthetic PD-1 sequence shown in Table 8. In some embodiments, the iCAR construct comprises an optional synthetic LIR1 sequence. In some embodiments, the iCAR comprises a synthetic LIR1 sequence shown in Table 8.

TABLE 8
synthetic PD-1 and LIR1 sequences

| SEQ | |
|---|---|
| ID NO | Sequence intracellular synPD-1 |
| 243 | CSRAARGTIGARRTGQPLKEDPSAVPVFSVDYGELVFPSGMGTSSPARR |
| | GSADGPRSAQPLRPEDGHCSWPL |
| 244 | CSRAARGTIGARRTGQPLKEDPSAVPVFSVDYGELDFQWREKTPEPPVP |
| | CVPEQVDYGELVFPSGMGTSSPARRGSADGPRSAQPLRPEDGHCSWPL |
| 245 | CSRAARGTIGARRTGQPLKEDPSAVPVFSVDYGELDFQWREKTPEPPVP |
| | CVPEQVDYGELDFQWREKTPEPPVPCVPEQVDYGELVFPSGMGTSSPAR |
| | RGSADGPRSAQPLRPEDGHCSWPL |
| 246 | CSRAARGTIGARRTGQPLKEDPSAVPVFSVDYGELDFQWREKTPEPPVP |
| | CVPEQVDYGELDFQWREKTPEPPVPCVPEQVDYGELDFQWREKTPEPP |
| | VPCVPEQVDYGELVFPSGMGTSSPARRGSADGPRSAQPLRPEDGHCSWP |
| | L |
| 247 | CSRAARGTIGARRTGQPLKEDPSAVPVFSVDYGELDFQWREKTPEPPVP |
| | CVPEQVDYGELDFQWREKTPEPPVPCVPEQVDYGELDFQWREKTPEPP |
| | VPCVPEQVDYGELDFQWREKTPEPPVPCVPEQVDYGELVFPSGMGTSSP |
| | ARRGSADGPRSAQPLRPEDGHCSWPL |
| 248 | CSRAARGTIGARRTGQPLKEDPSAVPVFSTEYATIVFPSGMGTSSPARRG |
| | SADGPRSAQPLRPEDGHCSWPL |
| 249 | CSRAARGTIGARRTGQPLKEDPSAVPVFSTEYATIDFQWREKTPEPPVPC |
| | VPEQTEYATIVFPSGMGTSSPARRGSADGPRSAQPLRPEDGHCSWPL |
| 250 | CSRAARGTIGARRTGQPLKEDPSAVPVFSTEYATIDFQWREKTPEPPVPC |
| | VPEQTEYATIDFQWREKTPEPPVPCVPEQTEYATIVFPSGMGTSSPARRG |
| | SADGPRSAQPLRPEDGHCSWPL |
| 251 | CSRAARGTIGARRTGQPLKEDPSAVPVFSTEYATIDFQWREKTPEPPVPC |
| | VPEQTEYATIDFQWREKTPEPPVPCVPEQTEYATIDFQWREKTPEPPVPC |
| | VPEQTEYATIVFPSGMGTSSPARRGSADGPRSAQPLRPEDGHCSWPL |
| 252 | CSRAARGTIGARRTGQPLKEDPSAVPVESTEYATIDFQWREKTPEPPVPC |
| | VPEQTEYATIDFQWREKTPEPPVPCVPEQTEYATIDFQWREKTPEPPVPC |
| | VPEQTEYATIDFQWREKTPEPPVPCVPEQTEYATIVFPSGMGTSSPARRG |
| | SADGPRSAQPLRPEDGHCSWPL |
| 253 | CSRAARGTIGARRTGQPLKEDPSAVPVFSVDYGELDFQWREKTPEPPVP |
| | CVPEQTEYATIVFPSGMGTSSPARRGSADGPRSAQPLRPEDGHCSWPLG |
| | GGGSGGGGSCSRAARGTIGARRTGQPLKEDPSAVPVFSVDYGELDFQW |
| | REKTPEPPVPCVPEQTEYATIVFPSGMGTSSPARRGSADGPRSAQPLRPE |
| | DGHCSWPL |
| 254 | CSRAARGTIGARRTGQPLKEDPSAVPVFSVDYGELDFQWREKTPEPPVP |
| | CVPEQTEYATIDFQWREKTPEPPVPCVPEQVDYGELDFQWREKTPEPPV |
| | PCVPEQTEYATIVFPSGMGTSSPARRGSADGPRSAQPLRPEDGHCSWPL |
| 296 | LRHRRQGKHWTSTQRKADFQHPAGAVGPEPTDRGLQWRSSPAADAQE |
| | ENLYAAVKHTQPEDGVEMDTRSPHDEDPQANLYAAVKHSRPRREMAS |
| | PPSPLSGEFLDTKDRQAEEDRQMDTEAAASEAPQDNLYAAVHSLTLRRE |
| | ATEPPPSQEGPSPAVPNLYAAVAIH |
| 297 | LRHRRQGKHWTSTQRKADFQHPAGAVGPEPTDRGLQWRSSPAADAQE |
| | EVTYAEVKHTQPEDGVEMDTRSPHDEDPQAVTYAEVKHSRPRREMASP |
| | PSPLSGEFLDTKDRQAEEDRQMDTEAAASEAPQDVTYAEVHSLTLRREA |
| | TEPPPSQEGPSPAVPVTYAEVAIH |
| 298 | LRHRRQGKHWTSTQRKADFQHPAGAVGPEPTDRGLQWRSSPAADAQE |
| | EVTYAQLKHTQPEDGVEMDTRSPHDEDPQAVTYAQLKHSRPRREMASP |
| | PSPLSGEFLDTKDRQAEEDRQMDTEAAASEAPQDVTYAQLHSLTLRREA |
| | TEPPPSQEGPSPAVPVTYAQLAIH |
| 299 | LRHRRQGKHWTSTQRKADFQHPAGAVGPEPTDRGLQWRSSPAADAQE |
| | ESIYATLKHTQPEDGVEMDTRSPHDEDPQASIYATLKHSRPRREMASPPS |
| | PLSGEFLDTKDRQAEEDRQMDTEAAASEAPQDSIYATLHSLTLRREATE |
| | PPPSQEGPSPAVPSIYATLAIH |
| 300 | LRHRRQGKHWTSTQRKADFQHPAGAVGPEPTDRGLQWRSSPAADAQE |
| | EVTYAQLKHTQPEDGVEMDTRSPHDEDPQASIYATLKHSRPRREMASPP |
| | SPLSGEFLDTKDRQAEEDRQMDTEAAASEAPQDVTYAQLHSLTLRREAT |
| | EPPPSQEGPSPAVPSIYATLAIH |
| 301 | LRHRRQGKHWTSTQRKADFQHPAGAVGPEPTDRGLQWRSSPAADAQE |
| | ETEYATIKHTQPEDGVEMDTRSPHDEDPQATEYATIKHSRPRREMASPPS |
| | PLSGEFLDTKDRQAEEDRQMDTEAAASEAPQDVTYAQLHSLTLRREAT |
| | EPPPSQEGPSPAVPSIYATLAIH |
| 302 | LRHRRQGKHWTSTQRKADFQHPAGAVGPEPTDRGLQWRSSPAADAQE |
| | EVTYAQLKHTQPEDGVEMDTRSPHDEDPQASIYATLKHSRPRREMASPP |
| | SPLSGEFLDTKDRQAEEDRQMDTEAAASEAPQDTEYATIHSLTLRREAT |
| | EPPPSQEGPSPAVPTEYATIAIH |
| 304 | LRHRRQGKHWTSTQRKADFQHPAGAVGPEPTDRGLQWRSSPAADAQE |
| | EVTYAQLKHTQPEDGVEMDTRSPHDEDPQATEYATIKHSRPRREMASPP |
| | SPLSGEFLDTKDRQAEEDRQMDTEAAASEAPQDTEYATIHSLTLRREAT |
| | EPPPSQEGPSPAVPSIYATLAIH |

6. Exemplary iCARs

In some embodiments, the iCAR comprises an scFv component comprising the VL and VH sequences of BB7.2 (SEQ ID NOs: 37 and 38). In some embodiments, the orientation of the iCAR VH and VL regions is VH-VL. In some embodiments, the orientation of the iCAR VH and VL regions is VL-VH. In some embodiments, the iCAR scFv comprises a (G₄S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa (SEQ ID NO: 289). In some embodiments, the iCAR comprises PD-1 (47) (SEQ ID NO: 290). In some embodiments, the iCAR comprises PD-1 (42) (SEQ ID NO: 291). In some embodiments, the iCAR comprises PD-1 (36) (SEQ ID NO: 292). In some embodiments, the iCAR comprises PD-1 (30) (SEQ ID NO: 293). In some embodiments, the iCAR comprises PD-1 (26) (SEQ ID NO: 294). In some embodiments, the iCAR comprises PD-1 (20) (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO: 112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:]122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306). In some embodiments, the iCAR comprises an scFv component comprising the VL and VH sequences of 3PF12/C4 (SEQ ID NOs: 39 and 40). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO: 114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of 3PF12/F12 (SEQ ID NOs: 41 and 42). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of 3PF12/B11 (SEQ ID NOs: 43 and 44). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO: 114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of W6/32 (SEQ ID NOs: 45 and 46). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO:117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of BBM.1 (SEQ ID NOs: 47 and 48). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO: 114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of SN66E3.1 (SEQ ID NOs: 49 and 50). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO:117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcβRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Ha5C2.A2 (SEQ ID NOs: 51 and 52). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO: 114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of MWB1 (SEQ ID NOs: 53 and 54). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO: 111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of MWB1-mod (MWB1.1) (SEQ ID NOs: 55 and 56). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO: 114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-69_A18VK (SEQ ID NOs: 57 and 58). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-69 (27,30)_A18 (SEQ ID NOs: 59 and 60). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2VH1-69 (27,30,48)_A18 (SEQ ID NOs: 61 and 62). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO:117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-69 (27,30,67)_A18 (SEQ ID NOs: 63 and 64). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-69 (27,30,69)_A18 (SEQ ID NOs: 65 and 66). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-69 (27,30,67,69)_A18 (SEQ ID NOs: 67 and 68). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-3_A18 (SEQ ID NOs: 69 and 70). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-3(48)_A18 (SEQ ID NOs: 71 and 72). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-3(67)_A18 (SEQ ID NOs: 73 and 74). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO:117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.Bb7.2 VH1-3(69)_A18 (SEQ ID NOs: 75 and 76). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO:117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the scFv has the VL and VH sequences of Hz.BB7.2 VH1-3(71)_A18 (SEQ ID NOs: 77 and 78). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz. BB7.2VH1-3(73)_A18 (SEQ ID NOs: 79 and 80). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the scFv has the VL and VH sequences of MWB1.2 (SEQ ID NOs: 163 and 164). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO: 139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the scFv has the VL and VH sequences of SN66E3.2 (SEQ ID NOs: 165 and 166). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the scFv has the VL and VH sequences of MWB1.1 (SEQ ID NOs: 273). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO:116). In some embodiments, the iCAR comprises a SIGLEC9 inhibitory domain (SEQ ID NO: 117). In some embodiments, the iCAR comprises a SIGLEC10 inhibitory domain (SEQ ID NO: 118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the scFv has the VL and VH sequences of MWB1.2 (SEQ ID NOs: 274). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO:117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the scFv has the VL and VH sequences of SN66E3.3 (SEQ ID NOs: 283 and 284). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises aLIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO: 113). In some embodiments, the iCAR comprises a SIGLEC6 inhibitory domain (SEQ ID NO:114). In some embodiments, the iCAR comprises a SIGLEC7 inhibitory domain (SEQ ID NO: 115). In some embodiments, the iCAR comprises a SIGLEC8 inhibitory domain (SEQ ID NO: 116). In some embodiments, the iCAR comprises a SIGLEC9 inhibitory domain (SEQ ID NO:117). In some embodiments, the iCAR comprises a SIGLEC10 inhibitory domain (SEQ ID NO: 118). In some embodiments, the iCAR comprises a SIGLEC11 inhibitory domain (SEQ ID NO: 119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2xPD1(G4S) inhibitory domain (SEQ ID NO:149). In some embodiments, the iCAR comprises a 2xPD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR has a set of components shown in Tables 9-10 and/or an amino acid sequence shown in Tables 11-12.

TABLE 9
iCAR constructs

| | | VH | VL | | | | | |
|---|---|---|---|---|---|---|---|---|
| | | SEQ | SEQ | | | | | |
| | | ID | ID | Signal | scFv | | | |
| Construct | scFv | NO | NO | peptide | Linker | Hinge | TM | Signaling |
| | BB7.2 | 38 | 37 | CD8alpha | (G4S) × 3 | PD-1 | PD-1 | PD-1 |
| MC0096 | 3PF12/C4 | 40 | 39 | CD8alpha | (G4S) × 3 | PD-1 | PD-1 | PD-1 |
| (VR96) | | | | | | | | |
| MC0274 | 3PF12/F12 | 42 | 41 | CD8alpha | (G4S) × 3 | PD-1 | PD-1 | PD-1 |
| (VR274) | | | | | | | | |
| MC0276 | 3PF12/B11 | 44 | 43 | CD8alpha | (G4S) × 3 | PD-1 | PD-1 | PD-1 |
| (VR276) | | | | | | | | |
| MC0097 | W6/32 | 46 | 45 | CD8alpha | (G4S) × 3 | PD-1 | PD-1 | PD-1 |
| (VR97) | | | | | | | | |
| MC0098 | BBM.1 | 48 | 47 | CD8alpha | (G4S) × 3 | PD-1 | PD-1 | PD-1 |
| (VR98) | | | | | | | | |
| MC0099 | SN66E3.1 | 50 | 49 | CD8alpha | (G4S) × 3 | PD-1 | PD-1 | PD-1 |
| (VR99) | | | | | | | | |
| MC0100 | Ha5C2.A2 | 52 | 51 | CD8alpha | (G4S) × 3 | PD-1 | PD-1 | PD-1 |
| (VR100) | | | | | | | | |
| MC0101 | MWB1 | 54 | 53 | CD8alpha | (G4S) × 3 | PD-1 | PD-1 | PD-1 |
| (VR101) | | | | | | | | |
| MC0102 | MWB1.1d | 56 | 55 | CD8alpha | (G4S) × 3 | PD-1 | PD-1 | PD-1 |
| (VR102) | | | | | | | | |
| MC0372 | Hz.BB7.2 VH1-69_A18VK | 58 | 57 | CD8 alpha | (G4S) × 3 | CD8 alpha | CD8 alpha | 41BBz |
| (VR372) | | | | | | | | |
| MC0373 | Hz.BB7.2 VH1-69 (27, 30)_A18 | 60 | 59 | CD8 alpha | (G4S) × 3 | CD8 alpha | CD8 alpha | 41BBz |
| (VR373) | | | | | | | | |
| MC0374 | Hz.BB7.2 VH1-69 (27, 30, 48)_A18 | 62 | 61 | CD8 alpha | (G4S) × 3 | CD8 alpha | CD8 alpha | 41BBz |
| (VR374) | | | | | | | | |
| MC0375 | Hz.BB7.2 VH1-69 (27, 30, 67)_A18 | 64 | 63 | CD8 alpha | (G4S) × 3 | CD8 alpha | CD8 alpha | 41BBz |
| (VR375) | | | | | | | | |
| MC0376 | Hz.BB7.2 VH1-69 (27, 30, 69)_A18 | 66 | 65 | CD8 alpha | (G4S) × 3 | CD8 alpha | CD8 alpha | 41BBz |
| (VR376) | | | | | | | | |
| MC0377 | Hz.BB7.2 VH1-69 (27, 30, 67, 69)_A18 | 68 | 67 | CD8 alpha | (G4S) × 3 | CD8 alpha | CD8 alpha | 41BBz |
| (VR377) | | | | | | | | |
| MC0378 | Hz.BB7.2 VH1-3_A18 | 70 | 69 | CD8 alpha | (G4S) × 3 | CD8alpha | CD8 alpha | 41BBz |
| (VR378) | | | | | | | | |
| MC0379 | Hz.BB7.2 VH1-3(48)_A18 | 72 | 71 | CD8 alpha | (G4S) × 3 | CD8 alpha | CD8 alpha | 41BBz |
| (VR379) | | | | | | | | |
| MC0380 | Hz.BB7.2 VH1-3(67)_A18 | 74 | 73 | CD8 alpha | (G4S) × 3 | CD8 alpha | CD8 alpha | 41BBz |
| (VR380) | | | | | | | | |
| MC0381 | Hz.BB7.2 VH1-3(69)_A18 | 76 | 75 | CD8 alpha | (G4S) × 3 | CD8 alpha | CD8 alpha | 41BBz |
| (VR381) | | | | | | | | |
| MC0382 | Hz.BB7.2 VH1-3(71)_A18 | 78 | 77 | CD8 alpha | (G4S) × 3 | CD8 alpha | CD8 alpha | 41BBz |
| (VR382) | | | | | | | | |
| MC0383 | Hz.BB7.2 VH1-3(73)_A18 | 80 | 79 | CD8 alpha | (G4S) × 3 | CD8 alpha | CD8 alpha | 41BBz |
| (VR383) | | | | | | | | |
| MC0384 | 3PF12_274_LIR1_HER2_shRNA(A2) | 40 | 41 | CD8 alpha | 3PF12_274 | (G4S) × 3 | PD-1 | LIR-1 |
| (VR384) | | | | | | | | |
| MC0385 | 3PF12_276_LIR1_HER2_shRNA(A2) | 44 | 43 | CD8 alpha | 3PF12_276 | (G4S) × 3 | PD-1 | LIR-1 |
| (VR385) | | | | | | | | |
| MC0386 | MWB1.1_HL_LIR1_HER2_shRNA(A2) | 56 | 55 | CD8 alpha | MWB1.1_HL | (G4S) × 3 | PD-1 | LIR-1 |
| (VR386) | | | | | | | | |
| MC0387 | MWB1.1_LH_LIR1_HER2_shRNA(A2) | 56 | 55 | CD8 alpha | MWB1.1_LH | (G4S) × 3 | PD-1 | LIR-1 |
| (VR387) | | | | | | | | |
| MC0388 | MWB1.2_HL_LIR1_HER2_shRNA(A2) | 164 | 163 | CD8 alpha | MWB1.2_HL | (G4S) × 3 | PD-1 | LIR-1 |
| (VR388) | | | | | | | | |
| MC0389 | MWB1.2_LH_LIR1_HER2_shRNA(A2) | 164 | 163 | CD8 alpha | MWB1.2_LH | (G4S) × 3 | PD-1 | LIR-1 |
| (VR389) | | | | | | | | |
| MC0390 | SN66E3.1_HL_LIR1_HER2_shRNA(A2) | 50 | 49 | CD8 alpha | SN66E3.1_HL | (G4S) × 3 | PD-1 | LIR-1 |
| (VR390) | | | | | | | | |
| MC0391 | SN66E3.1_LH_LIR1_HER2_shRNA(A2) | 50 | 49 | CD8 alpha | SN66E3.1_LH | (G4S) × 3 | PD-1 | LIR-1 |
| (VR391) | | | | | | | | |
| MC0446 | SN66E3.2_HL_LIR1_HER2_{—} | 166 | 165 | CD8 alpha | SN66E3.2_HL | (G4S) × 3 | LIR1 | LIR-1 |
| (VR446) | | | | | | | | |
| MC0447 | SN66E3.2_LH_LIR1_HER2_{—} | 166 | 165 | CD8 alpha | SN66E3.2_LH | (G4S) × 3 | LIR1 | LIR-1 |
| (VR447) | | | | | | | | |
| MC0448 | SN66E3.3(HL)_LIR1(26)_HER2 | 284 | 283 | CD8 alpha | SN66E.3.3_HL | | LIR1 | LIR1 |
| (VR448) | | | | | | | | |
| MC449 | SN66E3.3(LH)_LIR1(26)_HER2 | 284 | 283 | CD8 alpha | SN66E3.3_LH | (G4S) × 3 | LIR1 | LIR1 |
| (VR449) | | | | | | | | |
| MC0428 | HzBB7.2.1_H69_LIR1_H | 64 | 63 | CD8 alpha | HzBB7.2_H69 | (G4S) × 3 | LIR1 | LIR-1 |
| (VR428) | | | | | | | | |
| MC0421 | HzBB7.2.2_H3_LIR1_) | 72 | 71 | CD8 alpha | HzBB7.2_H3 | (G4S) × 3 | LIR1 | LIR-1 |
| (VR421) | | | | | | | | |

TABLE 10
iCAR constructs

| | Construct | Signal | | scFv | | | |
|---|---|---|---|---|---|---|---|
| Construct | Name | Peptide | scFv | Linker | Hinge | TM | Signaling |
| MC0058 | 1 × ITIM | CD8alpha | BB7.2 | (G4S) × 3 | PD-1 | PD-1 | 1 × ITIM |
| (VR58) | | | VH VL | | | | PD-1 |
| MC0059 | 2 × ITIM | CD8alpha | BB7.2 | (G4S) × 3 | PD-1 | PD-1 | 2 × ITIM |
| (VR59) | | | VH VL | | | | PD-1 |
| MC0060 | 3 × ITIM | CD8alpha | BB7.2 | (G4S) × 3 | PD-1 | PD-1 | 3 × ITIM |
| (VR60) | | | VH VL | | | | PD-1 |
| MC0061 | 4 × ITIM | CD8alpha | BB7.2 | (G4S) × 3 | PD-1 | PD-1 | 4 × ITIM |
| (VR61) | | | VH VL | | | | PD-1 |
| MC0062 | 5 × ITIM | CD8alpha | BB7.2 | (G4S) × 3 | PD-1 | PD-1 | 5 × ITIM |
| (VR62) | | | VH VL | | | | PD-1 |
| MC0063 | 1 × ITSM | CD8alpha | BB7.2 | (G4S) × 3 | PD-1 | PD-1 | 1 × ITSM |
| (VR63) | | | VH VL | | | | PD-1 |
| MC0064 | 2 × ITSM | CD8alpha | BB7.2 | (G4S) × 3 | PD-1 | PD-1 | 2 × ITSM |
| (VR64) | | | VH VL | | | | PD-1 |
| MC0065 | 3 × ITSM | CD8alpha | BB7.2 | (G4S) × 3 | PD-1 | PD-1 | 3 × ITSM |
| (VR65) | | | VH VL | | | | PD-1 |
| MC0066 | 4 × ITSM | CD8alpha | BB7.2 | (G4S) × 3 | PD-1 | PD-1 | 4 × ITSM |
| (VR66) | | | VH VL | | | | PD-1 |
| MC0067 | 5 × ITSM | CD8alpha | BB7.2 | (G4S) × 3 | PD-1 | PD-1 | 5 × ITSM |
| (VR67) | | | VH VL | | | | PD-1 |
| MC0068 | 2 × PD1(G4S) | CD8alpha | BB7.2 | (G4S) × 3 | PD-1 | PD-1 | 2 × PD-1 |
| (VR68) | | | VH VL | | | | (G4S) × 2 |
| MC0069 | 2 × PD1(PD1) | CD8alpha | BB7.2 | (G4S) × 3 | PD-1 | PD-1 | 2 × PD-1 |
| (VR69) | | | VH VL | | | | (PD1 linker) |

TABLE 11
iCAR constructs

| | | SEQ | |
|---|---|---|---|
| | | ID | |
| Construct | scFv | NO | Amino acid sequence |
| MC0387 | MWB1.1_ | 255 | MALPVTALLLPLALLLHAARPQSALTQPPSASGSPGQSV |
| (VR387) | LH_LIR1_ | | TISCTGTSSDVGGYKYVSWYQHHPDKAPKLMIYEVNK |
| | HER2_ | | RPSGVPDRFSGSKSDNTASLTVSGLQAEDEADYYCSSY |
| | shRNA(A2) | | AGSNNWVFGGGTKLTVLGGGGSGGGGSGGGGSQVQL |
| | | | VESGGGVVQPGGSLRLSCAASGFTFSTYGMHWVRQAP |
| | | | GKGLEWVASISYDGSNKYYADSGQGRFTISRDTSKNSL |
| | | | YLQMNSLRAEDTAVYYCAIGIYGAYSFDYWGQGTLVT |
| | | | VSSTERRAEVPTAHPSPSPRPAGQFQTLVVGVVGGLLGS |
| | | | LVLLVWVLAVILRHRRQGKHWTSTQRKADFQHPAGAV |
| | | | GPEPTDRGLQWRSSPAADAQEENLYAAVKHTQPEDGV |
| | | | EMDTRSPHDEDPQAVTYAEVKHSRPRREMASPPSPLSG |
| | | | EFLDTKDRQAEEDRQMDTEAAASEAPQDVTYAQLHSL |
| | | | TLRREATEPPPSQEGPSPAVPSIYATLAIH |
| MC0389 | MWB1.2_ | 256 | MALPVTALLLPLALLLHAARPQSALTQPPSASGSPGQSV |
| (VR389) | LH_LIR1_ | | TISCTGTSSDVGGYKYVSWYQQHPGKAPKLMIYEVNK |
| | HER2_ | | RPSGVPDRFSGSKSGNTASLTVSGLQAEDEADYYCSSY |
| | shRNA(A2) | | AGSNNWVFGGGTKLTVLGGGGSGGGGSGGGGSQVQL |
| | | | VESGGGVVQPGGSLRLSCAASGFTFSTYGMHWVRQAP |
| | | | GKGLEWVASISYDGSNKYYADSGQGRFTISRDTSKNSL |
| | | | YLQMNSLRAEDTAVYYCAIGIYGAYSFDYWGQGTLVT |
| | | | VSSTERRAEVPTAHPSPSPRPAGQFQTLVVGVVGGLLGS |
| | | | LVLLVWVLAVILRHRRQGKHWTSTQRKADFQHPAGAV |
| | | | GPEPTDRGLQWRSSPAADAQEENLYAAVKHTQPEDGV |
| | | | EMDTRSPHDEDPQAVTYAEVKHSRPRREMASPPSPLSG |
| | | | EFLDTKDRQAEEDRQMDTEAAASEAPQDVTYAQLHSL |
| | | | TLRREATEPPPSQEGPSPAVPSIYATLAIH |
| MC0391 | SN66E3.1_ | 257 | MALPVTALLLPLALLLHAARPDIVMTQSPDSLAVSLGE |
| (VR391) | LH_LIR1_ | | RATISCKSSQSVLYSSNNKNYLAWYQQKLGQPPKLLIY |
| | HER2_ | | WASTRESGVPDRFSGSGSGTNFTLTISSLQAENVAVYYC |
| | shRNA(A2) | | QQYYGTPFTFGGGTKVEIKGGGGSGGGGSGGGGSQVQ |
| | | | LVQSGAEVKKPGASVKVSCKASGYTFTDYYLHWVRQA |
| | | | PGQGLEWMGWINPYTGGTNYAQKFQGRVTMTRDASIS |
| | | | TVYMELSGLTSDDTAVHFCARAGASYYDFWSGWVED |
| | | | YWGQGTLVTVSSTERRAEVPTAHPSPSPRPAGQFQTLV |
| | | | VGVVGGLLGSLVLLVWVLAVILRHRRQGKHWTSTQRK |
| | | | ADFQHPAGAVGPEPTDRGLQWRSSPAADAQEENLYAA |
| | | | VKHTQPEDGVEMDTRSPHDEDPQAVTYAEVKHSRPRR |
| | | | EMASPPSPLSGEFLDTKDRQAEEDRQMDTEAAASEAPQ |
| | | | DVTYAQLHSLTLRREATEPPPSQEGPSPAVPSIYATLAIH |
| MC0447 | SN66E3.2 | 258 | MALPVTALLLPLALLLHAARPDIVMTQSPDSLAVSLGE |
| (VR447) | (LH)_LIR1 | | RATISCKSSQSVLYSSNNKNYLAWYQQKPGQPPKLLIY |
| | (30)_HER2 | | WASTRESGVPDRFSGSGSGTDFTLTISSLQAEDVAVYYC |
| | | | QQYYGTPFTFGGGTKVEIKGGGGSGGGGSGGGGSQVQ |
| | | | LVQSGAEVKKPGASVKVSCKASGYTFTDYYLHWVRQA |
| | | | PGQGLEWMGWINPYTGGTNYAQKFQGRVTMTRDTSIS |
| | | | TAYMELSGLTSDDTAVYYCARAGASYYDFWSGWVED |
| | | | YWGQGTLVTVSSGPTSTSGPEDQPLTPTGSDPQSGLGR |
| | | | HLGVVIGILVAVILLLLLLLLLFLILRHRRQGKHWTSTQ |
| | | | RKADFQHPAGAVGPEPTDRGLQWRSSPAADAQEENLY |
| | | | AAVKHTQPEDGVEMDTRSPHDEDPQAVTYAEVKHSRP |
| | | | RREMASPPSPLSGEFLDTKDRQAEEDRQMDTEAAASEA |
| | | | PQDVTYAQLHSLTLRREATEPPPSQEGPSPAVPSIYATL |
| | | | AIH |
| MC0449 | SN66E3.3 | 305 | MALPVTALLLPLALLLHAARPDIVMTQSPDSLAVSLGE |
| (VR449) | (LH)_LIR1 | | RATISCKSSQSVLYSSNNKNYLAWYQQKPGQPPKLLIY |
| | (26)_HER2 | | WASTRESGVPDRFSGSGSGTDFTLTISSLQAEDVAVYYC |
| | | | QQYYGTPFTFGGGTKVEIKGGGGSGGGGSGGGGSQVQ |
| | | | LVQSGAEVKKPGASVKVSCKASGYTFTDYYLHWVRQA |
| | | | PGQGLEWMGWINPYTGGTNYAQKFQGRVTMTRDTSIS |
| | | | TAYMELSRLRSEDTAVYYCARAGASYYDFWSGWVFD |
| | | | YWGQGTLVTVSSTSGPEDQPLTPTGSDPQSGLGRHLGV |
| | | | VIGILVAVILLLLLLLLLFLILRHRRQGKHWTSTQRKADF |
| | | | QHPAGAVGPEPTDRGLQWRSSPAADAQEENLYAAVKH |
| | | | TQPEDGVEMDTRSPHDEDPQAVTYAEVKHSRPRREMA |
| | | | SPPSPLSGEFLDTKDRQAEEDRQMDTEAAASEAPQDVT |
| | | | YAQLHSLTLRREATEPPPSQEGPSPAVPSIYATLAIH |
| MC0428 | HzBB7.2.1_ | 259 | MALPVTALLLPLALLLHAARPQVQLVQSGAEVKKPGSS |
| (VR428) | _LIR1 | | VKVSCKASGYTFTSYHIQWVRQAPGQGLEWMGWIYPG |
| | (52)_HER2 | | DGSTQYNEKFKGRTTITADKSTSTAYMELSSLRSEDTA |
| | | | VYYCAREGTYYAMDYWGQGTLVTVSSGGGGSGGGGS |
| | | | GGGGSDVVMTQTPLSLSVTPGQPASISCRSSQSIVHSNG |
| | | | NTYLEWYLQKPGQSPQLLIYKVSNRFSGVPDRFSGSGS |
| | | | GTDFTLKISRVEAEDVGVYYCFQGSHVPRTFGGGTKVE |
| | | | IKHPSDPLELVVSGPSGGPSSPTTGPTSTSGPEDQPLTPT |
| | | | GSDPQSGLGRHLGVVIGILVAVILLLLLLLLLFLILRHRR |
| | | | QGKHWTSTQRKADFQHPAGAVGPEPTDRGLQWRSSPA |
| | | | ADAQEENLYAAVKHTQPEDGVEMDTRSPHDEDPQAVT |
| | | | YAEVKHSRPRREMASPPSPLSGEFLDTKDRQAEEDRQM |
| | | | DTEAAASEAPQDVTYAQLHSLTLRREATEPPPSQEGPSP |
| | | | AVPSIYATLAIH |
| MC0421 | HzBB7.2.2_ | 260 | MALPVTALLLPLALLLHAARPQVQLVQSGAEVKKPGA |
| (VR421) | _H3_LIR1_ | | SVKVSCKASGYTFTSYHIQWVRQAPGQRLEWIGWIYPG |
| | HER2_ | | DGSTQYNEKFKGRVTITRDTSASTAYMELSSLRSEDTA |
| | | | VYYCAREGTYYAMDYWGQGTLVTVSSGGGGSGGGGS |
| | | | GGGGSDVVMTQTPLSLSVTPGQPASISCRSSQSIVHSNG |
| | | | NTYLEWYLQKPGQSPQLLIYKVSNRFSGVPDRFSGSGS |
| | | | GTDFTLKISRVEAEDVGVYYCFQGSHVPRTFGGGTKVE |
| | | | IKHPSDPLELVVSGPSGGPSSPTTGPTSTSGPEDQPLTPT |
| | | | GSDPQSGLGRHLGVVIGILVAVILLLLLLLLLFLILRHRR |
| | | | QGKHWTSTQRKADFQHPAGAVGPEPTDRGLQWRSSPA |
| | | | ADAQEENLYAAVKHTQPEDGVEMDTRSPHDEDPQAVT |
| | | | YAEVKHSRPRREMASPPSPLSGEFLDTKDRQAEEDRQM |
| | | | DTEAAASEAPQDVTYAQLHSLTLRREATEPPPSQEGPSP |
| | | | AVPSIYATLAIH |

TABLE 12
iCAR constructs

| | | SEQ | |
|---|---|---|---|
| | Construct | ID | |
| Construct | Name | NO | full length iCAR sequence |
| MC0058 | 1xITIM | 261 | MALPVTALLLPLALLLHAARPEQKLISEEDLQVQLQQSGP |
| (VR58) | | | ELVKPGASVKMSCKASGYTFTSYHIQWVKQRPGQGLEWI |
| | | | GWIYPGDGSTQYNEKFKGKTTLTADKSSSTAYMLLSSLT |
| | | | SEDSAIYFCAREGTYYAMDYWGQGTSVTVSSGGGGSGG |
| | | | GGSGGGGSDVLMTQTPLSLPVSLGDQVSISCRSSQSIVHS |
| | | | NGNTYLEWYLQKPGQSPKLLIYKVSNRFSGVPDRESGSG |
| | | | SGTDFTLKISRVEAEDLGVYYCFQGSHVPRTFGGGTKLEI |
| | | | KTERRAEVPTAHPSPSPRPAGQFQTLVVGVVGGLLGSLV |
| | | | LLVWVLAVICSRAARGTIGARRTGQPLKEDPSAVPVFSV |
| | | | DYGELVFPSGMGTSSPARRGSADGPRSAQPLRPEDGHCS |
| | | | WPL |
| MC0059 | 2xITIM | 262 | MALPVTALLLPLALLLHAARPEQKLISEEDLQVQLQQSGP |
| (VR59) | | | ELVKPGASVKMSCKASGYTFTSYHIQWVKQRPGQGLEWI |
| | | | GWIYPGDGSTQYNEKFKGKTTLTADKSSSTAYMLLSSLT |
| | | | SEDSAIYFCAREGTYYAMDYWGQGTSVTVSSGGGGSGG |
| | | | GGSGGGGSDVLMTQTPLSLPVSLGDQVSISCRSSQSIVHS |
| | | | NGNTYLEWYLQKPGQSPKLLIYKVSNRFSGVPDRESGSG |
| | | | SGTDFTLKISRVEAEDLGVYYCFQGSHVPRTFGGGTKLEI |
| | | | KTERRAEVPTAHPSPSPRPAGQFQTLVVGVVGGLLGSLV |
| | | | LLVWVLAVICSRAARGTIGARRTGQPLKEDPSAVPVFSV |
| | | | DYGELDFQWREKTPEPPVPCVPEQVDYGELVFPSGMGTS |
| | | | SPARRGSADGPRSAQPLRPEDGHCSWPL |
| MC0060 | 3xITIM | 263 | MALPVTALLLPLALLLHAARPEQKLISEEDLQVQLQQSGP |
| (VR60) | | | ELVKPGASVKMSCKASGYTFTSYHIQWVKQRPGQGLEWI |
| | | | GWIYPGDGSTQYNEKFKGKTTLTADKSSSTAYMLLSSLT |
| | | | SEDSAIYFCAREGTYYAMDYWGQGTSVTVSSGGGGSGG |
| | | | GGSGGGGSDVLMTQTPLSLPVSLGDQVSISCRSSQSIVHS |
| | | | NGNTYLEWYLQKPGQSPKLLIYKVSNRFSGVPDRESGSG |
| | | | SGTDFTLKISRVEAEDLGVYYCFQGSHVPRTFGGGTKLEI |
| | | | KTERRAEVPTAHPSPSPRPAGQFQTLVVGVVGGLLGSLV |
| | | | LLVWVLAVICSRAARGTIGARRTGQPLKEDPSAVPVFSV |
| | | | DYGELDFQWREKTPEPPVPCVPEQVDYGELDFQWREKTP |
| | | | EPPVPCVPEQVDYGELVFPSGMGTSSPARRGSADGPRSA |
| | | | QPLRPEDGHCSWPL |
| MC0061 | 4xITIM | 264 | MALPVTALLLPLALLLHAARPEQKLISEEDLQVQLQQSGP |
| (VR61) | | | ELVKPGASVKMSCKASGYTFTSYHIQWVKQRPGQGLEWI |
| | | | GWIYPGDGSTQYNEKFKGKTTLTADKSSSTAYMLLSSLT |
| | | | SEDSAIYFCAREGTYYAMDYWGQGTSVTVSSGGGGSGG |
| | | | GGSGGGGSDVLMTQTPLSLPVSLGDQVSISCRSSQSIVHS |
| | | | NGNTYLEWYLQKPGQSPKLLIYKVSNRFSGVPDRESGSG |
| | | | SGTDFTLKISRVEAEDLGVYYCFQGSHVPRTFGGGTKLEI |
| | | | KTERRAEVPTAHPSPSPRPAGQFQTLVVGVVGGLLGSLV |
| | | | LLVWVLAVICSRAARGTIGARRTGQPLKEDPSAVPVFSV |
| | | | DYGELDFQWREKTPEPPVPCVPEQVDYGELDFQWREKTP |
| | | | EPPVPCVPEQVDYGELDFQWREKTPEPPVPCVPEQVDYG |
| | | | ELVFPSGMGTSSPARRGSADGPRSAQPLRPEDGHCSWPL |
| MC0062 | 5xITIM | 265 | MALPVTALLLPLALLLHAARPEQKLISEEDLQVQLQQSGP |
| (VR62) | | | ELVKPGASVKMSCKASGYTFTSYHIQWVKQRPGQGLEWI |
| | | | GWIYPGDGSTQYNEKFKGKTTLTADKSSSTAYMLLSSLT |
| | | | SEDSAIYFCAREGTYYAMDYWGQGTSVTVSSGGGGSGG |
| | | | GGSGGGGSDVLMTQTPLSLPVSLGDQVSISCRSSQSIVHS |
| | | | NGNTYLEWYLQKPGQSPKLLIYKVSNRFSGVPDRESGSG |
| | | | SGTDFTLKISRVEAEDLGVYYCFQGSHVPRTFGGGTKLEI |
| | | | KTERRAEVPTAHPSPSPRPAGQFQTLVVGVVGGLLGSLV |
| | | | LLVWVLAVICSRAARGTIGARRTGQPLKEDPSAVPVFSV |
| | | | DYGELDFQWREKTPEPPVPCVPEQVDYGELDFQWREKTP |
| | | | EPPVPCVPEQVDYGELDFQWREKTPEPPVPCVPEQVDYG |
| | | | ELDFQWREKTPEPPVPCVPEQVDYGELVFPSGMGTSSPA |
| | | | RRGSADGPRSAQPLRPEDGHCSWPL |
| MC0063 | 1xITSM | 266 | MALPVTALLLPLALLLHAARPEQKLISEEDLQVQLQQSGP |
| (VR63) | | | ELVKPGASVKMSCKASGYTFTSYHIQWVKQRPGQGLEWI |
| | | | GWIYPGDGSTQYNEKFKGKTTLTADKSSSTAYMLLSSLT |
| | | | SEDSAIYFCAREGTYYAMDYWGQGTSVTVSSGGGGSGG |
| | | | GGSGGGGSDVLMTQTPLSLPVSLGDQVSISCRSSQSIVHS |
| | | | NGNTYLEWYLQKPGQSPKLLIYKVSNRFSGVPDRESGSG |
| | | | SGTDFTLKISRVEAEDLGVYYCFQGSHVPRTFGGGTKLEI |
| | | | KTERRAEVPTAHPSPSPRPAGQFQTLVVGVVGGLLGSLV |
| | | | LLVWVLAVICSRAARGTIGARRTGQPLKEDPSAVPVFSTE |
| | | | YATIVFPSGMGTSSPARRGSADGPRSAQPLRPEDGHCSWP |
| | | | L |
| MC0064 | 2xITSM | 267 | MALPVTALLLPLALLLHAARPEQKLISEEDLQVQLQQSGP |
| (VR64) | | | ELVKPGASVKMSCKASGYTFTSYHIQWVKQRPGQGLEWI |
| | | | GWIYPGDGSTQYNEKFKGKTTLTADKSSSTAYMLLSSLT |
| | | | SEDSAIYFCAREGTYYAMDYWGQGTSVTVSSGGGGSGG |
| | | | GGSGGGGSDVLMTQTPLSLPVSLGDQVSISCRSSQSIVHS |
| | | | NGNTYLEWYLQKPGQSPKLLIYKVSNRFSGVPDRESGSG |
| | | | SGTDFTLKISRVEAEDLGVYYCFQGSHVPRTFGGGTKLEI |
| | | | KTERRAEVPTAHPSPSPRPAGQFQTLVVGVVGGLLGSLV |
| | | | LLVWVLAVICSRAARGTIGARRTGQPLKEDPSAVPVFSTE |
| | | | YATIDFQWREKTPEPPVPCVPEQTEYATIVFPSGMGTSSP |
| | | | ARRGSADGPRSAQPLRPEDGHCSWPL |
| MC0065 | 3xITSM | 268 | MALPVTALLLPLALLLHAARPEQKLISEEDLQVQLQQSGP |
| (VR65) | | | ELVKPGASVKMSCKASGYTFTSYHIQWVKQRPGQGLEWI |
| | | | GWIYPGDGSTQYNEKFKGKTTLTADKSSSTAYMLLSSLT |
| | | | SEDSAIYFCAREGTYYAMDYWGQGTSVTVSSGGGGSGG |
| | | | GGSGGGGSDVLMTQTPLSLPVSLGDQVSISCRSSQSIVHS |
| | | | NGNTYLEWYLQKPGQSPKLLIYKVSNRFSGVPDRESGSG |
| | | | SGTDFTLKISRVEAEDLGVYYCFQGSHVPRTFGGGTKLEI |
| | | | KTERRAEVPTAHPSPSPRPAGQFQTLVVGVVGGLLGSLV |
| | | | LLVWVLAVICSRAARGTIGARRTGQPLKEDPSAVPVFSTE |
| | | | YATIDFQWREKTPEPPVPCVPEQTEYATIDFQWREKTPEP |
| | | | PVPCVPEQTEYATIVFPSGMGTSSPARRGSADGPRSAQPL |
| | | | RPEDGHCSWPL |
| MC0066 | 4xITSM | 269 | MALPVTALLLPLALLLHAARPEQKLISEEDLQVQLQQSGP |
| (VR66) | | | ELVKPGASVKMSCKASGYTFTSYHIQWVKQRPGQGLEWI |
| | | | GWIYPGDGSTQYNEKFKGKTTLTADKSSSTAYMLLSSLT |
| | | | SEDSAIYFCAREGTYYAMDYWGQGTSVTVSSGGGGSGG |
| | | | GGSGGGGSDVLMTQTPLSLPVSLGDQVSISCRSSQSIVHS |
| | | | NGNTYLEWYLQKPGQSPKLLIYKVSNRFSGVPDRESGSG |
| | | | SGTDFTLKISRVEAEDLGVYYCFQGSHVPRTFGGGTKLEI |
| | | | KTERRAEVPTAHPSPSPRPAGQFQTLVVGVVGGLLGSLV |
| | | | LLVWVLAVICSRAARGTIGARRTGQPLKEDPSAVPVFSTE |
| | | | YATIDFQWREKTPEPPVPCVPEQTEYATIDFQWREKTPEP |
| | | | PVPCVPEQTEYATIDFQWREKTPEPPVPCVPEQTEYATIVF |
| | | | PSGMGTSSPARRGSADGPRSAQPLRPEDGHCSWPL |
| MC0067 | 5xITSM | 270 | MALPVTALLLPLALLLHAARPEQKLISEEDLQVQLQQSGP |
| (VR67) | | | ELVKPGASVKMSCKASGYTFTSYHIQWVKQRPGQGLEWI |
| | | | GWIYPGDGSTQYNEKFKGKTTLTADKSSSTAYMLLSSLT |
| | | | SEDSAIYFCAREGTYYAMDYWGQGTSVTVSSGGGGSGG |
| | | | GGSGGGGSDVLMTQTPLSLPVSLGDQVSISCRSSQSIVHS |
| | | | NGNTYLEWYLQKPGQSPKLLIYKVSNRFSGVPDRESGSG |
| | | | SGTDFTLKISRVEAEDLGVYYCFQGSHVPRTFGGGTKLEI |
| | | | KTERRAEVPTAHPSPSPRPAGQFQTLVVGVVGGLLGSLV |
| | | | LLVWVLAVICSRAARGTIGARRTGQPLKEDPSAVPVFSTE |
| | | | YATIDFQWREKTPEPPVPCVPEQTEYATIDFQWREKTPEP |
| | | | PVPCVPEQTEYATIDFQWREKTPEPPVPCVPEQTEYATIDF |
| | | | QWREKTPEPPVPCVPEQTEYATIVFPSGMGTSSPARRGSA |
| | | | DGPRSAQPLRPEDGHCSWPL |
| MC0068 | 2xPD1(G4S) | 271 | MALPVTALLLPLALLLHAARPEQKLISEEDLQVQLQQSGP |
| (VR68) | | | ELVKPGASVKMSCKASGYTFTSYHIQWVKQRPGQGLEWI |
| | | | GWIYPGDGSTQYNEKFKGKTTLTADKSSSTAYMLLSSLT |
| | | | SEDSAIYFCAREGTYYAMDYWGQGTSVTVSSGGGGSGG |
| | | | GGSGGGGSDVLMTQTPLSLPVSLGDQVSISCRSSQSIVHS |
| | | | NGNTYLEWYLQKPGQSPKLLIYKVSNRFSGVPDRFSGSG |
| | | | SGTDFTLKISRVEAEDLGVYYCFQGSHVPRTFGGGTKLEI |
| | | | KTERRAEVPTAHPSPSPRPAGQFQTLVVGVVGGLLGSLV |
| | | | LLVWVLAVICSRAARGTIGARRTGQPLKEDPSAVPVFSV |
| | | | DYGELDFQWREKTPEPPVPCVPEQTEYATIVFPSGMGTSS |
| | | | PARRGSADGPRSAQPLRPEDGHCSWPLGGGGSGGGGSCS |
| | | | RAARGTIGARRTGQPLKEDPSAVPVFSVDYGELDFQWRE |
| | | | KTPEPPVPCVPEQTEYATIVFPSGMGTSSPARRGSADGPRS |
| | | | AQPLRPEDGHCSWPL |
| MC0069 | 2xPD1(PD1) | 272 | MALPVTALLLPLALLLHAARPEQKLISEEDLQVQLQQSGP |
| (VR69) | | | ELVKPGASVKMSCKASGYTFTSYHIQWVKQRPGQGLEWI |
| | | | GWIYPGDGSTQYNEKFKGKTTLTADKSSSTAYMLLSSLT |
| | | | SEDSAIYFCAREGTYYAMDYWGQGTSVTVSSGGGGSGG |
| | | | GGSGGGGSDVLMTQTPLSLPVSLGDQVSISCRSSQSIVHS |
| | | | NGNTYLEWYLQKPGQSPKLLIYKVSNRFSGVPDRESGSG |
| | | | SGTDFTLKISRVEAEDLGVYYCFQGSHVPRTFGGGTKLEI |
| | | | KTERRAEVPTAHPSPSPRPAGQFQTLVVGVVGGLLGSLV |
| | | | LLVWVLAVICSRAARGTIGARRTGQPLKEDPSAVPVFSV |
| | | | DYGELDFQWREKTPEPPVPCVPEQTEYATIDFQWREKTP |
| | | | EPPVPCVPEQVDYGELDFQWREKTPEPPVPCVPEQTEYAT |
| | | | IVFPSGMGTSSPARRGSADGPRSAQPLRPEDGHCSWPL |
| MC0456 | LIR1(ITIM1) | 327 | MALPVTALLLPLALLLHAARPQVQLVQSGAEVKKPGASV |
| (VR456) | X4 | | KVSCKASGYTFTSYHIQWVRQAPGQRLEWIGWIYPGDGS |
| | | | TQYNEKFKGRVTITRDTSASTAYMELSSLRSEDTAVYYC |
| | | | AREGTYYAMDYWGQGTLVTVSSGGGGSGGGGSGGGGS |
| | | | DVVMTQTPLSLSVTPGQPASISCRSSQSIVHSNGNTYLEW |
| | | | YLQKPGQSPQLLIYKVSNRFSGVPDRFSGSGSGTDFTLKIS |
| | | | RVEAEDVGVYYCFQGSHVPRTFGGGTKVEIKTERRAEVP |
| | | | TAHPSPSPRPAGQFQTLVVGVVGGLLGSLVLLVWVLAVI |
| | | | LRHRRQGKHWTSTQRKADFQHPAGAVGPEPTDRGLQWR |
| | | | SSPAADAQEENLYAAVKHTQPEDGVEMDTRSPHDEDPQ |
| | | | ANLYAAVKHSRPRREMASPPSPLSGEFLDTKDRQAEEDR |
| | | | QMDTEAAASEAPQDNLYAAVHSLTLRREATEPPPSQEGP |
| | | | SPAVPNLYAAVAIH |
| MC0457 | LIR1(ITIM2) | 328 | MALPVTALLLPLALLLHAARPQVQLVQSGAEVKKPGASV |
| (VR457) | X4 | | KVSCKASGYTFTSYHIQWVRQAPGQRLEWIGWIYPGDGS |
| | | | TQYNEKFKGRVTITRDTSASTAYMELSSLRSEDTAVYYC |
| | | | AREGTYYAMDYWGQGTLVTVSSGGGGSGGGGSGGGGS |
| | | | DVVMTQTPLSLSVTPGQPASISCRSSQSIVHSNGNTYLEW |
| | | | YLQKPGQSPQLLIYKVSNRFSGVPDRFSGSGSGTDFTLKIS |
| | | | RVEAEDVGVYYCFQGSHVPRTFGGGTKVEIKTERRAEVP |
| | | | TAHPSPSPRPAGQFQTLVVGVVGGLLGSLVLLVWVLAVI |
| | | | LRHRRQGKHWTSTQRKADFQHPAGAVGPEPTDRGLQWR |
| | | | SSPAADAQEEVTYAEVKHTQPEDGVEMDTRSPHDEDPQ |
| | | | AVTYAEVKHSRPRREMASPPSPLSGEFLDTKDRQAEEDR |
| | | | QMDTEAAASEAPQDVTYAEVHSLTLRREATEPPPSQEGP |
| | | | SPAVPVTYAEVAIH |
| MC0458 | LIR1(ITIM3) | 329 | MALPVTALLLPLALLLHAARPQVQLVQSGAEVKKPGASV |
| (VR458) | X4 | | KVSCKASGYTFTSYHIQWVRQAPGQRLEWIGWIYPGDGS |
| | | | TQYNEKFKGRVTITRDTSASTAYMELSSLRSEDTAVYYC |
| | | | AREGTYYAMDYWGQGTLVTVSSGGGGSGGGGSGGGGS |
| | | | DVVMTQTPLSLSVTPGQPASISCRSSQSIVHSNGNTYLEW |
| | | | YLQKPGQSPQLLIYKVSNRFSGVPDRFSGSGSGTDFTLKIS |
| | | | RVEAEDVGVYYCFQGSHVPRTFGGGTKVEIKTERRAEVP |
| | | | TAHPSPSPRPAGQFQTLVVGVVGGLLGSLVLLVWVLAVI |
| | | | LRHRRQGKHWTSTQRKADFQHPAGAVGPEPTDRGLQWR |
| | | | SSPAADAQEEVTYAQLKHTQPEDGVEMDTRSPHDEDPQ |
| | | | AVTYAQLKHSRPRREMASPPSPLSGEFLDTKDRQAEEDR |
| | | | QMDTEAAASEAPQDVTYAQLHSLTLRREATEPPPSQEGP |
| | | | SPAVPVTYAQLAIH |
| MC0459 | LIR1(ITM4) | 330 | MALPVTALLLPLALLLHAARPQVQLVQSGAEVKKPGASV |
| (VR459) | X4 | | KVSCKASGYTFTSYHIQWVRQAPGQRLEWIGWIYPGDGS |
| | | | TQYNEKFKGRVTITRDTSASTAYMELSSLRSEDTAVYYC |
| | | | AREGTYYAMDYWGQGTLVTVSSGGGGSGGGGSGGGGS |
| | | | DVVMTQTPLSLSVTPGQPASISCRSSQSIVHSNGNTYLEW |
| | | | YLQKPGQSPQLLIYKVSNRFSGVPDRFSGSGSGTDFTLKIS |
| | | | RVEAEDVGVYYCFQGSHVPRTFGGGTKVEIKTERRAEVP |
| | | | TAHPSPSPRPAGQFQTLVVGVVGGLLGSLVLLVWVLAVI |
| | | | LRHRRQGKHWTSTQRKADFQHPAGAVGPEPTDRGLQWR |
| | | | SSPAADAQEESIYATLKHTQPEDGVEMDTRSPHDEDPQA |
| | | | SIYATLKHSRPRREMASPPSPLSGEFLDTKDRQAEEDRQM |
| | | | DTEAAASEAPQDSIYATLHSLTLRREATEPPPSQEGPSPAV |
| | | | PSIYATLAIH |
| MC0460 | LIR1ITIM | 331 | MALPVTALLLPLALLLHAARPQVQLVQSGAEVKKPGASV |
| (VR460) | (3-4) | | KVSCKASGYTFTSYHIQWVRQAPGQRLEWIGWIYPGDGS |
| | | | TQYNEKFKGRVTITRDTSASTAYMELSSLRSEDTAVYYC |
| | | | AREGTYYAMDYWGQGTLVTVSSGGGGSGGGGSGGGGS |
| | | | DVVMTQTPLSLSVTPGQPASISCRSSQSIVHSNGNTYLEW |
| | | | YLQKPGQSPQLLIYKVSNRFSGVPDRFSGSGSGTDFTLKIS |
| | | | RVEAEDVGVYYCFQGSHVPRTFGGGTKVEIKTERRAEVP |
| | | | TAHPSPSPRPAGQFQTLVVGVVGGLLGSLVLLVWVLAVI |
| | | | LRHRRQGKHWTSTQRKADFQHPAGAVGPEPTDRGLQWR |
| | | | SSPAADAQEEVTYAQLKHTQPEDGVEMDTRSPHDEDPQ |
| | | | ASIYATLKHSRPRREMASPPSPLSGEFLDTKDRQAEEDRQ |
| | | | MDTEAAASEAPQDVTYAQLHSLTLRREATEPPPSQEGPSP |
| | | | AVPSIYATLAIH |
| MC0461 | PD-1ITSM | 332 | MALPVTALLLPLALLLHAARPQVQLVQSGAEVKKPGASV |
| (VR461) | LIR1 | | KVSCKASGYTFTSYHIQWVRQAPGQRLEWIGWIYPGDGS |
| | (ITIM3-4) | | TQYNEKFKGRVTITRDTSASTAYMELSSLRSEDTAVYYC |
| | | | AREGTYYAMDYWGQGTLVTVSSGGGGSGGGGSGGGGS |
| | | | DVVMTQTPLSLSVTPGQPASISCRSSQSIVHSNGNTYLEW |
| | | | YLQKPGQSPQLLIYKVSNRFSGVPDRFSGSGSGTDFTLKIS |
| | | | RVEAEDVGVYYCFQGSHVPRTFGGGTKVEIKTERRAEVP |
| | | | TAHPSPSPRPAGQFQTLVVGVVGGLLGSLVLLVWVLAVI |
| | | | LRHRRQGKHWTSTQRKADFQHPAGAVGPEPTDRGLQWR |
| | | | SSPAADAQEETEYATIKHTQPEDGVEMDTRSPHDEDPQA |
| | | | TEYATIKHSRPRREMASPPSPLSGEFLDTKDRQAEEDRQM |
| | | | DTEAAASEAPQDVTYAQLHSLTLRREATEPPPSQEGPSPA |
| | | | VPSIYATLAIH |
| MC0462 | LIR1 | 333 | MALPVTALLLPLALLLHAARPQVQLVQSGAEVKKPGASV |
| (VR462) | (ITIM3-4) | | KVSCKASGYTFTSYHIQWVRQAPGQRLEWIGWIYPGDGS |
| | PD- | | TQYNEKFKGRVTITRDTSASTAYMELSSLRSEDTAVYYC |
| | 1ITSMX2 | | AREGTYYAMDYWGQGTLVTVSSGGGGSGGGGSGGGGS |
| | | | DVVMTQTPLSLSVTPGQPASISCRSSQSIVHSNGNTYLEW |
| | | | YLQKPGQSPQLLIYKVSNRFSGVPDRESGSGSGTDFTLKIS |
| | | | RVEAEDVGVYYCFQGSHVPRTFGGGTKVEIKTERRAEVP |
| | | | TAHPSPSPRPAGQFQTLVVGVVGGLLGSLVLLVWVLAVI |
| | | | LRHRRQGKHWTSTQRKADFQHPAGAVGPEPTDRGLQWR |
| | | | SSPAADAQEEVTYAQLKHTQPEDGVEMDTRSPHDEDPQ |
| | | | ASIYATLKHSRPRREMASPPSPLSGEFLDTKDRQAEEDRQ |
| | | | MDTEAAASEAPQDTEYATIHSLTLRREATEPPPSQEGPSP |
| | | | AVPTEYATIAIH |
| MC0463 | LIR1 | 334 | MALPVTALLLPLALLLHAARPQVQLVQSGAEVKKPGASV |
| (VR463) | ITIM3, | | KVSCKASGYTFTSYHIQWVRQAPGQRLEWIGWIYPGDGS |
| | PD-1 | | TQYNEKFKGRVTITRDTSASTAYMELSSLRSEDTAVYYC |
| | (ITSM)X2, | | AREGTYYAMDYWGQGTLVTVSSGGGGSGGGGSGGGGS |
| | LIR1 ITIM4 | | DVVMTQTPLSLSVTPGQPASISCRSSQSIVHSNGNTYLEW |
| | | | YLQKPGQSPQLLIYKVSNRFSGVPDRFSGSGSGTDFTLKIS |
| | | | RVEAEDVGVYYCFQGSHVPRTFGGGTKVEIKTERRAEVP |
| | | | TAHPSPSPRPAGQFQTLVVGVVGGLLGSLVLLVWVLAVI |
| | | | LRHRRQGKHWTSTQRKADFQHPAGAVGPEPTDRGLQWR |
| | | | SSPAADAQEEVTYAQLKHTQPEDGVEMDTRSPHDEDPQ |
| | | | ATEYATIKHSRPRREMASPPSPLSGEFLDTKDRQAEEDRQ |
| | | | MDTEAAASEAPQDTEYATIHSLTLRREATEPPPSQEGPSP |
| | | | AVPSIYATLAIH |

In some embodiments, the iCAR comprises an amino acid sequence selected from the group consisting of SEQ ID NO: 255, SEQ ID NO:256, SEQ ID NO:257, SEQ ID NO:258, SEQ ID NO:305, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:327, SEQ ID NO:328, SEQ ID NO:329, SEQ ID NO:330, SEQ ID NO:331, SEQ ID NO:332, SEQ ID NO:333, and SEQ ID NO:334.

7. iCAR Portion/aCAR Portion: Linker

In some embodiments, the iCAR portion is covalently linked to the aCAR portion via a linker. In a certain embodiment, the linker is a gly-ser polypeptide linker, i.e., a peptide that consists of glycine and serine residues. Exemplary gly-ser polypeptide linkers comprise the amino acid sequence Ser(Gly₄Ser)ₙ, as well as (Gly₄Ser)ₙ and/or (Gly₄Ser₃)ₙ. In some embodiments, n=1. In some embodiments, n=2. In some embodiments, n=3, i.e., Ser(Gly₄Ser)₃. In some embodiments, n=4, i.e., Ser(Gly₄Ser)₄. In some embodiments, n=5. In some embodiments, n=6. In some embodiments, n=7. In some embodiments, n=8. In some embodiments, n=9. In some embodiments, n=10. Another exemplary gly-ser polypeptide linker comprises the amino acid sequence Ser(Gly₄Ser)ₙ. In some embodiments, n=1. In some embodiments, n=2. In some embodiments, n=3. In another embodiment, n=4. In some embodiments, n=5. In some embodiments, n=6. Another exemplary gly-ser polypeptide linker comprises (Gly₄Ser)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6. Another exemplary gly-ser polypeptide linker comprises (Gly₄Ser₃)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6.

In some embodiments, the bicistronic construct comprises a linker that covalently connects the iCAR portion and the aCAR portion. In some embodiments, the bicistronic construct comprises a viral self-cleaving 2A peptide between the nucleic acid sequence encoding the iCAR portion and the nucleic acid sequence encoding the aCAR portion of the construct. In some embodiments, the viral self-cleaving 2A peptide includes T2A from Thosea asigna virus (TaV). In some embodiments, the iCAR portion is covalently linked to the aCAR portion via a linker. In some embodiments, the iCAR portion is covalently linked to the aCAR portion via a GSG. In some embodiments, the iCAR portion is covalently linked to the aCAR portion via a GGGGS linker (SEQ ID NO: 153). In some embodiments, the iCAR portion is covalently linked to the aCAR portion via a GGGGSGGGGSGGGGS linker (SEQ ID NO:154). In some embodiments, the iCAR is covalently linked to the aCAR portion via a T2A linker (SEQ ID NO: 155). In some embodiments, the iCAR is covalently linked to the aCAR portion via a F2A linker (SEQ ID NO: 156). In some embodiments, the iCAR is covalently linked to the aCAR portion via a P2A linker (SEQ ID NO:157). In some embodiments, the GAR is covalently linked to the aCAR portion via a E2A linker (SEQ ID NO:158). In some embodiments, the AR is covalently linked to the aCAR portion via a IRES long linker (SEQ ID NO:159). In some embodiments, the iCAR is covalently linked to the aCAR portion via a IRES short linker (SEQ ID NO: 160).

TABLE 13
iCAR portion/aCAR portion linker sequences

| Sequence | | |
|---|---|---|
| Information | SEQ ID NO | Amino acid sequence |
| G₄S | 153 | GGGGS |
| (G₄S)X3 | 154 | GGGGSGGGGSGGGGS |
| T2A | 155 | GSGEGRGSLLTCGDVEENPGP |
| F2A | 156 | GSGVKQTLNFDLLKLAGDVESNPGP |
| P2A | 157 | GSGATNFSLLKQAGDVEENPGP |
| E2A | 158 | GSGQCTNYALLKLAGDVESNPGP |
| IRES long | 159 | CCCCCCCCCCTAACGTTACTGGCCGAAGCCGCTT |
| | | GGAATAAGGCCGGTGTGCGTTTGTCTATATGTTA |
| | | TTTTCCACCATATTGCCGTCTTTTGGCAATGTGAG |
| | | GGCCCGGAAACCTGGCCCTGTCTTCTTGACGAGC |
| | | ATTCCTAGGGGTCTTTCCCCTCTCGCCAAAGGAA |
| | | TGCAAGGTCTGTTGAATGTCGTGAAGGAAGCAGT |
| | | TCCTCTGGAAGCTTCTTGAAGACAAACAACGTCT |
| | | GTAGCGACCCTTTGCAGGCAGCGGAACCCCCCAC |
| | | CTGGCGACAGGTGCCTCTGCGGCCAAAAGCCACG |
| | | TGTATAAGATACACCTGCAAAGGCGGCACAACCC |
| | | CAGTGCCACGTTGTGAGTTGGATAGTTGTGGAAA |
| | | GAGTCAAATGGCTCTCCTCAAGCGTATTCAACAA |
| | | GGGGCTGAAGGATGCCCAGAAGGTACCCCATTGT |
| | | ATGGGATCTGATCTGGGGCCTCGGTGCACATGCT |
| | | TTACATGTGTTTAGTCGAGGTTAAAAAAACGTCT |
| | | AGGCCCCCCGAACCACGGGGACGTGGTTTTCCTT |
| | | TGAAAAACACGATGATAATATG |
| IRES short | 160 | CCCCTCTCGCCAAAGGAATGCAAGGTCTGTTGAA |
| | | TGTCGTGAAGGAAGCAGTTCCTCTGGAAGCTTCT |
| | | TGAAGACAAACAACGTCTGTAGCGACCCTTTGCA |
| | | GGCAGCGGAACCCCCCACCTGGCGACAGGTGCCT |
| | | CTGCGGCCAAAAGCCACGTGTATAAGATACACCT |
| | | GCAAAGGCGGCACAACCCCAGTGCCACGTTGTG |
| | | AGTTGGATAGTTGTGGAAAGAGTCAAATGGCTCT |
| | | CCTCAAGCGTATTCAACAAGGGGCTGAAGGATGC |
| | | CCAGAAGGTACCCCATTGTATGGGATCTGATCTG |
| | | GGGCCTCGGTGCACATGCTTTACATGTGTTTAGT |
| | | CGAGGTTAAAAAAACGTCTAGGCCCCCCGAACC |
| | | ACGGGGACGTGGTTTTCCTTTGAAAAACACGATG |
| | | ATAATATG |

8. iCAR Portion/aCAR Portion: Signal Peptide

In some embodiments, the bicistronic construct comprises a signal peptide upstream of the iCAR and aCAR portions. In some embodiments, the signal peptide is a CD8 alpha signal peptide (SEQ ID NO: 161). In some embodiments, the signal peptide is a GM-CSF signal peptide (SEQ ID NO: 162). In some embodiments, the signal peptide is a mIgK signal peptide (SEQ ID NO: 306).

TABLE 14
iCAR/aCAR signal peptide sequences

| Sequence | | |
|---|---|---|
| Information | SEQ ID NO | Amino acid sequence |
| CD8 alpha | 161 | MALPVTALLLPLALLLHAARP |
| GM-CSF | 162 | MLLLVTSLLLCELPHPAFLLIP |
| mIgK | 306 | MSVPTQVLGLLLLWLTDARC |

9. aCAR Portion: aCAR Scfv

In some embodiments, the bicistronic construct comprises an aCAR portion comprising a single chain variable fragment (scFv) component. In some embodiments, the iCAR portion comprises an scFv component. In some embodiments, the scFv targets Her2, Mesothelin, or EGFR. In some embodiments, the scFv targets Her2. In some embodiments, the scFv targets Mesothelin. In some embodiments, the scFv targets EGFR. In some embodiments, the scFv is an scFv based on trastuzumab (anti-Her2 antibody, also referred to as HERCEPTIN*), pertuzumab (anti-Her2 antibody, also referred to as PERJETA*), another commercial anti-Her2 antibody including, but not limited to, FRP5, A21, XMT1517, XMT1518, XMT1519, FWP51, bioequivalents thereof, or biosimilars thereof. In some embodiments, the scFv has the VH and VL domains of trastuzumab, pertuzumab, FRP5, A21, XMT1517, XMT1518, XMT1519, FWP51, bioequivalents thereof, or biosimilars thereof. In some embodiments, the scFv is an scFv based on cetuximab (anti-EGFR antibody, also referred to as ERBITUX*), panitumumab (anti-EGFR antibody, also referred to as VECTIBIX*), another commercial anti-EGFR antibody including, but not limited to, Imgatuzumab, Nimotuzumab, Necitumumab, ICR62, Matuzumab, C10, Zalutumumab, PIX, P2X, P3X, EGFR-1a1-VHH, bioequivalents thereof, or biosimilars thereof. In some embodiments, the scFv has the VH and VL domains of cetuximab, panitumumab, Imgatuzumab, Nimotuzumab, Necitumumab, ICR62, Matuzumab, C10, Zalutumumab, PIX, P2X, P3X, EGFR-1a1-VHH, bioequivalents thereof, or biosimilars thereof. In some embodiments, the scFv is an scFv based on a commercial anti-Mesothelin antibody including, but not limited to, Amatuximab, P4, SS1, SD1, SD2, 1H7, 3C02, bioequivalents thereof, or biosimilars thereof. In some embodiments, the scFv has the VH and VL domains of Amatuximab, P4, SS1, SD1, SD2, 1H7, 3C02, bioequivalents thereof, or biosimilars thereof.

In some embodiments, the scFv targets Her2. In some embodiments, the Her2 scFv is based on the Vh and Vl from trastuzumab or pertuzumab. In some embodiments, the Her2 scFv is based on the Vh and Vl from trastuzumab. In some embodiments, the Her2 scFv is based on the Vh and Vl from pertuzumab. The Vh and VI chains for trastuzumab and pertuzumab are provided below in Tables 15 and 16. In some embodiments, the Her2 scFv is based on the Vh and Vl from FRP5. In some embodiments, the Her2 scFv is based on the Vh and Vl from A21. In some embodiments, the Her2 scFv is based on the Vh and Vl from XMT1517. In some embodiments, the Her2 scFv is based on the Vh and Vl from XMT1518. In some embodiments, the Her2 scFv is based on the Vh and Vl from XMT1519. In some embodiments, the Her2 scFv is based on the Vh and Vl from FWP51. In some embodiments, the Her2 scFv is based on the Vh and Vl from trastuzumab F9G.

TABLE 15
anti-Her2 sequences

| | SEQ | |
|---|---|---|
| Sequence | ID | |
| Information | NO | Amino acid sequence |
| trastuzumab | 170 | EVQLVESGGGLVQPGGSLRLSCAASGFNIKDTYIHWVRQAP |
| Variable heavy | | GKGLEWVARIYPTNGYTRYADSVKGRFTISADTSKNTAYLQ |
| chain | | MNSLRAEDTAVYYCSRWGGDGFYAMDYWGQGTLVTVSS |
| trastuzumab | 171 | DIQMTQSPSSLSASVGDRVTITCRASQDVNTAVAWYQQKPG |
| Variable light | | KAPKLLIYSASFLYSGVPSRESGSRSGTDFTLTISSLQPEDFAT |
| chain | | YYCQQHYTTPPTFGQGTKVEIK |
| trastuzumab | 172 | EVQLVESGGGLVQPGGSLRLSCAASGFNIKDTYIHWVRQAP |
| scFv | | GKGLEWVARIYPTNGYTRYADSVKGRFTISADTSKNTAYLQ |
| | | MNSLRAEDTAVYYCSRWGGDGFYAMDYWGQGTLVTVSS |
| | | GSTSGSGKPGSGEGSTKGDIQMTQSPSSLSASVGDRVTITC |
| | | RASQDVNTAVAWYQQKPGKAPKLLIYSASFLYSGVPSRFSG |
| | | SRSGTDFTLTISSLQPEDFATYYCQQHYTTPPTFGQGTKVEIK |
| Trastuzumab | 307 | DIQMTQSPSSLSASVGDRVTITCRASQDVNTAVAWYQQKPG |
| F9G variable | | KAPKLLIYSASFLYSGVPSRFSGSRSGTDFTLTISSLQPEDFAT |
| Heavy chain | | YYCQQHYTTPPTFGQGTKVEIK |
| Trastuzumab | 308 | EVQLVESGGGLVQPGGSLRLSCAASGFNIKDTYIHWVRQAP |
| F9G variable | | GKGLEWVARIYPTNGYTRYADSVKGRFTISADTSKNTAYLQ |
| Light chain | | MNSLRAEDTAVYYCSRWGGDGGYAMDYWGQGTLVTVSS |
| pertuzumab | 173 | EVQLVESGGGLVQPGGSLRLSCAASGFTFTDYTMDWVRQA |
| Variable heavy | | PGKGLEWVADVNPNSGGSIYNQRFKGRFTLSVDRSKNTLYL |
| chain | | QMNSLRAEDTAVYYCARNLGPSFYFDYWGQGTLVTVSS |
| pertuzumab | 174 | DIQMTQSPSSLSASVGDRVTITCKASQDVSIGVAWYQQKPG |
| Variable light | | KAPKLLIYSASYRYTGVPSRFSGSGSGTDFTLTISSLQPEDFA |
| chain | | TYYCQQYYIYPYTFGQGTKVEIK |
| pertuzumab scFv | 175 | DIQMTQSPSSLSASVGDRVTITCKASQDVSIGVAWYQQKPG |
| | | KAPKLLIYSASYRYTGVPSRFSGSGSGTDFTLTISSLQPEDFA |
| | | TYYCQQYYIYPYTFGQGTKVEIKGSTSGSGKPGSGEGSTKG |
| | | EVQLVESGGGLVQPGGSLRLSCAASGFTFTDYTMDWVRQA |
| | | PGKGLEWVADVNPNSGGSIYNQRFKGRFTLSVDRSKNTLYL |
| | | QMNSLRAEDTAVYYCARNLGPSFYFDYWGQGTLVTVSS |
| FRP5 variable | 176 | QVQLQQSGPELKKPGETVKISCKASGYPFTNYGMNWVKQA |
| heavy vhain | | PGQGLKWMGWINTSTGESTFADDFKGRFDFSLETSANTAYL |
| | | QINNLKSEDMATYFCARWEVYHGYVPYWGQGTTVTVSS |
| FRP5 variable | 177 | DIQLTQSHKFLSTSVGDRVSITCKASQDVYNAVAWYQQKPG |
| light vhain | | QSPKLLIYSASSRYTGVPSRFTGSGSGPDFTFTISSVQAEDLA |
| | | VYFCQQHFRTPFTFGSGTKLEIK |
| A21 variable | 178 | EVQLQQSGPEVVKTGASVKISCKASGYSFTGYFINWVKKNS |
| heavy vhain | | GKSPEWIGHISSSYATSTYNQKFKNKAAFTVDTSSSTAFMQL |
| | | NSLTSEDSAVYYCVRSGNYEEYAMDYWGQGTSVTVSS |
| A21 variable | 179 | DIVLTQTPSSLPVSVGEKVTMTCKSSQTLLYSNNQKNYLAW |
| light vhain | | YQQKPGQSPKLLISWAFTRKSGVPDRFTGSGSGTDFTLTIGS |
| | | VKAEDLAVYYCQQYSNYPWTFGGGTKLEIK |
| XMT1517 | 180 | QVQLVESGGGVVQPGRSLRLSCAASGFTFSSYGMHWVRQA |
| variable heavy | | PGKGLEWVAVIWYDGSNKYYADSVKGRFTISRDNSKNTLY |
| vhain | | LQMNSLRAEDTAVYYCAKEAPYYAKDYMDVWGKGTTVT |
| | | VSS |
| XMT1517 | 181 | EIVLTQSPGTLSLSPGERATLSCRASQSVSSDYLAWYQQKPG |
| variable light | | QAPRLLIYGASSRATGIPDRESGSGSGTDFTLTISRLEPEDFA |
| vhain | | VYYCQQYVSYWTFGGGTKVEIK |
| XMT1518 | 182 | QVQLVESGGGVVQPGRSLRLSCAASGFTFSSYGMHWVRQA |
| variable heavy | | PGKGLEWVAGIWWDGSNEKYADSVKGRFTISRDNSKNTLY |
| vhain | | LQMNSLRAEDTAVYYCAKEAPYYAKDYMDVWGKGTTVT |
| | | VSS |
| XMT1518 | 183 | EIVLTQSPGTLSLSPGERATLSCRASQSVSSDYLAWYQQKPG |
| variable light | | QAPRLLIYGASRRATGIPDRESGSGSGTDFTLTISRLEPEDFA |
| vhain | | VYYCQQYVSYWTFGGGTKVEIK |
| XMT1519 | 184 | EVQLVESGGGLVQPGGSLRLSCAASGFTFSSYSMNWVRQA |
| variable heavy | | PGKGLEWVSYISSSSSTIYYADSVKGRFTISRDNAKNSLYLQ |
| vhain | | MNSLRAEDTAVYYCARGGHGYFDLWGRGTLVTVSS |
| XMT1519 | 185 | EIVLTQSPGTLSLSPGERATLSCRASQSVSSSYLAWYQQKPG |
| variable light | | QAPRLLIYGASSRATGIPDRFSGSGSGTDFTLTISRLEPEDFA |
| vhain | | VYYCQQYHHSPLTFGGGTKVEIK |
| FWP51 variable | 186 | QVQLQQSGAELVRPGTSVKLSCKASDYTFTSYWMNWVKQ |
| heavy vhain | | RPGQGLEWIGMIDPSDSETQYNQMFKDKAALTVDKSSNTA |
| | | YMQLSSLTSEDSAVYYCAKGGASGDWYFDVWGQGTTVT |
| FWP51 variable | 187 | DIQLTQSPSSLSASLGGEVTITCKASQDIKKYIAWYQHKPGK |
| light vhain | | SPRLLIHYTSVLQPGIPSRESGSGSGRDYSFSIHNLEPEDIATY |
| | | YCLHYDYLYTFGGGTKLEI |
| FWP51 VL VH | 188 | MLLLVTSLLLCELPHPAFLLIPDYKDDDDKQVQLQQSGAEL |
| | | VRPGTSVKLSCKASDYTFTSYWMNWVKQRPGQGLEWIGMI |
| | | DPSDSETQYNQMFKDKAALTVDKSSNTAYMQLSSLTSEDS |
| | | AVYYCAKGGASGDWYFDVWGQGTTVTGSTSGSGKPGSGE |
| | | GSTKGDIQLTQSPSSLSASLGGEVTITCKASQDIKKYIAWYQ |
| | | HKPGKSPRLLIHYTSVLQPGIPSRFSGSGSGRDYSFSIHNLEPE |
| | | DIATYYCLHYDYLYTFGGGTKLEI |
| Anti HER2 VHH | 309 | QVQLVQSGGGLVQAGGSLRLSCAASGRTESSYAMAWFRQA |
| | | PGKEREFVAAISWSGANIYVADSVKGRFTISRDNAKDTVYL |
| | | QMNSLKPEDTAVYYCAVKLGFAPVEERQYDYWGQGTQVT |
| | | VSS |

In some embodiments, the scFv targets EGFR. In some embodiments, the EGFR scFv is based on the Vh and Vl from cetuximab, panitumumab, Imgatuzumab, Nimotuzumab, Necitumumab, ICR62, Matuzumab, C10, Zalutumumab, P1X, P2X, P3X, or EGFR-1a1-VHH. In some embodiments, the EGFR scFv is based on the Vh and Vl from cetuximab. In some embodiments, the EGFR scFv is based on the Vh and Vl from panitumumab. In some embodiments, the EGFR scFv is based on the Vh and Vl from Imgatuzumab. In some embodiments, the EGFR scFv is based on the Vh and Vl from Nimotuzumab. In some embodiments, the EGFR scFv is based on the Vh and Vl from Nimotuzumab (K5). In some embodiments, the EGFR scFv is based on the Vh and VI from Necitumumab. In some embodiments, the EGFR scFv is based on the Vh and VI from ICR62. In some embodiments, the EGFR scFv is based on the Vh and Vl from Matuzumab. In some embodiments, the EGFR scFv is based on the Vh and Vl from C10. In some embodiments, the EGFR scFv is based on the Vh and Vl from Zalutumumab. In some embodiments, the EGFR scFv is based on the Vh and Vl from PIX. In some embodiments, the EGFR scFv is based on the Vh and Vl from P2X. In some embodiments, the EGFR scFv is based on the Vh and Vl from P3X. In some embodiments, the EGFR scFv is based on EGFR-1a1-VHH. In some embodiments, the EGFR scFv is based on EGFR-VHH.

TABLE 16
anti-EGFR sequences

| Sequence | SEQ | |
|---|---|---|
| Information | ID NO | Amino acid sequence |
| cetuximab | 189 | QVQLKQSGPGLVQPSQSLSITCTVSGFSLTNYGVHWVRQSPG |
| Variable heavy | | KGLEWLGVIWSGGNTDYNTPFTSRLSINKDNSKSQVFFKMN |
| chain | | SLQSNDTAIYYCARALTYYDYEFAYWGQGTLVTVS |
| cetuximab | 190 | DILLTQSPVILSVSPGERVSFSCRASQSIGTNIHWYQQRTNGSP |
| Variable light | | RLLIKYASESISGIPSRFSGSGSGTDFTLSINSVESEDIADYYCQ |
| chain | | QNNNWPTTFGAGTKLELK |
| cetuximab scFv | 191 | QVQLKQSGPGLVQPSQSLSITCTVSGFSLTNYGVHWVRQSPG |
| (SEQ ID NO:) | | KGLEWLGVIWSGGNTDYNTPFTSRLSINKDNSKSQVFFKMN |
| | | SLQSNDTAIYYCARALTYYDYEFAYWGQGTLVTVSGSTSGS |
| | | GKPGSGEGSTKGDILLTQSPVILSVSPGERVSFSCRASQSIGTN |
| | | IHWYQQRTNGSPRLLIKYASESISGIPSRFSGSGSGTDFTLSINS |
| | | VESEDIADYYCQQNNNWPTTFGAGTKLELK |
| panitumumab | 192 | QVQLQESGPGLVKPSETLSLTCTVSGGSVSSGDYYWTWIRQS |
| Variable heavy | | PGKGLEWIGHIYYSGNTNYNPSLKSRLTISIDTSKTQFSLKLSS |
| chain | | VTAADTAIYYCVRDRVTGAFDIWGQGTMVTVSS |
| panitumumab | 193 | DIQMTQSPSSLSASVGDRVTITCQASQDISNYLNWYQQKPGK |
| Variable light | | APKLLIYDASNLETGVPSRFSGSGSGTDFTFTISSLQPEDIATY |
| chain | | FCQHFDHLPLAFGGGTKVEIK |
| panitumumab | 194 | DIQMTQSPSSLSASVGDRVTITCQASQDISNYLNWYQQKPGK |
| scFv | | APKLLIYDASNLETGVPSRFSGSGSGTDFTFTISSLQPEDIATY |
| | | FCQHFDHLPLAFGGGTKVEIKGSTSGSGKPGSGEGSTKGQVQ |
| | | LQESGPGLVKPSETLSLTCTVSGGSVSSGDYYWTWIRQSPGK |
| | | GLEWIGHIYYSGNTNYNPSLKSRLTISIDTSKTQFSLKLSSVTA |
| | | ADTAIYYCVRDRVTGAFDIWGQGTMVTVSS |
| Imgatuzuma | 195 | QVQLVQSGAEVKKPGSSVKVSCKASGFTFTDYKIHWVRQAP |
| variable heavy | | GQGLEWMGYFNPNSGYSTYAQKFQGRVTITADKSTSTAYM |
| chain | | ELSSLRSEDTAVYYCARLSPGGYYVMDAWGQGTTVTVSS |
| Imgatuzumab | 196 | DIQMTQSPSSLSASVGDRVTITCRASQGINNYLNWYQQKPGK |
| variable light | | APKRLIYNTNNLQTGVPSRFSGSGSGTEFTLTISSLQPEDFATY |
| chain | | YCLQHNSFPTFGQGTKLEIK |
| Nimotuzumab | 197 | QVQLQQSGAEVKKPGSSVKVSCKASGYTFTNYYIYWVRQAP |
| variable heavy | | GQGLEWIGGINPTSGGSNFNEKFKTRVTITVDESTNTAYMEL |
| chain | | SSLRSEDTAFYFCARQGLWFDSDGRGFDFWGQGSTVTVSS |
| Nimotuzumab | 198 | DIQMTQSPSSLSASVGDRVTITCRSSQNIVHSNGNTYLDWYQ |
| variable light | | QTPGKAPKLLIYKVSNRFSGVPSRFSGSGSGTDFTFTISSLQPE |
| chain | | DIATYYCFQYSHVPWTFGQGTKLQIT |
| Nimotuzumab | 310 | DIQMTQSPSSLSASVGDRVTITCRSSQNIVHSNGNTYLDWYQ |
| (K5) variable | | QTPGKAPKLLIYKVSNRFSGVPSRFSGSGSGTDFTFTISSLQPE |
| light chain | | DIATYYCFQYSHVPWTFGQGTKLQIT |
| Nimotuzumab | 311 | QVQLQQSGAEVKKPGSSVKVSCKASGYTFTDYYIYWVRQAP |
| (K5) variable | | GQGLEWIGGINPVTQRPVFNEKFKTRVTITVDESTNTAYMEL |
| Heavy chain | | SSLRSEDTAFYFCARQGLWFDSDGRGFDFWGQGSTVTVSS |
| Necitumumab | 199 | QVQLQESGPGLVKPSQTLSLTCTVSGGSISSGDYYWSWIRQP |
| variable heavy | | PGKGLEWIGYIYYSGSTDYNPSLKSRVTMSVDTSKNQFSLKV |
| chain | | NSVTAADTAVYYCARVSIFGVGTFDYWGQGTLVTVSS |
| Necitumumab | 200 | EIVMTQSPATLSLSPGERATLSCRASQSVSSYLAWYQQKPGQ |
| variable light | | APRLLIYDASNRATGIPARFSGSGSGTDFTLTISSLEPEDFAVY |
| chain | | YCHQYGSTPLTFGGGTKAEIK |
| ICR62 variable | 201 | QVNLLQSGAALVKPGASVKLSCKGSGFTFTDYKIHWVKQSH |
| heavy chain | | GKSLEWIGYFNPNSGYSTYNEKFKSKATLTADKSTDTAYME |
| | | LTSLTSEDSATYYCTRLSPGGYYVMDAWGQGASVTVSS |
| ICR62 variable | 202 | DIQMTQSPSFLSASVGDRVTINCKASQNINNYLNWYQQKLG |
| light chain | | EAPKRLIYNTNNLQTGIPSRFSGSGSGTDYTLTISSLQPEDFAT |
| | | YFCLQHNSFPTFGAGTKLELK |
| ICR62 VL VH | 203 | MLLLVTSLLLCELPHPAFLLIPDIQMTQSPSFLSASVGDRVTIN |
| | | CKASQNINNYLNWYQQKLGEAPKRLIYNTNNLQTGIPSRFSG |
| | | SGSGTDYTLTISSLQPEDFATYFCLQHNSFPTFGAGTKLELKG |
| | | STSGSGKPGSGEGSTKGQVNLLQSGAALVKPGASVKLSCKG |
| | | SGFTFTDYKIHWVKQSHGKSLEWIGYFNPNSGYSTYNEKFKS |
| | | KATLTADKSTDTAYMELTSLTSEDSATYYCTRLSPGGYYVM |
| | | DAWGQGASVTVSS |
| Matuzumab | 204 | QVQLVQSGAEVKKPGASVKVSCKASGYTFTSHWMHWVRQ |
| variable heavy | | APGQGLEWIGEFNPSNGRTNYNEKFKSKATMTVDTSTNTAY |
| chain | | MELSSLRSEDTAVYYCASRDYDYDGRYFDYWGQGTLVTVS |
| | | S |
| Matuzumab | 205 | DIQMTQSPSSLSASVGDRVTITCSASSSVTYMYWYQQKPGK |
| variable light | | APKLLIYDTSNLASGVPSRESGSGSGTDYTFTISSLQPEDIATY |
| chain | | YCQQWSSHIFTFGQGTKVEIK |
| C10 variable | 206 | EVQLVQSGAEVKKPGSSVKVSCKASGGTFSSYAIGWVRQAP |
| heavy chain | | GQGLEWMGGIIPIFGIANYAQKFQGRVTITADESTSSAYMELS |
| | | SLRSEDTAVYYCAREEGPYCSSTSCYAAFDIWGQGTLVTLSS |
| C10 variable | 207 | QSVLTQDPAVSVALGQTVKITCQGDSLRSYFASWYQQKPGQ |
| light chain | | APTLVMYARNDRPAGVPDRESGSKSGTSASLSAISGLQPEDE |
| | | AYYCAAWDDSLNGYLFGAGTKLTVL |
| Zalutumumab | 208 | QVQLVESGGGVVQPGRSLRLSCAASGFTFSTYGMHWVRQA |
| variable heavy | | PGKGLEWVAVIWDDGSYKYYGDSVKGRFTISRDNSKNTLYL |
| chain | | QMNSLRAEDTAVYYCARDGITMVRGVMKDYFDYWGQGTL |
| | | VTVSS |
| Zalutumumab | 209 | AIQLTQSPSSLSASVGDRVTITCRASQDISSALVWYQQKPGK |
| variable light | | APKLLIYDASSLESGVPSRFSGSESGTDFTLTISSLQPEDFATY |
| chain | | YCQQFNSYPLTFGGGTKVEIK |
| P1X variable | 210 | QVQLVQSGAEVKKPGSSVKVSCKASGGTFSSYAISWVRQAP |
| heavy chain | | GQGLEWMGSIIPIFGTVNYAQKFQGRVTITADESTSTAYMEL |
| | | SSLRSEDTAVYYCARDPSVNLYWYFDLWGRGTLVTVSS |
| P1X variable | 211 | DIQMTQSPSTLSASVGDRVTITCRASQSISSWWAWYQQKPG |
| light chain | | KAPKLLIYDASSLESGVPSRFSGSGSGTEFTLTISSLQPDDFAT |
| | | YYCQQYHAHPTTFGGGTKVEIK |
| P2X variable | 212 | QVQLVQSGAEVKKPGSSVKVSCKASGGTFGSYAISWVRQAP |
| heavy chain | | GQGLEWMGSIIPIFGAANPAQKSQGRVTITADESTSTAYMEL |
| | | SSLRSEDTAVYYCAKMGRGKVAFDIWGQGTMVTVSS |
| P2X variable | 213 | DIVMTQSPDSLAVSLGERATINCKSSQSVLYSPNNKNYLAWY |
| light chain | | QQKPGQPPKLLIYWASTRESGVPDRFSGSGSGTDFTLTISSLQ |
| | | AEDVAVYYCQQYYGSPITFGGGTKVEIK |
| P3X variable | 214 | QVQLVQSGAEVKKPGASVKVSCKASGYAFTSYGINWVRQA |
| heavy chain | | PGQGLEWMGWISAYNGNTYYAQKLRGRVTMTTDTSTSTAY |
| | | MELRSLRSDDTAVYYCARDLGGYGSGSVPFDPWGQGTLVT |
| | | VSS |
| P3X variable | 215 | EIVMTQSPATLSVSPGERATLSCRASQSVSSNLAWYQQKPGQ |
| light chain | | APRLLIYGASTRATGIPARFSGSGSGTEFTLTISSLQSEDFAVY |
| | | YCQDYRTWPRRVFGGGTKVEIK |
| EGFR-la1- | 216 | QVQLQESGGGLVQAGGSLLLSCAASGRTFSSYAMGWFRQAP |
| VHH variable | | GKEREFVAAINWSGGSTSYADSVKGRFTISRDNTKNTVYLQ |
| heavy chain | | MNSLKPEDTAAFYCAATYNPYSRDHYFPRMTTEYDYWGQG |
| | | TQVTVSS |
| EGFR-VHH | 312 | EVQQASGGGLVQAGGSLRLSCAASGRTETTSAIAWFRQAPG |
| variable heavy | | KEREFVAQISASGLGINYSGTVKGRFTISRDADKTTVYLQMN |
| chain | | SLTPEDTAVYYCAAGFHYIAAIRRTTDFHFWGPGTLVTVSS |

In some embodiments, the scFv targets Mesothelin. In some embodiments, the Mesothelin scFv is based on the Vh and VI from Amatuximab, P4, SS1, SD1, SD2, 1H7, or 3C02. In some embodiments, the Mesothelin scFv is based on the Vh and VI from Amatuximab. In some embodiments, the Mesothelin scFv is based on the Vh and VI from P4. In some embodiments, the Mesothelin scFv is based on the Vh and VI from SS1. In some embodiments, the Mesothelin scFv is based on SD1. In some embodiments, the Mesothelin scFv is based on SD2. In some embodiments, the Mesothelin scFv is based on the Vh and VI from 1H7. In some embodiments, the Mesothelin scFv is based on the Vh and VI from 3C02.

TABLE 17
anti-Mesothelin sequences

| Sequence | SEQ | |
|---|---|---|
| Information | ID NO | Amino acid sequence |
| Amatuximab | 217 | QVQLVQSGAEVKKPGASVKVSCKASGYSFTGYTMNWV |
| variable heavy | | RQAPGQGLEWMGLITPYNGASSYNQKFRGKATMTVDTS |
| chain | | TSTVYMELSSLRSEDTAVYYCARGGYDGRGFDYWGQGT |
| | | LVTVSS |
| Amatuximab | 218 | DIQMTQSPSSLSASVGDRVTITCSASSSVSYMHWYQQKS |
| variable light | | GKAPKLLIYDTSKLASGVPSRFSGSGSGTDFTLTISSLQPE |
| chain | | DFATYYCQQWSKHPLTFGQGTKLEIK |
| P4 variable | 219 | QVQLQQSGPGLVTPSQTLSLTCAISGDSVSSNSATWNWIR |
| heavy chain | | QSPSRGLEWLGRTYYRSKWYNDYAVSVKSRMSINPDTS |
| | | KNQFSLQLNSVTPEDTAVYYCARGMMTYYYGMDVWGQ |
| | | GTTVTVSS |
| P4 variable light | 220 | QPVLTQSSSLSASPGASASLTCTLRSGINVGPYRIYWYQQ |
| chain | | KPGSPPQYLLNYKSDSDKQQGSGVPSRFSGSKDASANAG |
| | | VLLISGLRSEDEADYYCMIWHSSAAVFGGGTQLTVL |
| P4 VL VH | 221 | MLLLVTSLLLCELPHPAFLLIPQPVLTQSSSLSASPGASASL |
| | | TCTLRSGINVGPYRIYWYQQKPGSPPQYLLNYKSDSDKQ |
| | | QGSGVPSRFSGSKDASANAGVLLISGLRSEDEADYYCMI |
| | | WHSSAAVFGGGTQLTVLGSTSGSGKPGSGEGSTKGQVQL |
| | | QQSGPGLVTPSQTLSLTCAISGDSVSSNSATWNWIRQSPS |
| | | RGLEWLGRTYYRSKWYNDYAVSVKSRMSINPDTSKNQF |
| | | SLQLNSVTPEDTAVYYCARGMMTYYYGMDVWGQGTTV |
| | | TVSS |
| SS1 variable | 222 | QVQLQQSGPELEKPGASVKISCKASGYSFTGYTMNWVK |
| heavy chain | | QSHGKSLEWIGLITPYNGASSYNQKFRGKATLTVDKSSST |
| | | AYMDLLSLTSEDSAVYFCARGGYDGRGFDYWGSGTPVT |
| | | VSS |
| SS1 variable | 223 | DIELTQSPAIMSASPGEKVTMTCSASSSVSYMHWYQQKS |
| light chain | | GTSPKRWIYDTSKLASGVPGRFSGSGSGNSYSLTISSVEAE |
| | | DDATYYCQQWSKHPLTFGSGTKVEIK |
| SS1 VL VH | 224 | MLLLVTSLLLCELPHPDIELTQSPAIMSASPGEKVTMTCSA |
| | | SSSVSYMHWYQQKSGTSPKRWIYDTSKLASGVPGRESGS |
| | | GSGNSYSLTISSVEAEDDATYYCQQWSKHPLTFGSGTKV |
| | | EIKGSTSGSGKPGSGEGSTKGQVQLQQSGPELEKPGASVK |
| | | ISCKASGYSFTGYTMNWVKQSHGKSLEWIGLITPYNGAS |
| | | SYNQKFRGKATLTVDKSSSTAYMDLLSLTSEDSAVYFCA |
| | | RGGYDGRGFDYWGSGTPVTVSS |
| SD1 VHH | 225 | QVQLVQSGGGLVQPGGSLRLSCAASDFDFAAYEMSWVR |
| | | QSAPGQGLEWVAIISHDGIDKYYTDSVKGRFTISRDNSKN |
| | | TLYLQMNTLRAEDTATYYCLRLGAVGQGTLVTVSSS |
| SD2 VHH | 226 | QVQLVQSGGGLVQPGGSLRLSCAASDFAFDDYEMSWVR |
| | | QAPGKALEWIGDINHSGTTIYNPSLKSRVTISRDNSKNTL |
| | | YLQMNTLRAEDTAIYYCARPHYGDYSDAFDIWGQGTMV |
| | | TVSS |
| 1H7 variable | 227 | EVQLQQSGTVLARPGASVKMSCKASGYSFTNYRMNWV |
| heavy chain | | KQRPGQGLEWIGGIYPGNRDTTYNQKFKDKAKLTAVTSA |
| | | NTAYMELSSLTNEDSAVYYCTRGVIGIYFDYWGQGTTLT |
| | | VSS |
| 1H7 variable | 228 | DIVMTQSPASLAVSLGQRATISCKASQSVDYDGDSYMNW |
| light chain | | YQQKPGQPPKLLIYAASNLESGIPARFSGSGSGTDFTLNIH |
| | | PVEEEDAATYYCQQNNEAPLTFGAGTKLELK |
| 1H7 VL VH | 229 | MLLLVTSLLLCELPHPAFLLIPDIVMTQSPASLAVSLGQRA |
| | | TISCKASQSVDYDGDSYMNWYQQKPGQPPKLLIYAASNL |
| | | ESGIPARFSGSGSGTDFTLNIHPVEEEDAATYYCQQNNEA |
| | | PLTFGAGTKLELKGSTSGSGKPGSGEGSTKGEVQLQQSGT |
| | | VLARPGASVKMSCKASGYSFTNYRMNWVKQRPGQGLE |
| | | WIGGIYPGNRDTTYNQKFKDKAKLTAVTSANTAYMELSS |
| | | LTNEDSAVYYCTRGVIGIYFDYWGQGTTLTVSS |
| 3C02 variable | 230 | QVQLQQSGTVLARPGASVKMSCKASGYSFTNYRMYWV |
| heavy chain | | KQRPGQGLEWIGAIYPGNSDTTYKQKFKGKAKLTAVTSA |
| | | STAYMELSSLTNEDSAVYYCTRGIRGSYFDVWGAGTTVT |
| | | VSS |
| 3C02 variable | 231 | DIVMTQSPASLAVSLGQRATISCKASQSVDYDGDSYMNW |
| light chain | | YQQKPGQPPKLLIYAASNLESGIPARFSGSGSGTDFTLNIH |
| | | PVEEEDAATYYCQQSNEDPYTFGGGTKLEIK |
| 3C02 VL VH | 232 | MLLLVTSLLLCELPHPAFLLIPDIVMTQSPASLAVSLGQRA |
| | | TISCKASQSVDYDGDSYMNWYQQKPGQPPKLLIYAASNL |
| | | ESGIPARFSGSGSGTDFTLNIHPVEEEDAATYYCQQSNEDP |
| | | YTFGGGTKLEIKGSTSGSGKPGSGEGSTKGQVQLQQSGT |
| | | VLARPGASVKMSCKASGYSFTNYRMYWVKQRPGQGLE |
| | | WIGAIYPGNSDTTYKQKFKGKAKLTAVTSASTAYMELSS |
| | | LTNEDSAVYYCTRGIRGSYFDVWGAGTTVTVSS |
| M1 variable | 313 | EIVLTQSPATLSLSPGERATISCRASQSVSSNFAWYQQRPG |
| Ligh Chain | | QAPRLLIYDASNRATGIPPRFSGSGSGTDFTLTISSLEPEDF |
| | | AAYYCHQRSNWLYTFGQGTKVDIK |
| M1 variable | 314 | QVQLQQSGAEVKKPGASVKVSCKASGYTFTGYYMHWV |
| Heavy Chain | | RQAPGQGLEWMGRINPNSGGTNYAQKFQGRVTMTRDTS |
| | | ISTAYMELSRLRSEDTAVYYCARGRYYGMDVWGQGTM |
| | | VTVSS |
| M5 Variable | 315 | DIVMTQSPSSLSASVGDRVTITCRASQSIRYYLSWYQQKP |
| Light chain | | GKAPKLLIYTASILQNGVPSRESGSGSGTDFTLTISSLQPED |
| | | FATYYCLQTYTTPDFGPGTKVEIK |
| M5 Variable | 316 | QVQLVQSGAEVEKPGASVKVSCKASGYTFTDYYMHWV |
| Heavy chain | | RQAPGQGLEWMGWINPNSGGTNYAQKFQGRVTMTRDT |
| | | SISTAYMELSRLRSDDTAVYYCASGWDFDYWGQGTLVT |
| | | VSS |
| VD9.V3 | 317 | DIQMTQSPSSLSASVGDRVTITCKSSQSVLYSSNQKNYLA |
| Variable light | | WFQQKPGKAPKLLIYWASTRESGVPSRFSGSGSGTDFTLT |
| chain | | ISSLQPEDFATYFCHQYLSSYTFGQGTKVEIK |
| VD9.V3 | 318 | EVQLVESGGGLVQPGGSLRLSCAASGYTFTTYWMHWVR |
| Variable Heavy | | QAPGKGLEWVGYIRPSTGYTEYNQKFKDRFTISADTSKN |
| chain | | TAYLQMNSLRAEDTAVYYCARSRWLLDYWGQGTLVTV |
| | | SS |

In some embodiments, the orientation of the aCAR VH and VL regions is VH-VL. In some embodiments, the orientation of the aCAR VH and VL regions is VL-VH.

In some embodiments, the aCAR scFv comprises a linker that covalently connects the VH and the VL to form the aCAR scFv. In some embodiments, the aCAR comprises a GS based linker sequence, connecting the VH and VL to form the scFv. In some embodiments, the GS linker comprises GGGGS (SEQ ID NO:81). In some embodiments, the aCAR comprises a Whitlow linker sequence, e.g., GSTSGSGKPGSGEGSTKG (SEQ ID NO:82).

10. aCAR Portion: Hinge and Transmembrane Domain

In some embodiments, the bicistronic construct comprises an aCAR portion comprising a hinge transmembrane (TM) domain component. In some embodiments, the aCAR portion comprises a hinge TM domain. In some embodiments, the hinge TM domain comprises a hinge TM domain selected from the group consisting of a CD28 hinge TM domain and a CD8 hinge TM domain (including a CD8a hinge TM domain). In some embodiments, the hinge TM domain is a CD28 hinge TM domain. In some embodiments, the vector comprises a CD8 hinge TM domain. In some embodiments, the vector comprises a CD8a hinge TM domain. In some embodiments, the hinge domain comprises a hinge domain selected from the group consisting of a CD28 hinge domain and a CD8 hinge domain (including a CD8a hinge domain). In some embodiments, the hinge domain is a CD28 hinge domain. In some embodiments, the vector comprises a CD8 hinge domain. In some embodiments, the vector comprises a CD8a hinge domain. In some embodiments, the TM domain comprises a TM domain selected from the group consisting of a CD28 TM domain and a CD8 TM domain (including a CD8a TM domain). In some embodiments, the TM domain is a CD28 TM domain. In some embodiments, the vector comprises a CD8 TM domain. In some embodiments, the vector comprises a CD8a TM domain. In some embodiments, the hinge domain is a CD28 hinge domain of SEQ ID NO:85. In some embodiments, the vector comprises a CD8a hinge domain of SEQ ID NO:84. In some embodiments, the TM domain is a CD28 TM domain of SEQ ID NO:319. In some embodiments, the vector comprises a CD8a TM domain of SEQ ID NO:320.

TABLE 18
aCAR hinge and TM domain sequences

| Sequence | SEQ ID | |
|---|---|---|
| Information | NO | Amino acid sequence |
| CD28 hinge | 85 | IEVMYPPPYLDNEKSNGTIIHVKGKHLCPSP |
| | | LFPGPSKP |
| CD28 TM | 319 | FWVLVVVGGVLACYSLLVTVAFIIFWV |
| CD8alpha hinge | 84 | TTTPAPRPPTPAPTIASQPLSLRPEACRPAAG |
| | | GAVHTRGLDFACD |
| CD8alpha TM | 320 | IYIWAPLAGTCGVLLLSLVITLYC |

11. aCAR Portion: Co-Stimulatory and Activation Signaling Domain

In some embodiments, the bicistronic construct comprises an aCAR portion comprising co-stimulatory domain component. In some embodiments, the aCAR portion comprises a co-stimulatory domain. In some embodiments, the co-stimulatory domain is selected from the group consisting of CD137 (4-1BB) or CD28 or both 4-1BB and CD28 (28BB). In some embodiments, the co-stimulatory domain is a CD137 (4-1BB) co-stimulatory domain. In some embodiments, the co-stimulatory domain is a CD28 co-stimulatory domain. In some embodiments, the activation signaling domain is CD3z domain. In some embodiments, the co-stimulatory domain is a 28BB co-stimulatory domain. In some embodiments, the co-stimulatory domain is 4-1BB (SEQ ID NO:233). In some embodiments, the co-stimulatory domain is CD28 (SEQ ID NO:234). In some embodiments, the activation signaling domain is CD3z (SEQ ID NO:235).

TABLE 19
aCAR co-stimulatory and activation signaling domain sequences

| Sequence | SEQ | |
|---|---|---|
| Information | ID NO | Amino acid sequence |
| 4-1BB costim | 233 | KRGRKKLLYIFKQPFMRPVQTTQEEDGCSCRFPEEEEG |
| | | GCEL |
| CD28 costim | 234 | RSKRSRLLHSDYMNMTPRRPGPTRKHYQPYAPPRDFA |
| | | AY |
| CD3z activation | 235 | RVKFSRSADAPAYQQGQNQLYNELNLGRREEYDVLD |
| signaling | | KRRGRDPEMGGKPRRKNPQEGLYNELQKDKMAEAYS |
| | | EIGMKGERRRGKGHDGLYQGLSTATKDTYDALHMQA |
| | | LPPR |

12. aCAR Portion: Immunoreceptor Tyrosine-Based Activation Motif (ITAM)

In some embodiments, the aCAR portion comprises an Immunoreceptor Tyrosine-Based Activation Motif (ITAM). In some embodiments, the ITAM is a CD3 zeta domain. In some embodiments, the ITAM is a CD3 zeta domain of SEQ ID NO:236. In some embodiments, the ITAM is a CD3 zeta 3F domain of SEQ ID NO:237. In some embodiments, the ITAM is a CD3 zeta 4F domain of SEQ ID NO:238. In some embodiments, the ITAM is a CD3 zeta 40F domain of SEQ ID NO:239.

TABLE 20
aCAR ITAM domain sequences

| Sequence | SEQ | |
|---|---|---|
| Information | ID NO | Amino acid sequence |
| CD3 zeta domain | 236 | RVKFSRSADAPAYQQGQNQLYNELNLGRREEYDVLD |
| | | KRRGRDPEMGGKPRRKNPQEGLYNELQKDKMAEAY |
| | | SEIGMKGERRRGKGHDGLYQGLSTATKDTYDALHMQ |
| | | ALPPR |
| CD3 Zeta 3F | 237 | RVKFSRSADAPAYQQGQNQLFNELNLGRREEYDVLD |
| | | KRRGRDPEMGGKPRRKNPQEGLYNELQKDKMAEAFS |
| | | EIGMKGERRRGKGHDGLFQGLSTATKDTYDALHMQA |
| | | LPPR |
| CD3 Zeta 4F | 238 | RVKFSRSADAPAYQQGQNQLFNELNLGRREEFDVLD |
| | | KRRGRDPEMGGKPRRKNPQEGLYNELQKDKMAEAFS |
| | | EIGMKGERRRGKGHDGLFQGLSTATKDTYDALHMQA |
| | | LPPR |
| CD3 Zeta 4OF | 239 | RVKFSRSADAPAYQQGQNQLFNELNLGRREEFDVLD |
| | | KRRGRDPEMGGKPRRKNPQEGLFNELQKDKMAEAYS |
| | | EIGMKGERRRGKGHDGLYQGLSTATKDTFDALHMQA |
| | | LPPR |

13. Exemplary aCARs

In some embodiments, the aCAR comprises an scFv component comprising the VL and VH sequences of trastuzumab (SEQ ID NOs: 170 and 171). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z activation signaling domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of trastuzumab F9G (SEQ ID NOs: 307 and 308). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z activation signaling domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of pertuzumab (SEQ ID NOs: 173 and 174). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of FRP5 (SEQ ID NOs: 176 and 177). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of A21 (SEQ ID NOs: 178 and 179). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of XMT1517 (SEQ ID NOs: 180 and 181). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of XMT1518 (SEQ ID NOs: 182 and 183). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of XMT1519 (SEQ ID NOs: 184 and 185). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of FWP51 (SEQ ID NOs: 186 and 187). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the anti-HER2 VHH (SEQ ID NO: 309). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239).

In some embodiments, the aCAR comprises an scFv component comprising the VL and VH sequences of Cetuximab (SEQ ID NOs: 189 and 190). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Panitumumab (SEQ ID NOs: 192 and 193). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Imgatuzumab (SEQ ID NOs: 195 and 196). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Nimotuzumab (SEQ ID NOs: 197 and 198). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Nimotuzumab (K5) (SEQ ID NOs: 310 and 311). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Necitumumab (SEQ ID NOs: 199 and 200). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of ICR62 (SEQ ID NOs: 201 and 202). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Matuzumab (SEQ ID NOs: 204 and 205). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of C10 (SEQ ID NOs: 206 and 207). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Zalutumumab (SEQ ID NOs: 208 and 209). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of P1X (SEQ ID NOs: 210 and 211). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of P2X (SEQ ID NOs: 212 and 213). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of P3X (SEQ ID NOs: 214 and 215). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VHH sequence of EGFR-1a1-VHH (SEQ ID NO: 216). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VHH sequence of EGFR-VHH (SEQ ID NO: 312). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Amatuximab (SEQ ID NOs: 217 and 218). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of P4 (SEQ ID NOs: 219 and 220). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of SS1 (SEQ ID NOs: 222 and 223). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VHH sequence of SD1 (SEQ ID NO: 225). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VHH sequence of SD2 (SEQ ID NO: 226). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of 1H7 (SEQ ID NOs: 227 and 228). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of 3C02 (SEQ ID NOs: 230 and 231). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 40F domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR has a set of components shown in Table 21.

TABLE 21
aCAR constructs

| | | | | | | | |
|---|---|---|---|---|---|---|---|
| | Signal | | scFv | | | Co- | |
| Construct | Peptide | scFv | Linker | Hinge | TM | stimulatory | Signaling |

Anti-EGFR

| | | | | | | | |
|---|---|---|---|---|---|---|---|
| MC0001 | CD8 | Imgatuzumab | Whitlow | CD8 | CD8 | 4-1BB | CD3 zeta |
| (VR1) | alpha | VL_VH | | alpha | alpha | | |
| MC0002 | CD8 | Cextuximab | Whitlow | CD8 | CD8 | 4-1BB | CD3 zeta |
| (VR2) | alpha | VL_VH | | alpha | alpha | | |
| MC0003 | CD8 | Panitumumab | Whitlow | CD8 | CD8 | 4-1BB | CD3 zeta |
| (VR3) | alpha | VL_VH | | alpha | alpha | | |
| MC0004 | CD8 | Nimotuzumab | Whitlow | CD8 | CD8 | 4-1BB | CD3 zeta |
| (VR4) | alpha | VL_VH | | alpha | alpha | | |
| MC0005 | CD8 | Necitumumab | Whitlow | CD8 | CD8 | 4-1BB | CD3 zeta |
| (VR5) | alpha | VL_VH | | alpha | alpha | | |
| MC0163 | GM- | ICR62 VH | Whitlow | CD8 | CD8 | 4-1BB | CD3 zeta |
| (VR163) | CSF | VL | | alpha | alpha | | |
| MC0164 | GM- | ICR62 VL | Whitlow | CD8 | CD8 | 4-1BB | CD3 zeta |
| (VR164) | CSF | VH | | alpha | alpha | | |
| MC0165 | GM- | Matuzumab | Whitlow | CD8 | CD8 | 4-1BB | CD3 zeta |
| (VR165) | CSF | VH VL BBz | | alpha | alpha | | |
| MC0166 | GM- | Matuzumab | Whitlow | CD8 | CD8 | 4-1BB | CD3 zeta |
| (VR166) | CSF | VL VH BBz | | alpha | alpha | | |
| MC0167 | GM- | C10 VH VL | Whitlow | CD8 | CD8 | 4-1BB | CD3 zeta |
| (VR167) | CSF | BBz | | alpha | alpha | | |
| MC0168 | GM- | C10 VL VH | Whitlow | CD8 | CD8 | 4-1BB | CD3 zeta |
| (VR168) | CSF | BBz | | alpha | alpha | | |
| MC0169 | GM- | Zalutumumab | Whitlow | CD8 | CD8 | 4-1BB | CD3 zeta |
| (VR169) | CSF | VH VL BBz | | alpha | alpha | | |
| MC0170 | GM- | Zalutumumab | Whitlow | CD8 | CD8 | 4-1BB | CD3 zeta |
| (VR170) | CSF | VL VH BBz | | alpha | alpha | | |
| MC0171 | GM- | P1X VH VL | Whitlow | CD8 | CD8 | 4-1BB | CD3 zeta |
| (VR171) | CSF | BBz | | alpha | alpha | | |
| MC0172 | GM- | P1X VL VH | Whitlow | CD8 | CD8 | 4-1BB | CD3 zeta |
| (VR172) | CSF | BBz | | alpha | alpha | | |
| MC0173 | GM- | P2X VH VL | Whitlow | CD8 | CD8 | 4-1BB | CD3 zeta |
| (VR173) | CSF | BBz | | alpha | alpha | | |
| MC0174 | GM- | P2X VL VH | Whitlow | CD8 | CD8 | 4-1BB | CD3 zeta |
| (VR174) | CSF | BBz | | alpha | alpha | | |
| MC0175 | GM- | P3X VH VL | Whitlow | CD8 | CD8 | 4-1BB | CD3 zeta |
| (VR175) | CSF | BBz | | alpha | alpha | | |
| MC0176 | GM- | P3X VL VH | Whitlow | CD8 | CD8 | 4-1BB | CD3 zeta |
| (VR176) | CSF | BBz | | alpha | alpha | | |
| MC0177 | GM- | EGFR-la1- | Whitlow | CD8 | CD8 | 4-1BB | CD3 zeta |
| (VR177) | CSF | VHH BBz | | alpha | alpha | | |
| N/A | CD8 | ICR62 | Whitlow | CD8 | CD8 | 4-1BB | CD3 zeta |
| | alpha | VH_VL | | alpha | alpha | | |
| N/A | CD8 | ICR62 | Whitlow | CD8 | CD8 | 4-1BB | CD3 zeta |
| | alpha | VL_VH | | alpha | alpha | | |
| N/A | CD8 | Matuzumab | Whitlow | CD8 | CD8 | 4-1BB | CD3 zeta |
| | alpha | VH_VL | | alpha | alpha | | |
| N/A | CD8 | Matuzumab | Whitlow | CD8 | CD8 | 4-1BB | CD3 zeta |
| | alpha | VL_VH | | alpha | alpha | | |
| N/A | CD8 | C10 VH VL | Whitlow | CD8 | CD8 | 4-1BB | CD3 zeta |
| | alpha | BBz | | alpha | alpha | | |
| N/A | CD8 | C10 VL VH | Whitlow | CD8 | CD8 | 4-1BB | CD3 zeta |
| | alpha | BBz | | alpha | alpha | | |
| N/A | CD8 | Zalutumumab | whitlow | CD8 | CD8 | 4-1BB | CD3 zeta |
| | alpha | VH VL | | alpha | alpha | | |
| MC0483 | CD8 | Zalutumumab | whitlow | CD8 | CD8 | 4-1BB | CD3 zeta |
| (VR483) | alpha | VL_VH | | alpha | alpha | | |
| N/A | CD8 | P1X VH VL | Whitlow | CD8 | CD8 | 4-1BB | CD3 zeta |
| | alpha | BBz | | alpha | alpha | | |
| N/A | CD8 | P1X VL VH | Whitlow | CD8 | CD8 | 4-1BB | CD3 zeta |
| | alpha | BBz | | alpha | alpha | | |
| N/A | CD8 | P2X VH VL | Whitlow | CD8 | CD8 | 4-1BB | CD3 zeta |
| | alpha | BBz | | alpha | alpha | | |
| N/A | CD8 | P2X VL VH | Whitlow | CD8 | CD8 | 4-1BB | CD3 zeta |
| | alpha | BBz | | alpha | alpha | | |
| N/A | CD8 | P3X VH VL | Whitlow | CD8 | CD8 | 4-1BB | CD3 zeta |
| | alpha | BBz | | alpha | alpha | | |
| N/A | CD8 | P3X VL VH | Whitlow | CD8 | CD8 | 4-1BB | CD3 zeta |
| | alpha | BBz | | alpha | alpha | | |
| MC0484 | CD8 | EGFR-l1a- | whitlow | CD8 | CD8 | 4-1BB | CD3 zeta |
| (VR484) | alpha | VHH | | alpha | alpha | | |

Anti-HER2

| | | | | | | | |
|---|---|---|---|---|---|---|---|
| MC0006 | CD8 | Trastuzumab | Whitlow | CD8 | CD8 | 4-1BB | CD3 zeta |
| (VR6) | alpha | VL_VH | | alpha | alpha | | |
| MC0007 | CD8 | Pertuzumab | Whitlow | CD8 | CD8 | 4-1BB | CD3 zeta |
| (VR7) | alpha | VL_VH | | alpha | alpha | | |
| MC0008 | CD8 | FRP5 VL VH | Whitlow | CD8 | CD8 | 4-1BB | CD3 zeta |
| (VR8) | alpha | | | alpha | alpha | | |
| MC0009 | CD8 | A21 VL VH | Whitlow | CD8 | CD8 | 4-1BB | CD3 zeta |
| (VR9) | alpha | | | alpha | alpha | | |
| MC0178 | GM- | XMT1517 | Whitlow | CD8 | CD8 | 4-1BB | CD3 zeta |
| (VR178) | CSF | VH VL | | alpha | alpha | | |
| MC0179 | GM- | XMT1517 | Whitlow | CD8 | CD8 | 4-1BB | CD3 zeta |
| (VR179) | CSF | VL VH | | alpha | alpha | | |
| MC0180 | GM- | XMT1518 | Whitlow | CD8 | CD8 | 4-1BB | CD3 zeta |
| (VR180) | CSF | VH VL | | alpha | alpha | | |
| MC0181 | GM- | XMT1518 | Whitlow | CD8 | CD8 | 4-1BB | CD3 zeta |
| (VR181) | CSF | VL VH | | alpha | alpha | | |
| MC0182 | GM- | XMT1519 | Whitlow | CD8 | CD8 | 4-1BB | CD3 zeta |
| (VR182) | CSF | VH VL | | alpha | alpha | | |
| MC0183 | GM- | XMT1519 | Whitlow | CD8 | CD8 | 4-1BB | CD3 zeta |
| (VR183) | CSF | VL VH | | alpha | alpha | | |
| MC0184 | GM- | FWP51 | Whitlow | CD8 | CD8 | 4-1BB | CD3 zeta |
| (VR184) | CSF | VH VL | | alpha | alpha | | |
| MC0185 | GM- | FWP51 | Whitlow | CD8 | CD8 | 4-1BB | CD3 zeta |
| (VR185) | CSF | VL VH | | alpha | alpha | | |
| N/A | GM- | Trastuzumab | Whitlow | CD8 | CD8 | 4-1BB | CD3 zeta |
| | CSF | VL_VH | | alpha | alpha | | |
| N/A | GM- | Pertuzumab | Whitlow | CD8 | CD8 | 4-1BB | CD3 zeta |
| | CSF | VL_VH | | alpha | alpha | | |
| N/A | GM- | FRP5 VL VH | Whitlow | CD8 | CD8 | 4-1BB | CD3 zeta |
| | CSF | | | alpha | alpha | | |
| N/A | GM- | A21 VL VH | Whitlow | CD8 | CD8 | 4-1BB | CD3 zeta |
| | CSF | | | alpha | alpha | | |
| N/A | CD8 | XMT1517 | Whitlow | CD8 | CD8 | 4-1BB | CD3 zeta |
| | alpha | VH VL | | alpha | alpha | | |
| N/A | CD8 | XMT1517 | Whitlow | CD8 | CD8 | 4-1BB | CD3 zeta |
| | alpha | VL VH | | alpha | alpha | | |
| N/A | CD8 | XMT1518 | Whitlow | CD8 | CD8 | 4-1BB | CD3 zeta |
| | alpha | VH VL | | alpha | alpha | | |
| N/A | CD8 | XMT1518 | Whitlow | CD8 | CD8 | 4-1BB | CD3 zeta |
| | alpha | VL VH | | alpha | alpha | | |
| N/A | CD8 | XMT1519 | Whitlow | CD8 | CD8 | 4-1BB | CD3 zeta |
| | alpha | VH VL | | alpha | alpha | | |
| N/A | CD8 | XMT1519 | Whitlow | CD8 | CD8 | 4-1BB | CD3 zeta |
| | alpha | VL VH | | alpha | alpha | | |
| N/A | CD8 | FWP51 | Whitlow | CD8 | CD8 | 4-1BB | CD3 zeta |
| | alpha | VH VL | | alpha | alpha | | |
| N/A | CD8 | FWP51 | Whitlow | CD8 | CD8 | 4-1BB | CD3 zeta |
| | alpha | VL VH | | alpha | alpha | | |

Anti-Mesothelin

| | | | | | | | |
|---|---|---|---|---|---|---|---|
| MC0159 | GM- | Amatuximab | Whitlow | CD8 | CD8 | 4-1BB | CD3 zeta |
| (VR159) | CSF | VH VL | | alpha | alpha | | |
| MC0160 | GM- | Amatuximab | Whitlow | CD8 | CD8 | 4-1BB | CD3 zeta |
| (VR160) | CSF | VL VL | | alpha | alpha | | |
| MC0161 | GM- | P4 VH VL | Whitlow | CD8 | CD8 | 4-1BB | CD3 zeta |
| (VR161) | CSF | | | alpha | alpha | | |
| MC0162 | GM- | P4 VL VH | Whitlow | CD8 | CD8 | 4-1BB | CD3 zeta |
| (VR162) | CSF | | | alpha | alpha | | |
| MC0186 | GM- | SS1 VH VL | Whitlow | CD8 | CD8 | 4-1BB | CD3 zeta |
| (VR186) | CSF | | | alpha | alpha | | |
| MC0187 | GM- | SS1 VL VH | Whitlow | CD8 | CD8 | 4-1BB | CD3 zeta |
| (VR187) | CSF | | | alpha | alpha | | |
| MC0188 | GM- | SD1 VHH | Whitlow | CD8 | CD8 | 4-1BB | CD3 zeta |
| (VR188) | CSF | | | alpha | alpha | | |
| MC0189 | GM- | SD2 VHH | Whitlow | CD8 | CD8 | 4-1BB | CD3 zeta |
| (VR189) | CSF | | | alpha | alpha | | |
| MC0190 | GM- | 1H07 VH VL | Whitlow | CD8 | CD8 | 4-1BB | CD3 zeta |
| (VR190) | CSF | | | alpha | alpha | | |
| MC0191 | GM- | 1H07 VL VH | Whitlow | CD8 | CD8 | 4-1BB | CD3 zeta |
| (VR191) | CSF | | | alpha | alpha | | |
| MC0192 | GM- | 3C02 VH VL | Whitlow | CD8 | CD8 | 4-1BB | CD3 zeta |
| (VR192) | CSF | | | alpha | alpha | | |
| MC0193 | GM- | 3C02 VL VH | Whitlow | CD8 | CD8 | 4-1BB | CD3 zeta |
| (VR193) | CSF | | | alpha | alpha | | |
| N/A | CD8 | Amatuximab | Whitlow | CD8 | CD8 | 4-1BB | CD3 zeta |
| | alpha | VH VL | | alpha | alpha | | |
| MC0485 | CD8 | Amatuximab | Whitlow | CD8 | CD8 | 4-1BB | CD3 zeta |
| (VR485) | alpha | VL VH | | alpha | alpha | | |
| N/A | CD8 | P4 VH VL | Whitlow | CD8 | CD8 | 4-1BB | CD3 zeta |
| | alpha | | | alpha | alpha | | |
| MC0487 | CD8 | P4 VL VH | Whitlow | CD8 | CD8 | 4-1BB | CD3 zeta |
| (VR487) | alpha | | | alpha | alpha | | |
| N/A | CD8 | SS1 VH VL | Whitlow | CD8 | CD8 | 4-1BB | CD3 zeta |
| | alpha | | | alpha | alpha | | |
| MC0488 | CD8 | SS1 VL VH | Whitlow | CD8 | CD8 | 4-1BB | CD3 zeta |
| (VR488) | alpha | | | alpha | alpha | | |
| N/A | CD8 | SD1 VHH | Whitlow | CD8 | CD8 | 4-1BB | CD3 zeta |
| | alpha | | | alpha | alpha | | |
| N/A | CD8 | SD2 VHH | Whitlow | CD8 | CD8 | 4-1BB | CD3 zeta |
| | alpha | | | alpha | alpha | | |
| N/A | CD8 | 1H07 VH VL | Whitlow | CD8 | CD8 | 4-1BB | CD3 zeta |
| | alpha | | | alpha | alpha | | |
| MC0490 | CD8 | 1H07 VL VH | Whitlow | CD8 | CD8 | 4-1BB | CD3 zeta |
| (VR490) | alpha | | | alpha | alpha | | |
| N/A | CD8 | 3C02 VH VL | Whitlow | CD8 | CD8 | 4-1BB | CD3 zeta |
| | alpha | | | alpha | alpha | | |
| N/A | CD8 | 3C02 VL VH | Whitlow | CD8 | CD8 | 4-1BB | CD3 zeta |
| | alpha | | | alpha | alpha | | |
| MC0486 | CD8 | M1 VL VH | Whitlow | CD8 | CD8 | 4-1BB | CD3 zeta |
| (VR486) | alpha | | | alpha | alpha | | |
| MC0498 | CD8 | M5 VL VH | Whitlow | CD8 | CD8 | 4-1BB | CD3 zeta |
| (VR498) | alpha | | | alpha | alpha | | |
| MC0489 | CD8 | 7D9.V3 VL | Whitlow | CD8 | CD8 | 4-1BB | CD3 zeta |
| (VR489) | alpha | VH | | alpha | alpha | | |

14. Optional shRNA

In some embodiments, the bicistronic construct comprises an optional short hairpin RNA (shRNA). In some embodiments, the bicistronic construct comprises an HLA-A2 shRNA. In some embodiments, the bicistronic construct comprises an HLA-A2 shRNA having a sequence of SEQ ID NO:240. In some embodiments, the bicistronic construct comprises an HLA-A2 shRNA having a sequence of SEQ ID NO:241. In some embodiments, the bicistronic construct comprises an HLA-beta2 shRNA. In some embodiments, the bicistronic construct comprises an HLA-beta2 shRNA having a sequence of SEQ ID NO:242. In some embodiments, the bicistronic construct comprises an HLA-A2 shRNA having both sequences of SEQ ID NO:240 and SEQ ID NO:242. In some embodiments, the bicistronic construct comprises an HLA-A2 shRNA having both sequences of SEQ ID NO:241 and SEQ ID NO:242.

TABLE 22
shRNA sequences

| | SEQ | |
|---|---|---|
| Sequence | ID | |
| Information | NO | Amino acid sequence |
| HLA-A2-shRNA | 240 | GGATTACATCGCCCTGAAAGTTCAAGAGACTTTCAGGGC |
| 1 | | GATGTAATCCTTTTTT |
| HLA-A2-shRNA | 241 | CACCTGCCATGTGCAGCATGATTTGTGTAGTCATGCTGC |
| 2 | | ACATGGCAGGTG |
| HLA-beta2- | 242 | GAATGGAGAGAGAATTGAATTCAAGAGATTCAATTCTCT |
| shRNA | | CTCCATTC |

15. Monocistronic Constructs

In some embodiments, the iCAR and aCAR constructs are expressed by separate vectors, and the iCAR/aCAR pairs are co-expressed in cells. Methods of co-expressing multiple constructs in the same cell are well known in the art and include, e.g., co-transfection of two or more expression vectors, integration of the constructs into the same or different loci within a cell, optionally followed by enrichment for co-expression.

III. CAR-T Bicistronic ICAR/aCAR Vector Construction

In some embodiments, the bicistronic construct or co-transduction of monocistronic aCAR and iCAR constructs allows for the iCAR and the aCAR to be encoded by a single nucleic acid vector. In some embodiments, the present invention provides a vector comprising a nucleic acid molecule of the invention as defined in any one of the above embodiments, and at least one control element, such as a promoter, operably linked to the nucleic acid molecule.

In some embodiments, the vector is a lentiviral (LV) vector. In some embodiments, the LV vector is a commercially available LV vector. In some embodiments, the LV vector includes but is not limited to pLenti, pLVX-Puro, pLVX-IRES-Puro/Neo/Hygro, pLVx-EF1a-IRES (TAKARA), and/or pcLV-EF1a (Sirion). In some embodiments, the LV vector is pLVX-Puro. In some embodiments, the LV vector is pLVX-IRES-Puro/Neo/Hygro. In some embodiments, the LV vector is pLVx-EF1a-IRES (TAKARA). In some embodiments, the LV vector is pcLV-EF1a (Sirion).

In some embodiments, the vector comprises an EF1 promoter. In some embodiments, the vector comprises a CMV promoter. In some embodiments, the vector comprises a PGK promoter.

In some embodiments, the nucleotide sequence of the vector comprises an internal ribosome entry site (IRES) between the nucleotide sequence encoding for the aCAR and the nucleotide sequence encoding for the iCAR. In general, the nucleotide sequence encoding for the aCAR and the nucleotide sequence encoding for the iCAR can be in any sequential order, but in particular embodiments, the nucleotide sequence encoding for the aCAR is downstream of the nucleotide sequence encoding for the iCAR.

In some embodiments, the nucleotide sequences encoding for the aCAR and the iCAR are encoded on a single vector. In some embodiments, the vector comprises an internal ribosome entry site (IRES) between the nucleotide sequence encoding for the aCAR and the nucleotide sequence encoding for the iCAR. In some embodiments, the nucleotide sequence encoding for the aCAR is downstream of the nucleotide sequence encoding for the iCAR. In some embodiments, the nucleotide sequence comprises a viral self-cleaving 2A peptide located between the nucleotide sequence encoding for the aCAR and the nucleotide sequence encoding for the iCAR. In some embodiments, the nucleotide sequence of the vector comprises a viral self-cleaving 2A peptide between the nucleotide sequence encoding for the aCAR and the nucleotide sequence encoding for the iCAR. In some embodiments, the viral self-cleaving 2A peptide includes is the T2A from Thosea asigna virus (TaV). In some embodiments, the vector comprises a nucleotide sequence encoding the constitutive aCAR linked via a flexible linker to said iCAR.

The immune cells may be transfected with the appropriate nucleic acid molecule described herein by e.g., RNA transfection or by incorporation in a plasmid fit for replication and/or transcription in a eukaryotic cell or a viral vector. In some embodiments, the vector is selected from a retroviral or lentiviral vector.

Combinations of retroviral vector and an appropriate packaging line can also be used, where the capsid proteins will be functional for infecting human cells. Several amphotropic virus-producing cell lines are known, including PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Nati. Acad. Sci. USA 85:6460-6464). Alternatively, non-amphotropic particles can be used, such as, particles pseudotyped with VSVG, RD 114 or GAL V envelope and in some embodiments produced in a PG13 cell line. Cells can further be transduced by direct co-culture with producer cells, e.g., by the method of Bregni, et al. (1992) Blood 80: 1418-1422, or culturing with viral supernatant alone or concentrated vector stocks, e.g., by the method of Xu, et al. (1994) Exp. Hemat. 22:223-230; and Hughes, et al. (1992) J Clin. Invest. 89: 1817.

In some embodiments, the iCAR and aCAR are encoded by different constructs, for example as separate monocistronic aCAR and iCAR constructs. In some embodiments, the iCAR and aCAR are encoded by a single construct, for example as separate monocistronic aCAR and iCAR constructs within a single expression vector.

In some embodiments, the iCAR and aCAR are encoded by the same expression vector. In some embodiments, the expression vector comprises a nucleic acid sequence that encodes a bicistronic iCAR/aCAR selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the expression vector comprises a bicistronic iCAR/aCAR nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the expression vector comprises a bicistronic iCAR/aCAR nucleic acid that encodes an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments, the nucleic acid sequence that encodes a bicistronic iCAR/aCAR exhibits 75%, 80%, 85%, 88%, 90%, 91%, 92%, 93%, 94%, 95%, 96%, 97%, 98%, 99% or 100% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 75% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 80% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 85% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 90% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 91% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 92% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 93% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 94% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 95% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 96% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 97% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 98% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 99% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits 100% sequence identity to a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

As used herein, sequence identity can include the identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. Methods of alignment of sequences for comparison are well known in the art. Various programs and alignment algorithms are described in, for example but not limited to Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations. The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site. For example, BLASTN can be used to compare nucleic acid sequences, while BLASTP can be used to compare amino acid sequences. To compare two nucleic acid sequences, the options can be set as follows: -i is set to a file containing the first nucleic acid sequence to be compared (such as C:\seq1.txt); --j is set to a file containing the second nucleic acid sequence to be compared (such as C:\seq2.txt); --p is set to blastn; --o is set to any desired file name (such as C:\output.txt); --q is set to --1; --r is set to 2; and all other options are left at their default setting. For example, the following command can be used to generate an output file containing a comparison between two sequences: C:\B12seq --i c:\seq1.txt --j c:\seq2.txt --p blastn --o c:\output.txt --q --1 --r 2.

IV. Construction of Effector Cells

In still another aspect, the present invention provides a method for preparing a safe effector immune cell comprising: (i) transfecting an effector immune cell directed to a tumor-associated antigen with a nucleic acid molecule comprising a nucleotide sequence encoding a bicistronic iCAR/aCAR construct or monocistronic aCAR and iCAR constructs for co-transduction as defined herein above or transducing the cells with a vector or (ii) transfecting a naïve effector immune cell with a nucleic acid molecule comprising a nucleotide sequence encoding a bicistronic iCAR/aCAR construct as defined herein above; or transducing an effector immune cell with a vector as defined herein above. In some embodiments, the bicistronic iCAR/aCAR construct is encoded a single vector.

In still another aspect, the present invention provides a method for preparing a safe effector immune cell comprising: (i) transfecting a TCR-engineered effector immune cell directed to a tumor-associated antigen with a nucleic acid molecule comprising a nucleotide sequence encoding a bicistronic iCAR/aCAR construct or monocistronic aCAR and iCAR constructs for co-transduction as defined herein above or transducing the cells with a vector or (ii) transfecting a naïve effector immune cell with a nucleic acid molecule comprising a nucleotide sequence encoding a bicistronic iCAR/aCAR construct as defined herein above; or transducing an effector immune cell with a vector as defined herein above. In some embodiments, the bicistronic iCAR/aCAR construct is encoded a single vector. In some embodiments, the bicistronic iCAR and aCAR constructs are encoded on different/separate vectors. In some embodiments, the monocistronic aCAR and iCAR constructs for co-transduction are encoded on a single vector. In some embodiments, the monocistronic aCAR and iCAR constructs for co-transduction are encoded on different/separate vectors.

In some embodiments, the immune cell for use in engineering includes but is not limited to a T-cell, a natural killer cell, or a cytokine-induced killer cell. In some embodiments, the immune cell for use in engineering includes but is not limited to a Jurkat T-cell, a Jurkat-NFAT T-cell, and/or a peripheral blood mononuclear cell (PBMC).

In some embodiments, the immune cell is modified such that is a safe effector immune cell. In yet another aspect, the present invention provides a safe effector immune cell obtained by the method of the present invention as described above. The safe effector immune cell may be a redirected T cell expressing an exogenous T cell receptor (TCR) and a bicistronic iCAR/aCAR construct or monocistronic aCAR and iCAR constructs for co-transduction, wherein the exogenous TCR is directed to a non-polymorphic cell surface epitope of an antigen or a single allelic variant of a polymorphic cell surface epitope, wherein said epitope is a tumor-associated antigen or is shared at least by cells of related tumor and normal tissue, and a bicistronic iCAR/aCAR construct or monocistronic aCAR and iCAR constructs for co-transduction is as defined above; or the safe effector immune cell is a redirected effector immune cell such as a natural killer cell or a T cell expressing a bicistronic iCAR/aCAR construct or monocistronic aCAR and iCAR constructs for co-transduction as defined above.

In some embodiments, the safe effector immune cell expresses on its surface an aCAR comprising an extracellular domain that specifically binds to a non-polymorphic cell surface epitope of an antigen and an iCAR comprising an extracellular domain that specifically binds a single allelic variant of a polymorphic cell surface epitope of a different antigen to which the extracellular domain of said aCAR binds. In some embodiments, the extracellular domain of the iCAR specifically binds a single allelic variant of a different polymorphic cell surface epitope are of the same antigen to which the extracellular domain of said aCAR binds; or the extracellular domain of the iCAR specifically binds a different single allelic variant of the same polymorphic cell surface epitope area to which the extracellular domain of said aCAR binds.

In some embodiments, the aCAR and the iCAR are present on the cell surface as separate proteins. In some embodiments, the expression level on the cell surface of the iCAR is greater than or equal to the expression level of the aCAR. In some embodiments, the extracellular domain of the iCAR expressed on the cell surface is directed against or specifically binds to a single allelic variant of an at least one extracellular polymorphic epitope.

In some embodiments, the extracellular domain of the iCAR expressed on the cell surface is directed against or specifically binds to a single allelic variant of HLA-A2. In some embodiments, the iCAR will be directed toward HLA-A2. In some embodiments, the aCAR with be directed toward EGFR. In some embodiments, the aCAR with be directed toward HER2. In some embodiments, the iCAR/aCAR set will be HLA-A2 and EGFR respectively. In some embodiments, the iCAR/aCAR set will be HLA-A2 and HER2 respectively.

In some embodiments, the safe effector immune cell comprises a bicistronic iCAR/aCAR nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the safe effector immune cell comprises and expression vector comprising a bicistronic iCAR/aCAR nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the safe effector immune cell comprises a bicistronic iCAR/aCAR nucleic acid that encodes an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments, the safe effector immune cell comprises expression vector comprises a bicistronic iCAR/aCAR nucleic acid that encodes an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments, EGFR is the aCAR target and HLA is the iCAR target. In some embodiments, HER2 is the aCAR target and HLA is the iCAR target. In some embodiments, the safe effector immune cells used for treating cancer as defined comprises an expression vector. In some embodiments, the iCAR and aCAR are encoded by a bicistronic nucleic acid based expression vector. In some embodiments, the expression vector comprises a nucleic acid sequence a sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:3, SEQ ID NO:275, SEQ ID NO:277, SEQ ID NO:279, SEQ ID NO:281, SEQ ID NO:321, SEQ ID NO:323, and SEQ ID NO:325. In some embodiments, the expression vector comprises a nucleic acid sequence that codes for an amino sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments, the safe effector immune cells used for treating cancer comprises an expression vector that comprises a bicistronic iCAR/aCAR nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the safe effector immune cells used for treating cancer comprises a bicistronic iCAR/aCAR nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the safe effector immune cells used for treating cancer as comprises a bicistronic iCAR/aCAR nucleic acid that encodes an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments, the safe effector immune cells used for treating cancer as comprises an expression vector that comprises a bicistronic iCAR/aCAR nucleic acid that encodes an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

A. In Vitro Assays

In some embodiments, the bicistronic iCAR/aCAR constructs will be tested for activity effects, including effectiveness and ability to inhibit, using a variety of assays. In some embodiments, the bicistronic iCAR/aCAR constructs or monocistronic aCAR and iCAR constructs for co-transduction will be tested in-vitro and/or in-vivo. In some embodiments, the bicistronic iCAR/aCAR constructs or monocistronic aCAR and iCAR constructs for co-transduction will be tested in-vitro. In some embodiments, the bicistronic iCAR/aCAR constructs or monocistronic aCAR and iCAR constructs for co-transduction will be tested in-vivo. In some embodiments, the in vitro assays measure cytokine secretion and/or cytotoxicity effects. In some embodiments, the in vivo assays will evaluate the bicistronic iCAR/aCAR constructs or monocistronic aCAR and iCAR constructs for co-transduction inhibition and protection to on-target off tumor xenografts. In some embodiments, the in vivo assays will evaluate the bicistronic iCAR/aCAR constructs or monocistronic aCAR and iCAR constructs for co-transduction inhibition and protection to on-target off tumor tissue and/or viral organs.

i. Luciferase Cytotoxicity Assay

In some embodiments, bicistronic iCAR/aCAR constructs or monocistronic aCAR and iCAR constructs for co-transduction are evaluated using a luciferase cytotoxicity assay. Generally, for a luciferase cytotoxic assay, target tumor cells (which can be referred to as “T”) are engineered to express firefly luciferase. In some embodiments, commercially available ATCC cell lines are used. In some embodiments, H1703 cells were used. In some embodiments, H1650 cells were used. In some embodiments, H1792 cells were used. In some embodiments, H292 cells were used. The in vitro luciferase assay can be performed according to the Bright-Glo Luciferase assay (commercially available from Promega or BPS Biosciences or other commercial vendors). Transduced effector (E) T cells (which have been transduced with bicistronic iCAR/aCAR constructs or mock/control construct) can be incubated for 18-48 hrs with recombinant target cells expressing the iCAR or aCAR target to be tested in different effector to target ratios. In some embodiments, the iCAR/aCAR pair comprises any of aCAR and/or iCAR with the components as described above. In some embodiments, the bicistronic iCAR/aCAR constructs described above are to be tested. In some embodiments, the bicistronic iCAR/aCAR comprises an amino acid sequence encoded by a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325. In some embodiments, the bicistronic iCAR/aCAR comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326. Cell killing can be quantified indirectly by estimating the number of live cells with the Bright-Glo Luciferase system. Cell killing can also be measured using an IncuCyte cytotoxicity assay.

In some embodiments, the ‘off-tumor’ cytotoxicity can be manipulated by sorting transduced T cell populations according to iCAR/aCAR expression level or by selecting a sub population of recombinant target cells according to their target expression, including for example, expression of the gene product encoding for at least one extracellular polymorphic epitope. In some embodiments, the aCAR and iCAR target is any target with an extracellular domain. In some embodiments, the sorting is based on EGFR, HER2, or HLA-A2 expression level.

In some embodiments, the bicistronic iCAR/aCAR construct or monocistronic aCAR and iCAR constructs for co-transduction is examined to determine whether the iCAR transduced T cells can discriminate between the ‘on-tumor’ cells (e.g., tumor cells) and ‘off-tumor’ cells (e.g., non-tumor cells) in vitro. Generally, this is tested by examining the killing effect of transduced T cells incubated with a mix of ‘on-tumor’ and ‘off-tumor’ cells at a ratio of 1:1 to 1:10. In some embodiments, the ratio Target cells to Effector T cells (T:E ratio) is 1:0.02, 1:0.04, 1:0.06, 1:0.08, 1:0.1, 1:0.12, 1:0.12, 1:0.14, 1:0.16, 1:0.18, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the E:T ratio (Effector T cells to Target cells) is 0.02:1, 0.04:1, 0.06:1, 0.08:1, 0.1:1, 0.12:1, 0.12:1, 0.14:1, 0.16:1, 0.18:1, 2:1, 3:1, 4:1, 5:1:1, 6:1, 7:1, 8:1, 9:1, 10:1, 11:1, 12:1, 13:1, 14:1, 15:1, 16:1, 17:1, 18:1, 19:1, or 20:1. The on tumor recombinant cells can be distinguished from the ‘off-tumor’ recombinant cells by luciferase expression in embodiments where only one cell population will be engineered to express the luciferase gene at a time). Killing can be quantified after 24-48 hrs of co-incubation using the Bright-Glo Luciferase assay (Promega). Killing can also be quantified using an IncCyte cytotoxicity assay. In some embodiments, transduced cells were only used in the assay of transduction efficiency was greater than 10% and expression was observed for both aCAR and iCAR.

In some embodiments, the bicistronic iCAR/aCAR construct or monocistronic aCAR and iCAR constructs for co-transduction transduced T cells exhibit about 10%, about 20%, about 30%, about 40%, about 50%, about 60%, about 70%, about 80%, about 90%, and/or about 95% less off-tumor cell killing as compared to T cells transduced with aCAR (or other control) but not transduced with the bicistronic iCAR/aCAR construct or monocistronic aCAR and iCAR constructs for co-transduction. In some embodiments, the bicistronic iCAR/aCAR construct or monocistronic aCAR and iCAR constructs for co-transduction transduced T cells exhibit about 1-fold, about 2-fold, about 3-fold, about 4-fold, about 5-fold, or about 10-fold less off-tumor cell killing as compared to T cells transduced with aCAR (or other control) but not transduced with the bicistronic iCAR/aCAR construct.

ii. Caspase 3

In some embodiments, caspase 3-detection assays are employed to determine the level of apoptosis of the ‘on-tumor’ cells (e.g., tumor cells) and ‘off-tumor’ cells (e.g., non-tumor cells) in vitro. In some embodiments, caspase 3-detection of cytotoxic lymphocyte (CTL) induced apoptosis by an antibody to activated cleaved caspase 3 is examined.

Generally, one of the pathways by which CTLs kill target cells is by inducing apoptosis through the Fas ligand. The CASP3 protein is a member of the cysteine-aspartic acid protease (caspase) family. Typically, sequential activation of caspases plays a significant role in the execution-phase of cell apoptosis and as such, cleavage of pro-caspase 3 to caspase 3 results in conformational change and expression of catalytic activity. The cleaved activated form of caspase 3 can be recognized specifically by a monoclonal antibody.

In some embodiments, transduced T cells can be incubated with either ‘on-tumor’ (e.g., mimicking tumor) and ‘off-tumor’ cells (e.g., mimicking non-tumor) recombinant cells. In some embodiments, the ‘on-tumor’ (e.g., tumor) and ‘off-tumor’ cells (e.g., non-tumor) recombinant cells have been previously labeled with CFSE ((5(6)-Carboxyfluorescein N-hydroxysuccinimidyl ester)) or other cell tracer dye (e.g., CellTrace Violet). In some embodiments, co-incubation of target cells with effector cells occurs for about 1 hour to 6 about hours, about 2 hours to about 5 hours, or about 2 to about 4 hrs. In some embodiments, target cell apoptosis is quantified by flow cytometry. Cells can be permeabilized and fixed by an inside staining kit (Miltenyi or BD bioscience) and stained with an antibody for activated caspase 3 (BD bioscience).

In some embodiments, the bicistronic iCAR/aCAR construct transduced T cells induce about 10%, about 20%, about 30%, about 40%, about 50%, about 60%, about 70%, about 80%, about 90%, and/or about 95% less off-tumor cell apoptosis as compared to T cells transduced with the bicistronic iCAR/aCAR construct but not transduced with the iCAR (or other appropriate control). In some embodiments, the bicistronic iCAR/aCAR construct transduced T cells induce about 1-fold, about 2-fold, about 3-fold, about 4-fold, about 5-fold, or about 10-fold less off-tumor cell apoptosis as compared to T cells transduced with aCAR (or other control) but not transduced with the bicistronic iCAR/aCAR construct.

iii. Time-Lapse Microscopy

Time lapse microscopy of the bicistronic iCAR/aCAR construct or monocistronic aCAR and iCAR constructs for co-transduction transduced T cells can be employed in order to discern target binding. In some embodiments, target cells will be labeled with a reporter gene (for example but not limited to a fluorescent protein such as mCherry). In some embodiments, transduced T cells are incubated with either ‘on-tumor’ or ‘off-tumor’ cells for up to 5 days. In some embodiments, time lapse microscopy can be used to visualize killing. In some embodiments, flow cytometry analysis using viable cell number staining and CountBright™ beads (commercially available from Thermofisher/Invitrogen) for determining target cell number at end-point time will be conducted.

In some embodiments, in order to determine if the bicistronic iCAR/aCAR construct or monocistronic aCAR and iCAR constructs for co-transduction transduced T cells can discern targets in vitro, each recombinant target cells (‘on-tumor’ or ‘off-tumor’) is labeled with a different reporter protein (for example GFP and mCherry). In some embodiments, any report protein pair would work, so long as the reporter pair contains two reporters which are easily distinguishable. In some embodiments, transduced T cells (Effector cells) will be co-incubated with the recombinant cells (target cells) at a 1:1 ratio of E/T. In some embodiments, the ration of effector to target (E/T) includes but is not limited to 16:1, 15:1, 14:1, 13:1, 12:1, 11:1, 10:1, 9:1, 8:1, 6:1, 4:1, 2:1, or 1:1. In some embodiments, the cell fate is then examined by microscopy imaging.

iv. Cytokine Expression Intra Cellular Staining

Cytokine expression and/or release can be examined in order to determine T cells activation. In some embodiments, a bicistronic iCAR/aCAR construct transduced T cells are incubated with the recombinant target cells and cytokine production for one or more cytokines is quantified, for example, either by measuring cytokine secretion in cell culture supernatant according to or by flow cytometry analysis, or by Luminex and/or MSD. For the flow cytometry analysis, a Golgi stop can be employed to prevent the secretion of the cytokines. In some embodiments, following a 6 hour and 18 hour to 24 hour incubation of the transduced T cells with target cells, T cells will be permeabilized and fixed by an intracellular staining kit (Miltenyi) and stained with antibodies for the T cell markers (CD3 and CD8) and for one or more cytokines. In some embodiments, the cytokines include but are not limited to IL-2, INFγ, and/or TNFα. In some embodiments, the cytokines are secreted and include but are not limited to IL-2, INFγ, and/or TNFα. In some embodiments, the cytokines are intracellular and include but are not limited to IL-2, INFγ, and/or TNFα.

v. T Cell Degranulation Assay Measured by CD107a Staining

Staining for CD107a can also be examined as a surrogate for cytolytic activity of the transduced T cells. Generally, degranulating of T cells can be identified by the surface expression of CD107a, a lysosomal associated membrane protein (LAMP-1), and surface expression of LAMP-1 has been shown to correlate with CD8 T cell cytotoxicity. Further, this molecule is located on the luminal side of lysosomes. Typically, upon activation, CD107a is transferred to the cell membrane surface of activated lymphocytes. Moreover, CD107a is expressed on the cell surface transiently and is rapidly re-internalized via the endocytic pathway. Therefore, while not being bound by theory, CD107a detection is maximized by antibody staining during cell stimulation and by the addition of monensin (for example, to prevent acidification and subsequent degradation of endocytosed CD107a antibody complexes).

In some embodiments, the bicistronic iCAR/aCAR construct transduced T cells are incubated with the target cells for about 6 hours to about 24 hours and CD107a expression on the CD8 T cells is examined. In some embodiments, the target cells expresso only one target protein recognized by aCAR (as in tumor cells) or target cells expressing both target proteins recognized by aCAR and iCAR (as in normal cells). In some embodiments, the bicistronic iCAR/aCAR construct transduced T cells are incubated with the target cells for about 6 ours to about 24 hrs in the presence of monensin and CD107a expression on the CD8 T cells is followed by flow cytometry using conjugated antibodies against the T cell surface markers (for example, CD3 and CD8) and a conjugated antibody for CD107a.

vi. Quantitation of Secreted Cytokines by ELISA/Luminex

In some embodiments, following co-cultivation of bicistronic iCAR/aCAR construct transduced T-cells (Jurkat, or primary T-cells) expressing iCAR or aCAR or both aCAR and iCAR with modified target cells, expressing iCAR or aCAR or both aCAR and iCAR antigens on their cell surface, conditioned medium will be collected, and cytokine's concentration will be measured by cytokine ELISA or by Luminex xMAP Multiplex Assay technology (Luminex). In some embodiments, the cytokine is selected from the group consisting of IL-2, INFγ and/or TNF□. In some embodiments, the cytokine is selected from the group consisting of IL-2. In some embodiments, the cytokine is selected from the group consisting of INFγ. In some embodiments, the cytokine is selected from the group consisting of TNF□. In some embodiments, a decrease of about 20%, about 25%, about 30%, about 35%, about 40%, about 45%, about 50%, about 55%, about 60%, about 65%, about 70%, about 75%, about 80%, about 85%, about 90%, about 95%, or about 99% is demonstrated with bicistronic iCAR/aCAR construct transduced cells.

vii. Cytokines Secretion Measured by Cytometric Bead Array (CBA) Assay

Cytometric Bead Array (CBA) is used to measure a variety of soluble and intracellular proteins, including cytokines, chemokines and growth factors. In some embodiments, T-cells (primary T-cells or Jurkat cells) transduced with aCAR or both aCAR and iCAR constructs (Effector cells) are stimulated with modified target cells expressing both iCAR and aCAR or aCAR or iCAR target antigens on their cell surface. In some embodiments, the effector to target ratio ranges from 20:1 up to 1:1. In some embodiments, the effector to target ratio ranges from 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or 1:1. In some embodiments, following several hours of co-incubation the effector cells produce and secrete cytokines which indicate their effector state. In some embodiments, the supernatant of the reaction is collected, and secreted IL-2, IFN-γ, and/or TNFα were measured and quantified by multiplex CBA assay.

In some embodiments, a decrease of about 20%, about 25%, about 30%, about 35%, about 40%, about 45%, about 50%, about 55%, about 60%, about 65%, about 70%, about 75%, about 80%, about 85%, about 90%, about 95%, or about 99% is demonstrated with dual CAR (aCAR/iCAR) transduced cells were co-incubated with target cells expressing both target antigens as compared to IL-2, IFN-γ, and/or TNFα secretion resulted from co-incubation of the same effector cells with target cells expressing only one target. In some embodiments, a decrease of about 20%, about 25%, about 30%, about 35%, about 40%, about 45%, about 50%, about 55%, about 60%, about 65%, about 70%, about 75%, about 80%, about 85%, about 90%, about 95%, or about 99% in IL-2 IFN-γ, and/or TNFα secretion was demonstrated when bicistronic iCAR/aCAR construct transduced cells were co-incubated with target cells expressing both target antigens as compared to IL-2 IFN-γ, and/or TNFα secretion resulted from co-incubation of the same effector cells with target cells expressing only one target. In some embodiments, a decrease of 86%.

B. In Vivo Assays

In some embodiments, the bicistronic iCAR/aCAR construct are tested for effectiveness in vivo. In some embodiments, NOD/SCID/γc- or similar mice are inoculated subcutaneously or orthotopically with tumor cells. In some embodiments, the tumor cells are EGFR and HER2 positive cells lines A549, A431, Fadu, SK-OV-3, U-87, MCF7, NCI-H460, NCI-H1703, NCI-H1650, NCI-H1975, NCI-H292 (ATCC cell lines) cells. In some embodiments, for establishment of and/or differentiation between ‘on-target’ cells and ‘off-tumor’ cells, A549, A431, Fadu, SK-OV-3, U-87, MCF7, NCI-H460 NCI-H1703, NCI-H1650, NCI-H1975, NCI-H292 can be engineered to be deficient or express the iCAR epitope, thereby representing the healthy cells. In some embodiments, the iCAR epitope comprises at least one extracellular polymorphic epitope. In some embodiments, the iCAR epitope is from HLA (including, for example, HLA-A2, HLA-A3, HLA-A, HLA-B, HLA-C, HLA-G, HLA-E, HLA-F, HLA-DPA1, HLA-DQA1, HLA-DQB1, HLA-DQB2, HLA-DRB1, or HLA-DRB5). In some embodiments, the iCAR epitope is from HLA-A2. Other cells that could be employed in these assays include but are not limited to Raji or any other recombinant cell lines. In some embodiments, such assays can be in a PDX (patient derived xenograft) model.

For the assay, mice will be divided into study groups; one cohort will be injected with the A549, A431, Fadu, SK-OV-3, U-87, MCF7, NCI-H460 NCI-H1703, NCI-H1650, NCI-H1975, and/or NCI-H292 cells not expressing the iCAR epitope, while the other will be injected with the corresponding A549, A431, Fadu, SK-OV-3, U-87, MCF7, NCI-H460 NCI-H1703, NCI-H1650, NCI-H1975, NCI-H292 cells expressing the iCAR epitope. Following staging, mice will be infused intravenously with T cells transduced with aCAR, aCAR/iCAR and a control group of untransduced T cells or no T cells. Tumor burden will be measured by through measurement of the subcutaneous tumor volume.

According to one embodiment of the assay, in order to test whether the T cells expressing the bicistronic iCAR/aCAR constructs could discriminate between the target cells and off target cells in vivo within the same organism, mice are injected with a 1:1 mixture of the ‘on-tumor’/‘off-tumor A549, A431, Fadu, SK-OV-3, U-87, MCF7, NCI-H460 NCI-H1703, NCI-H1650, NCI-H1975, and/or NCI-H292 cells, followed by injection of transduced T cells expressing either the aCAR alone or both aCAR and iCAR (including as the bicistronic iCAR/aCAR constructs as described herein) after staging. With this embodiment, upon sacrifice of the mice the presence of the ‘on-tumor’ and ‘off-tumor cells Will be evaluated by immunohistochemical staining

According to one embodiment of the assay, in order to test whether the T cells expressing the bicistronic iCAR/aCAR constructs could discriminate between the target cells and off target cells in vivo within the same organism, mice are injected with a 1:10 mixture of the ‘on-tumor’/‘off-tumor NALM-6, A549, A431, Fadu, SK-OV-3, U-87, MCF7, and/or NCI-H460 NCI-H1703, NCI-H1650, NCI-H1975, NCI-H292 cells, followed by injection of transduced T cells expressing either the aCAR alone or both aCAR and iCAR. With this embodiment, upon sacrifice of the mice the presence of the ‘on-tumor’ and ‘off-tumor cells in the spleen and bone marrow will be analyzed by flow cytometry for iCAR and aCAR markers.

i. Tumor Growth Kinetics in Human Xenograft Mouse Models

In some embodiments, the tumor cells express either the iCAR target, aCAR target or both. In some embodiments, an aCAR tumor cell line could be the EGFR or HER2 positive cells lines A549, A431, Fadu, SK-OV-3 U-87, MCF7, and/or NCI-H460 (ATCC cell lines). In some embodiments, tumor cells that express both the aCAR and iCAR (i.e. ‘off-tumor’ cells) are NALM 6, A549, A431, Fadu, SK-OV-3, U-87, MCF7, MDA-MB-231, and/or NCI-H460 engineered to express the iCAR epitope (for example, HLA-A2) thereby representing the healthy cells. In some embodiments, NALM 6 and NALM 6-HLA-A2 can also be engineered to express a reporter gene (e.g., firefly luciferase, GFP, mCherry), for easy detection. In some embodiments, A549 and A549-HLA-A2 can also be engineered to express a reporter gene (e.g., firefly luciferase), for easy detection. In some embodiments, A431 and A431-HLA-A2 can also be engineered to express a reporter gene (e.g., firefly luciferase), for easy detection. In some embodiments, Fadu and Fadu-HLA-A2 can also be engineered to express a reporter gene (e.g., firefly luciferase), for easy detection. In some embodiments, SK-OV-3 and SK-OV-3-HLA-A2 can also be engineered to express a reporter gene (e.g., firefly luciferase), for easy detection. In some embodiments, NCI-H460 and NCI-H460-HLA-A2 can also be engineered to express a reporter gene (e.g., firefly luciferase), for easy detection. In some embodiments, U-87 and U-87-HLA-A2 can also be engineered to express a reporter gene (e.g., firefly luciferase), for easy detection. In some embodiments, MCF7 and MCF7-HLA-A2 can also be engineered to express a reporter gene (e.g., firefly luciferase), for easy detection. In some embodiments, can also be engineered to express a reporter gene (e.g., firefly luciferase), for easy detection. In some embodiments, NCI-H460 and NCI-H460-HLA-A2 can also be engineered to express a reporter gene (e.g., firefly luciferase), for easy detection.

In some embodiments, monitoring will be conducted by measuring tumor volume by mechanical means (caliper) and also by using in-vivo imaging systems (IVIS). In some embodiments, tumor burden can be quantified, and infiltrating T-cell populations can be analyzed by FACS.

C. Treatment Methods

The present invention provides methods for the treatment of cancers by employing the bicistronic iCAR/aCAR constructs or monocistronic aCAR and iCAR constructs for co-transduction as described herein. The methods of treatment for cancer as described herein can employ exploiting loss of heterozygosity, or other genetic loss or allelic imbalance phenotypes found in human tumors, including, without limitation, loss of function or expression, resulting from mutations affecting one or more nucleotides (for example, without limitation, in HLA-1 genes) by means of CAR-T therapy, or by modifying other cells of the immune system.

In yet another aspect, the present invention provides a method of selecting a personalized biomarker for a subject having a tumor characterized by loss of heterozygosity, or other genetic loss or allelic imbalance phenotypes found in human tumors, the method comprising (i) obtaining a tumor biopsy from the subject; (ii) obtaining a sample of normal tissue from the subject, e.g., PBMCs; (iii) identifying a single allelic variant of a polymorphic cell surface epitope that is not expressed by cells of the tumor due to loss of heterozygosity, or other genetic loss or allelic imbalance phenotypes found in human tumors, but that is expressed by the cells of the normal tissue, thereby identifying a personalized biomarker for the subject, and (iv) determining the appropriate bicistronic iCAR/aCAR construct or monocistronic aCAR and iCAR constructs for co-transduction as described herein for use in treatment.

In a further aspect, the present invention provides a method for treating cancer in a patient having a tumor characterized by loss of heterozygosity, or other genetic loss or allelic imbalance phenotypes found in human tumors, comprising administering to the patient an effector immune cell as defined above, wherein the iCAR is directed to a single allelic variant encoding a polymorphic cell surface epitope absent from cells of the tumor due to loss of heterozygosity, or other genetic loss or allelic imbalance phenotypes found in human tumors but present at least on all cells of related mammalian normal tissue of the patient. In some embodiments, the effector immune cell comprises a bicistronic iCAR/aCAR construct as described herein.

In some embodiments, the treating results in reduced on-target, off-tumor reactivity, as compared with a treatment comprising administering to the cancer patient at least one population of immune effector cells expressing a bicistronic iCAR/aCAR construct as described herein.

In some embodiments, the safe effector immune cells used for treating cancer as defined above express on their surface an aCAR comprising an extracellular domain that specifically binds to a tumor-associated antigen or a non-polymorphic cell surface epitope of an antigen and an iCAR comprising an extracellular domain that specifically binds a single allelic variant of a polymorphic cell surface epitope of an antigen expressed at least in a tissue of origin of the tumor or of a housekeeping protein, which is a different antigen than that to which the extracellular domain of said aCAR binds. In some embodiments, the effector immune cell expresses the components of a bicistronic iCAR/aCAR construct or monocistronic aCAR and iCAR constructs for co-transduction as described herein.

In some embodiments, the safe effector immune cells used for treating cancer as defined above express on their surface an aCAR comprising an extracellular domain that specifically binds to a tumor-associated antigen or a non-polymorphic cell surface epitope of an antigen and an iCAR comprising an extracellular domain that specifically binds a single allelic variant of a polymorphic cell surface epitope of an antigen expressed at least in a tissue of origin of the tumor or of a housekeeping protein, such as an HLA genes (including for example, HLA-A, HLA-B, HLA-C, HLA-G, HLA-E, HLA-F, HLA-K, HLA-L, HLA-DM, HLA-DO, HLA-DP, HLA-DQ, or HLA-DR) which is a different antigen than that to which the extracellular domain of said aCAR binds.

In some embodiments, the safe effector immune cells used for treating cancer as defined above express on their surface an aCAR comprising an extracellular domain that specifically binds to a tumor-associated antigen or a non-polymorphic cell surface epitope of an antigen and an iCAR comprising an extracellular domain that specifically binds a single allelic variant of a polymorphic cell surface epitope of an antigen expressed at least in a tissue of origin of the tumor, such as an HLA-A, which is a different antigen than that to which the extracellular domain of said aCAR binds.

In some embodiments, the safe effector immune cells used in the method of treating cancer are selected from T cells, natural killer cells or cytokine-induced killer cells. In some embodiments, the safe effector immune cell is autologous or universal (allogeneic) effector cells. In some embodiments, the iCAR used in any one of the methods of treating cancer defined above is directed to all tissues of the patient on which the target-antigen of the aCAR is present, wherein the target antigen of the aCAR is a non-polymorphic cell surface epitope of an antigen or a single allelic variant of a polymorphic cell surface epitope is present, and said epitope is a tumor-associated antigen or is shared at least by cells of related tumor and normal tissue.

In some embodiments, the cancer is selected from the group consisting of Acute Myeloid Leukemia [LAML], Adrenocortical carcinoma [ACC], Bladder Urothelial Carcinoma [BLCA], Brain Lower Grade Glioma [LGG], Breast invasive carcinoma [BRCA], Cervical squamous cell carcinoma and endocervical adenocarcinoma [CESC], Cholangiocarcinoma [CHOL], Colon adenocarcinoma [COAD], Esophageal carcinoma [ESCA], Glioblastoma multiforme [GBM], Head and Neck squamous cell carcinoma [HNSC], Kidney Chromophobe [KICH], Kidney renal clear cell carcinoma [KIRC], Kidney renal papillary cell carcinoma [KIRP], Liver hepatocellular carcinoma [LIHC], Lung adenocarcinoma [LUAD], Lung squamous cell carcinoma [LUSC], Lymphoid Neoplasm Diffuse Large B-cell Lymphoma [DLBC], Mesothelioma [MESO], Ovarian serous cystadenocarcinoma [OV], Pancreatic adenocarcinoma [PAAD], Pheochromocytoma and Paraganglioma [PCPG], Prostate adenocarcinoma [PRAD], Rectum adenocarcinoma [READ], Sarcoma [SARC], Skin Cutaneous Melanoma [SKCM], Stomach adenocarcinoma [STAD], Testicular Germ Cell Tumors [TGCT], Thymoma [THYM], Thyroid carcinoma [THCA], Uterine Carcinosarcoma [UCS], Uterine Corpus Endometrial Carcinoma [UCEC], Uveal Melanoma [UVM], Non-small cell lung carcinoma [NSCLC], and Small cell lung cancer [SCLC].

In some embodiments, the bicistronic iCAR/aCAR construct or monocistronic aCAR and iCAR constructs for co-transduction for use in the treatment of cancer is any bicistronic iCAR/aCAR construct described herein. In some embodiments, the bicistronic iCAR/aCAR construct used to treat the cancer, such as any one of the cancer types recited above, is directed against or specifically binds to a single allelic variant of an HLA genes (including for example, HLA-A, HLA-B, HLA-C, HLA-G, HLA-E, HLA-F, HLA-K, HLA-L, HLA-DM, HLA-DO, HLA-DP, HLA-DQ, or HLA-DR, HLA-B gene or HLA-C gene or against a single allelic variant. In some embodiments, the treatment method employs administration of a safe effector cell comprising the bicistronic iCAR/aCAR construct or monocistronic aCAR and iCAR constructs for co-transduction. In some embodiments, the treatment method employs administration of a safe effector cell expressing the bicistronic iCAR/aCAR construct or monocistronic aCAR and iCAR constructs for co-transduction.

In some embodiments, the bicistronic iCAR/aCAR or monocistronic aCAR and iCAR constructs for co-transduction for use in the treatment of cancer comprises an amino acid sequence encoded by a nucleic acid sequence selected from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325. In some embodiments, the bicistronic iCAR/aCAR construct or monocistronic aCAR and iCAR constructs for co-transduction comprises an amino acid sequence encoded by a nucleic acid sequence selected from the group consisting of SEQ ID NO:275, SEQ ID NO:277, SEQ ID NO:279, SEQ ID NO:281, SEQ ID NO:321, SEQ ID NO:323, and SEQ ID NO:325. In some embodiments, the treatment method employs administration of a safe effector cell comprising the bicistronic iCAR/aCAR construct or monocistronic aCAR and iCAR constructs for co-transduction. In some embodiments, the treatment method employs administration of a safe effector cell expressing the bicistronic iCAR/aCAR construct or monocistronic aCAR and iCAR constructs for co-transduction.

In some embodiments, the bicistronic iCAR/aCAR for use in the treatment of cancer comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326. In some embodiments, the bicistronic iCAR/aCAR construct or monocistronic aCAR and iCAR constructs for co-transduction comprises an amino acid sequence selected from the group consisting of SEQ ID NO:276, SEQ ID NO:278, SEQ ID NO:280, SEQ ID NO:282, SEQ ID NO:322, SEQ ID NO:324, and SEQ ID NO:326. In some embodiments, the treatment method employs administration of a safe effector cell comprising the bicistronic iCAR/aCAR construct or monocistronic aCAR and iCAR constructs for co-transduction. In some embodiments, the treatment method employs administration of a safe effector cell expressing the bicistronic iCAR/aCAR construct or monocistronic aCAR and iCAR constructs for co-transduction.

The compositions may be formulated for parenteral administration by injection, e.g., by bolus injection or continuous infusion. Formulations for injection may be presented in unit dosage form, e.g., in ampoules or in multidose containers, with an added pharmaceutically acceptable carrier and/or preservative. The compositions may take such forms as suspensions, solutions or emulsions in oily or aqueous vehicles, and may contain formulatory agents such as suspending, stabilizing and/or dispersing agents.

For purposes of clarity, and in no way limiting the scope of the teachings, unless otherwise indicated, all numbers expressing quantities, percentages or proportions, and other numerical values recited herein, should be interpreted as being preceded in all instances by the term “about.” Accordingly, the numerical parameters recited in the present specification are approximations that may vary depending on the desired outcome. For example, each numerical parameter may be construed in light of the number of reported significant digits and by applying ordinary rounding techniques.

### EXAMPLES

### Example 1. Development and Testing of Bicistronic Inhibitory Chimeric Antigen Receptor (iCAR)/Activating Chimeric Antigen Receptor (aCAR) Constructs

### INTRODUCTION

This example provides the results related to development and testing of bicistronic inhibitory chimeric antigen receptor (iCAR)/activating chimeric antigen receptor (aCAR) constructs in order to develop cancer therapeutics for use in safely target tumors that have lost genomic segments encoding cell-membrane proteins with polymorphic protein coding changes). Data provide in the example and figures include T-REP identification of new iCAR leads, new human HLA-A2 scFv constructs, and bicistronic LV transduction—FaDu/MCF7-Luc immune killing assay—including development of novel iCAR leads.

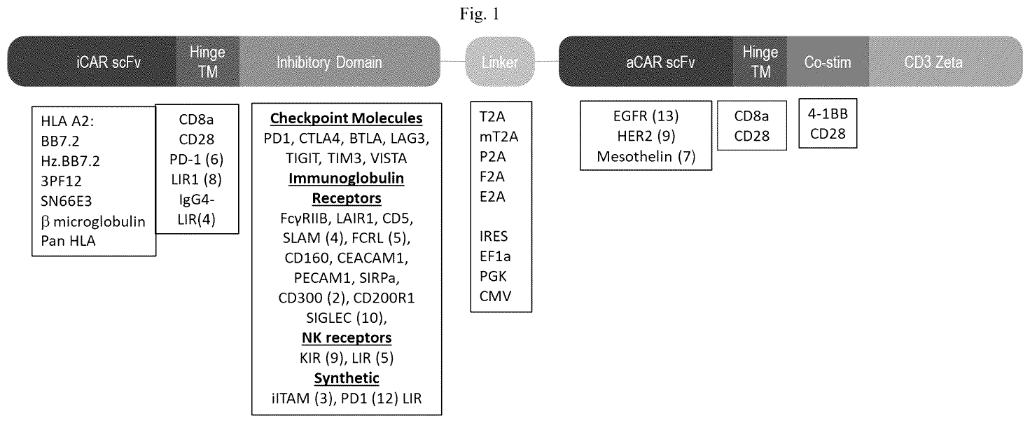

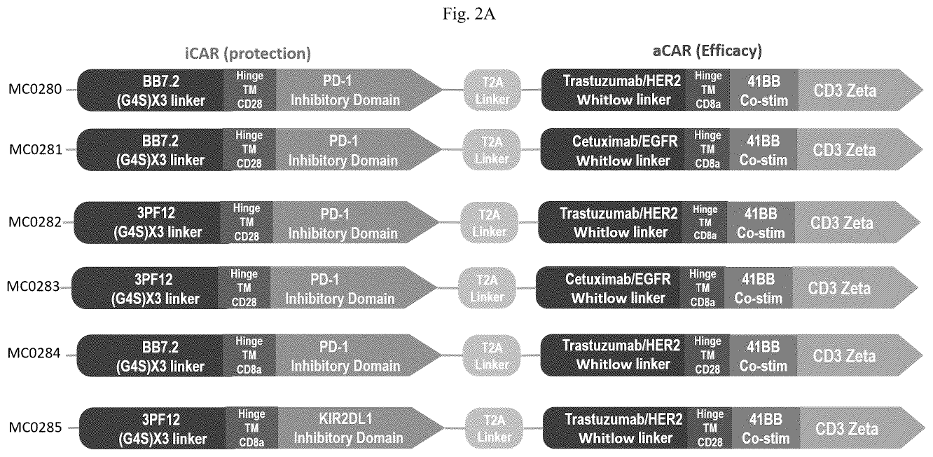

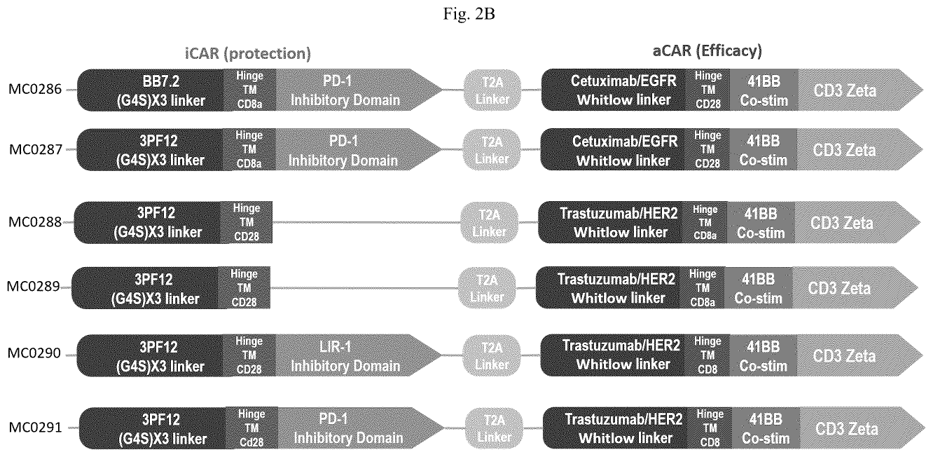

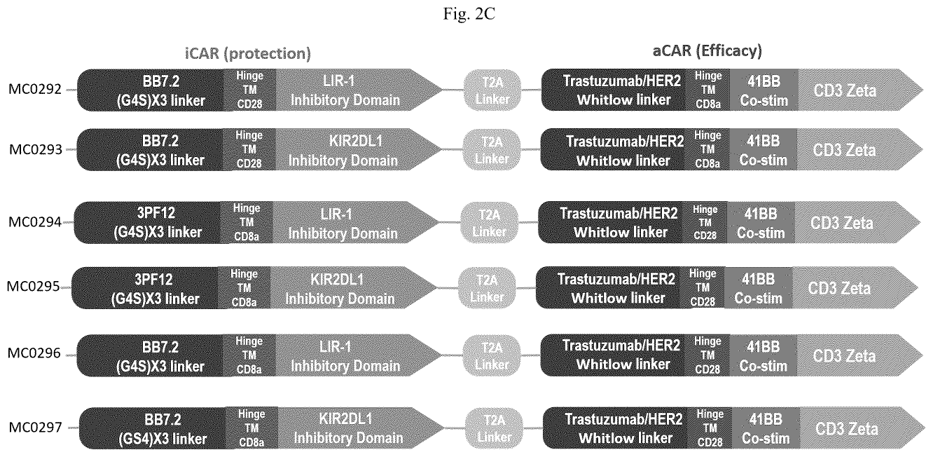

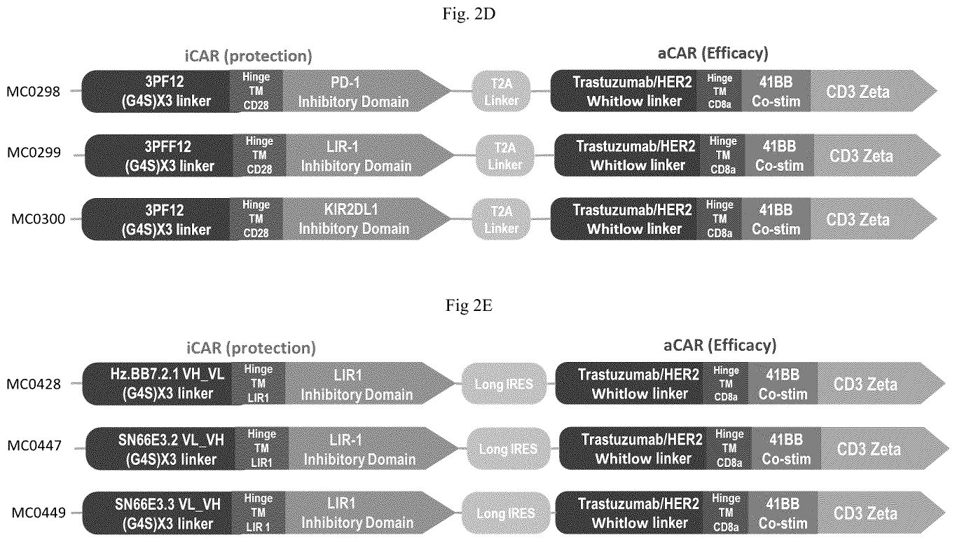

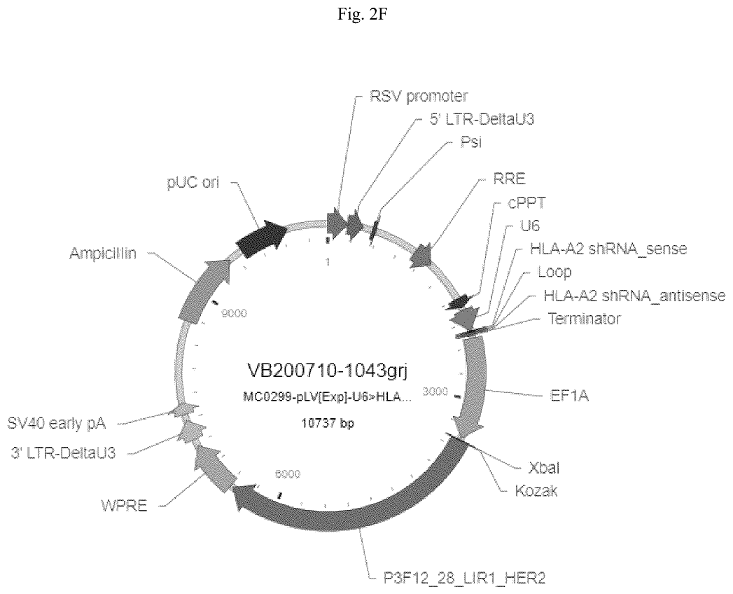

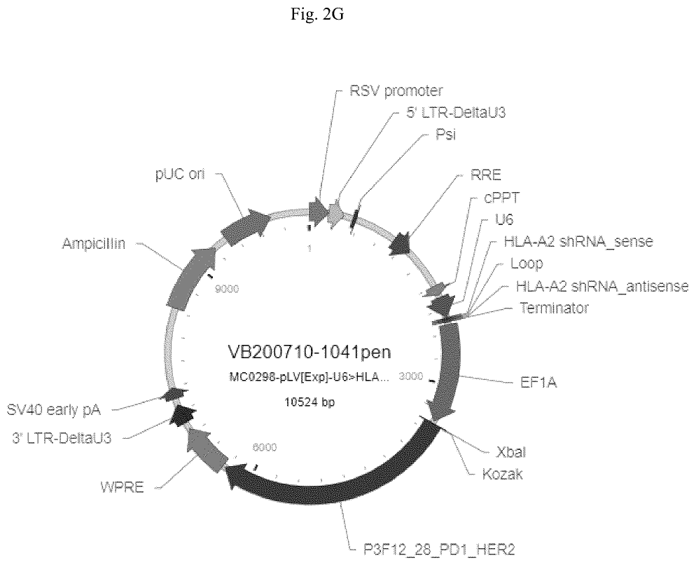

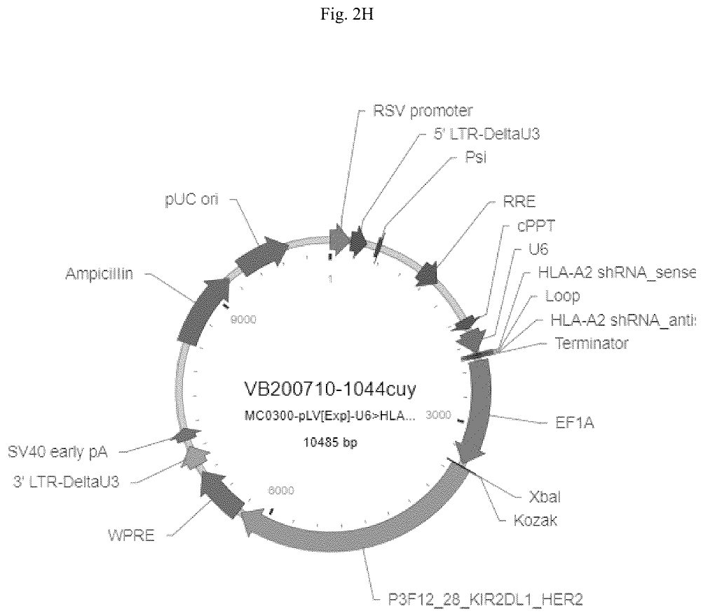

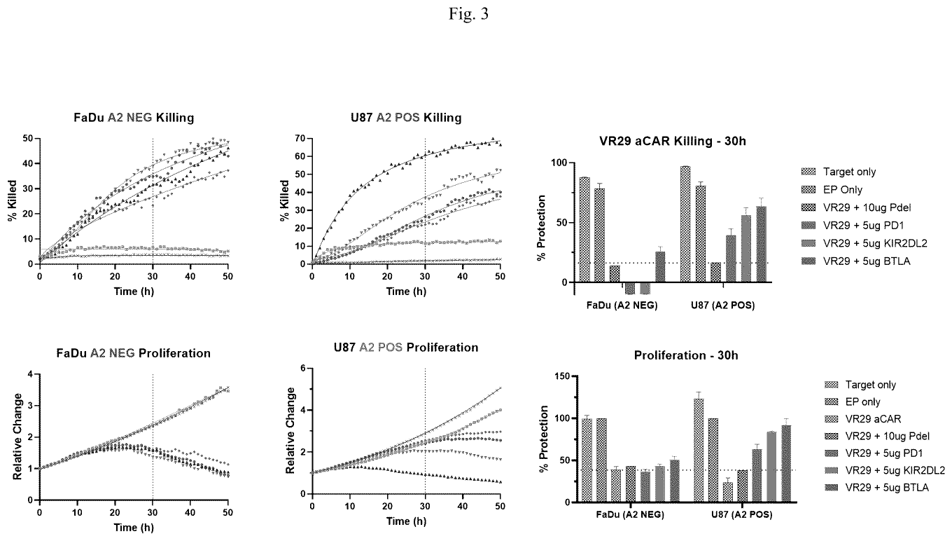

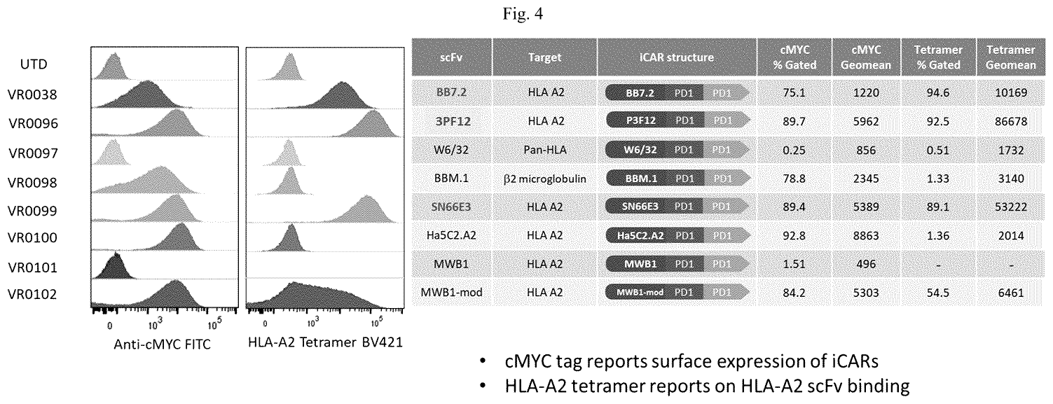

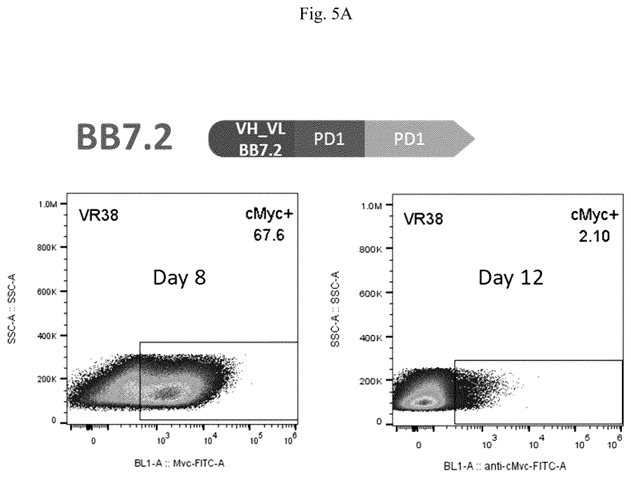

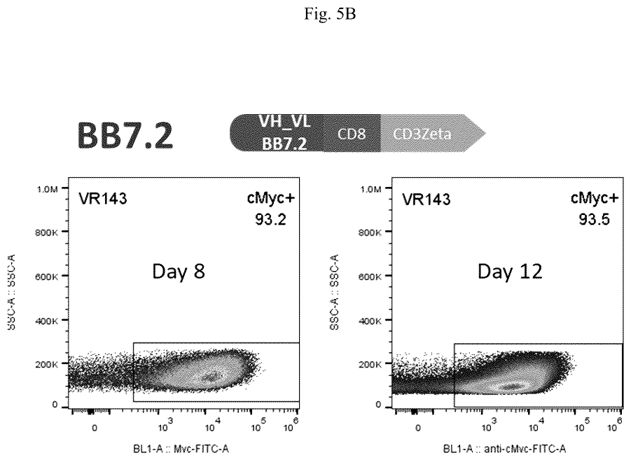

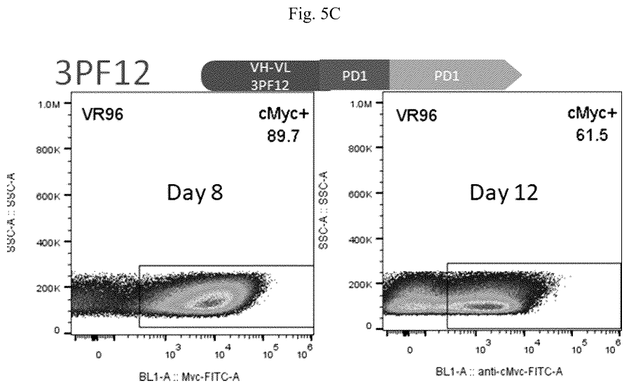

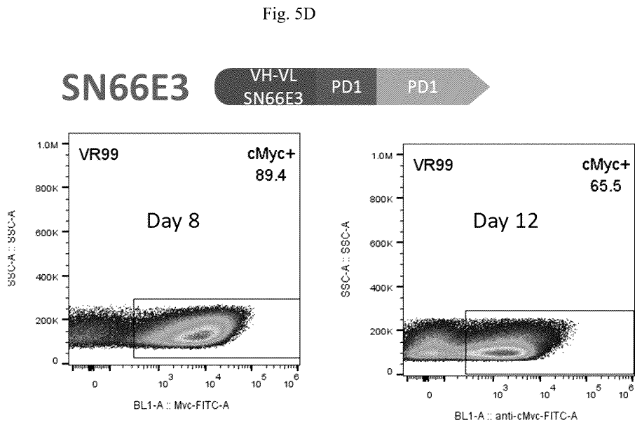

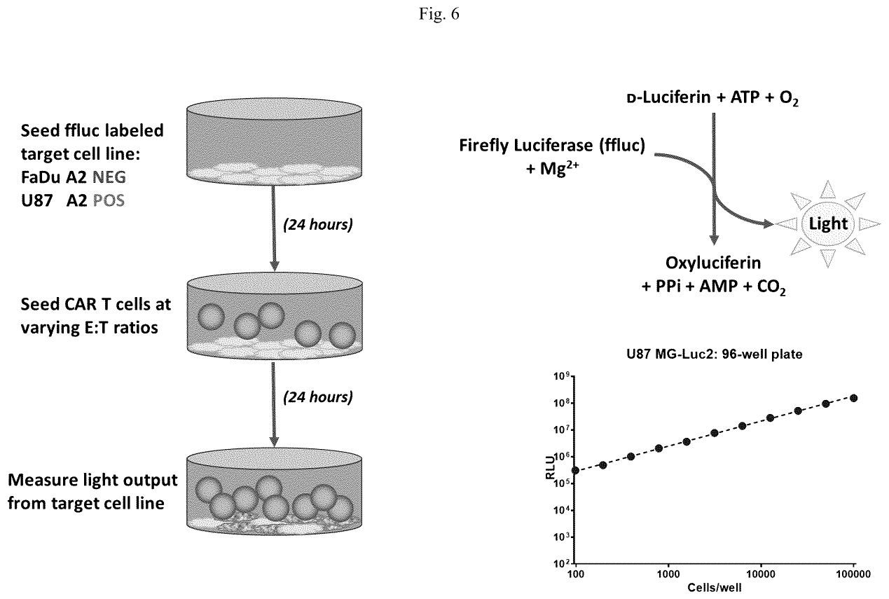

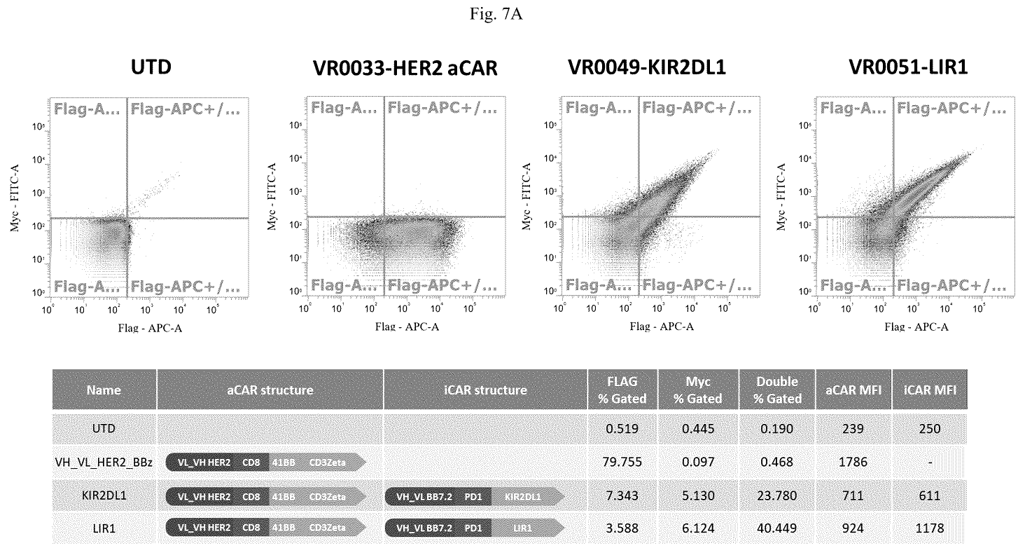

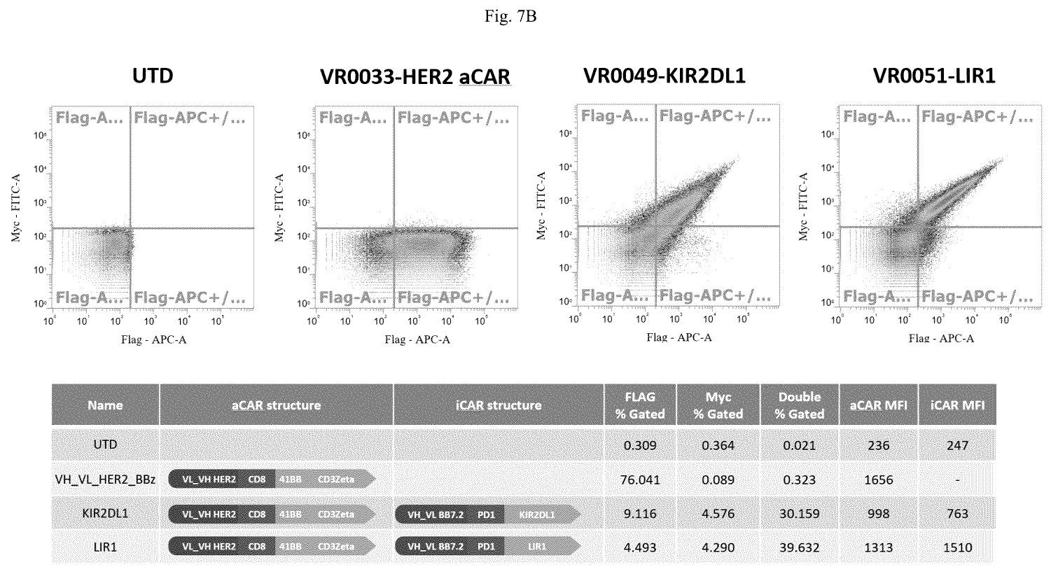

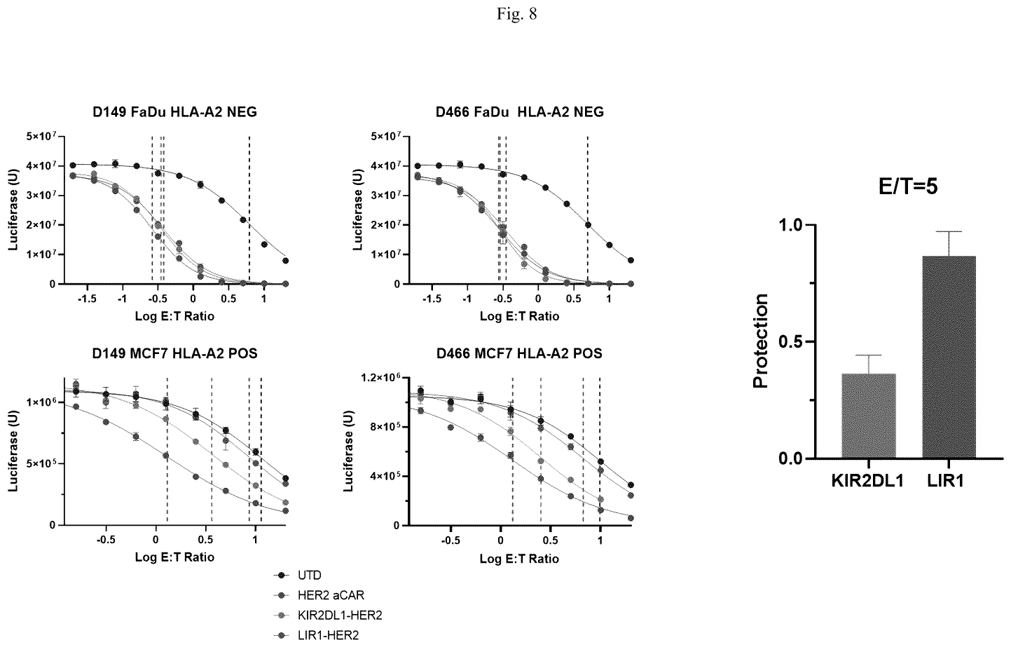

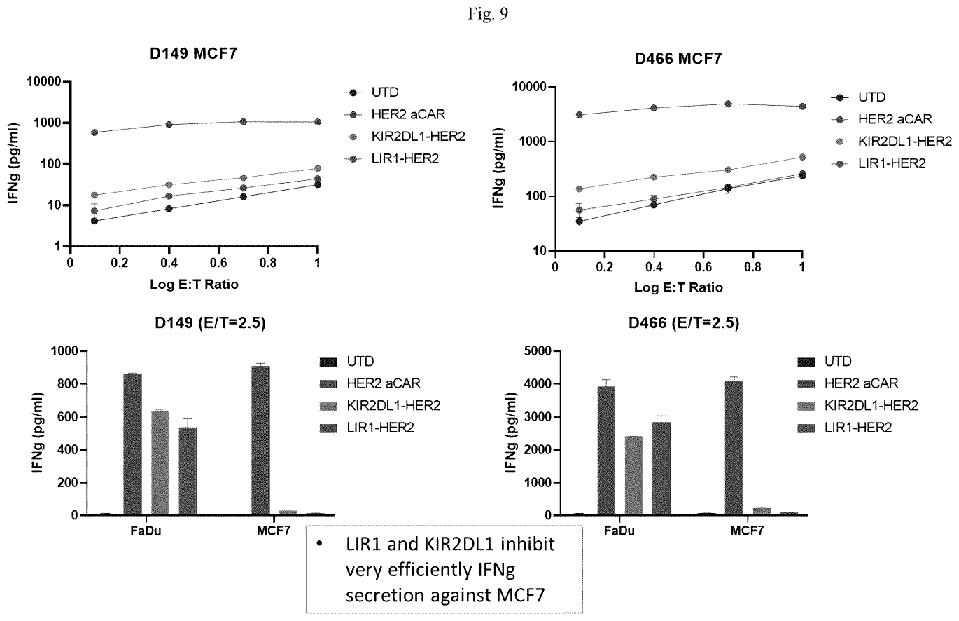

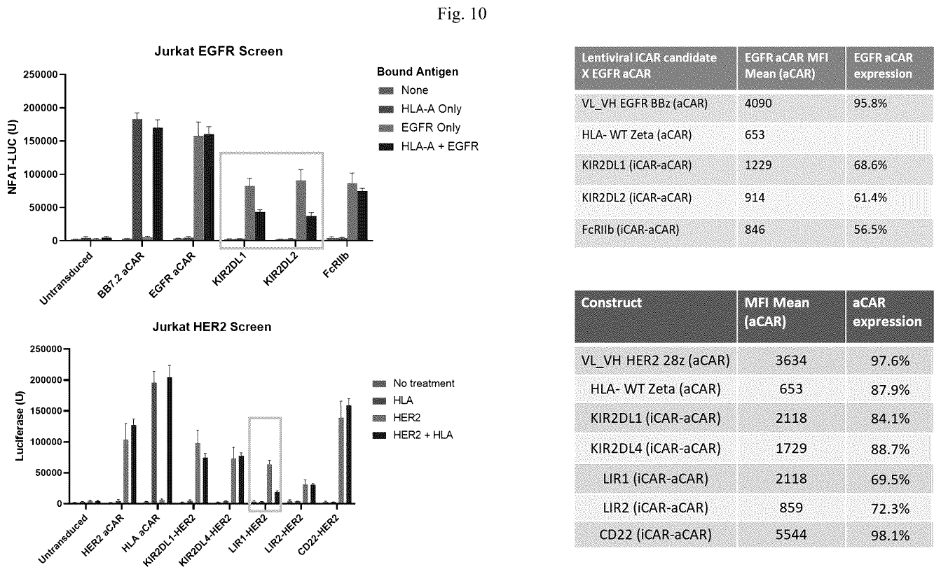

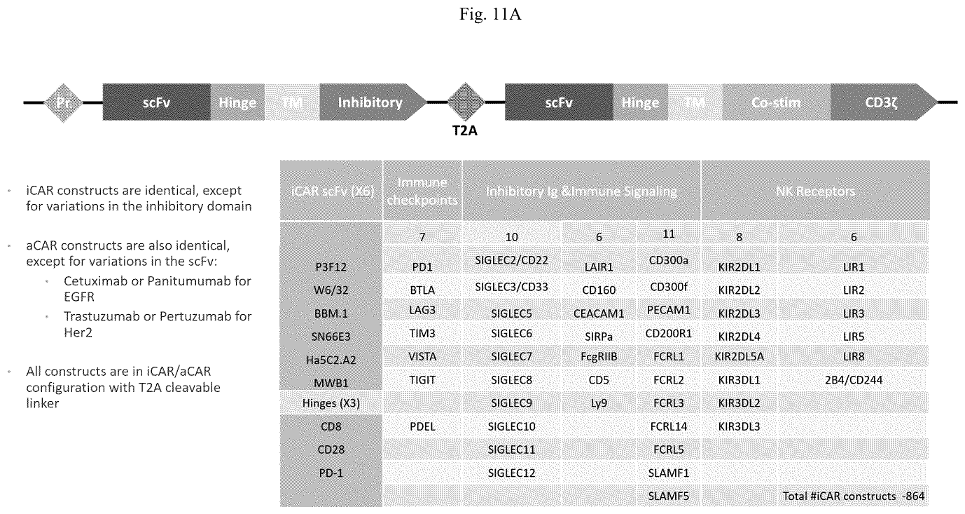

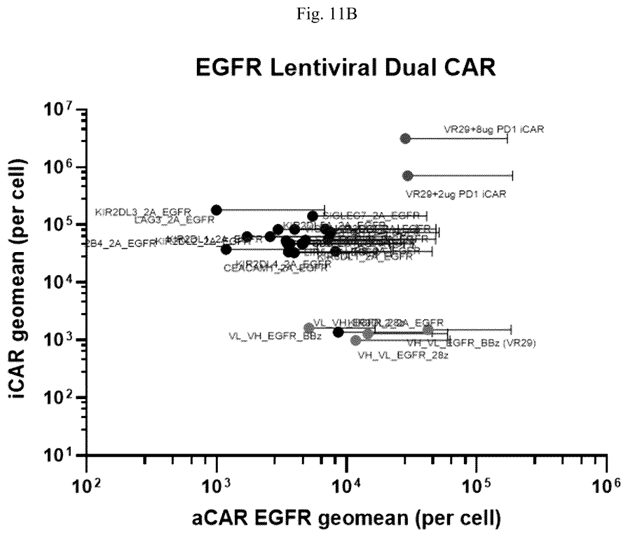

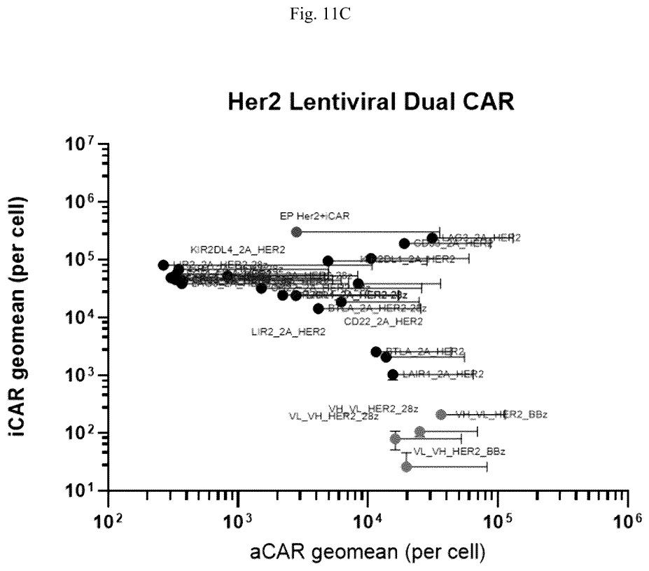

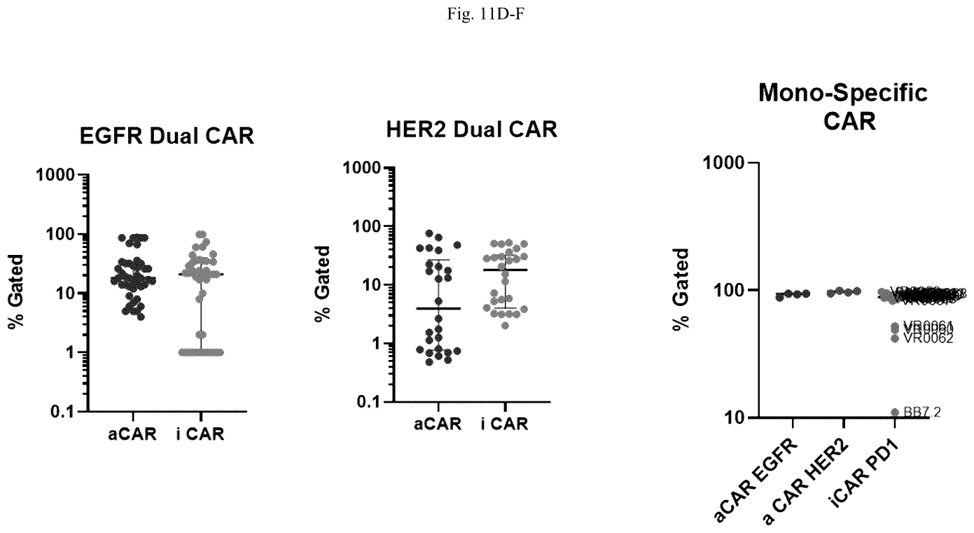

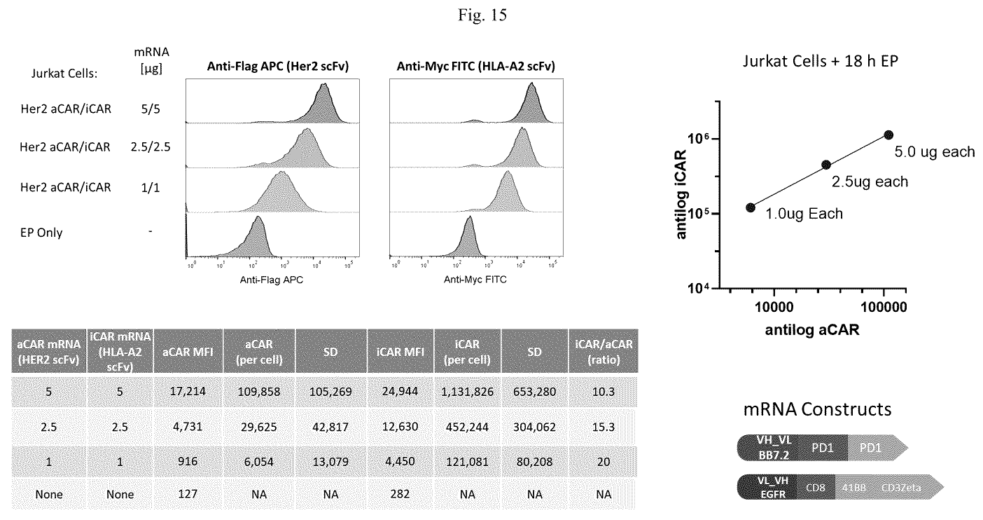

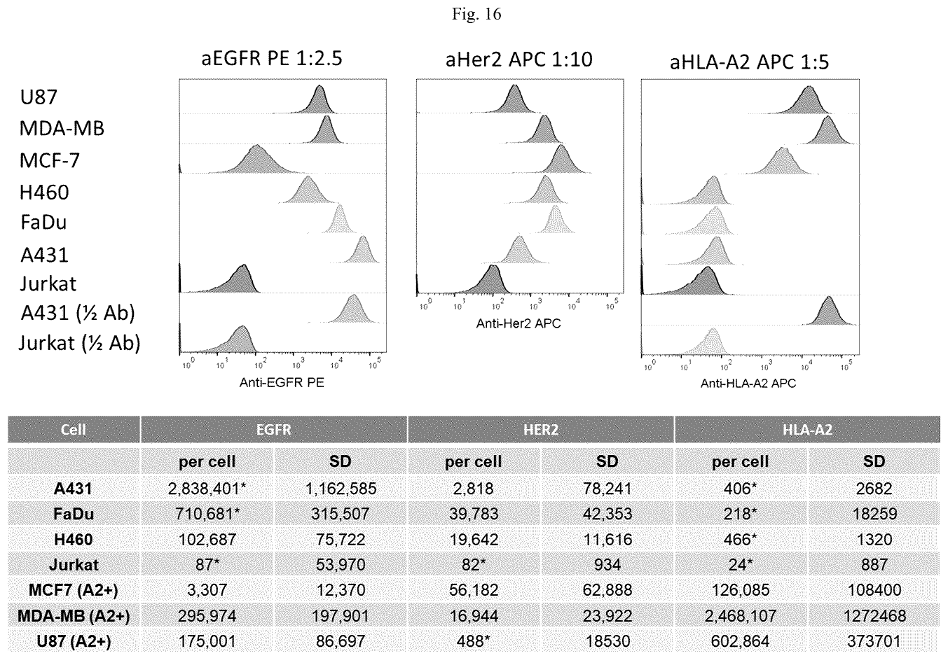

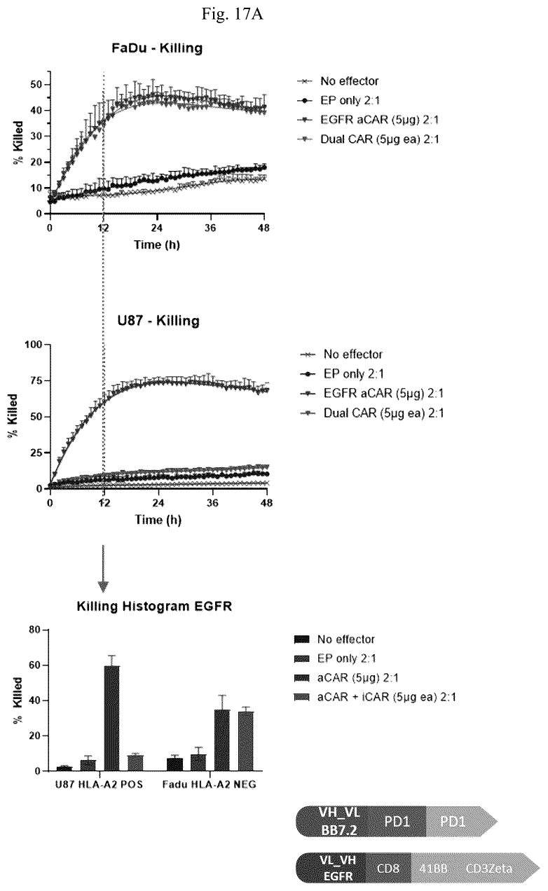

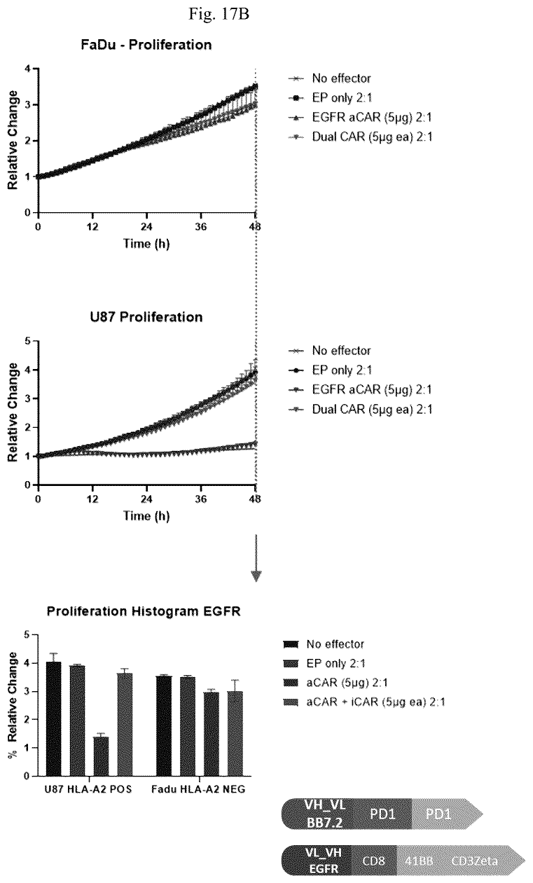

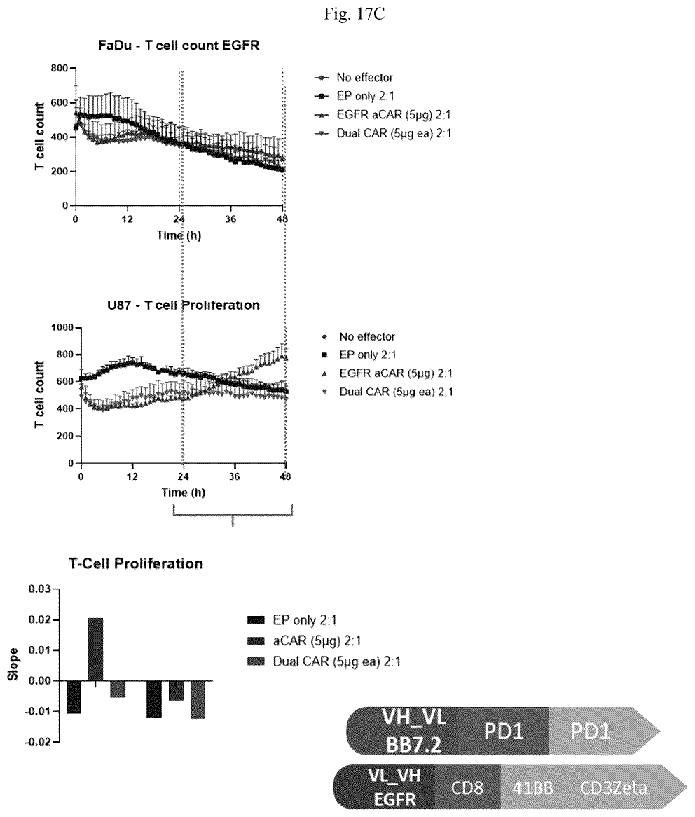

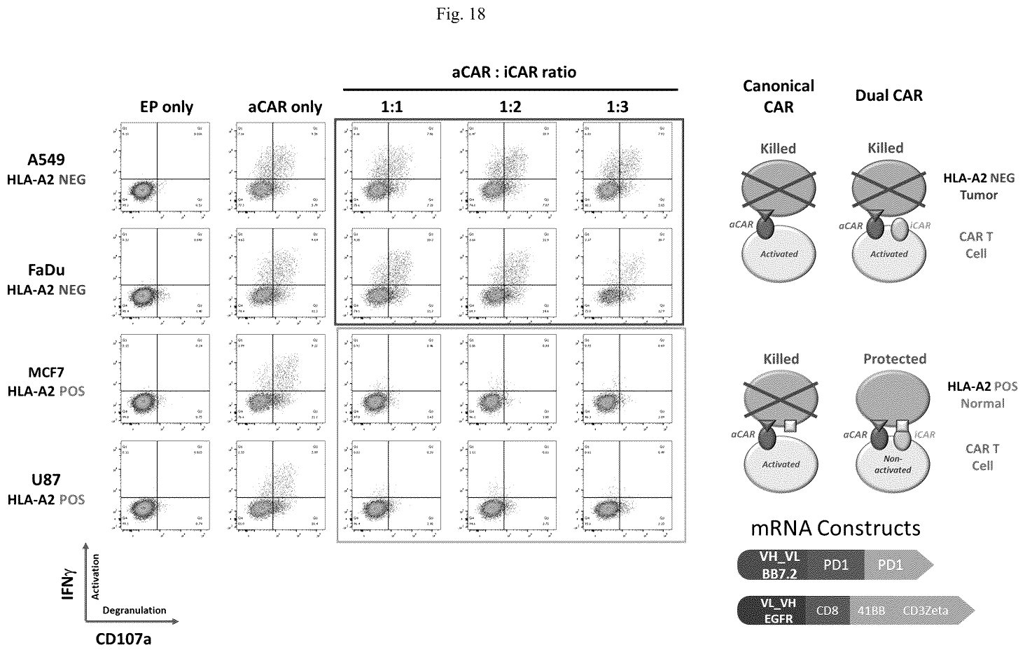

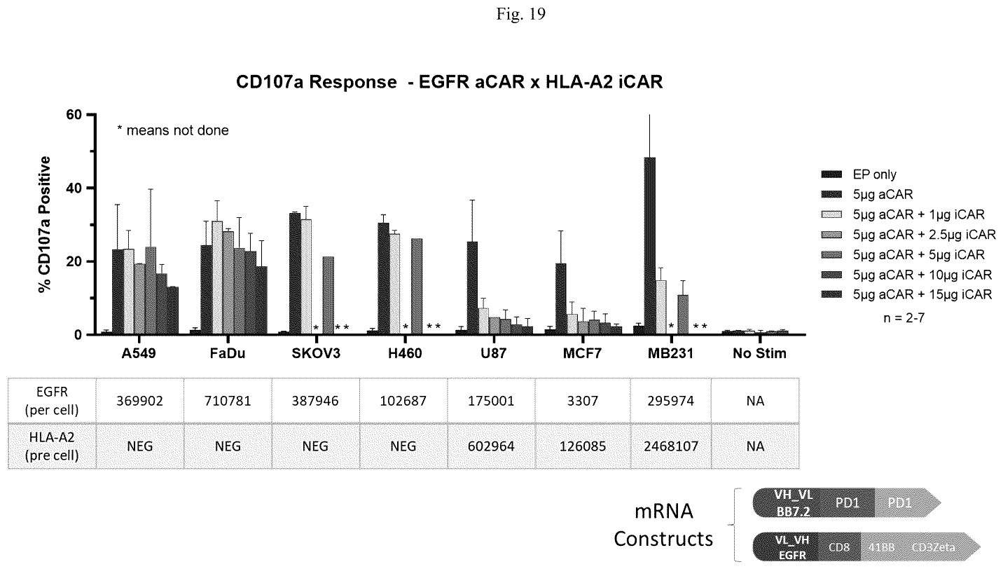

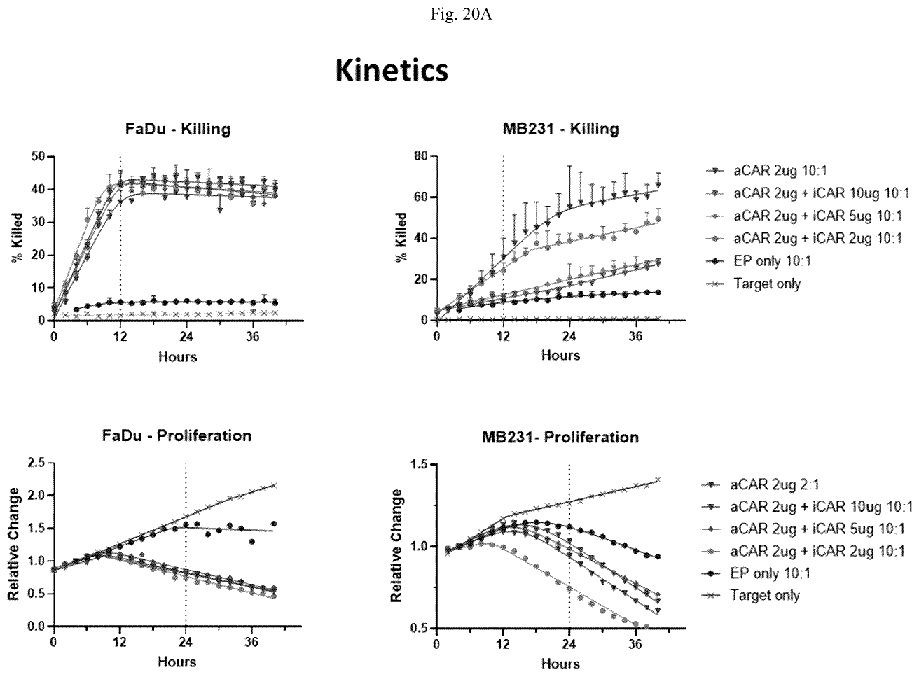

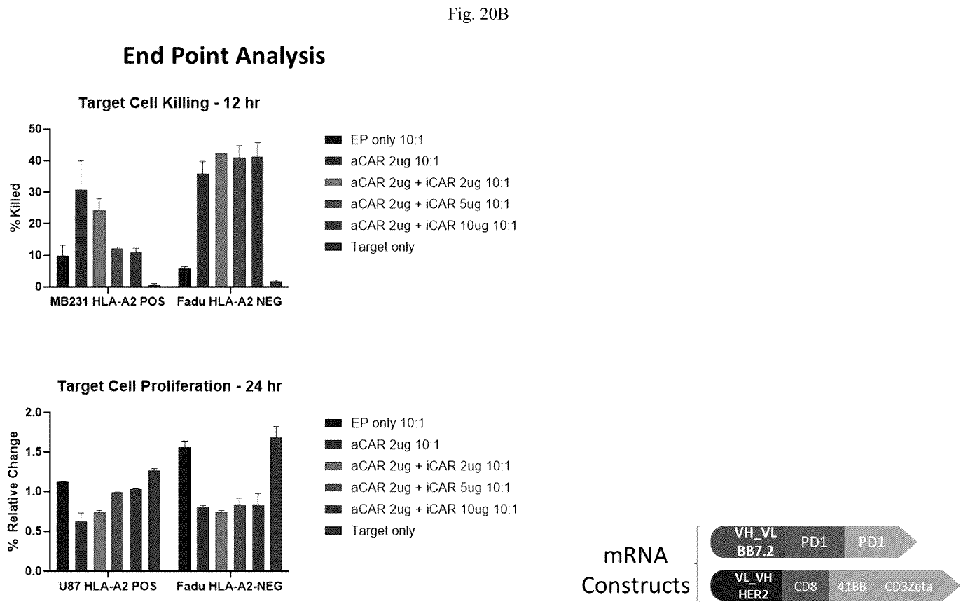

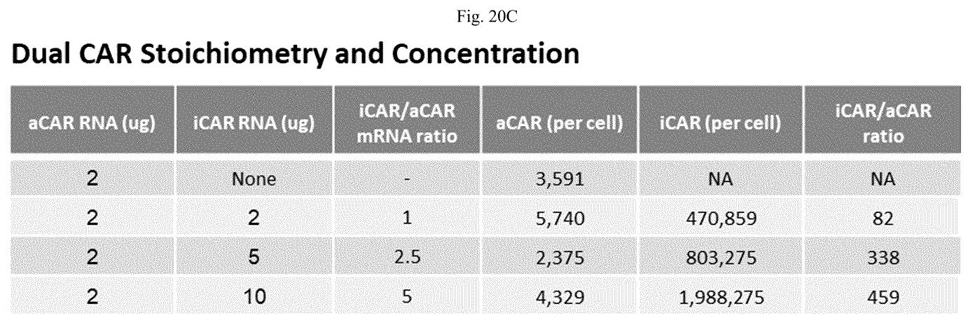

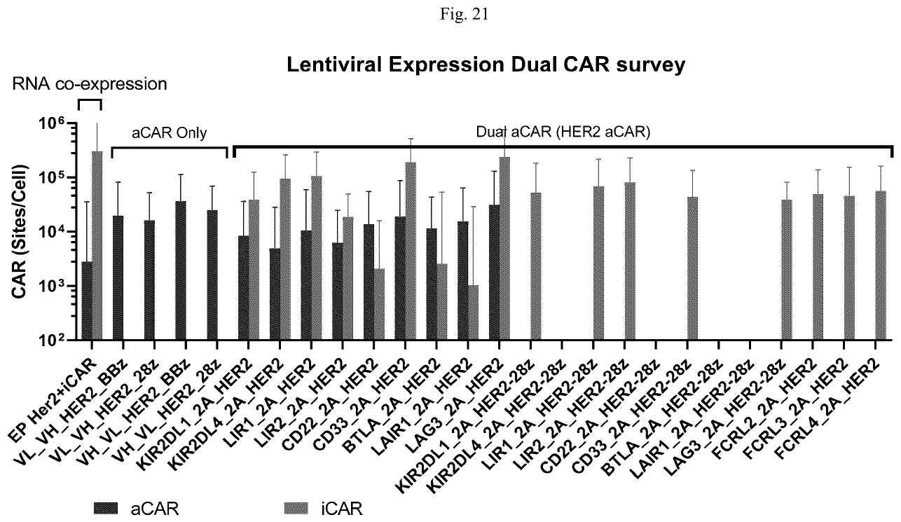

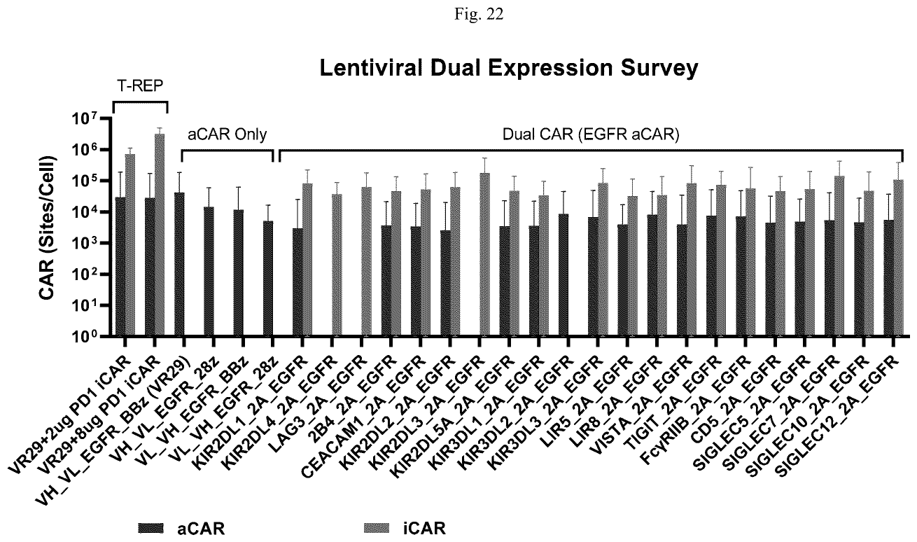

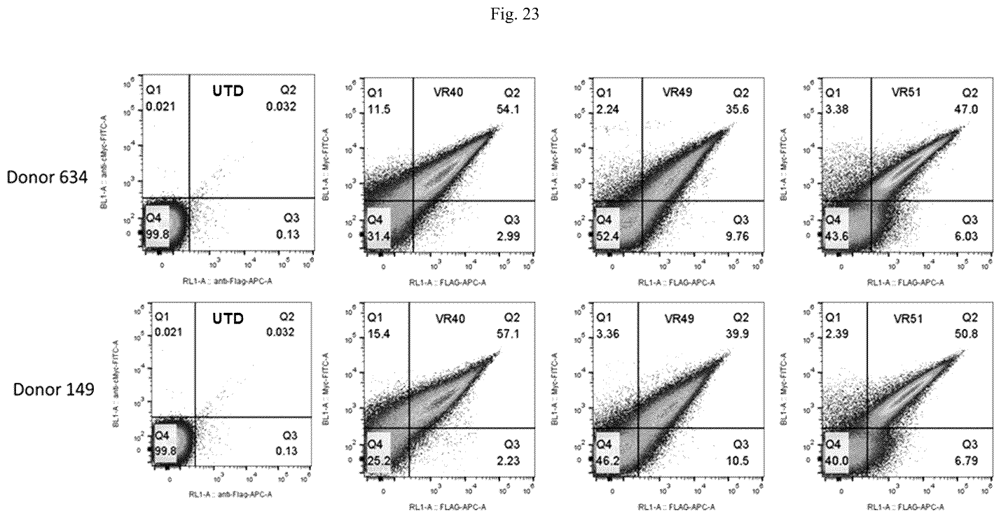

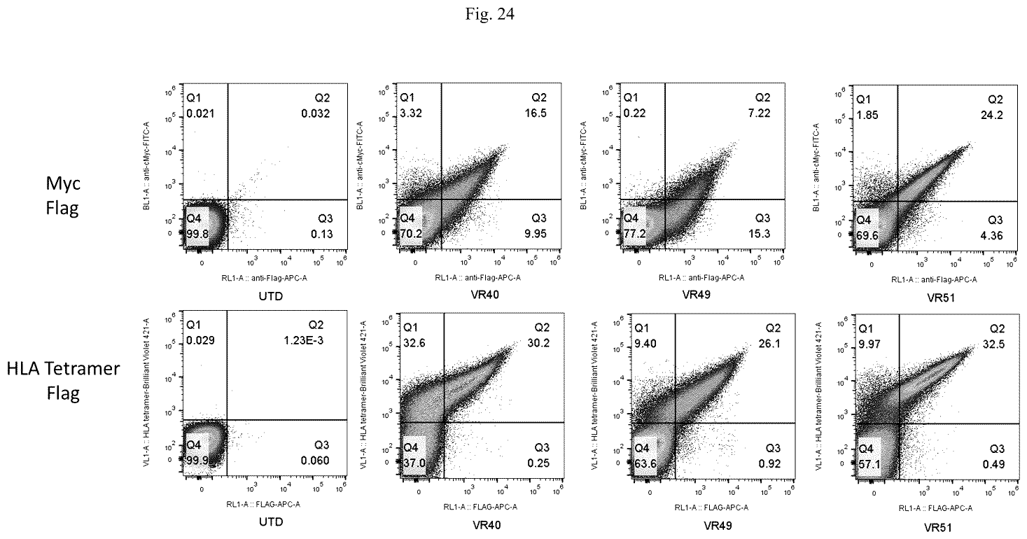

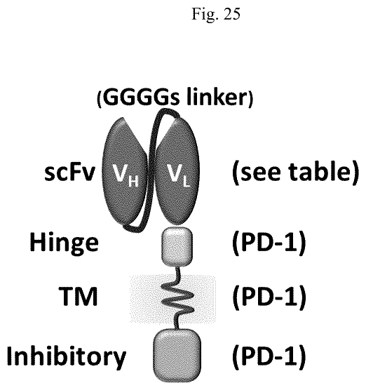

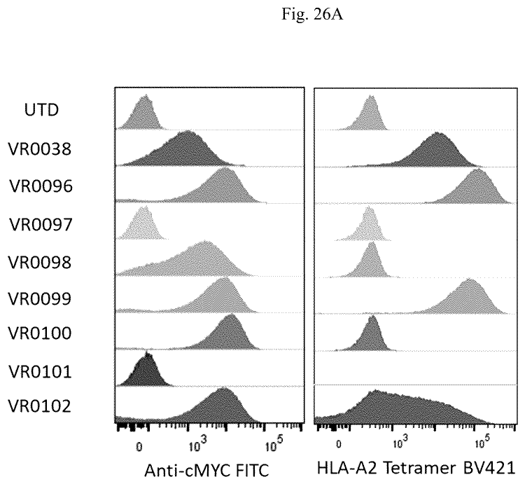

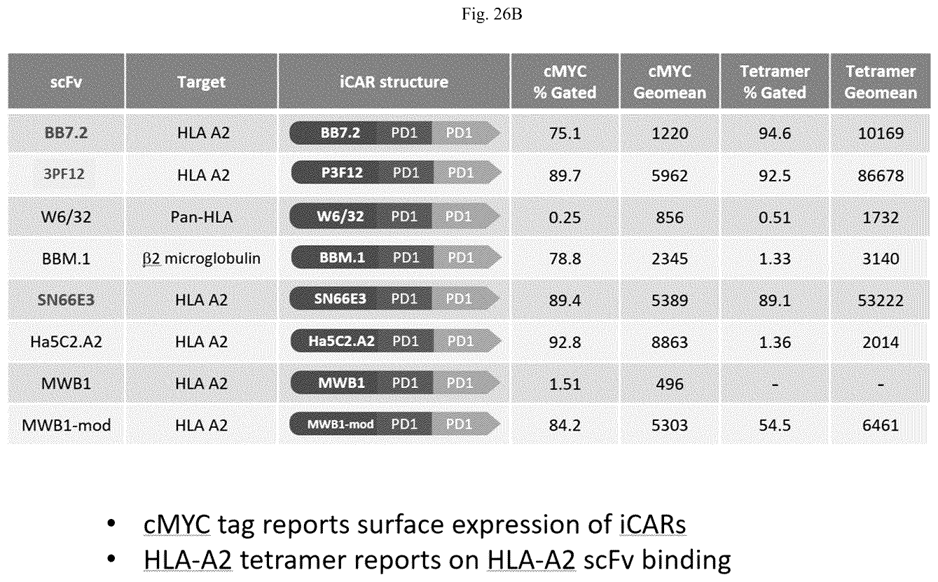

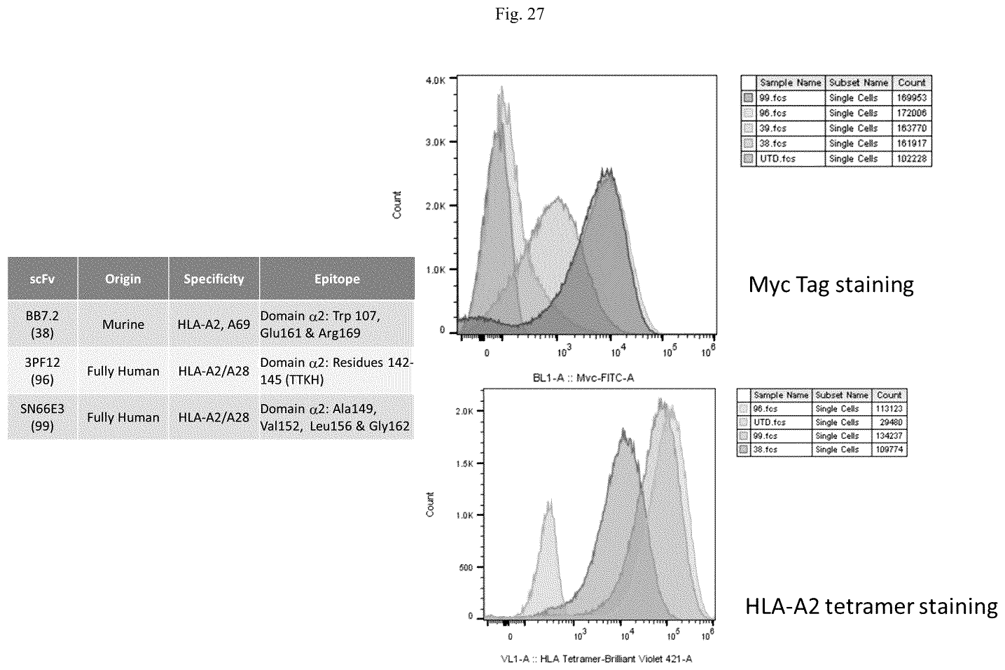

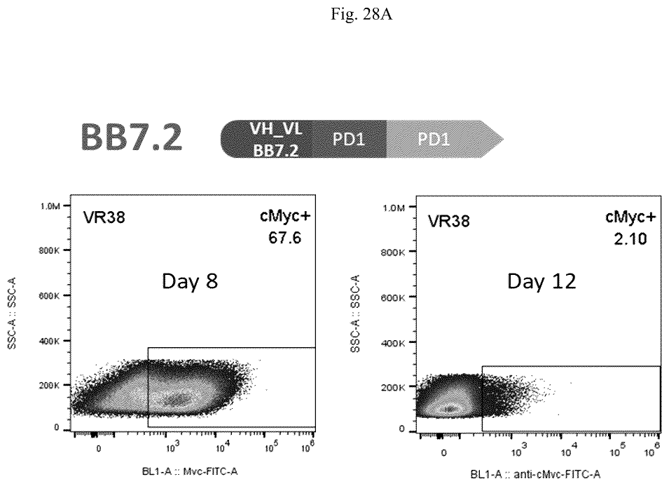

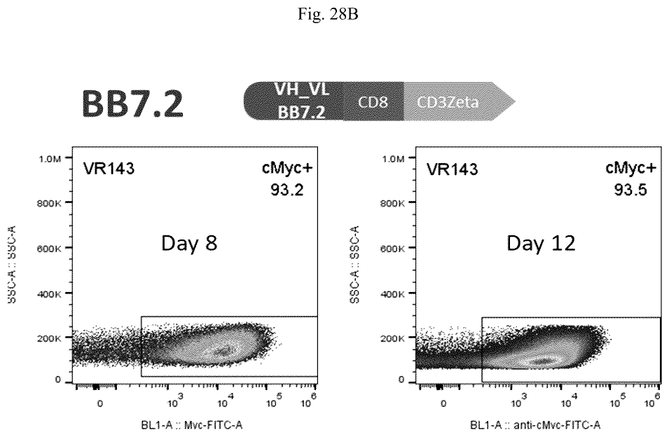

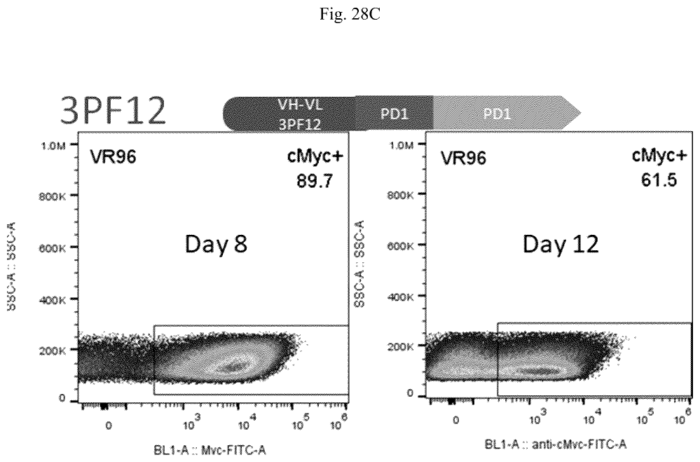

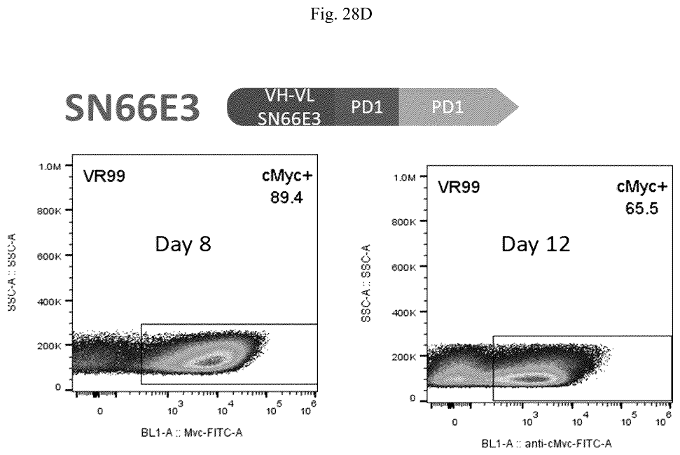

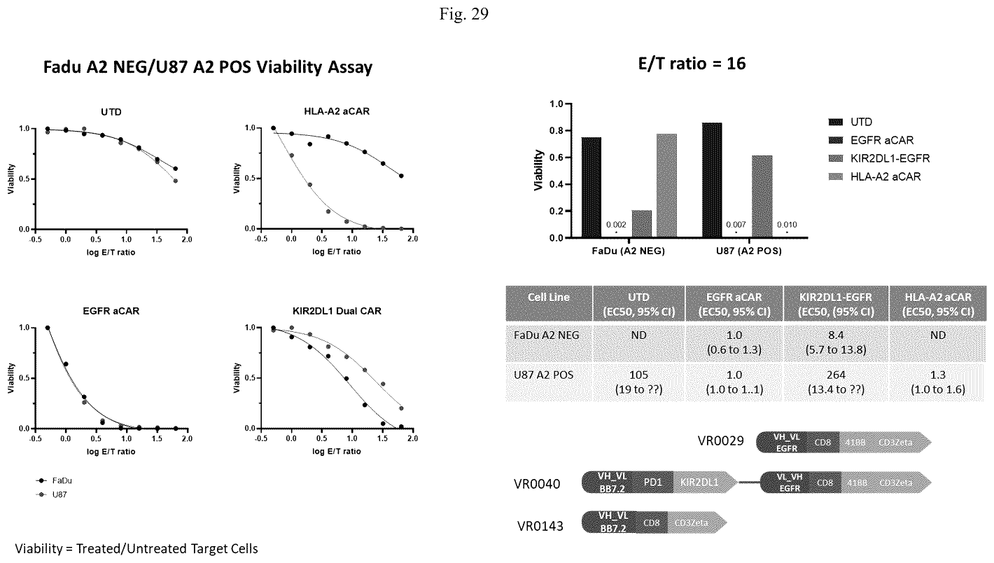

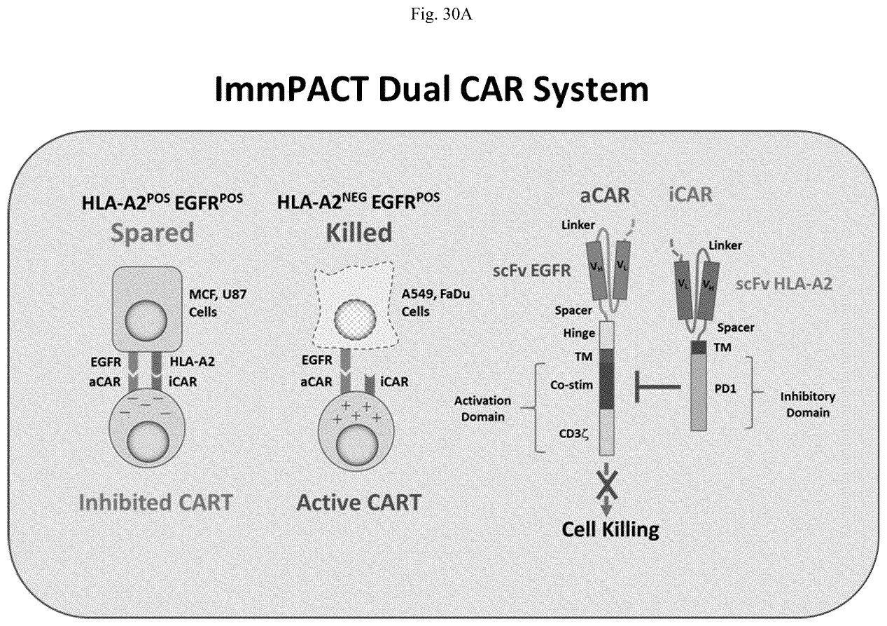

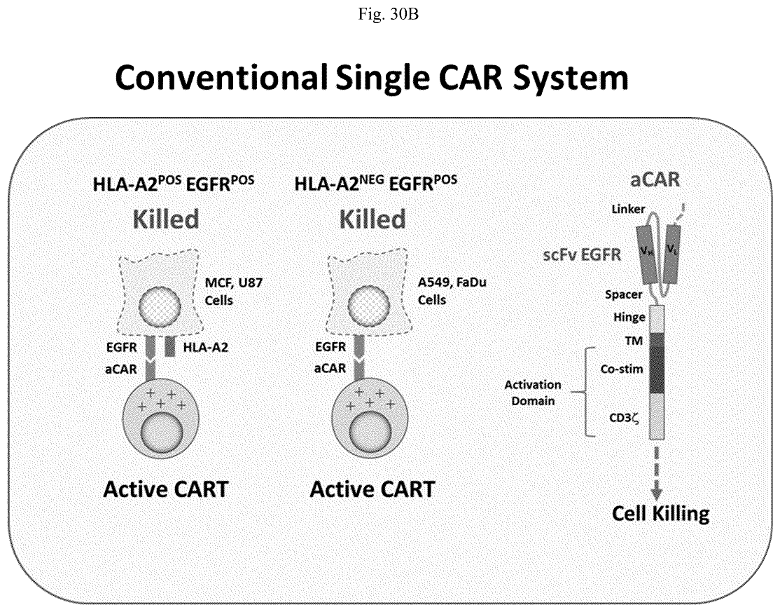

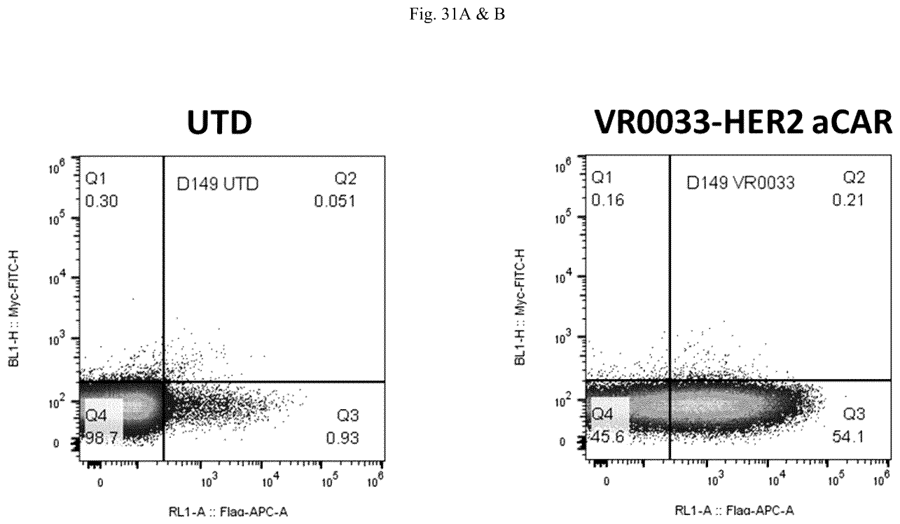

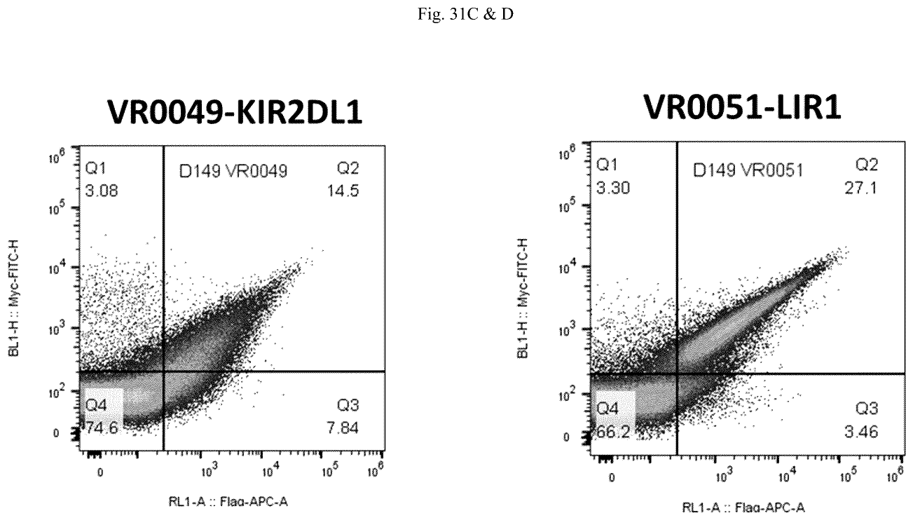

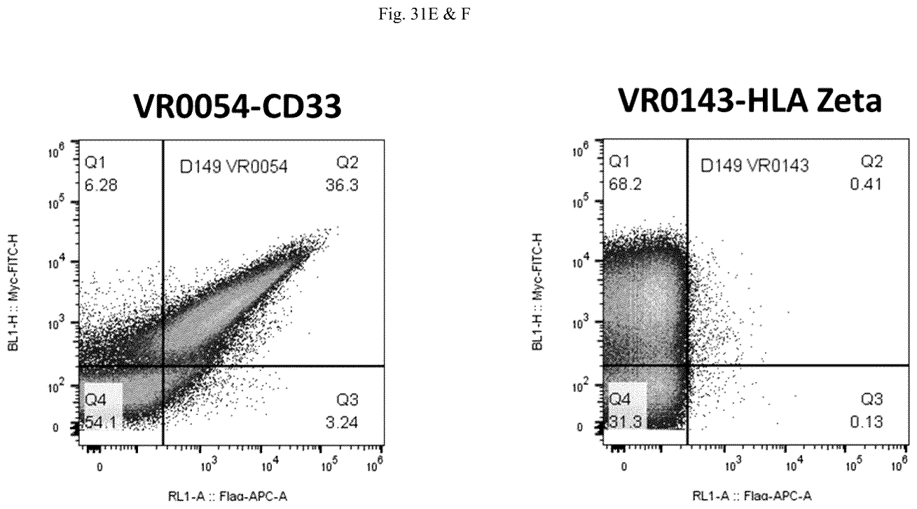

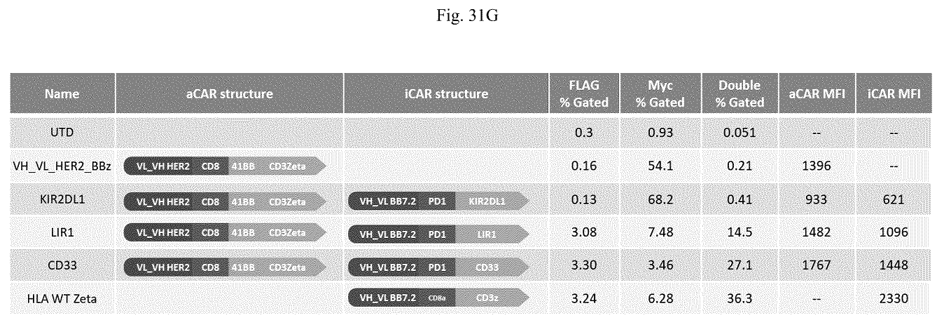

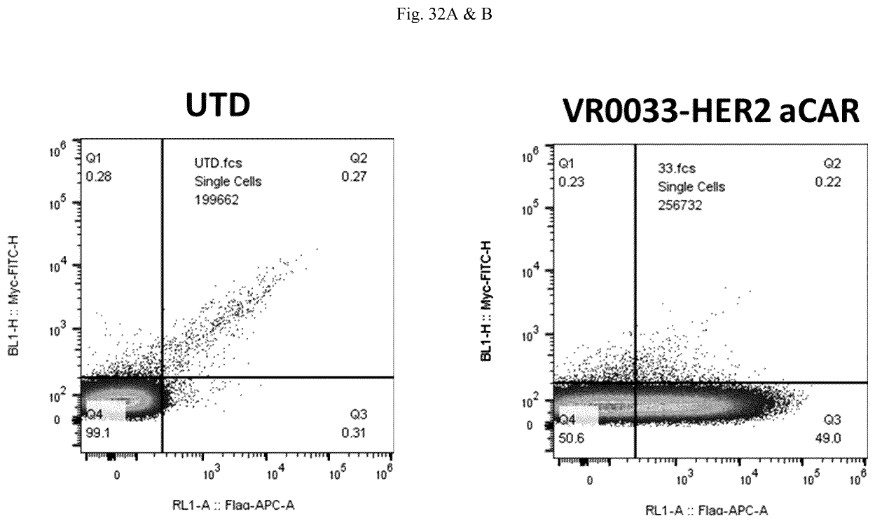

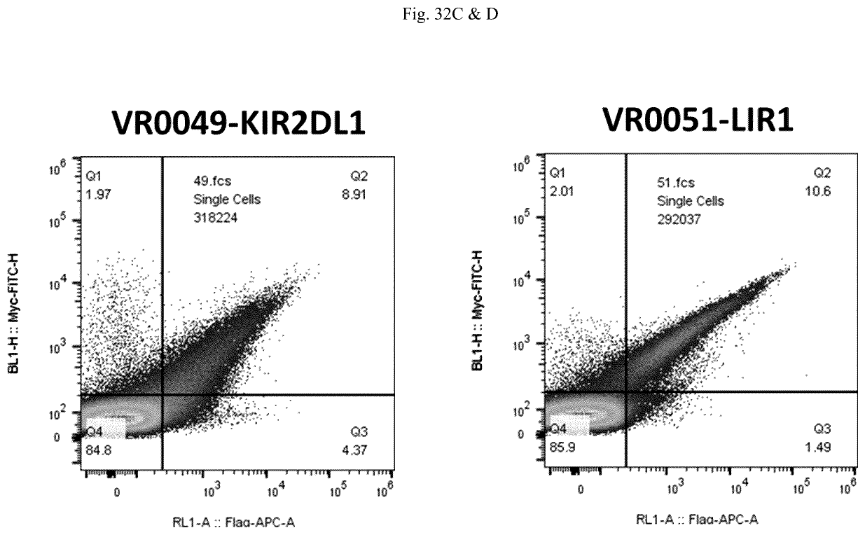

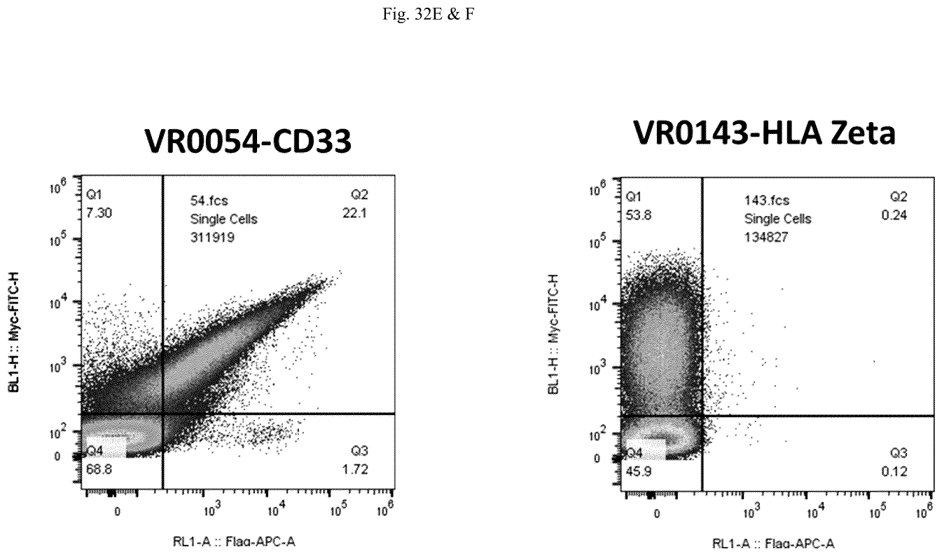

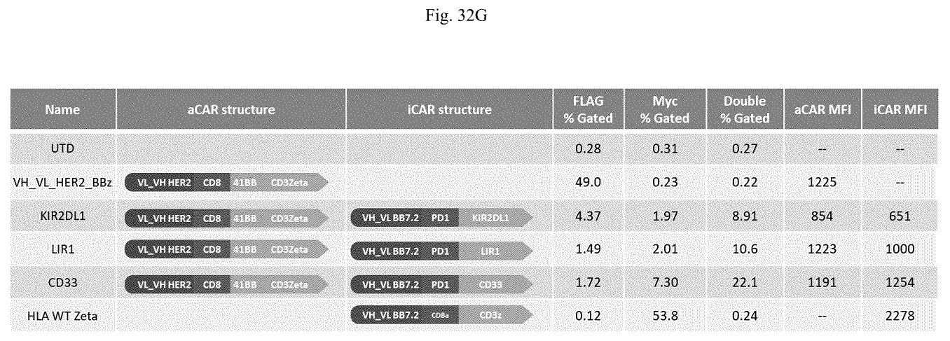

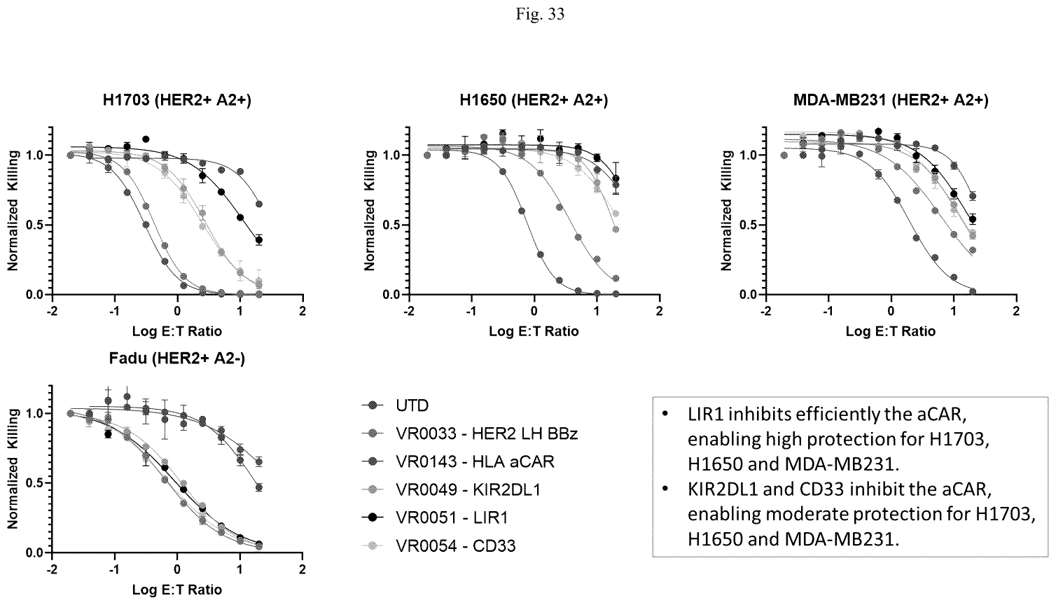

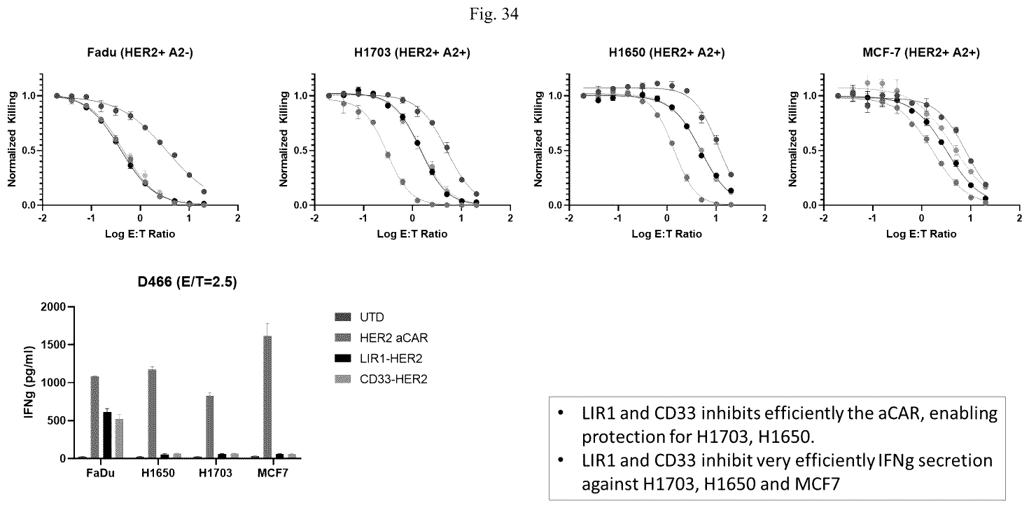

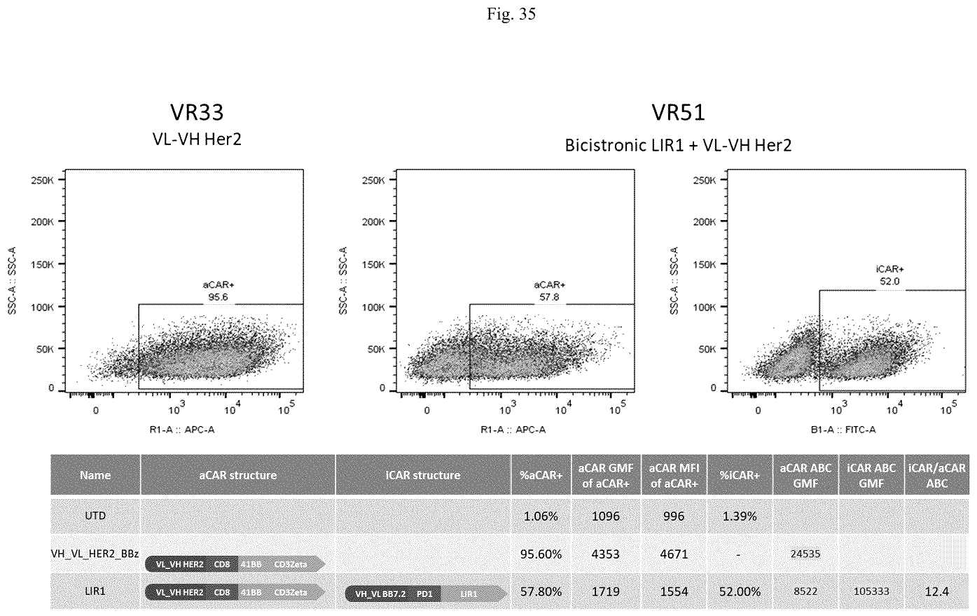

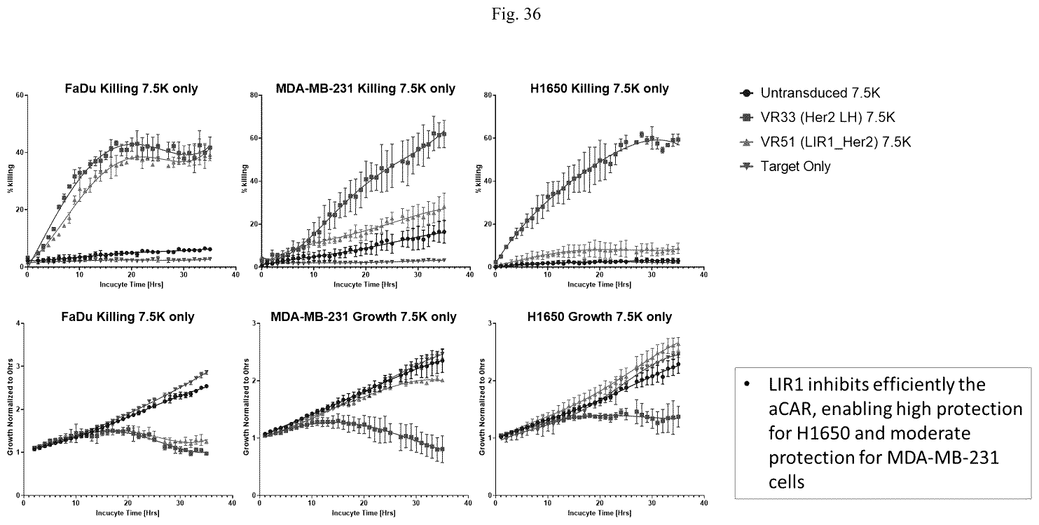

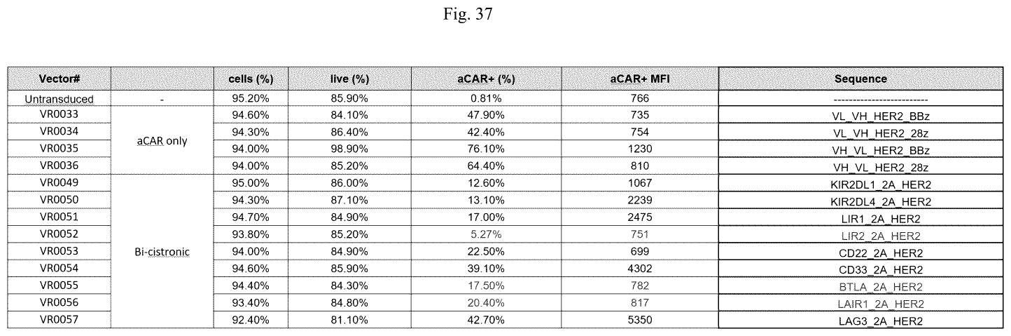

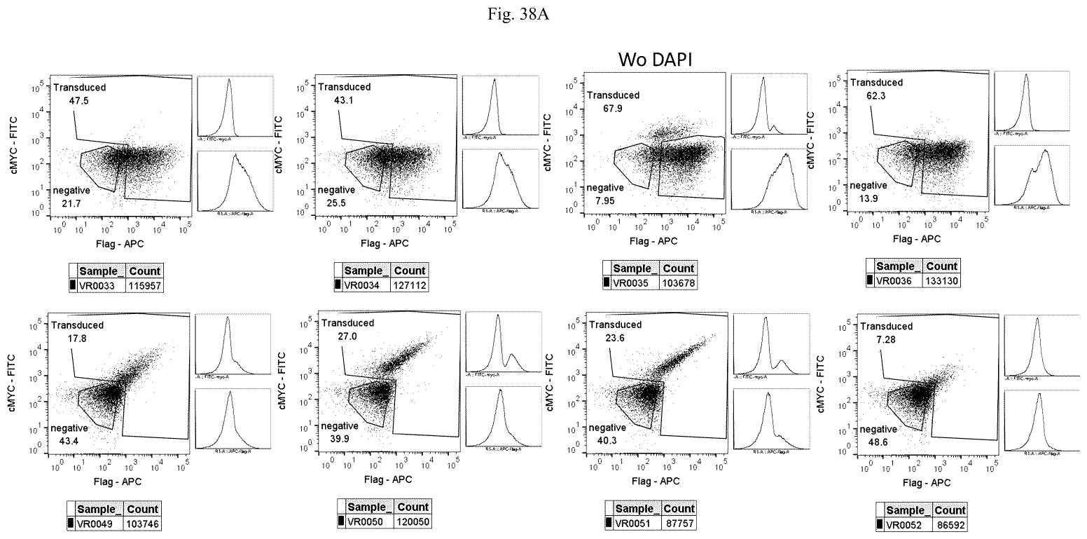

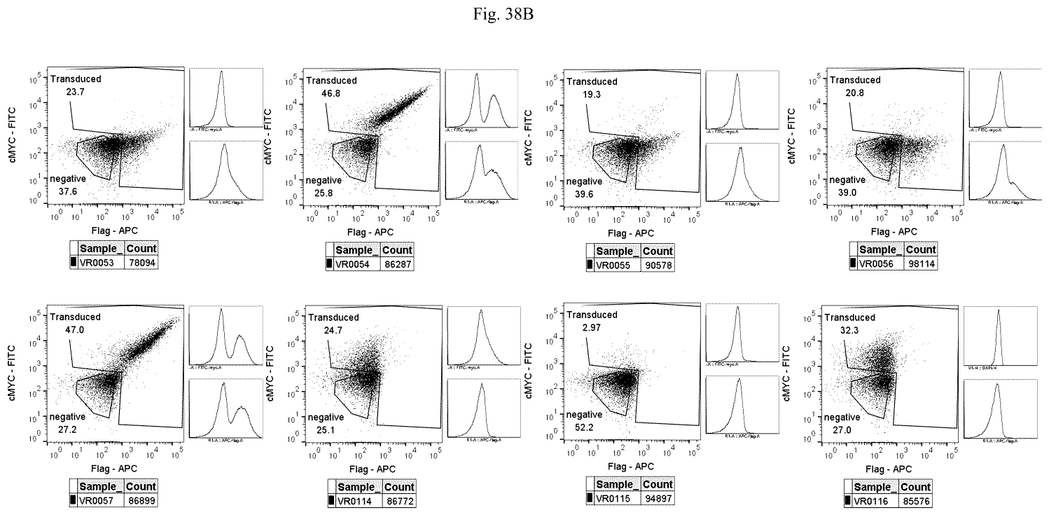

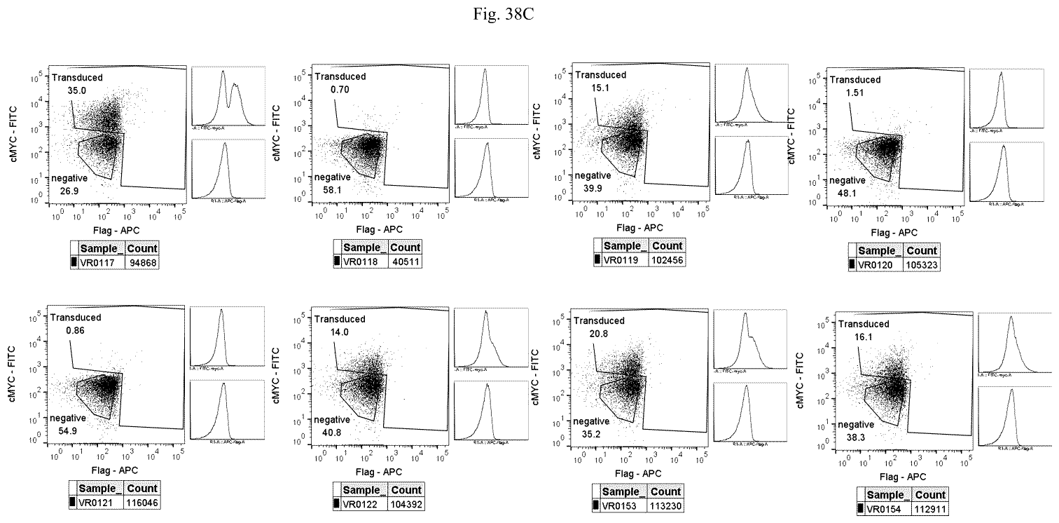

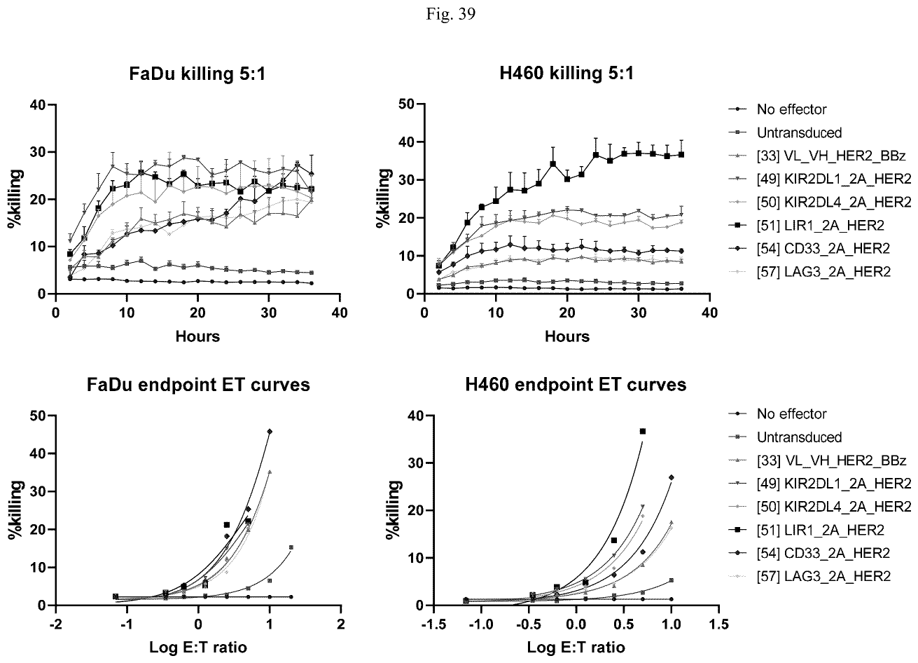

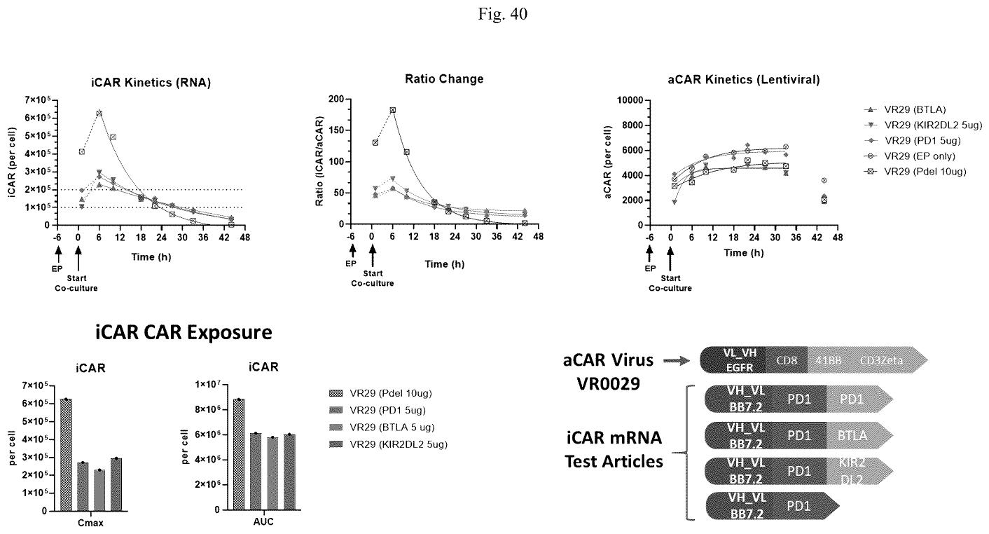

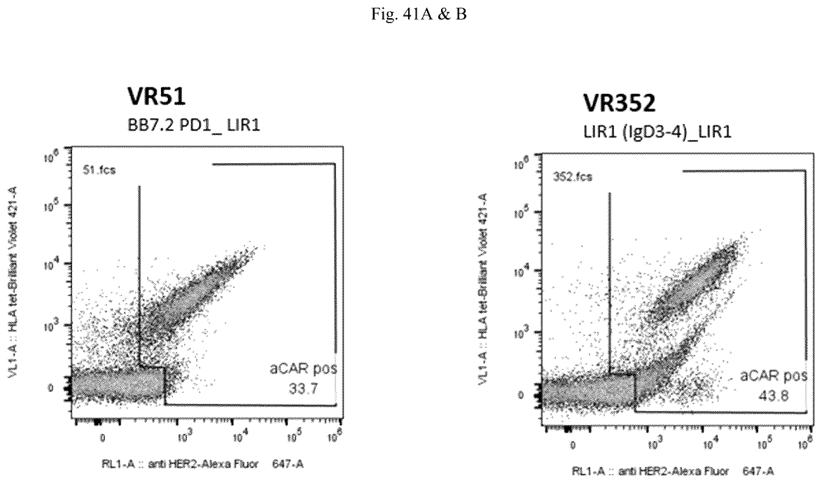

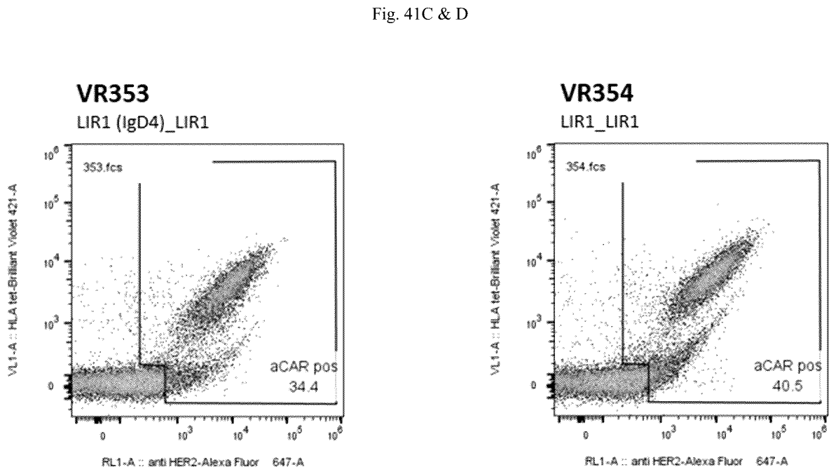

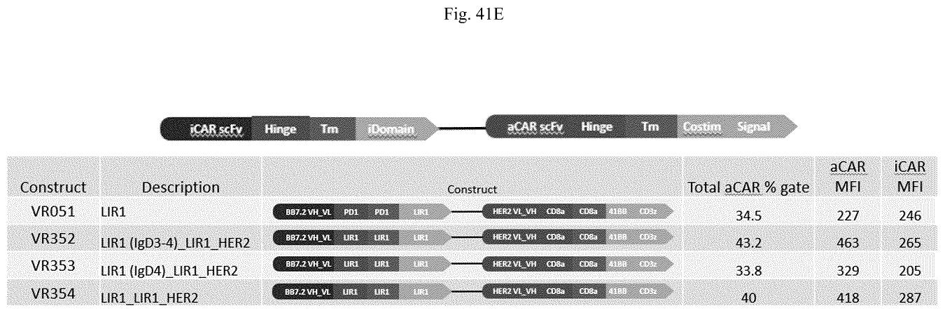

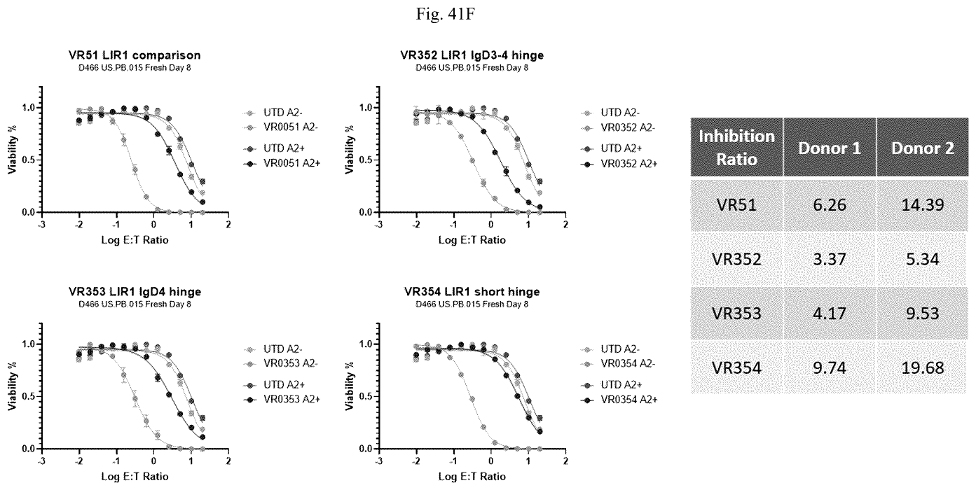

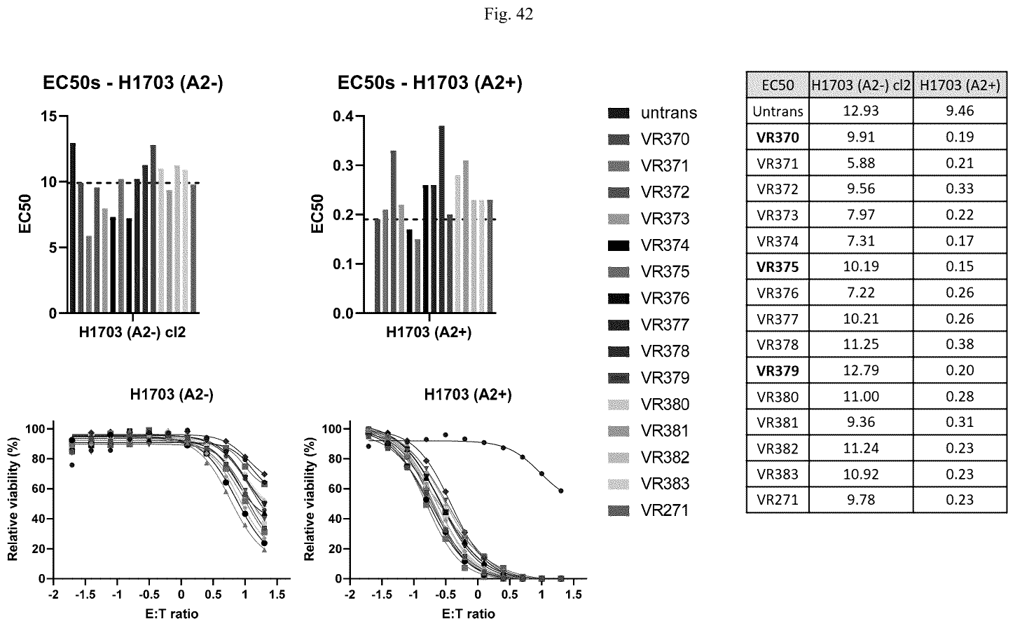

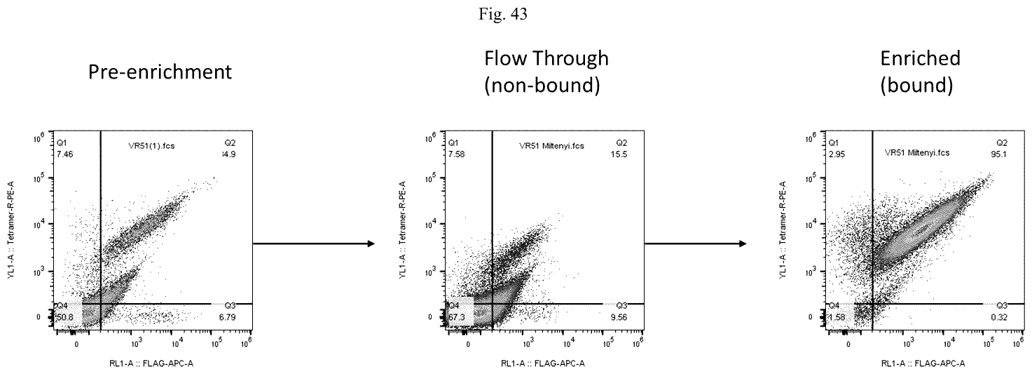

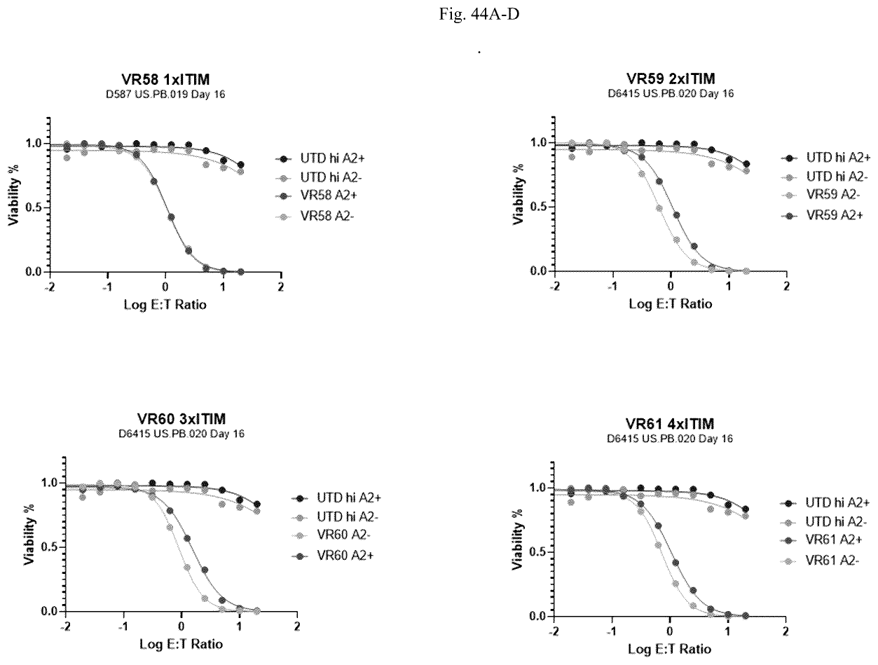

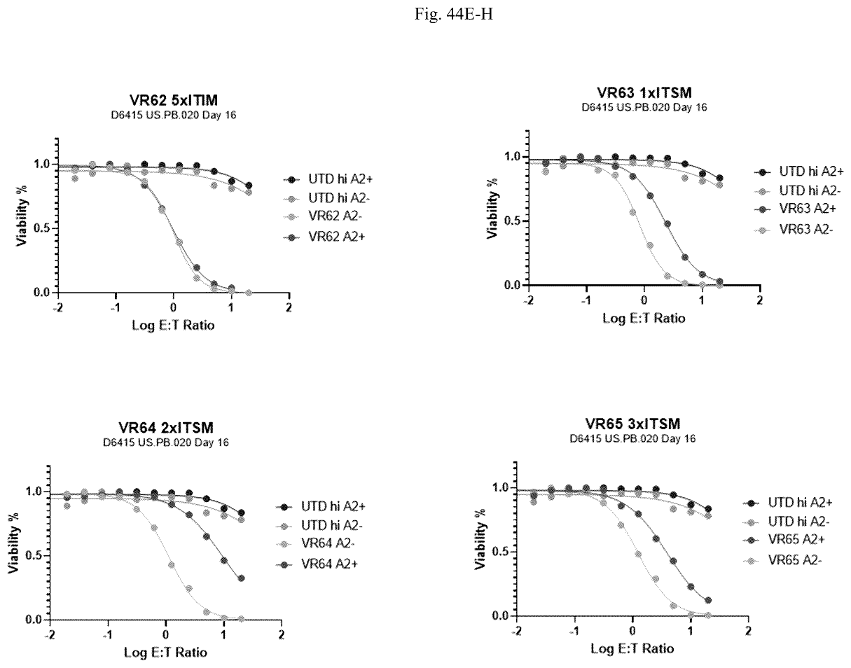

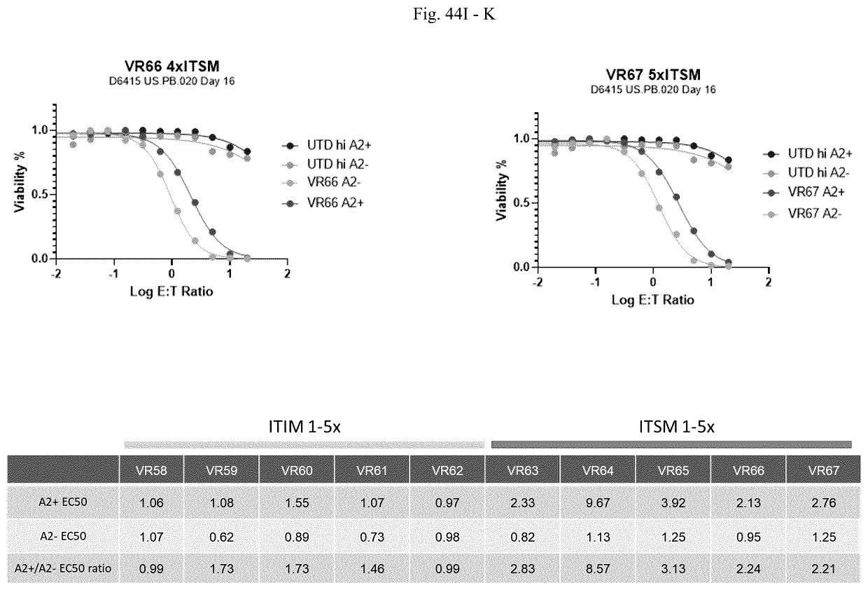

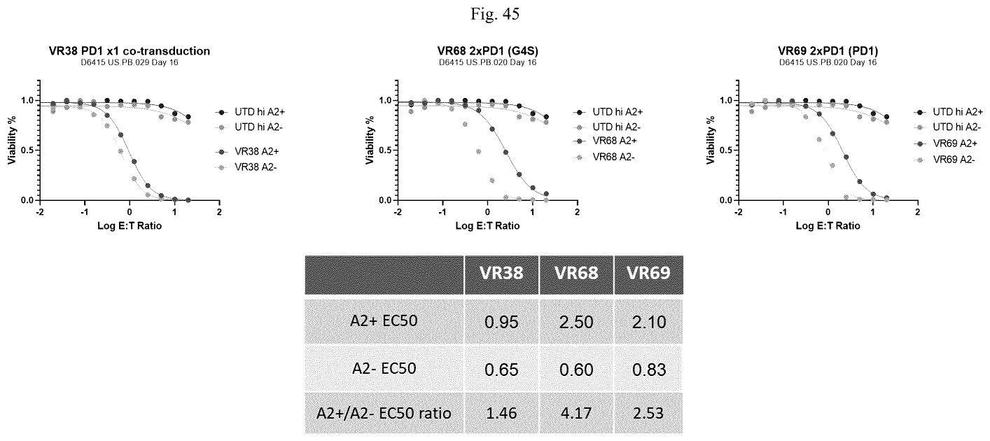

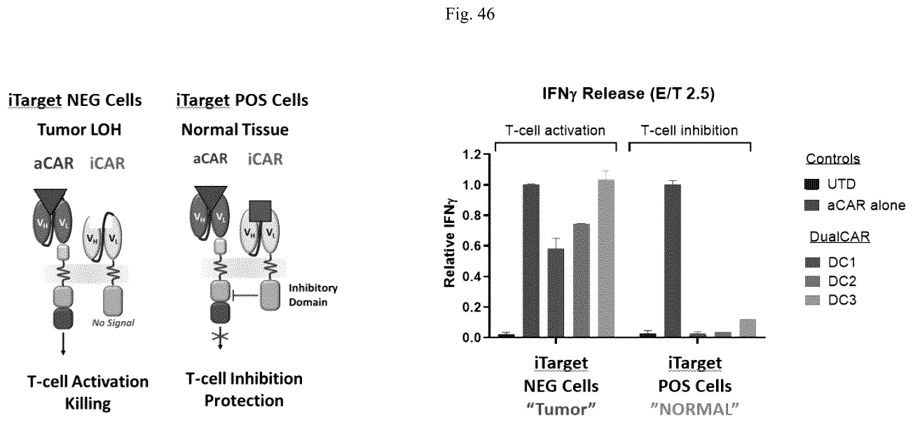

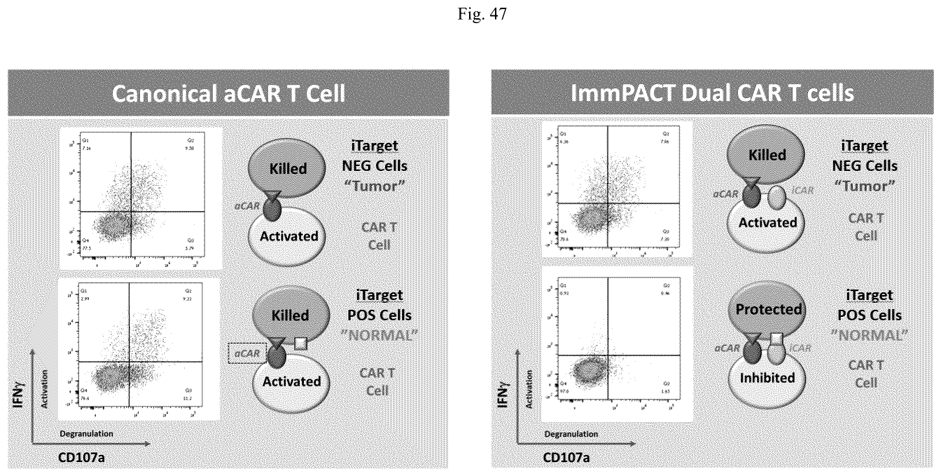

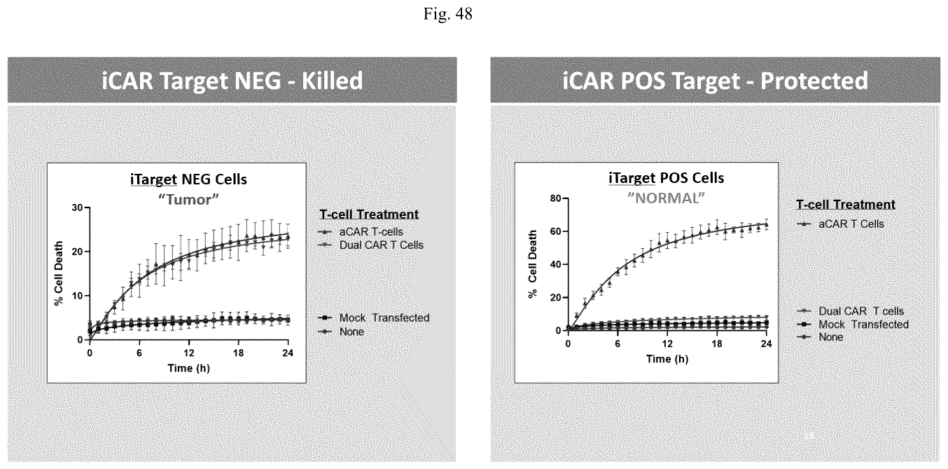

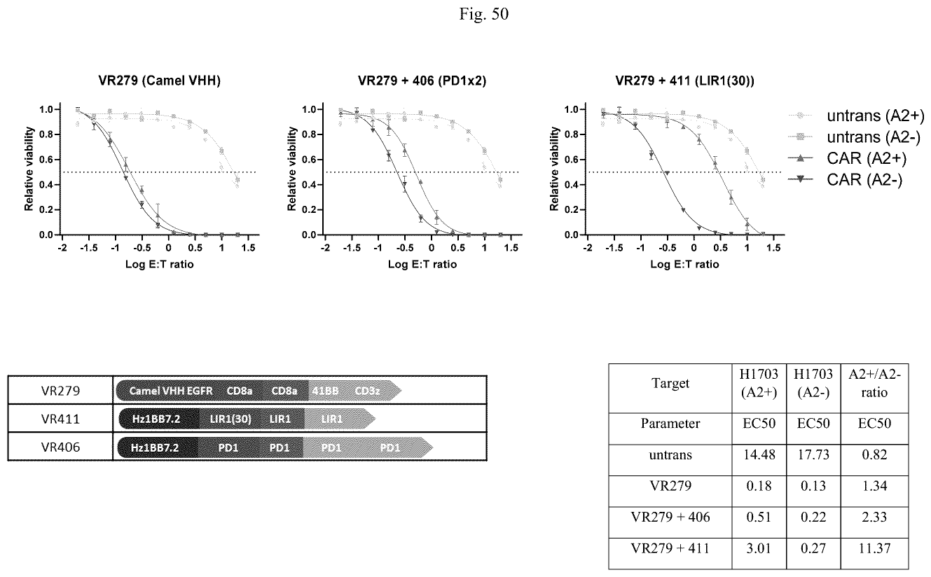

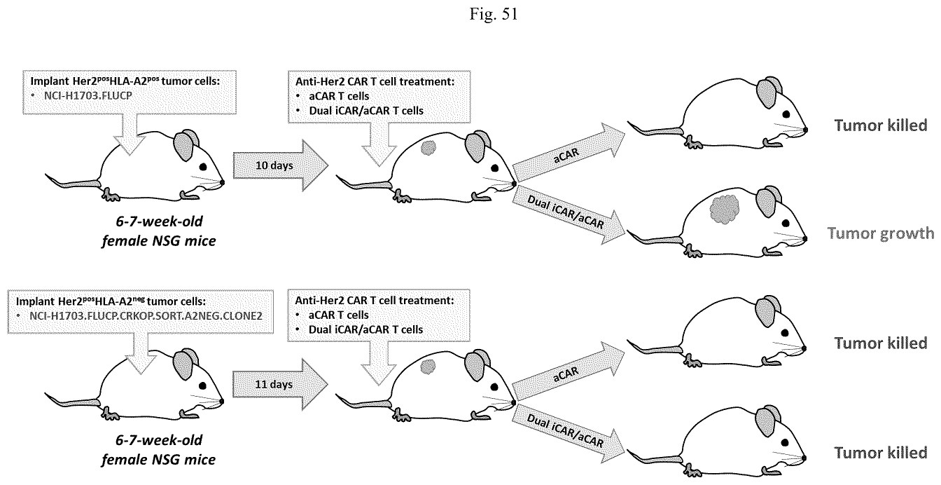

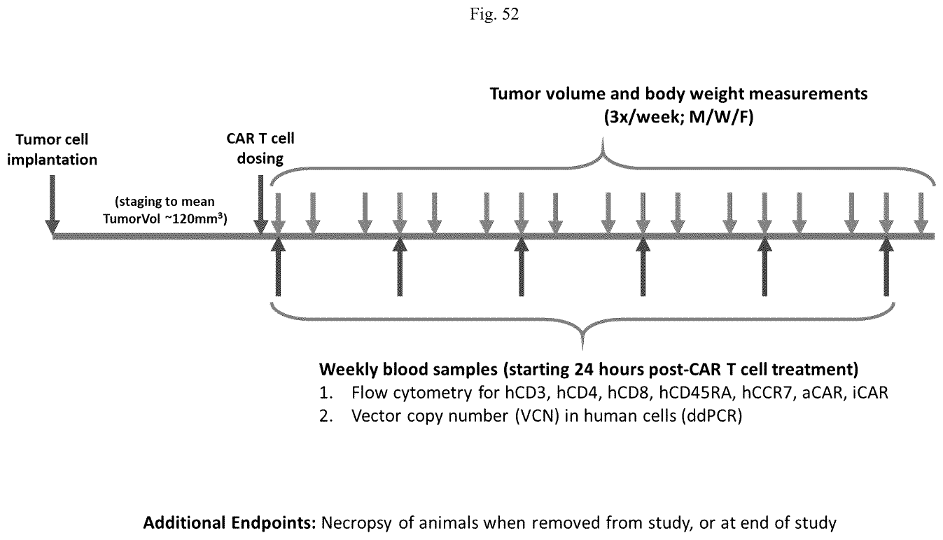

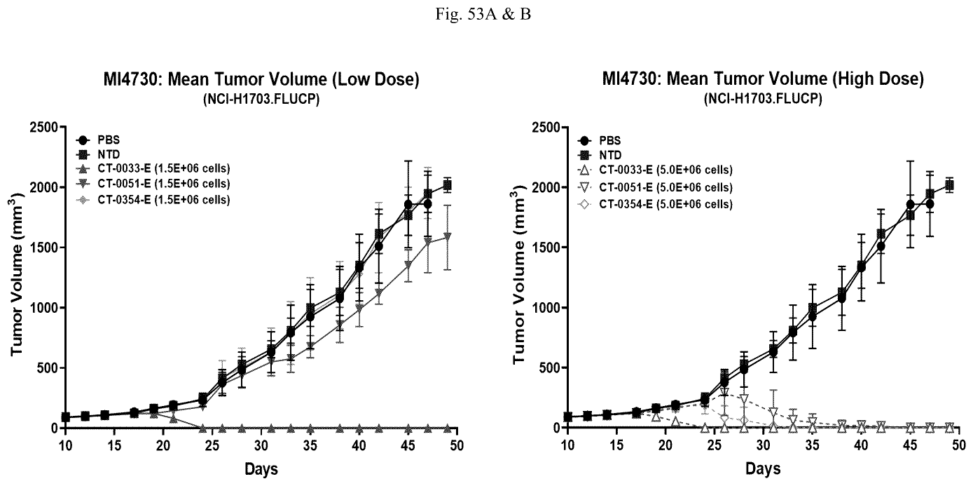

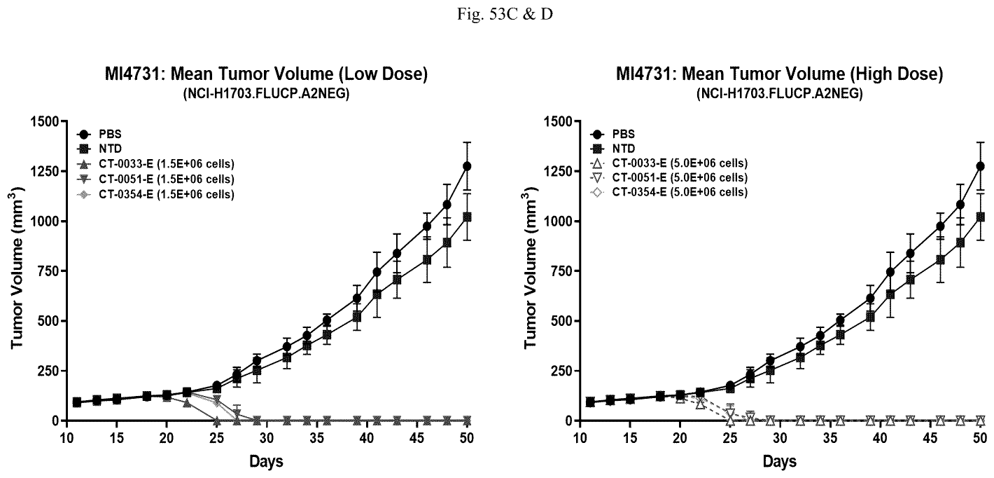

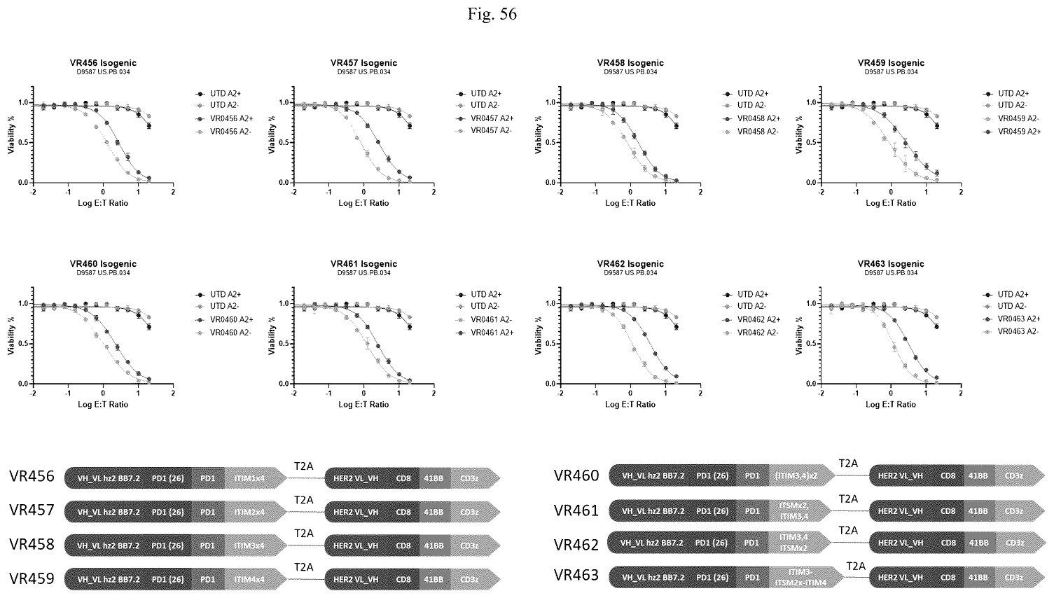

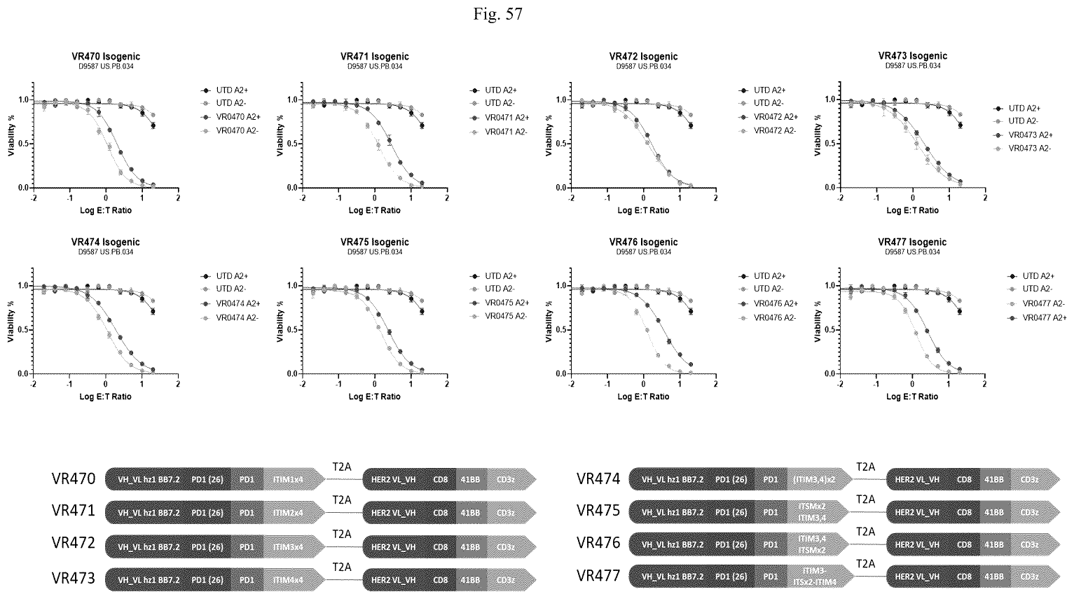

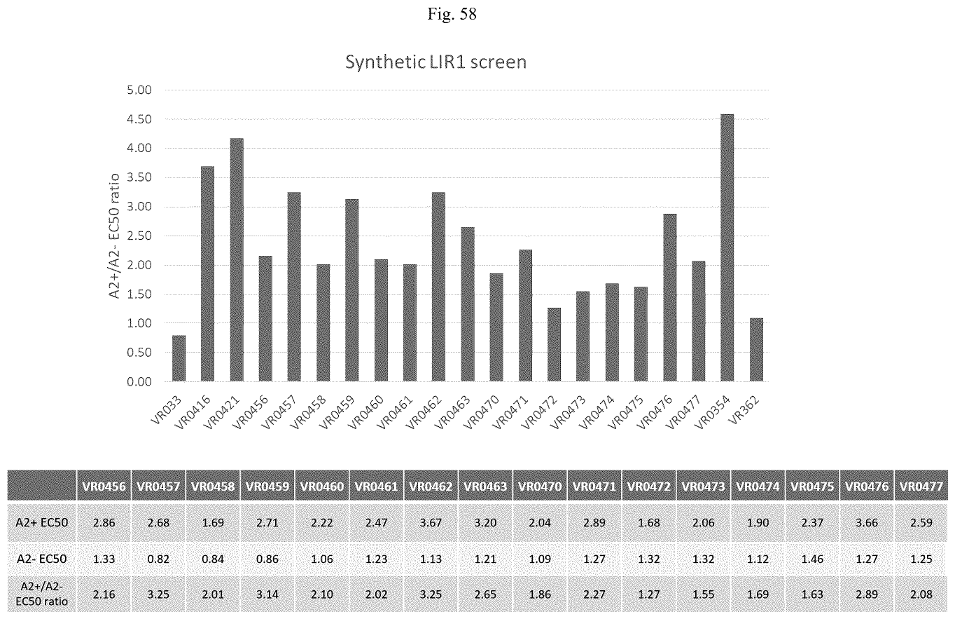

Bicistronic iCAR/aCAR constructs have been developed and preliminary testing performed in order to prepare and examine these constructs for use as cancer therapeutics. See, FIGS. 1-59 as well as Tables 1-22 for illustrative design and evaluation of examples of iCAR and aCAR constructs as described herein, as well as sequences thereof.

### Materials and Methods

mRNA Transcription In Vitro

Appropriate plasmids were linearized using SpeI or BamHI restriction enzymes. Linear plasmid was used to transcribe in-vitro mRNA using T7mScript Standard mRNA Production System (CELLSCRIPT, Madison, U.S.A.). The concentration and quality of the mRNA were assessed by spectrophotometry. Preparation was according to manufacturer's protocol.

PBMC Purification

Leukocyte enriched samples were acquired from The Sheba Medical Center blood bank, diluted with equal volumes of PBS and loaded on Ficoll-Paque PLUS (GE Healthcare) for density-based cell separation. Preparation was according to manufacturer's protocol. Mononuclear cells were collected from the plasma/Ficoll interface, washed several times and resuspended in Cryostor CS10 (Merck).

PBMC Culture and Transduction

PBMCs were thawed and seeded at a density of 1×10⁶ cells/ml in LymphoOne medium (Takara-Bio, Kusatsu, Japan) supplemented with 100 U/ml IL2 (Miltenyi Biotech, Bergisch Gladbach, Germany). The next day concentrated lentiviruses were added at an MOI of 5, 10, or 20 (according to prior calibrations). After 3 days cells were transferred to 24-well G-Rex plates (Wilson Wolf, Saint Paul, MN) containing LymphoOne medium supplemented with 1% human serum (Access Biologicals, Vista, CA) and 100 U/ml IL2. On day 7 post-thaw 100 U/ml IL2 was added, and on day 8 the medium was replaced. Functional assays were typically performed.

mRNA Electroporation

On day 8 or 10 of PBMC's culture, 2×10⁶ cells were washed twice with OptiMEM medium (GibcoBRL, Grand Island, NY). The cells were resuspended in 100 ul OptiMEM containing 1-10 ug mRNA and electroporated in 2 mm cuvette, using NepaGene21 electroporator (Nepa Gene Co., Ltd., Japan) at 200V, 2.5 ms, one pulse or using ECM830 electroporator (BTX.Ltd., US) at 300V, 2 ms, one pulse. The cells were resuspended in 5 ml growth medium and transferred into 6 well plates for further incubation.

### IncuCyte Cytotoxicity Assay

Target cells expressing nuclear-GFP (nGFP) were seeded in black-walled 384-well plates with microclear bottom (Greiner Bio-One, Kremsmunster, Austria), 1.5×10⁴ cells per well, in LymphoOne medium supplemented with 1% human serum. The next day, transduced or electroporated PBMCs were added to the wells at the desired E:T ratio. Annexin-V Red (Essen BioScience, Ann Arbor, MI) to detect apoptosis was added immediately before adding PBMCs). Plates were imaged for 3 days using the IncuCyte S3 (Essen BioScience) instrument at 37 C, 5% CO₂. Percent killing was calculated as nGFP+ Annexin-V-Red+ cell count divided by total nGFP+ cell count.

### ELISA

Target cells expressing nuclear-GFP (nGFP) were seeded in 96 well plates (Thermo, NU-167008), 5×10³ cells per well, in LymphoOne medium supplemented with 1% human serum. The next day, transduced or electroporated PBMCs were added to the wells at 5:1 E:T ratio. Cells are co-incubated for 15-18 hrs at 37 C, 5% CO₂. Following co-incubation, supernatant is harvested and transferred to non-binding 96-well plates (Greiner, #655901) at −200c. Supernatants are diluted 3 and 100-fold, ELISA performed as to manufactures instruction (Human IFN-gamma Quantikine, R&D, #SIF50) and quantified using Tecan plate reader.

### Quantification of Antigen Expression by Flow Cytometry

The MESF/“Antibody Binding Capacity” (ABC) ratio of a particular antibody can be used to quantify the number of antigen sites per cell. To establish the MESF/ABC ratio of each antibody Lot, MFIs of stained SCQ beads were correlated to the MFIs of MESF standards. The slope of the curve constitutes the ratio of fluorochrome label in MESF units per antibody. The MESF/ABC of every antibody Lot was measured using mouse/human/rat Simple Cellular Quantum (SCQ) Beads and MESF standards purchased from Bangs laboratories. Each of the 4 populations of SCQ beads has a known Antibody Binding Capacity (ABC), typically in the range of several thousands to 500-800K, so by staining these beads with an antibody at near saturation, one can correlate the fluorescence measurement (MFI) on a flow cytometer to the amount of bound antibody (ABC). MESF standard beads are composed of 4-5 different bead populations labeled with a known amount of fluorochrome molecules. By running MESF beads on a flow cytometer, one can correlate an MFI measurement to MESF units and compare between data that was collected on multiple different occasions, PMT voltages and instruments. When using HLA-A2/NYESO1-PE tetramers to stain tag-less iCAR constructs, the MESF/ABC ratio was established by staining control Jurkat cell lines that express a tagged aCAR and iCAR at high and low levels, with both quantifiable Anti-Myc Tag antibody and HLA-A2/NYESO1-PE tetramers. For each staining 100-200K positive cells were washed twice with 100 ul of cold FACS buffer (2% FCS in PBS×1) by centrifugation, 300 g for 5 min at 4° C. For Flag tagged aCAR and Myc tagged iCAR quantification, the cells were stained with 50 ul of APC (130-119-584, Miltenyi) and FITC (130-116-485, Miltenyi) labeled antibodies diluted 1/25 with FACS buffer. For un-tagged trastuzumab aCAR and Anti-HLA-A2 iCAR quantification, primary human Anti-Trastuzumab scFv69 (Ab00618-10.0, Absolute Antibody), HLA-A2/NYESO1-PE tetramers (TB-M105-1, MBL) and secondary Anti-human Fc APC (BLG-409306, biolegend) were diluted in FACS buffer, 1/25, 1/5 and 1/10 respectively. For target cell line antigen quantification, Anti-EGFR PE (FAB9577P-100, R&D), Anti-HER2 APC (130-106-696, Miltenyi) and Anti-HLA-A2 APC (17-9876-42, ebioscience) were diluted with FACS buffer, 1/2.5, 1/10 and 1/5 respectively. The cells were incubated at 4° C. in the dark for 45-60 min and washed thrice with 100 ul cold FACS buffer as described previously. The cells were resuspended with 150 ul of FACS buffer or PBS×1 containing 0.5-1 ug/ml DAPI (MBD0015-1, Merck-Sigma). The cells were analyzed by flow cytometry (BD FACS Celesta or MACSQuant Analyzer 10) collecting 10K-50K double positive events from each sample. Next, without changing the PMT voltages on the instrument, 5-10K events of each population of relevant MESF standard beads (FITC 555P-5ML, APC 823-5ML, PE 827-5ML, Bangs), were collected. FlowJo software was used to gate and calculate MFIs (Geometric Mean Fluorescence) and MESF beads QuickCal files, provided by the manufacturer, were used to convert the MFIs in to MESF units. Next, the values were converted to ABC units Using the MESF/ABC curves of the specific antibody lots used.

### Discussion

FaDu/MCF7-Luc Immune Killing Assay

Identification of novel iCAR using a nucGFP labeled target cells endpoint and bicistronic LV transduction.

The assay was useful regarding increasing the potency of iCAR inhibition (scFv avidity & activity) is necessary to decrease the iCAR/aCAR stoichiometry for efficient aCAR protection. Continued development and analysis related to dual differential expression in a lentiviral bicistronic format is ongoing and in progress.

Focused on HER2 (anti-Trastuzumab scFv) as an aCAR. Identified fully human or humanized scFv's to target HLA-A2. Identified novel iDomains (LIR1, KIR2DL1, KIR2DL2, and/or BTLA).

Lentiviral dualCAR Expression

Low transduction efficiency and variable differential expression.

iCAR constructs are identical, except for variations in the inhibitory domain.

aCAR constructs are also identical, except for variations in the scFv: Cetuximab or Panitumumab for EGFR and Trastuzumab or Pertuzumab for Her2.

All constructs are in iCAR/aCAR configuration with T2A cleavable linker.

IncuCyte Immune Cell Killing Assay: a cell imagining platform to monitor target killing and proliferation, and T-cell activation kinetics.

Quantum Bead Assay: a methodology to incorporate absolute aCAR & iCAR level, stoichiometry, and expression kinetics into screening and analysis.

IMPT001 GO: in vitro validation HLA-A2 scFv/PD-1 iCAR pairing with EGFR & HER2 scFv aCAR using mRNA co-electroporation of constructs into effector cells, using the Incucyte platform and FACS T-cell profiling IMPT001 go.

Lentiviral Technology: Design and evaluate the expression of mono- and dual lentiviral aCAR & iCAR to support IMPT001 and identify novel iCAR (64 constructs).

New Potent HLA-A2 scFv: Characterize a fully human HLA-A2 scFv alternative to murine BB7.2 as a lentiviral iCAR transduced into donor PBMC that appears to bind HLA-A tetramers more avidly.

FaDU/U87-Luc Immune Killing Assay: identification of novel iCAR using a Luciferase viability endpoint. LIR1 & KIR2DL1 iCARs identified.

### Validation of an IncuCyte Immune Cell Killing Assay

Dependence of target cell killing & proliferation on E/T ratio.

Implemented an immune cell killing assay that simultaneously images the kinetics of target cell killing and proliferation, and T-cell activation.

The technology is applicable to diverse adherent cancer cell lines partially circumventing the time and cost associated with engineering isogenic cell lines.

The kinetic and endpoints are a quantitative metrics that will allow dual CAR ranking, i.e., directly proportional to E/T ratio and aCAR and iCAR level.

The sensitivity (E/T EC50) of target cancer cell lines to EGFR and HER2 aCAR killing varies >5-fold and does not correlate with EGFR expression level.

### Cell-Surface Expression

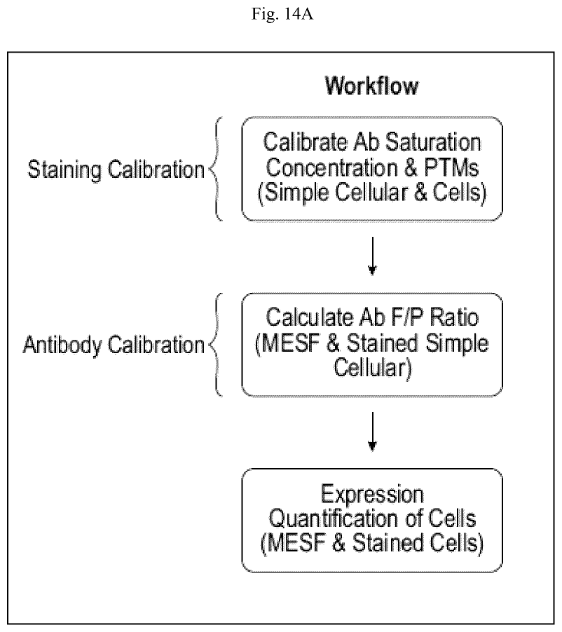

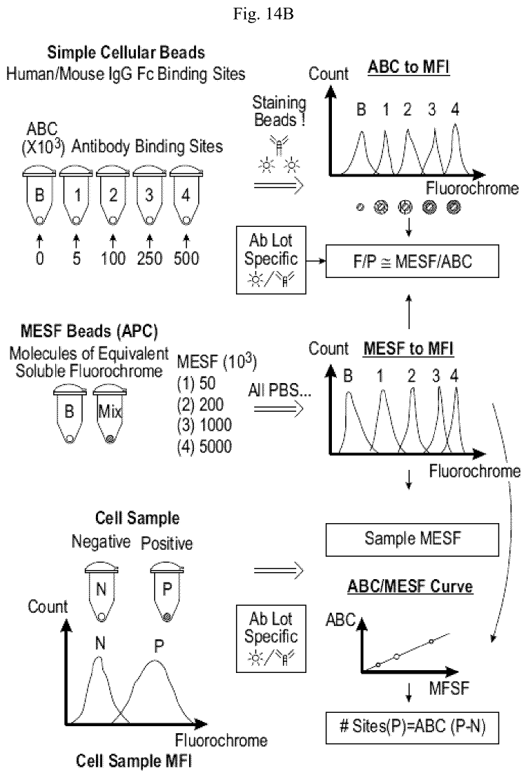

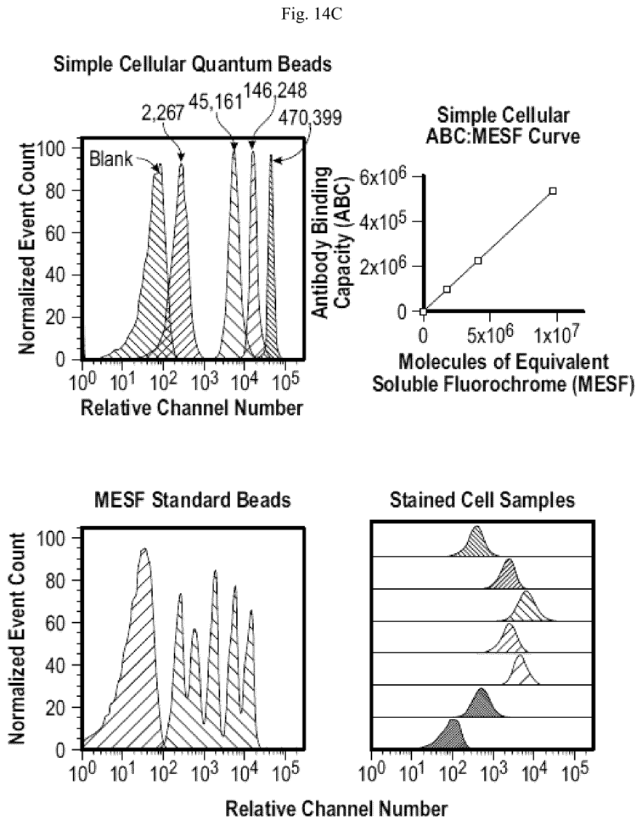

Absolute iCAR/aCAR level and stoichiometry—Effector cells. Absolute iCAR/aCAR antigen level and stoichiometry—Target cells. See, for example, FIG. 14.

A highly reproducible FACS based method has been implemented to quantify absolute CAR and target antigen levels)

The level of aCAR and iCAR expression obtained with mRNA co-electroporation are linearly dependent on mRNA amount.

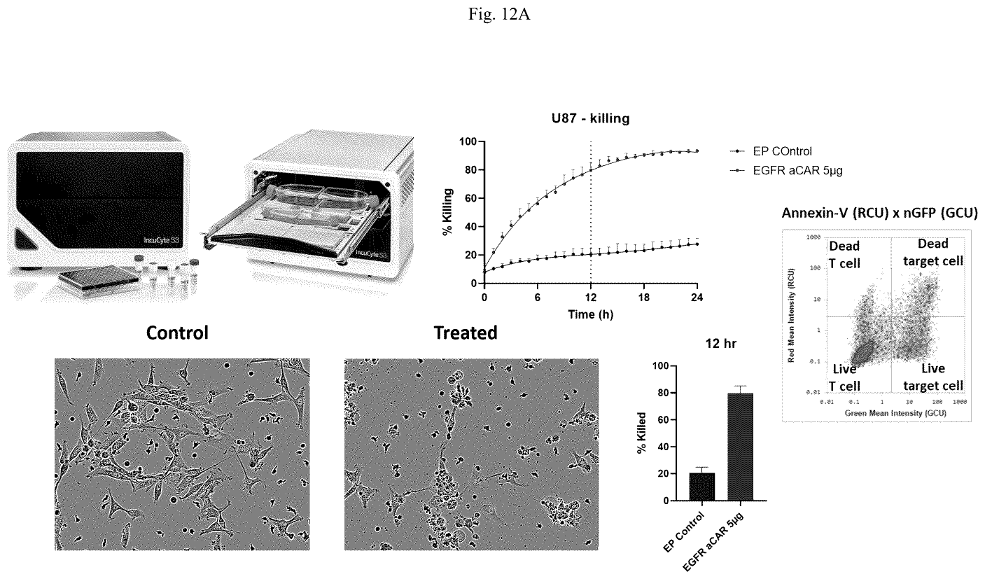

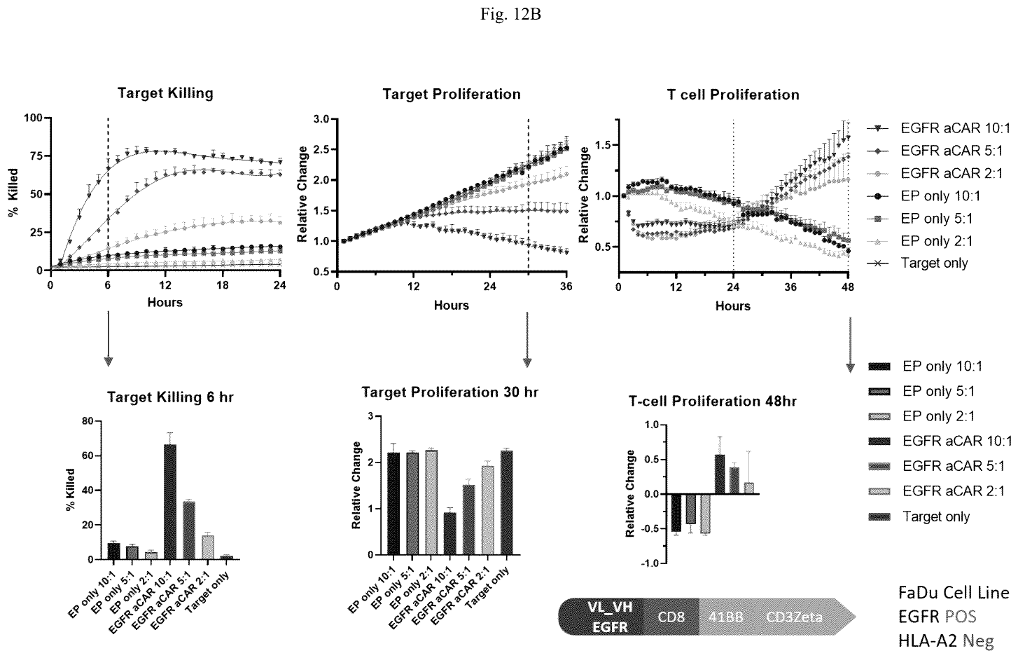

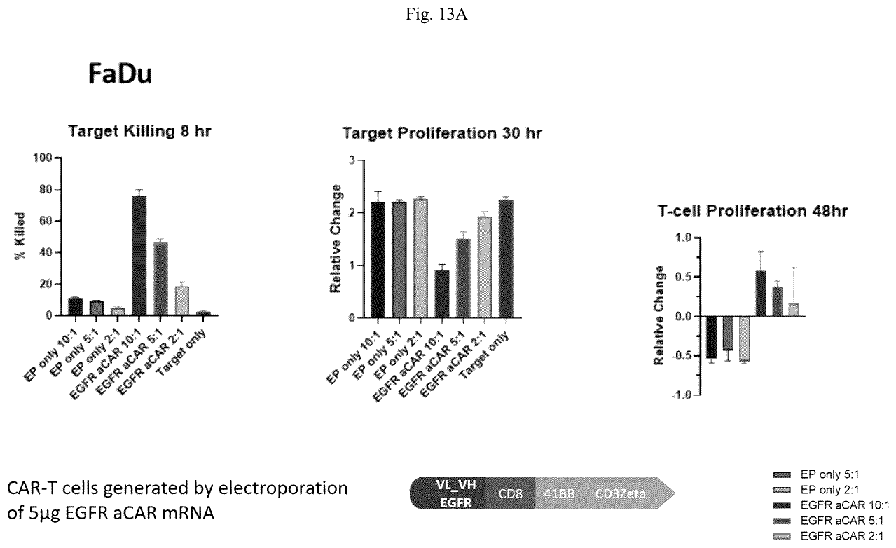

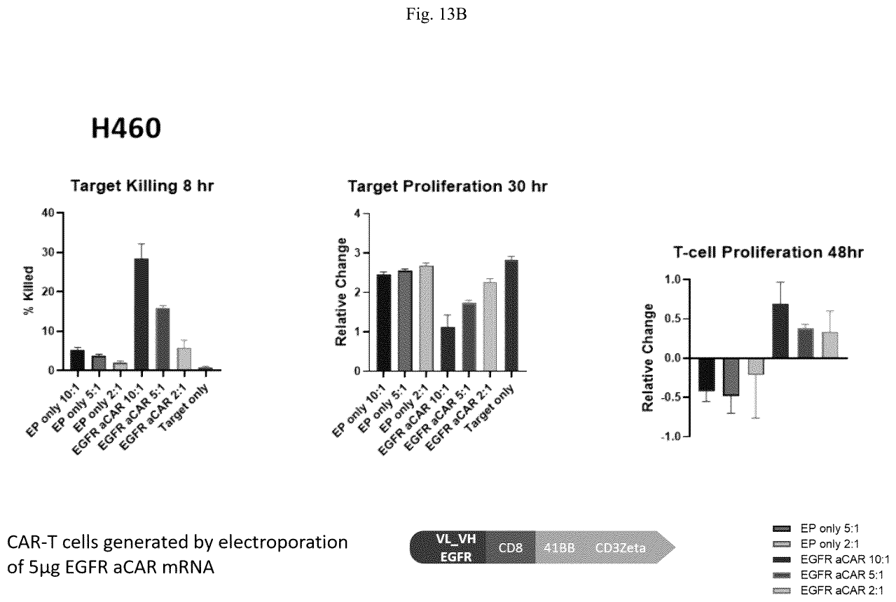

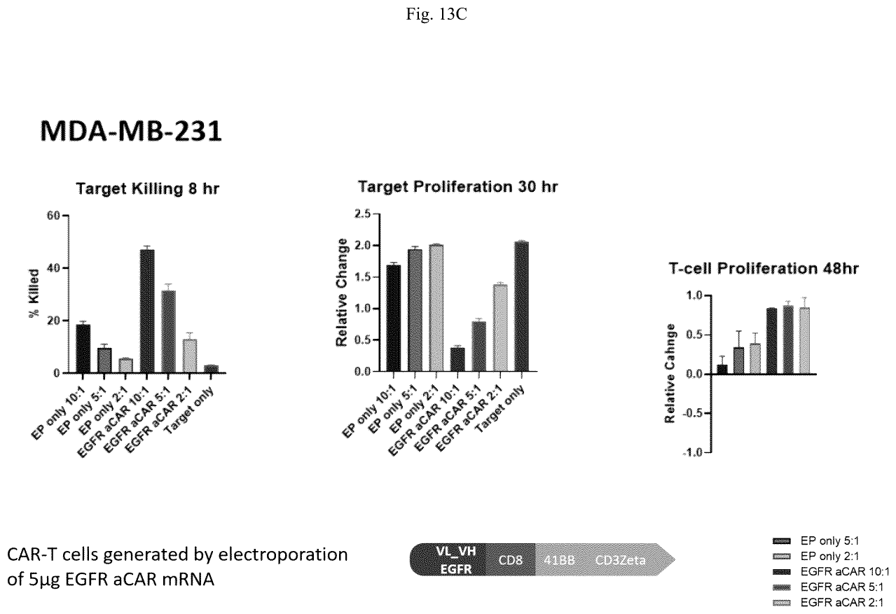

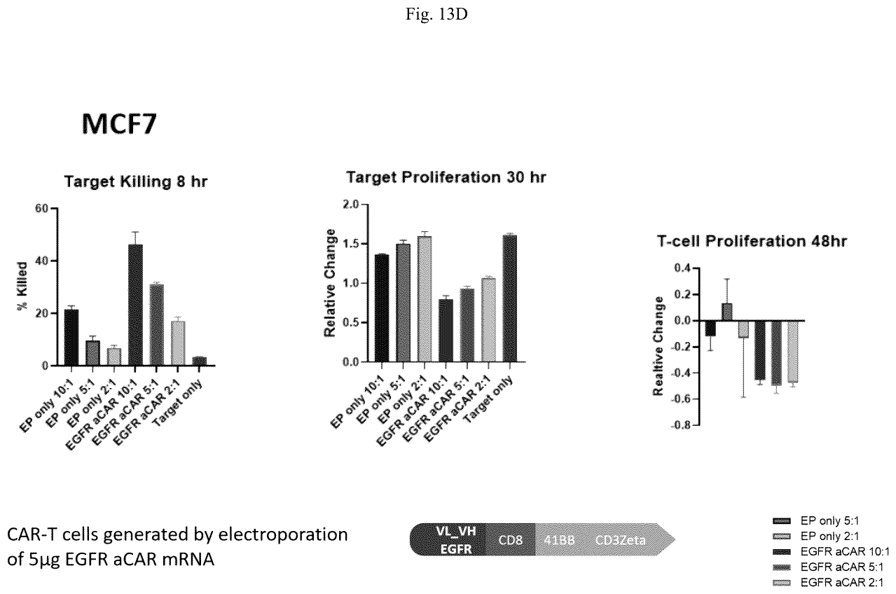

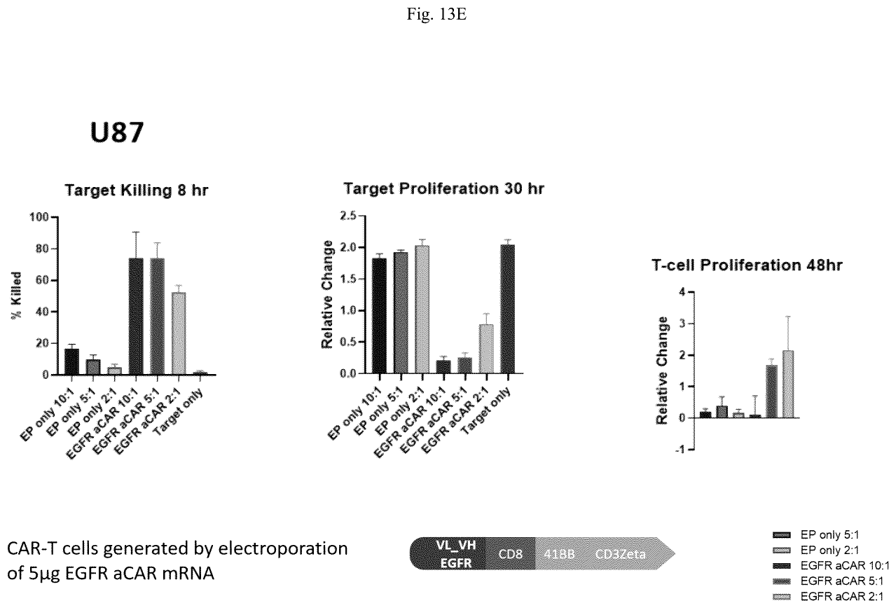

Stoichiometric expression by co-electroporation is heavily iCAR biased (e.g., iCAR/aCAR slope=6.0 on the Jurkat experiment (See, for example, FIGS. 12-13).

### An EGFR x HLA-A2 Dual CAR

Validation with mRNA co-electroporation studies.

Pairing of Cetuximab aCAR with a BB7.2 PD-1 iCAR was assessed by mRNA co-electroporation in HLA-A2 NEG and HLA-A2 POS adherent cancer cell-lines.

Killing of FaDu A2 NEG cells by dual CAR T-cells was obtained at low E/T ratios without apparent loss of aCAR activity, U87 A2 POS (EGFR aCAR sensitive) cancer cells were fully protected.

All HLA-A2 POS cancer cell lines tested inhibited T-cell activation (CD107a, IFNg) at low E/T regardless of HLA-A2 level (˜10⁵ to ˜10⁶ per cell) and target cell killing efficiency.

CAR quantification has not yet been performed, however HLA-A2 dependent protection is associated with excess iCAR exposure (>10-fold Cmax). See, for example, FIG. 12A.

Pairing of Trastuzumab scFv aCAR with a BB7.2 scFv PD-1 iCAR was assessed by mRNA co-electroporation in HLA-A2 NEG and HLA-A2 POS adherent cancer cell-lines.

Killing of FaDu A2 NEG cells by dual CAR T-cells was obtained at E/T=10 without apparent loss of aCAR activity.

Protection of MDA-MB-231 A2 POS cancer cells appeared to depend on a 300-fold excess of iCAR over aCAR expression (Cmax).

In contrast, lower iCAR levels were sufficient to inhibit T-cell activation (CD107a, IFNg, TNFα) regardless of target cell killing efficiency.

### Dual CAR Lentiviral Transduction

Absolute and stoichiometric expression in PBMCs.

iCAR constructs are identical, except for variations in the inhibitory domain.

aCAR constructs are also identical, except for variations in the scFv: Cetuximab or Panitumumab for EGFR and Trastuzumab or Pertuzumab for Her2.

All constructs are in iCAR/aCAR configuration with T2A cleavable linker.

The PBMC transduction efficiency (% gated double positive) of lentiviral bicistronic CARs was variable and most often too low (<20%) for IncuCyte co-culture assays.

iCAR expression (proximal gene) could exceed aCAR expression (distal gene) by 5-10 fold but exceptions and failures were not uncommon.

Identification of HUMAN Alternatives to BB7.2 HLA-A2 scFv

Mono-cistronic expression in PBMCs and HLA-A tetramer binding.

Binding to HLA-A2 tetramers was observed for BB7.2 (++), 3PF12 (+++), SN66E3 (+++), MBW1. Sequence Modifies (++). Binding to HLA-A2 tetramers was no observed for Ha5C2.A2 and murine BBM.1.

cMYC tag reports surface expression of iCARs.

HLA-A2 tetramer reports on HLA-A2 scFv binding.

Two fully human HLA-A2 scFv were identified as potential alternatives to BB7.2 (murine) that appear to bind HLA-A2 tetramer with higher avidity: 3PF12 and SN66E3.

### FaDu/U87-Luc Immune Killing Assay

Identification of novel iCAR using a Luciferase viability endpoint.

FaDu/U87-Luc Assay was developed and internal controls were validated. EGFR aCARs show robust specific killing of FaDu and U87 cells. HLA-A2 aCAR shows specific killing in U87 HLA-A2 POS cells.

Achieves high E/T ratios without assay interference (E/T=64).

HLA-A2 dependent protection observed KIR2DL1 is consistent with T-REP (KIR2DL2) and Jurkat NFAT-Luc FA experiments (KIR2DL1+KIR2DL2).

### FaDu/MCF7-Luc Immune Killing Assay

Identification of novel iCAR using a Luciferase viability endpoint.

FaDu/MCF7-Luc Assay was developed and internal controls were validated. HER2 aCARs show robust specific killing of FaDu and MCF7 cells. Achieves high E/T ratios without assay interference (E/T=20).

HLA-A2 dependent protection observed with KIR2DL1 is consistent with T-REP (KIR2DL2) and Jurkat NFAT-Luc FA experiments (KIR2DL1+KIR2DL2).

HLA-A2 dependent protection observed with LIR1 is consistent with Jurkat NFAT-Luc FA experiments.

### SUMMARY

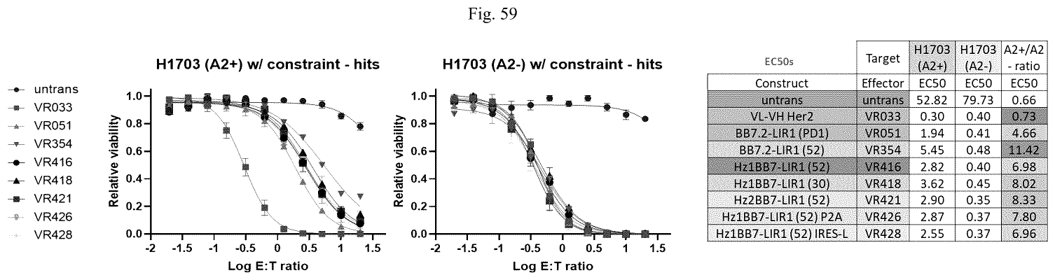

The data provide herein supports in vitro validation of a humanized BB7.2 iCAR scFv (see, for example, FIG. 59). This data confirmed that efficacy was observed for all constructs with a Hz BB7.2 version. This data also demonstrated that protection was observed for all constructs with a Hz BB7.2 version. VR428 and VR421 were identified as exemplary constructs.

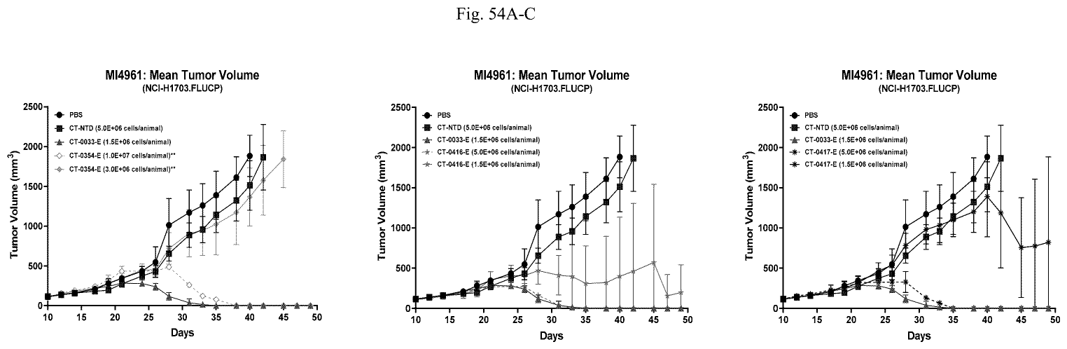

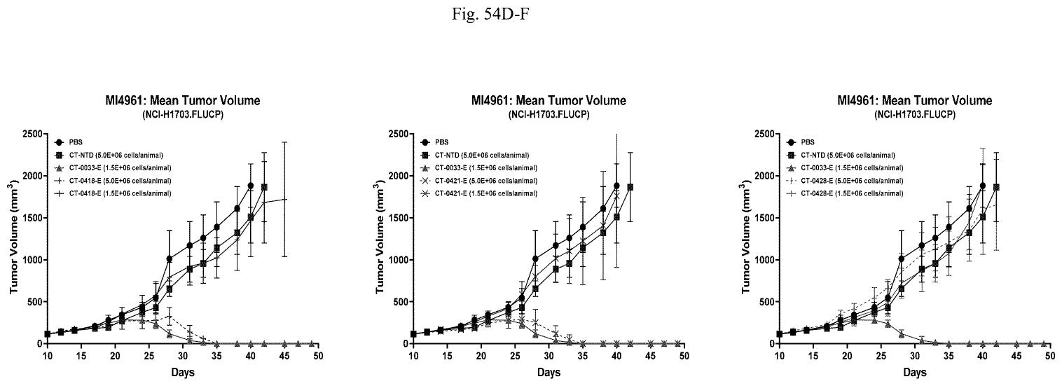

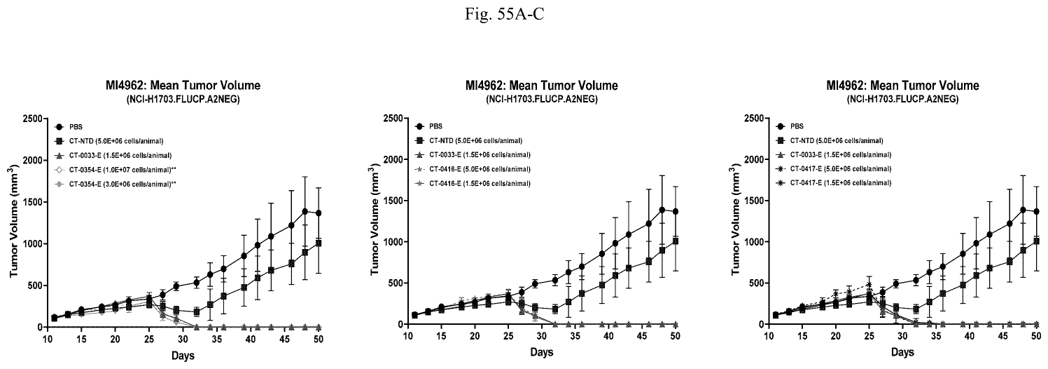

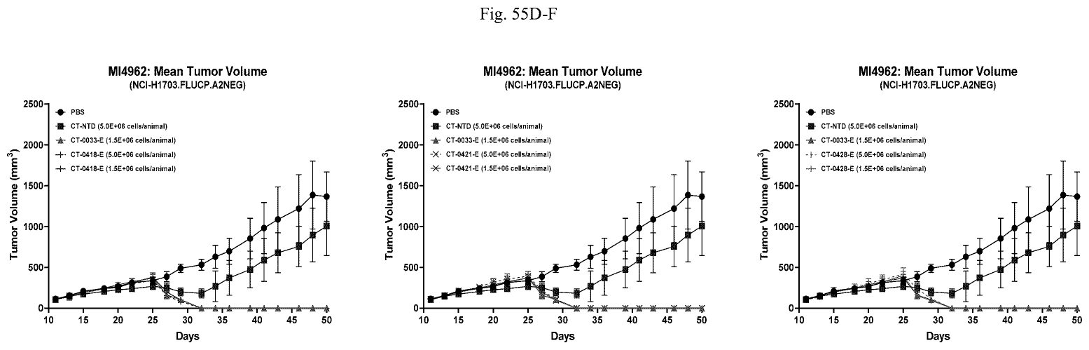

Also provided by the data was in vivo validation of HzBB7.2 iCAR scFv (see, for example, FIG. 54 and FIG. 55). Both efficacy and protection were demonstrated in an in vivo study for low and high dose with VR428 administration. VR428 was identified as an exemplary construct.

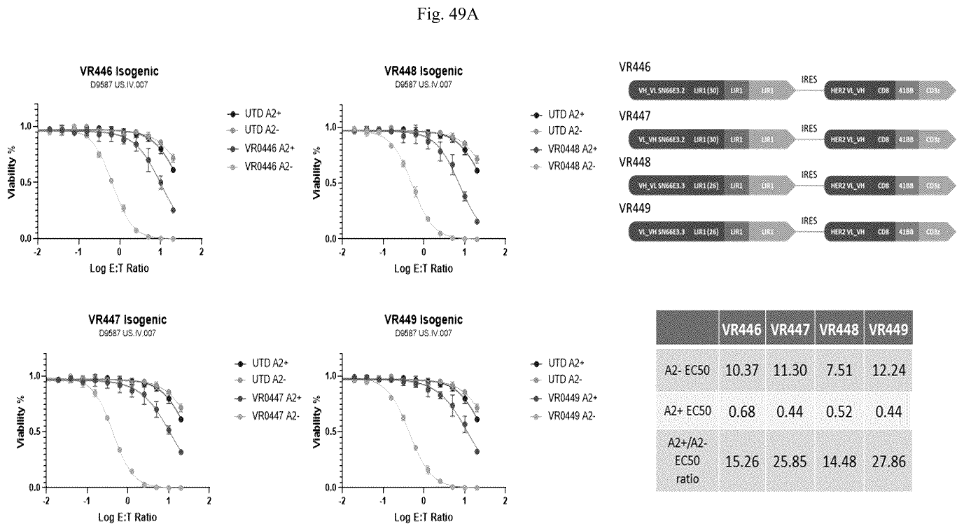

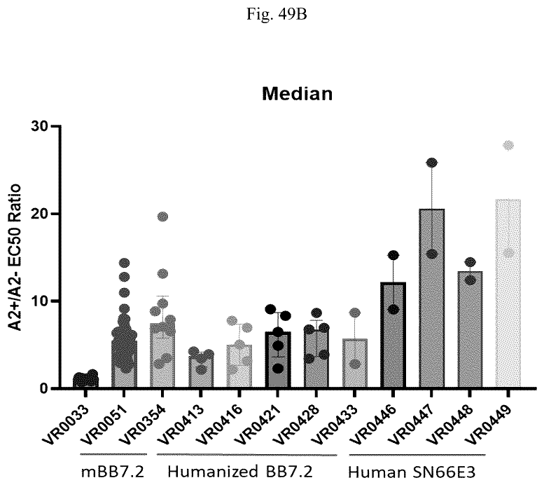

Also provided by the data was in vitro validation of a fully human SN66E3.3 iCAR scFv (see, for example, FIG. 49). This data confirmed that efficacy was observed for all constructs with a fully human SN66E3 version. This data also demonstrated that protection was observed for all constructs with a fully human SN66E3 versions. VR447 and VR449 were identified as exemplary constructs.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A bicistronic iCAR/aCAR construct or monocistronic aCAR and iCAR constructs for co-transduction comprising:
an iCAR portion, wherein the iCAR portion comprises:
an iCAR single chain variable fragment (scFv) component optionally in the VH-VL or VL-VH orientation;
an iCAR hinge domain component;
an iCAR transmembrane (TM) domain component;
an iCAR inhibitory domain component; and
an aCAR portion, wherein the iCAR portion comprises:
an aCAR single chain variable fragment (scFv) component optionally in the VH-VL or VL-VH orientation;
an aCAR hinge domain component;
an aCAR co-stimulatory domain component an aCAR activation signaling domain; and
a linker that connects the iCAR portion in (i) and the aCAR portion in (ii).

2. The bicistronic iCAR/aCAR construct or monocistronic aCAR and iCAR constructs for co-transduction according to claim 1, wherein the linker connecting the VH-VL or VL-VH in either orientation comprises one or more linker selected from the group consisting of (G4S)X3 linker (SEQ ID NO:81), G4S (SEQ ID NO:153), (G4S)X3 (SEQ ID NO:154), and Whitlow linker (SEQ ID NO:82).

3. The bicistronic iCAR/aCAR construct or monocistronic aCAR and iCAR constructs for co-transduction according to claim 1 or 2, wherein the iCAR scFv component targets an HLA antigen.

4. The bicistronic iCAR/aCAR construct or monocistronic aCAR and iCAR constructs for co-transduction according to any one of claims 1 to 3, wherein the HLA antigen is selected from the group consisting of HLA-A2, HLA-A3, HLA-A, HLA-B, HLA-C, HLA-G, HLA-E, HLA-F, HLA-DPA1, HLA-DQA1, HLA-DQB1, HLA-DQB2, HLA-DRB1, and HLA-DRB5.

5. The bicistronic iCAR/aCAR construct or monocistronic aCAR and iCAR constructs for co-transduction according to any one of claims 1 to 4, wherein the iCAR scFv component is selected from the group consisting of BB7.2, 3PF12, 3PF12/C4, 3PF12/F12, 3PF12/B11, W6/32, BBM.1, SN66E3, Ha5C2.A2, MWB1, MWB1-mod, Hz.BB7.2 VH1-69_A18VK, Hz.BB7.2 VH1-69 (27,30)_A18, Hz.BB7.2 VH1-69 (27,30,48)_A18, Hz.BB7.2 VH1-69 (27,30,67)_A18, Hz.BB7.2 VH1-69 (27,30,69)_A18, Hz.BB7.2 VH1-69 (27,30,67,69)_A18, Hz.BB7.2 VH1-3_A18, Hz.BB7.2 VH1-3(48)_A18, Hz.BB7.2 VH1-3(67)_A18, Hz.BB7.2 VH1-3(69)_A18, Hz.BB7.2 VH1-3(71)_A18, Hz.BB7.2 VH1-3(73)_A18, MWB1.2, SN66E3.2 and SN66E3.3.

6. The bicistronic iCAR/aCAR construct or monocistronic aCAR and iCAR constructs for co-transduction according to any one of claims 1 to 5, wherein the iCAR scFv component is BB7.2.

7. The bicistronic iCAR/aCAR construct or monocistronic aCAR and iCAR constructs for co-transduction according to claim 6, wherein the iCAR scFv comprises the Vh and Vl from BB7.2 (SEQ ID NOs: 37 and 38) or vhCDR1, vhCDR2, vhCDR3, vlCDR1, vlCDR2, and vlCDR3 from SEQ ID NOs: 37 and 38.

8. The bicistronic iCAR/aCAR construct or monocistronic aCAR and iCAR constructs for co-transduction according to claim 6, wherein the iCAR scFv comprises the Vh and Vl from Hz.BB7.2 VH1-69_A18VK (SEQ ID NOs: 57 and 58) or vhCDR1, vhCDR2, vhCDR3, vlCDR1, vlCDR2, and vlCDR3 from SEQ ID NOs: 57 and 58.

9. The bicistronic iCAR/aCAR construct or monocistronic aCAR and iCAR constructs for co-transduction according to claim 6, wherein the iCAR scFv comprises the Vh and Vl from Hz.BB7.2 VH1-69 (27,30)_A18 (SEQ ID NOs: 59 and 60) or vhCDR1, vhCDR2, vhCDR3, vlCDR1, vlCDR2, and vlCDR3 from SEQ ID NOs: 59 and 60.

10. The bicistronic iCAR/aCAR construct or monocistronic aCAR and iCAR constructs for co-transduction according to claim 6, wherein the iCAR scFv comprises the Vh and Vl from Hz.BB7.2 VH1-69 (27,30,48)_A18 (SEQ ID NOs: 61 and 62) or vhCDR1, vhCDR2, vhCDR3, vlCDR1, vlCDR2, and vlCDR3 from SEQ ID NOs: 61 and 62.

11. The bicistronic iCAR/aCAR construct or monocistronic aCAR and iCAR constructs for co-transduction according to claim 6, wherein the iCAR scFv comprises the Vh and Vl from Hz.BB7.2 VH1-69 (27,30,67)_A18 (SEQ ID NOs: 63 and 64) or vhCDR1, vhCDR2, vhCDR3, vlCDR1, vlCDR2, and vlCDR3 from SEQ ID NOs: 63 and 64.

12. The bicistronic iCAR/aCAR construct or monocistronic aCAR and iCAR constructs for co-transduction according to claim 6, wherein the iCAR scFv comprises the Vh and Vl from Hz.BB7.2 VH1-69 (27,30,69)_A18 (SEQ ID NOs: 65 and 66) or vhCDR1, vhCDR2, vhCDR3, vlCDR1, vlCDR2, and vlCDR3 from SEQ ID NOs: 65 and 66.

13. The bicistronic iCAR/aCAR construct or monocistronic aCAR and iCAR constructs for co-transduction according to claim 6, wherein the iCAR scFv comprises the Vh and Vl from Hz.BB7.2 VH1-69 (27,30,67,69)_A18 (SEQ ID NOs: 67 and 68) or vhCDR1, vhCDR2, vhCDR3, vlCDR1, vlCDR2, and vlCDR3 from SEQ ID NOs: 67 and 68.

14. The bicistronic iCAR/aCAR construct or monocistronic aCAR and iCAR constructs for co-transduction according to claim 6, wherein the iCAR scFv comprises the Vh and Vl from Hz.BB7.2 VH1-3_A18 (SEQ ID NOs: 69 and 70) or vhCDR1, vhCDR2, vhCDR3, vlCDR1, vlCDR2, and vlCDR3 from SEQ ID NOs: 69 and 70.

15. The bicistronic iCAR/aCAR construct or monocistronic aCAR and iCAR constructs for co-transduction according to claim 6, wherein the iCAR scFv comprises the Vh and Vl from Hz.BB7.2 VH1-3(48)_A18 (SEQ ID NOs: 71 and 72) or vhCDR1, vhCDR2, vhCDR3, vlCDR1, vlCDR2, and vlCDR3 from SEQ ID NOs: 71 and 72.

16. The bicistronic iCAR/aCAR construct or monocistronic aCAR and iCAR constructs for co-transduction according to claim 6, wherein the iCAR scFv comprises the Vh and Vl from Hz.BB7.2 VH1-3(67)_A18 (SEQ ID NOs: 73 and 74) or vhCDR1, vhCDR2, vhCDR3, vlCDR1, vlCDR2, and vlCDR3 from SEQ ID NOs: 73 and 74.

17. The bicistronic iCAR/aCAR construct or monocistronic aCAR and iCAR constructs for co-transduction according to claim 6, wherein the iCAR scFv comprises the Vh and Vl from Hz.BB7.2 VH1-3(69)_A18 (SEQ ID NOs: 75 and 76) or vhCDR1, vhCDR2, vhCDR3, vlCDR1, vlCDR2, and vlCDR3 from SEQ ID NOs: 75 and 76.

18. The bicistronic iCAR/aCAR construct or monocistronic aCAR and iCAR constructs for co-transduction according to claim 6, wherein the iCAR scFv comprises the Vh and Vl from Hz.BB7.2 VH1-3(71)_A18 (SEQ ID NOs: 77 and 78) or vhCDR1, vhCDR2, vhCDR3, vlCDR1, vlCDR2, and vlCDR3 from SEQ ID NOs: 77 and 78.

19. The bicistronic iCAR/aCAR construct or monocistronic aCAR and iCAR constructs for co-transduction according to claim 6, wherein the iCAR scFv comprises the Vh and Vl from Hz.BB7.2 VH1-3(73)_A18 (SEQ ID NOs: 79 and 80) or vhCDR1, vhCDR2, vhCDR3, vlCDR1, vlCDR2, and vlCDR3 from SEQ ID NOs: 79 and 80.

20. The bicistronic iCAR/aCAR construct or monocistronic aCAR and iCAR constructs for co-transduction according to claim 6, wherein the iCAR scFv is BB7.2 of SEQ ID NO:167.

21. The bicistronic iCAR/aCAR construct or monocistronic aCAR and iCAR constructs for co-transduction according to any one of claims 1 to 4, wherein the iCAR scFv component is 3PF12.

22. The bicistronic iCAR/aCAR construct or monocistronic aCAR and iCAR constructs for co-transduction according to claim 21, wherein the iCAR scFv comprises the Vh and Vl from 3PF12/C4 (SEQ ID NOs: 39 and 40) or vhCDR1, vhCDR2, vhCDR3, vlCDR1, vlCDR2, and vlCDR3 from SEQ ID NOs: 39 and 40.

23. The bicistronic iCAR/aCAR construct or monocistronic aCAR and iCAR constructs for co-transduction according to claim 21, wherein the iCAR scFv comprises the Vh and Vl from 3PF12/F12 (SEQ ID NOs: 41 and 42) or vhCDR1, vhCDR2, vhCDR3, vlCDR1, vlCDR2, and vlCDR3 from SEQ ID NOs: 41 and 42.

24. The bicistronic iCAR/aCAR construct or monocistronic aCAR and iCAR constructs for co-transduction according to claim 21, wherein the iCAR scFv comprises the Vh and Vl from 3PF12/B11 (SEQ ID NOs: 43 and 44) or vhCDR1, vhCDR2, vhCDR3, vlCDR1, vlCDR2, and vlCDR3 from SEQ ID NOs: 43 and 44.

25. The bicistronic iCAR/aCAR construct or monocistronic aCAR and iCAR constructs for co-transduction according to claim 21, wherein the iCAR scFv is 3PF12 of SEQ ID NO:168.

26. The bicistronic iCAR/aCAR construct or monocistronic aCAR and iCAR constructs for co-transduction according to any one of claims 1 to 2, wherein the iCAR scFv component is SN66E3.

27. The bicistronic iCAR/aCAR construct or monocistronic aCAR and iCAR constructs for co-transduction according to claim 26, wherein the iCAR scFv comprises the Vh and Vl from SN66E3.1 (SEQ ID NOs: 49 and 50) or vhCDR1, vhCDR2, vhCDR3, vlCDR1, vlCDR2, and vlCDR3 from SEQ ID NOs: 49 and 50.

28. The bistronic iCAR/aCAR construct or monocistronic aCAR and iCAR constructs for co-transduction according to claim 26, wherein the iCAR scFv is SN66E3.1 of SEQ ID NO:169.

29. The bicistronic iCAR/aCAR construct or monocistronic aCAR and iCAR constructs for co-transduction according to claim 26, wherein the iCAR scFv comprises the Vh and Vl from SN66E3.2 (SEQ ID NOs: 165 and 166) or vhCDR1, vhCDR2, vhCDR3, vlCDR1, vlCDR2, and vlCDR3 from SEQ ID NOs: 165 and 166.

30. The bicistronic iCAR/aCAR construct or monocistronic aCAR and iCAR constructs for co-transduction according to claim 26, wherein the iCAR scFv is SN66E3.2 of SEQ ID NO:285.

31. The bicistronic iCAR/aCAR construct or monocistronic aCAR and iCAR constructs for co-transduction according to claim 26, wherein the iCAR scFv comprises the Vh and Vl from SN66E3.3 (SEQ ID NOs: 283 and 284) or vhCDR1, vhCDR2, vhCDR3, vlCDR1, vlCDR2, and vlCDR3 from SEQ ID NOs: 283 and 284.

32. The bicistronic iCAR/aCAR construct or monocistronic aCAR and iCAR constructs for co-transduction according to claim 26, wherein the iCAR scFv is SN66E3.3 of SEQ ID NO:286.

33. The bicistronic iCAR/aCAR construct or monocistronic aCAR and iCAR constructs for co-transduction according to any one of claims 1 to 4, wherein the iCAR scFv component is W6/32.

34. The bicistronic iCAR/aCAR construct or monocistronic aCAR and iCAR constructs for co-transduction according to claim 33, wherein the iCAR scFv comprises the Vh and Vl from W6/32 (SEQ ID NOs: 45 and 46) or vhCDR1, vhCDR2, vhCDR3, vlCDR1, vlCDR2, and vlCDR3 from SEQ ID NOs: 45 and 46.

35. The bicistronic iCAR/aCAR construct or monocistronic aCAR and iCAR constructs for co-transduction according to any one of claims 1 to 5, wherein the iCAR scFv component is BBM.1.

36. The bicistronic iCAR/aCAR construct or monocistronic aCAR and iCAR constructs for co-transduction according to claim 35, wherein the iCAR scFv comprises the Vh and Vl from BBM.1 (SEQ ID NOs: 47 and 48) or vhCDR1, vhCDR2, vhCDR3, vlCDR1, vlCDR2, and vlCDR3 from SEQ ID NOs: 47 and 48.

37. The bicistronic iCAR/aCAR construct or monocistronic aCAR and iCAR constructs for co-transduction according to any one of claims 1 to 5, wherein the iCAR scFv component is Ha5C2.A2.

38. The bicistronic iCAR/aCAR construct or monocistronic aCAR and iCAR constructs for co-transduction according to claim 33, wherein the iCAR scFv comprises the Vh and Vl from Ha5C2.A2 (SEQ ID NOs: 51 and 52) or vhCDR1, vhCDR2, vhCDR3, vlCDR1, vlCDR2, and vlCDR3 from SEQ ID NOs: 51 and 52.

39. The bicistronic iCAR/aCAR construct or monocistronic aCAR and iCAR constructs for co-transduction according to any one of claims 1 to 5, wherein the iCAR scFv component is MWB1.

40. The bicistronic iCAR/aCAR construct or monocistronic aCAR and iCAR constructs for co-transduction according to claim 39, wherein the iCAR scFv comprises the Vh and Vl from MWB1 (SEQ ID NOs: 53 and 54) or vhCDR1, vhCDR2, vhCDR3, vlCDR1, vlCDR2, and vlCDR3 from SEQ ID NOs: 53 and 54.

41. The bicistronic iCAR/aCAR construct or monocistronic aCAR and iCAR constructs for co-transduction according to claim 35, wherein the iCAR scFv comprises the Vh and Vl from MWB1-mod (MWB1.1) (SEQ ID NOs: 55 and 56) or vhCDR1, vhCDR2, vhCDR3, vlCDR1, vlCDR2, and vlCDR3 from SEQ ID NOs: 55 and 56.

42. The bicistronic iCAR/aCAR construct or monocistronic aCAR and iCAR constructs for co-transduction according to claim 35, wherein the iCAR scFv comprises the Vh and Vl from MWB1.2 (SEQ ID NOs: 163 and 164).

43. The bicistronic iCAR/aCAR construct or monocistronic aCAR and iCAR constructs for co-transduction according to any one of claims 1 to 4, wherein the iCAR scFv is MWB1.1 scFvVH_VL (SEQ ID NO:273).

44. The bicistronic iCAR/aCAR construct or monocistronic aCAR and iCAR constructs for co-transduction according to any one of claims 1 to 5, wherein the iCAR scFv is MWB1.2 scFvVH_VL (SEQ ID NO:274).

45. The bicistronic iCAR/aCAR construct or monocistronic aCAR and iCAR constructs for co-transduction according to any one of claims 1 to 44, wherein the iCAR hinge domain component is selected from a PD-1 hinge, a CD28 hinge, and a CD8 hinge (including a CD8a hinge), a LIR1 Ig3-4 hinge, a LIR1 Ig-4 hinge, a LIR1 52 aa hinge, a LIR1 36 aa hinge, a LIR1 30 aa hinge, a LIR1 26 aa hinge, a LIR1 8 aa hinge, a CD33 hinge, a KIR2DL1 hinge, a PD-1 (47) hinge, a PD-1 (42) hinge, a PD-1 (36) hinge, a PD-1 (30) hinge, a PD-1 (26) hinge, and a PD-1 (20) hinge.

46. The bicistronic iCAR/aCAR construct or monocistronic aCAR and iCAR constructs for co-transduction according to claim 45, wherein the iCAR hinge domain component is a PD-1 hinge (SEQ ID NO:86).

47. The bicistronic iCAR/aCAR construct or monocistronic aCAR and iCAR constructs for co-transduction according to claim 45, wherein the iCAR hinge domain component is a CD28 hinge (SEQ ID NO:85).

48. The bicistronic iCAR/aCAR construct or monocistronic aCAR and iCAR constructs for co-transduction according to claim 45, wherein the iCAR hinge domain component is a CD8 alpha hinge (SEQ ID NO:84).

49. The bicistronic iCAR/aCAR construct or monocistronic aCAR and iCAR constructs for co-transduction according to claim 45, wherein the iCAR hinge domain component is a LIR1 Ig3-4 hinge (SEQ ID NO:87).

50. The bicistronic iCAR/aCAR construct or monocistronic aCAR and iCAR constructs for co-transduction according to claim 45, wherein the iCAR hinge domain component is a LIR1 Ig-4 hinge (SEQ ID NO:88).

51. The bicistronic iCAR/aCAR construct or monocistronic aCAR and iCAR constructs for co-transduction according to claim 45, wherein the iCAR hinge domain component is a LIR1 52 aa hinge (SEQ ID NO:89).

52. The bicistronic iCAR/aCAR construct or monocistronic aCAR and iCAR constructs for co-transduction according to claim 45, wherein the iCAR hinge domain component is a LIR1 36 aa hinge (SEQ ID NO:90).

53. The bicistronic iCAR/aCAR construct or monocistronic aCAR and iCAR constructs for co-transduction according to claim 45, wherein the iCAR hinge domain component is a LIR1 30 aa hinge (SEQ ID NO:91).

54. The bicistronic iCAR/aCAR construct or monocistronic aCAR and iCAR constructs for co-transduction according to claim 45, wherein the iCAR hinge domain component is a LIR1 26 aa hinge (SEQ ID NO:289).

55. The bicistronic iCAR/aCAR construct or monocistronic aCAR and iCAR constructs for co-transduction according to claim 45, wherein the iCAR hinge domain component is a LIR1 8 aa hinge (SEQ ID NO:92).

56. The bicistronic iCAR/aCAR construct or monocistronic aCAR and iCAR constructs for co-transduction according to claim 45, wherein the iCAR hinge domain component is a CD33 hinge (SEQ ID NO:93).

57. The bicistronic iCAR/aCAR construct or monocistronic aCAR and iCAR constructs for co-transduction according to claim 45, wherein the iCAR hinge domain component is a KIR2DL1 hinge (SEQ ID NO:94).

58. The bicistronic iCAR/aCAR construct or monocistronic aCAR and iCAR constructs for co-transduction according to claim 45, wherein the iCAR hinge domain component is a PD-1 (47) hinge (SEQ ID NO:290).

59. The bicistronic iCAR/aCAR construct or monocistronic aCAR and iCAR constructs for co-transduction according to claim 45, wherein the iCAR hinge domain component is a PD-1 (42) hinge (SEQ ID NO:291).

60. The bicistronic iCAR/aCAR construct or monocistronic aCAR and iCAR constructs for co-transduction according to claim 45, wherein the iCAR hinge domain component is a PD-1 (36) hinge (SEQ ID NO:292).

61. The bicistronic iCAR/aCAR construct or monocistronic aCAR and iCAR constructs for co-transduction according to claim 45, wherein the iCAR hinge domain component is a PD-1 (30) hinge (SEQ ID NO:293).

62. The bicistronic iCAR/aCAR construct or monocistronic aCAR and iCAR constructs for co-transduction according to claim 45, wherein the iCAR hinge domain component is a PD-1 (26) hinge (SEQ ID NO:294).

63. The bicistronic iCAR/aCAR construct or monocistronic aCAR and iCAR constructs for co-transduction according to claim 45, wherein the iCAR hinge domain component is a PD-1 (20) hinge (SEQ ID NO:295).

64. The bicistronic iCAR/aCAR construct or monocistronic aCAR and iCAR constructs for co-transduction according to any one of claims 1 to 63, wherein the iCAR TM domain component is selected from a PD-1 TM domain, a CD28 TM domain, a CD8 TM domain (including a CD8a TM domain), a LIR1 TM domain, a CD33 TM domain, and a KIR2DL1 TM domain.

65. The bicistronic iCAR/aCAR construct or monocistronic aCAR and iCAR constructs for co-transduction according to claim 57, wherein the iCAR TM domain component is a PD-1 TM domain (SEQ ID NO:97).

66. The bicistronic iCAR/aCAR construct or monocistronic aCAR and iCAR constructs for co-transduction according to claim 57, wherein the iCAR TM domain component is a CD28 TM domain (SEQ ID NO:96).

67. The bicistronic iCAR/aCAR construct or monocistronic aCAR and iCAR constructs for co-transduction according to claim 57, wherein the iCAR TM domain component is a CD8 alpha TM domain (SEQ ID NO:95).

68. The bicistronic iCAR/aCAR construct or monocistronic aCAR and iCAR constructs for co-transduction according to claim 57, wherein the iCAR TM domain component is a LIR1 TM domain (SEQ ID NO:98).

69. The bicistronic iCAR/aCAR construct or monocistronic aCAR and iCAR constructs for co-transduction according to claim 57, wherein the iCAR TM domain component is a CD33 TM domain (SEQ ID NO:99).

70. The bicistronic iCAR/aCAR construct or monocistronic aCAR and iCAR constructs for co-transduction according to claim 57, wherein the iCAR TM domain component is a KIR2DL1 TM domain (SEQ ID NO:100).

71. The bicistronic iCAR/aCAR construct or monocistronic aCAR and iCAR constructs for co-transduction according to any one of claims 1 to 63, wherein the iCAR inhibitory domain component is an inhibitory domain from a protein selected from the group consisting of PD-1, KIR2DL1, KIR2DL2, KIR2DL3, KIR2DL4, KIR2DL5A, KIR3DL1, KIR3DL2, KIR3DL3, LAIR1, CD22, CD33, SIGLEC5, SIGLEC6, SIGLEC7, SIGLEC8, SIGLEC9, SIGLEC10, SIGLEC11, SIGLEC12, PECAM1/CD31, CD200R1, FCRL1, FCRL2, FCRL3, FCRL4, FCRL5, SLAMF1, SLAMF5, BTLA, LAGS, 2B4, CD160, CEACAM1, TIM3, VISTA, TIGIT, SIRPalpha, FcγRIIB, CD5, CD300a, CD300f, LIR1, LIR2, LIR3, LIR5, LIRE, Ly9, 2xPD1(G4S), 2xPD1(PD1), PVRIg, and AA2AR.

72. The bicistronic iCAR/aCAR construct or monocistronic aCAR and iCAR constructs for co-transduction according to claim 64, wherein the iCAR inhibitory domain component is a PD-1 inhibitory domain (SEQ ID NO:101).

73. The bicistronic iCAR/aCAR construct or monocistronic aCAR and iCAR constructs for co-transduction according to claim 64, wherein the iCAR component is a KIR2DL1 inhibitory domain (SEQ ID NO:102).

74. The bicistronic iCAR/aCAR construct or monocistronic aCAR and iCAR constructs for co-transduction according to claim 64, wherein the iCAR component is a KIR2DL2 inhibitory domain (SEQ ID NO:103).

75. The bicistronic iCAR/aCAR construct or monocistronic aCAR and iCAR constructs for co-transduction according to claim 64, wherein the iCAR component is a KIR2DL3 inhibitory domain (SEQ ID NO:104).

76. The bicistronic iCAR/aCAR construct or monocistronic aCAR and iCAR constructs for co-transduction according to claim 64, wherein the iCAR inhibitory domain component is a KIR2DL4 inhibitory domain (SEQ ID NO:105).

77. The bicistronic iCAR/aCAR construct or monocistronic aCAR and iCAR constructs for co-transduction according to claim 64, wherein the iCAR inhibitory domain component is a KIR2DL5A inhibitory domain (SEQ ID NO:106).

78. The bicistronic iCAR/aCAR construct or monocistronic aCAR and iCAR constructs for co-transduction according to claim 64, wherein the iCAR inhibitory domain component is a KIR3DL1 inhibitory domain (SEQ ID NO:107).

79. The bicistronic iCAR/aCAR construct or monocistronic aCAR and iCAR constructs for co-transduction according to claim 64, wherein the iCAR inhibitory domain component is a KIR3DL2 inhibitory domain (SEQ ID NO:108).

80. The bicistronic iCAR/aCAR construct or monocistronic aCAR and iCAR constructs for co-transduction according to claim 64, wherein the iCAR inhibitory domain component is a KIR3DL3 inhibitory domain (SEQ ID NO:109).

81. The bicistronic iCAR/aCAR construct or monocistronic aCAR and iCAR constructs for co-transduction according to claim 64, wherein the iCAR inhibitory domain component is a LAIR1 inhibitory domain (SEQ ID NO:110).

82. The bicistronic iCAR/aCAR construct or monocistronic aCAR and iCAR constructs for co-transduction according to claim 64, wherein the iCAR inhibitory domain component is a CD22 inhibitory domain (SEQ ID NO:111).

83. The bicistronic iCAR/aCAR construct or monocistronic aCAR and iCAR constructs for co-transduction according to claim 64, wherein the iCAR inhibitory domain component is a CD33 inhibitory domain (SEQ ID NO:112).

84. The bicistronic iCAR/aCAR construct or monocistronic aCAR and iCAR constructs for co-transduction according to claim 64, wherein the iCAR inhibitory domain component is a SIGLEC5 inhibitory domain (SEQ ID NO:113).

85. The bicistronic iCAR/aCAR construct or monocistronic aCAR and iCAR constructs for co-transduction according to claim 64, wherein the iCAR inhibitory domain component is a SIGLEC6 inhibitory domain (SEQ ID NO:114).

86. The bicistronic iCAR/aCAR construct or monocistronic aCAR and iCAR constructs for co-transduction according to claim 64, wherein the iCAR inhibitory domain component is a SIGLEC7 inhibitory domain (SEQ ID NO:115).

87. The bicistronic iCAR/aCAR construct or monocistronic aCAR and iCAR constructs for co-transduction according to claim 64, wherein the iCAR inhibitory domain component is a SIGLEC8 inhibitory domain (SEQ ID NO:116).

88. The bicistronic iCAR/aCAR construct or monocistronic aCAR and iCAR constructs for co-transduction according to claim 64, wherein the iCAR inhibitory domain component is a SIGLEC9 inhibitory domain (SEQ ID NO:117).

89. The bicistronic iCAR/aCAR construct or monocistronic aCAR and iCAR constructs for co-transduction according to claim 64, wherein the iCAR inhibitory domain component is a SIGLEC10inhibitory domain (SEQ ID NO:118).

90. The bicistronic iCAR/aCAR construct or monocistronic aCAR and iCAR constructs for co-transduction according to claim 64, wherein the iCAR inhibitory domain component is a SIGLEC11inhibitory domain (SEQ ID NO:119).

91. The bicistronic iCAR/aCAR construct or monocistronic aCAR and iCAR constructs for co-transduction according to claim 64, wherein the iCAR inhibitory domain component is a SIGLEC12inhibitory domain (SEQ ID NO:120).

92. The bicistronic iCAR/aCAR construct or monocistronic aCAR and iCAR constructs for co-transduction according to claim 64, wherein the iCAR inhibitory domain component is a PECAM1/CD31 inhibitory domain (SEQ ID NO:121).

93. The bicistronic iCAR/aCAR construct or monocistronic aCAR and iCAR constructs for co-transduction according to claim 64, wherein the iCAR inhibitory domain component is a CD200R1inhibitory domain (SEQ ID NO:122).

94. The bicistronic iCAR/aCAR construct or monocistronic aCAR and iCAR constructs for co-transduction according to claim 64, wherein the iCAR inhibitory domain component is a FCRL1inhibitory domain (SEQ ID NO:123).

95. The bicistronic iCAR/aCAR construct or monocistronic aCAR and iCAR constructs for co-transduction according to claim 64, wherein the iCAR inhibitory domain component is a FCRL2inhibitory domain (SEQ ID NO:124).

96. The bicistronic iCAR/aCAR construct or monocistronic aCAR and iCAR constructs for co-transduction according to claim 64, wherein the iCAR inhibitory domain component is a FCRL3inhibitory domain (SEQ ID NO:125).

97. The bicistronic iCAR/aCAR construct or monocistronic aCAR and iCAR constructs for co-transduction according to claim 64, wherein the iCAR inhibitory domain component is a FCRL4 inhibitory domain (SEQ ID NO:126).

98. The bicistronic iCAR/aCAR construct or monocistronic aCAR and iCAR constructs for co-transduction according to claim 64 wherein the iCAR inhibitory domain component is a FCRL5 inhibitory domain (SEQ ID NO:127).

99. The bicistronic iCAR/aCAR construct or monocistronic aCAR and iCAR constructs for co-transduction according to claim 64, wherein the iCAR inhibitory domain component is a SLAMF1 inhibitory domain (SEQ ID NO:128).

100. The bicistronic iCAR/aCAR construct or monocistronic aCAR and iCAR constructs for co-transduction according to claim 64, wherein the iCAR inhibitory domain component is a SLAMF5 inhibitory domain (SEQ ID NO:129).

101. The bicistronic iCAR/aCAR construct or monocistronic aCAR and iCAR constructs for co-transduction according to claim 64, wherein the iCAR inhibitory domain component is a BTLA inhibitory domain (SEQ ID NO:130).

102. The bicistronic iCAR/aCAR construct or monocistronic aCAR and iCAR constructs for co-transduction according to claim 64, wherein the iCAR inhibitory domain component is a LAG3 inhibitory domain (SEQ ID NO:131).

103. The bicistronic iCAR/aCAR construct or monocistronic aCAR and iCAR constructs for co-transduction according to claim 64, wherein the iCAR inhibitory domain component is a 2B4inhibitory domain (SEQ ID NO:132).

104. The bicistronic iCAR/aCAR construct or monocistronic aCAR and iCAR constructs for co-transduction according to claim 64, wherein the iCAR inhibitory domain component is a CD160 inhibitory domain (SEQ ID NO:133).

105. The bicistronic iCAR/aCAR construct or monocistronic aCAR and iCAR constructs for co-transduction according to claim 64, wherein the iCAR inhibitory domain component is a CEACAM1 inhibitory domain (SEQ ID NO:134).

106. The bicistronic iCAR/aCAR construct or monocistronic aCAR and iCAR constructs for co-transduction according to claim 64, wherein the iCAR inhibitory domain component is a TIM3 inhibitory domain (SEQ ID NO:135).

107. The bicistronic iCAR/aCAR construct or monocistronic aCAR and iCAR constructs for co-transduction according to claim 64, wherein the iCAR inhibitory domain component is a VISTA inhibitory domain (SEQ ID NO:136).

108. The bicistronic iCAR/aCAR construct or monocistronic aCAR and iCAR constructs for co-transduction according to claim 64, wherein the iCAR inhibitory domain component is a TIGIT inhibitory domain (SEQ ID NO:137).

109. The bicistronic iCAR/aCAR construct or monocistronic aCAR and iCAR constructs for co-transduction according to claim 64, wherein the iCAR inhibitory domain component is a SIRPalpha inhibitory domain (SEQ ID NO:138).

110. The bicistronic iCAR/aCAR construct or monocistronic aCAR and iCAR constructs for co-transduction according to claim 64, wherein the iCAR inhibitory domain component is a FcγRIIB inhibitory domain (SEQ ID NO:139).

111. The bicistronic iCAR/aCAR construct or monocistronic aCAR and iCAR constructs for co-transduction according to claim 64, wherein the iCAR inhibitory domain component is a CD5 inhibitory domain (SEQ ID NO:140).

112. The bicistronic iCAR/aCAR construct or monocistronic aCAR and iCAR constructs for co-transduction according to claim 64, wherein the iCAR inhibitory domain component is a CD300a inhibitory domain (SEQ ID NO:141).

113. The bicistronic iCAR/aCAR construct or monocistronic aCAR and iCAR constructs for co-transduction according to claim 64, wherein the iCAR inhibitory domain component is a CD300f inhibitory domain (SEQ ID NO:142).

114. The bicistronic iCAR/aCAR construct or monocistronic aCAR and iCAR constructs for co-transduction according to claim 64, wherein the iCAR inhibitory domain component is a LIR1 inhibitory domain (SEQ ID NO:143).

115. The bicistronic iCAR/aCAR construct or monocistronic aCAR and iCAR constructs for co-transduction according to claim 64, wherein the iCAR inhibitory domain component is a LIR2 inhibitory domain (SEQ ID NO:144).

116. The bicistronic iCAR/aCAR construct or monocistronic aCAR and iCAR constructs for co-transduction according to claim 64, wherein the iCAR inhibitory domain component is a LIR3 inhibitory domain (SEQ ID NO:145).

117. The bicistronic iCAR/aCAR construct or monocistronic aCAR and iCAR constructs for co-transduction according to claim 64, wherein the iCAR inhibitory domain component is a LIR5 inhibitory domain (SEQ ID NO:146).

118. The bicistronic iCAR/aCAR construct or monocistronic aCAR and iCAR constructs for co-transduction according to claim 64, wherein the iCAR inhibitory domain component is a LIR8 inhibitory domain (SEQ ID NO:147).

119. The bicistronic iCAR/aCAR construct or monocistronic aCAR and iCAR constructs for co-transduction according to claim 64, wherein the iCAR inhibitory domain component is a Ly9 inhibitory domain (SEQ ID NO:148).

120. The bicistronic iCAR/aCAR construct or monocistronic aCAR and iCAR constructs for co-transduction according to claim 64, wherein the iCAR inhibitory domain component is a 2xPD1(G4S) inhibitory domain (SEQ ID NO:149).

121. The bicistronic iCAR/aCAR construct or monocistronic aCAR and iCAR constructs for co-transduction according to claim 64, wherein the iCAR inhibitory domain component is a 2xPD1(PD1) inhibitory domain (SEQ ID NO:150).

122. The bicistronic iCAR/aCAR construct or monocistronic aCAR and iCAR constructs for co-transduction according to claim 64, wherein the iCAR inhibitory domain component is a PVRIg inhibitory domain (SEQ ID NO:151).

123. The bicistronic iCAR/aCAR construct or monocistronic aCAR and iCAR constructs for co-transduction according to claim 64, wherein the iCAR inhibitory domain component is a AA2AR inhibitory domain (SEQ ID NO:152).

124. The bicistronic iCAR/aCAR construct or monocistronic aCAR and iCAR constructs for co-transduction according to any one of claims 1 to 123, wherein the aCAR single chain variable fragment (scFv) component targets Her2.

125. The bicistronic iCAR/aCAR construct or monocistronic aCAR and iCAR constructs for co-transduction according to any one of claims 1 to 124, wherein the aCAR scFv comprises the Vh and Vl from trastuzumab (SEQ ID NOs:170 and 171, respectively).

126. The bicistronic iCAR/aCAR construct or monocistronic aCAR and iCAR constructs for co-transduction according to claim 125, wherein the aCAR scFv is SEQ ID NO:172.

127. The bicistronic iCAR/aCAR construct or monocistronic aCAR and iCAR constructs for co-transduction according to any one of claims 1 to 124, wherein the aCAR scFv comprises the Vh and Vl from trastuzumab F9G (SEQ ID NOs: 307 and 308).

128. The bicistronic iCAR/aCAR construct or monocistronic aCAR and iCAR constructs for co-transduction according to any one of claims 1 to 124, wherein the aCAR scFv comprises the Vh and Vl from pertuzumab (SEQ ID NOs:173 and 174, respectively).

129. The bicistronic iCAR/aCAR construct or monocistronic aCAR and iCAR constructs for co-transduction according to claim 128, wherein the aCAR scFv is SEQ ID NO:175.

130. The bicistronic iCAR/aCAR construct or monocistronic aCAR and iCAR constructs for co-transduction according to any one of claims 1 to 124, wherein the aCAR scFv comprises the Vh and Vl from FRP5 (SEQ ID NOs:176 and 177, respectively).

131. The bicistronic iCAR/aCAR construct or monocistronic aCAR and iCAR constructs for co-transduction according to any one of claims 1 to 124, wherein the aCAR scFv comprises the Vh and Vl from A21 (SEQ ID NOs:178 and 179, respectively).

132. The bicistronic iCAR/aCAR construct or monocistronic aCAR and iCAR constructs for co-transduction according to any one of claims 1 to 124, wherein the aCAR scFv comprises the Vh and Vl from XMT1517 (SEQ ID NOs:180 and 181, respectively).

133. The bicistronic iCAR/aCAR construct or monocistronic aCAR and iCAR constructs for co-transduction according to any one of claims 1 to 124, wherein the aCAR scFv comprises the Vh and Vl from XMT1518 (SEQ ID NOs:182 and 183, respectively).

134. The bicistronic iCAR/aCAR construct or monocistronic aCAR and iCAR constructs for co-transduction according to any one of claims 1 to 124, wherein the aCAR scFv comprises the Vh and Vl from XMT1519 (SEQ ID NOs:184 and 185, respectively).

135. The bicistronic iCAR/aCAR construct or monocistronic aCAR and iCAR constructs for co-transduction according to any one of claims 1 to 124, wherein the aCAR scFv comprises the Vh and Vl from FWP51 (SEQ ID NOs:186 and 187, respectively).

136. The bicistronic iCAR/aCAR construct or monocistronic aCAR and iCAR constructs for co-transduction according to claim 135, wherein the aCAR scFv comprises SEQ ID NO:188.

137. The bicistronic iCAR/aCAR construct or monocistronic aCAR and iCAR constructs for co-transduction according to any one of claims 1 to 123, wherein the aCAR single chain variable fragment (scFv) component targets EGFR.

138. The bicistronic iCAR/aCAR construct or monocistronic aCAR and iCAR constructs for co-transduction according to any one of claim 1 to 123 or 137, wherein the aCAR scFv comprises the Vh and Vl from cetuximab (SEQ ID NOs:189 and 190, respectively).

139. The bicistronic iCAR/aCAR construct or monocistronic aCAR and iCAR constructs for co-transduction according to any one of claim 1 to 123 or 137, wherein the aCAR scFv is SEQ ID NO:191.

140. The bicistronic iCAR/aCAR construct or monocistronic aCAR and iCAR constructs for co-transduction according to any one of claim 1 to 123 or 137, wherein the aCAR scFv comprises the Vh and Vl from panitumumab (SEQ ID NOs:192 and 193, respectively).

141. The bicistronic iCAR/aCAR construct or monocistronic aCAR and iCAR constructs for co-transduction according to any one of claim 1 to 123 or 137, wherein the aCAR scFv is SEQ ID NO:194.

142. The bicistronic iCAR/aCAR construct or monocistronic aCAR and iCAR constructs for co-transduction according to any one of claim 1 to 123 or 137, wherein the aCAR scFv comprises the Vh and Vl from Imgatuzumab (SEQ ID NOs:195 and 196, respectively).

143. The bicistronic iCAR/aCAR construct or monocistronic aCAR and iCAR constructs for co-transduction according to any one of claim 1 to 123 or 137, wherein the aCAR scFv comprises the Vh and Vl from Nimotuzumab (SEQ ID NOs:197 and 198, respectively).

144. The bicistronic iCAR/aCAR construct or monocistronic aCAR and iCAR constructs for co-transduction according to any one of claim 1 to 123 or 137, wherein the aCAR scFv comprises the Vh and Vl from Nimotuzumab (K5) (SEQ ID NOs:310 and 311, respectively).

145. The bicistronic iCAR/aCAR construct or monocistronic aCAR and iCAR constructs for co-transduction according to any one of claim 1 to 123 or 137, wherein the aCAR scFv comprises the Vh and Vl from Necitumumab (SEQ ID NOs:199 and 200, respectively).

146. The bicistronic iCAR/aCAR construct or monocistronic aCAR and iCAR constructs for co-transduction according to any one of claim 1 to 123 or 137, wherein the aCAR scFv comprises the Vh and Vl from ICR62 (SEQ ID NOs:201 and 202, respectively).

147. The bicistronic iCAR/aCAR construct or monocistronic aCAR and iCAR constructs for co-transduction according to any one of claim 1 to 123 or 137, wherein the aCAR scFv comprises the Vh and Vl from Matuzumab (SEQ ID NOs:204 and 205, respectively).

148. The bicistronic iCAR/aCAR construct or monocistronic aCAR and iCAR constructs for co-transduction according to any one of claim 1 to 123 or 137, wherein the aCAR scFv comprises the Vh and Vl from C10 (SEQ ID NOs:206 and 207, respectively).

149. The bicistronic iCAR/aCAR construct or monocistronic aCAR and iCAR constructs for co-transduction according to any one of claim 1 to 123 or 137, wherein the aCAR scFv comprises the Vh and Vl from Zalutumumab (SEQ ID NOs:208 and 209, respectively).

150. The bicistronic iCAR/aCAR construct or monocistronic aCAR and iCAR constructs for co-transduction according to any one of claim 1 to 123 or 137, wherein the aCAR scFv comprises the Vh and Vl from P1X (SEQ ID NOs:210 and 211, respectively).

151. The bicistronic iCAR/aCAR construct or monocistronic aCAR and iCAR constructs for co-transduction according to any one of claim 1 to 123 or 137, wherein the aCAR scFv comprises the Vh and Vl from P2X (SEQ ID NOs:212 and 213, respectively).

152. The bicistronic iCAR/aCAR construct or monocistronic aCAR and iCAR constructs for co-transduction according to any one of claim 1 to 123 or 137, wherein the aCAR scFv comprises the Vh and Vl from P3X (SEQ ID NOs:214 and 215, respectively).

153. The bicistronic iCAR/aCAR construct or monocistronic aCAR and iCAR constructs for co-transduction according to any one of claim 1 to 123 or 137, wherein the aCAR scFv comprises the VH from EGFR-1a1-VHH (SEQ ID NO:216).

154. The bicistronic iCAR/aCAR construct or monocistronic aCAR and iCAR constructs for co-transduction according to any one of claim 1 to 123 or 137, wherein the aCAR scFv comprises the VH from EGFR-VHH (SEQ ID NO:312).

155. The bicistronic iCAR/aCAR construct or monocistronic aCAR and iCAR constructs for co-transduction according to any one of claims 1 to 123, wherein the aCAR single chain variable fragment (scFv) component targets Mesothelin.

156. The bicistronic iCAR/aCAR construct or monocistronic aCAR and iCAR constructs for co-transduction according to any one of claim 1 to 123 or 155, wherein the aCAR scFv comprise the Vh and Vl from Amatuximab (SEQ ID NOs:217 and 218, respectively).

157. The bicistronic iCAR/aCAR construct or monocistronic aCAR and iCAR constructs for co-transduction according to any one of claim 1 to 123 or 155, wherein the aCAR scFv comprise the Vh and Vl from P4 (SEQ ID NOs:219 and 220, respectively).

158. The bicistronic iCAR/aCAR construct or monocistronic aCAR and iCAR constructs for co-transduction according to any one of claim 1 to 123 or 155, wherein the aCAR scFv comprise the Vh and Vl from SS1 (SEQ ID NOs:222 and 223, respectively).

159. The bicistronic iCAR/aCAR construct or monocistronic aCAR and iCAR constructs for co-transduction according to any one of claim 1 to 123 or 155, wherein the aCAR scFv comprise the VHH from SD1 (SEQ ID NO:225).

160. The bicistronic iCAR/aCAR construct or monocistronic aCAR and iCAR constructs for co-transduction according to any one of claim 1 to 123 or 155, wherein the aCAR scFv comprise the VHH from SD2 (SEQ ID NO:226).

161. The bicistronic iCAR/aCAR construct or monocistronic aCAR and iCAR constructs for co-transduction according to any one of claim 1 to 123 or 155, wherein the aCAR scFv comprise the Vh and Vl from 1H7 (SEQ ID NOs:227 and 228, respectively).

162. The bicistronic iCAR/aCAR construct or monocistronic aCAR and iCAR constructs for co-transduction according to any one of claim 1 to 123 or 155, wherein the aCAR scFv comprise the Vh and Vl from 3C02 (SEQ ID NOs:230 and 231, respectively).

163. The bicistronic iCAR/aCAR construct or monocistronic aCAR and iCAR constructs for co-transduction according to any one of claims 1 to 162, wherein the hinge TM domain component is selected from the group consisting of a CD28 hinge and a CD8 hinge (including a CD8a hinge domain).

164. The bicistronic iCAR/aCAR construct or monocistronic aCAR and iCAR constructs for co-transduction according to claim 163, wherein the hinge TM domain component is a CD28 hinge domain (SEQ ID NO:85).

165. The bicistronic iCAR/aCAR construct or monocistronic aCAR and iCAR constructs for co-transduction according to claim 163, wherein the hinge TM domain component is a CD8 alpha hinge domain (SEQ ID NO:84).

166. The bicistronic iCAR/aCAR construct or monocistronic aCAR and iCAR constructs for co-transduction according to any one of claims 1 to 165, wherein the co-stimulatory domain component is selected from the group consisting of a CD137 (4-1BB) co-stimulatory domain, a CD28 co-stimulatory domain, a 28BB co-stimulatory domain, and a CD3z co-stimulatory domain.

167. The bicistronic iCAR/aCAR construct or monocistronic aCAR and iCAR constructs for co-transduction according to claim 166, wherein the co-stimulatory domain component is a CD137 (4-1BB) co-stimulatory domain (SEQ ID NO:233).

168. The bicistronic iCAR/aCAR construct or monocistronic aCAR and iCAR constructs for co-transduction according to claim 166, wherein the co-stimulatory domain component is a CD28 co-stimulatory domain (SEQ ID NO:234).

169. The bicistronic iCAR/aCAR construct or monocistronic aCAR and iCAR constructs for co-transduction according to claim 166, wherein the co-stimulatory domain component a CD3z activation signaling domain (SEQ ID NO:235).

170. The bicistronic iCAR/aCAR construct or monocistronic aCAR and iCAR constructs for co-transduction according to any one of claims 1 to 169, wherein the ITAM is a CD3 zeta domain.

171. The bicistronic iCAR/aCAR construct or monocistronic aCAR and iCAR constructs for co-transduction according to claim 170, wherein the ITAM is a CD3 zeta domain (SEQ ID NO:236).

172. The bicistronic iCAR/aCAR construct or monocistronic aCAR and iCAR constructs for co-transduction according to claim 170, wherein the ITAM is a CD3 zeta 3F domain (SEQ ID NO:237).

173. The bicistronic iCAR/aCAR construct or monocistronic aCAR and iCAR constructs for co-transduction according to claims 1 to 172, wherein the ITAM is a CD3 zeta 4F domain (SEQ ID NO:238).

174. The bicistronic iCAR/aCAR construct or monocistronic aCAR and iCAR constructs for co-transduction according to claim 170, wherein the ITAM is a CD3 zeta 4OF domain (SEQ ID NO:239).

175. The bicistronic iCAR/aCAR construct or monocistronic aCAR and iCAR constructs for co-transduction according to any one of claims 1 to 174, wherein the linker connecting the iCAR portion and the aCAR portion comprises one or more linker selected from the group consisting of T2A (SEQ ID NO:155), F2A (SEQ ID NO:156), P2A (SEQ ID NO:157), E2A (SEQ ID NO:158), and an IRES sequence (SEQ ID NO:159 or 160).

176. The bicistronic iCAR/aCAR construct or monocistronic aCAR and iCAR constructs for co-transduction according to any one of claims 1 to 175, wherein the linker connecting the iCAR portion and the aCAR portion is GSG T2A (SEQ ID NO:155).

177. The bicistronic iCAR/aCAR construct or monocistronic aCAR and iCAR constructs for co-transduction according to any one of claims 1 to 176, wherein the bicistronic iCAR/aCAR construct comprises an amino acid sequence encoded by a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

178. The bicistronic iCAR/aCAR construct or monocistronic aCAR and iCAR constructs for co-transduction according to any one of claims 1 to 176, wherein the bicistronic iCAR/aCAR construct comprises an amino acid sequence encoded by a nucleic acid sequence selected from the group consisting of SEQ ID NO:275, SEQ ID NO:277, SEQ ID NO:279, SEQ ID NO:281, SEQ ID NO:321, SEQ ID NO:323, and SEQ ID NO:325.

179. The bicistronic iCAR/aCAR construct or monocistronic aCAR and iCAR constructs for co-transduction according to any one of claims 1 to 177, wherein the bicistronic iCAR/aCAR construct comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

180. The bicistronic iCAR/aCAR construct or monocistronic aCAR and iCAR constructs for co-transduction according to any one of claims 1 to 177, wherein the bicistronic iCAR/aCAR construct comprises an amino acid sequence selected from the group consisting of SEQ ID NO:276, SEQ ID NO:278, SEQ ID NO:280, SEQ ID NO:282, SEQ ID NO:322, SEQ ID NO:324, and SEQ ID NO:326.

181. The bicistronic iCAR/aCAR construct or monocistronic aCAR and iCAR constructs for co-transduction according any of the preceding claims, wherein the bicistronic iCAR/aCAR construct further comprises a short hairpin RNA (shRNA).

182. The bicistronic iCAR/aCAR construct or monocistronic aCAR and iCAR constructs for co-transduction according any of the preceding claims, wherein the iCAR comprises a synthetic PD-1 or LIR1 sequence as shown in Table 8, including one selected from the group consisting of SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:296, SEQ ID NO:297, SEQ ID NO:298, SEQ ID NO:299, SEQ ID NO:300, SEQ ID NO:301, SEQ ID NO:302, and SEQ ID NO:304.

183. The bicistronic iCAR/aCAR construct or monocistronic aCAR and iCAR constructs for co-transduction according any of the preceding claims, wherein the iCAR/aCAR comprises a construct as described in Table 1.

184. The bicistronic iCAR/aCAR construct or monocistronic aCAR and iCAR constructs for co-transduction according any of the preceding claims, wherein the iCAR/aCAR comprises a nucleic acid sequence as described in Table 1, including SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

185. The bicistronic iCAR/aCAR construct or monocistronic aCAR and iCAR constructs for co-transduction according any of the preceding claims, wherein the iCAR/aCAR comprises an amino acid sequence as described in Table 1, including SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

186. The bicistronic iCAR/aCAR construct or monocistronic aCAR and iCAR constructs for co-transduction according any of the preceding claims, wherein the iCAR comprises an amino acid sequence selected from the group consisting of SEQ ID NO: 255, SEQ ID NO:256, SEQ ID NO:257, SEQ ID NO:258, SEQ ID NO:305, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:327, SEQ ID NO:328, SEQ ID NO:329, SEQ ID NO:330, SEQ ID NO:331, SEQ ID NO:332, SEQ ID NO:333, and SEQ ID NO:334.

187. The bicistronic iCAR/aCAR construct or monocistronic aCAR and iCAR constructs for co-transduction according any of the preceding claims, wherein the iCAR/aCAR comprises a construct as described in Table 1, Table 11 and/or Table 12.

188. The bicistronic iCAR/aCAR construct or monocistronic aCAR and iCAR constructs for co-transduction according any of the preceding claims, wherein the iCAR/aCAR comprises a construct or portion thereof as described in any one of Tables 1 to 22.

189. The bicistronic iCAR/aCAR construct or monocistronic aCAR and iCAR constructs for co-transduction according any of the preceding claims, wherein the aCAR comprises a construct as described in any one of Tables 15, 16, 17, and/or 21.

190. The bicistronic iCAR/aCAR construct or monocistronic aCAR and iCAR constructs for co-transduction according any of the preceding claims, wherein the iCAR comprises a construct as described in any one of Tables 1, 2, 4, 9, 10, 11 and/or 12.

191. A nucleic acid composition comprising a nucleic acid that encodes a bicistronic iCAR/aCAR construct or monocistronic aCAR and iCAR constructs for co-transduction according to any one of the preceding claims.

192. A vector comprising a nucleic acid sequence encoding for a bicistronic iCAR/aCAR construct or monocistronic aCAR and iCAR constructs for co-transduction according to any one of the preceding claims.

193. A vector composition comprising the vector according to claim 188.

194. The nucleic acid or vector according to claims 187 to 189, wherein the iCAR/aCAR construct or monocistronic aCAR and iCAR constructs for co-transduction comprises a signal peptide upstream of the iCAR and/or aCAR portions.

195. The nucleic acid or vector according to claim 190, wherein the signal peptide is a CD8 alpha signal peptide (SEQ ID NO: 161), a GM-CSF signal peptide (SEQ ID NO: 162), or a mIgK signal peptide (SEQ ID NO: 306).

196. A safe effector cell comprising a nucleic acid or nucleic acid sequence composition according to claim 187.

197. A safe effector cell comprising a vector or vector composition of according to claim 188 or 189.

198. A safe effector immune cell expressing a bicistronic iCAR/aCAR construct or monocistronic aCAR and iCAR constructs for co-transduction according to any one of the preceding claims.

199. A method for treating cancer in a patient having a tumor characterized by LOH, comprising administering to the patient a safe effector immune cell according to any one of claims 192 to 194.

200. A method for treating cancer in a patient having a tumor characterized by a genetic mutation resulting in a complete loss of expression of a target gene or target extracellular polymorphic epitope gene, comprising administering to the patient a safe effector immune cell according to any one of claims 192 to 194.

201. A method for treating cancer in a patient having a tumor characterized by loss of heterozygosity (LOH), or other genetic loss or allelic imbalance phenotypes including, without limitation, loss of function or expression, resulting from mutations affecting one or more nucleotides, comprising administering to the patient a safe effector immune cell according to any one of claims 188 to 190.

202. The method of claim 193, wherein the cancer is selected from the group consisting of Acute Myeloid Leukemia [LAML], Adrenocortical carcinoma [ACC], Bladder Urothelial Carcinoma [BLCA], Brain Lower Grade Glioma [LGG], Breast invasive carcinoma [BRCA], Cervical squamous cell carcinoma and endocervical adenocarcinoma [CESC], Cholangiocarcinoma [CHOL], Colon adenocarcinoma [COAD], Esophageal carcinoma [ESCA], Glioblastoma multiforme [GBM], Head and Neck squamous cell carcinoma [HNSC], Kidney Chromophobe [KICH], Kidney renal clear cell carcinoma [KIRC], Kidney renal papillary cell carcinoma [KIRP], Liver hepatocellular carcinoma [LIHC], Lung adenocarcinoma [LUAD], Lung squamous cell carcinoma [LUSC], Lymphoid Neoplasm Diffuse Large B-cell Lymphoma [DLBC], Mesothelioma [MESO], Ovarian serous cystadenocarcinoma [OV], Pancreatic adenocarcinoma [PAAD], Pheochromocytoma and Paraganglioma [PCPG], Prostate adenocarcinoma [PRAD], Rectum adenocarcinoma [READ], Sarcoma [SARC], Skin Cutaneous Melanoma [SKCM], Stomach adenocarcinoma [STAD], Testicular Germ Cell Tumors [TGCT], Thymoma [THYM], Thyroid carcinoma [THCA], Uterine Carcinosarcoma [UCS], Uterine Corpus Endometrial Carcinoma [UCEC], Uveal Melanoma [UVM], Non-small cell lung carcinoma [NSCLC], and Small cell lung cancer [SCLC].
